(19) United States
(12) Patent Application Publication
(10) Pub. No.: US 2014/0196161 A1
(43) Pub. Date: Jul. 10, 2014

(54) Transgenic Plants With Enhanced Agronomic Traits

(71) Applicant: Monsanto Technology LLC., St. Louis, MO (US)

(72) Inventors: Mark Scott Abad, Webster Grove, MO (US); Mary Fernandes, St. Louis, MO (US); Barry S. Goldman, St. Louis, MO (US)

(73) Assignee: Monsanto Technology LLC., St. Louis, MO (US)

(21) Appl. No.: 13/998,684

(22) Filed: Nov. 22, 2013

Related U.S. Application Data
(63) Continuation of application No. 13/289,012, filed on Nov. 4, 2011, now abandoned.
Application No. 13/289,012 is a continuation of application No. 12/074,197, filed on Feb. 29, 2008, now abandoned.
Application No. 12/074,197 is a continuation of application No. PCT/US2006/033989, filed on Aug. 30, 2005.
(60) Provisional application No. 60/713,150, filed on Aug. 30, 2005.

(51) Int. Cl. C12N 15/82; G01N 33/50; C12Q 1/68

(52) U.S. Cl. CPC C12N 15/8261; C12N 15/8274; C12Q 1/6895; G01N 33/5097; C12N 15/8273; C12N 15/8251; C12N 15/8247; C12N 15/8209
USPC 800/260; 435/419; 435/418; 800/298; 800/320.1; 800/312; 800/314; 800/306; 800/300.1; 800/320.3; 800/320.2; 506/9; 435/29

(57) ABSTRACT

This invention provides recombinant DNA for expression of proteins that are useful for imparting enhanced agronomic trait(s) to transgenic crop plants. Also provided by this invention is transgenic seed for growing a transgenic plant having recombinant DNA in its genome and exhibiting an enhance agronomic trait, i.e. enhanced nitrogen use efficiency, increased yield, enhanced water use efficiency, enhanced tolerance to cold stress and/or improved seed compositions. Also disclosed are methods for identifying such transgenic plants by screening for nitrogen use efficiency, yield, water use efficiency, growth under cold stress, and seed composition changes. This invention also discloses a method of identifying the target genes of a transcription factor.

### CROSS REFERENCE TO RELATED APPLICATIONS

This application is a continuation of U.S. application Ser. No. 13/289,012 (pending) which was filed on Nov. 4, 2011, which is a continuation of U.S. application Ser. No. 12/074,197 (abandoned), which was filed on Feb. 29, 2008, which claims benefit under 35 U.S.C. 119(e) of U.S. provisional application Ser. No. 60/713,150, filed Aug. 30, 2005, and International application PCT/US2006/033989, all of which are incorporated herein by reference in their entirety.

### INCORPORATION OF SEQUENCE LISTING

Two copies of the sequence listing (Copy 1 and Copy 2) in ASCII computer readable form (CRF) and a written copy (compact disc copy, in ASCII CRF) of the sequence listing, all on CD-Rs, each containing the text file named “3126014US3.TXT”, which is 35,160,466 bytes (measured in MS-WINDOWS) and created on Nov. 22, 2013 are incorporated herein by reference.

### FIELD OF THE INVENTION

Disclosed herein are inventions in the field of plant genetics and developmental biology. More specifically, the present inventions provide transgenic seeds for crops, wherein the genome of said seed comprises recombinant DNA, the expression of which results in the production of transgenic plants with enhanced agronomic traits.

### SUMMARY OF THE INVENTION

This invention employs recombinant DNA for expression of proteins that are useful for imparting enhanced agronomic traits to the transgenic plants. Recombinant DNA in this invention is provided in a construct comprising a promoter that is functional in plant cells and that is operably linked to DNA that encodes a protein having at least one amino acid domain in a sequence that exceeds the Pfam gathering cutoff for amino acid sequence alignment with a protein domain family identified by a Pfam name in the group of Pfam domain names as identified in Table 11. In more specific embodiments of the invention the protein expressed in plant cells has an amino acid sequence with at least 90% identity to a consensus amino acid sequence in the group of consensus amino acid sequences consisting of the consensus amino acid sequence constructed for SEQ ID NO: 194 and homologs thereof listed in Table 7 through the consensus amino acid sequence constructed for SEQ ID NO: 386 and homologs thereof listed in Table 7. In even more specific embodiments of the invention the protein expressed in plant cells is a protein selected from the group of proteins identified in Table 1.

Other aspects of the invention are specifically directed to transgenic plant cells comprising the recombinant DNA of the invention, transgenic plants comprising a plurality of such plant cells, progeny transgenic seed, embryo and transgenic pollen from such plants. Such plant cells are selected from a population of transgenic plants regenerated from plant cells transformed with recombinant DNA and that express the protein by screening transgenic plants in the population for an enhanced trait as compared to control plants that do not have said recombinant DNA, where the enhanced trait is selected from group of enhanced traits consisting of enhanced water use efficiency, enhanced cold tolerance, increased yield, enhanced nitrogen use efficiency, enhanced seed protein and enhanced seed oil.

In yet another aspect of the invention the plant cells, plants, seeds, embryo and pollen further comprise DNA expressing a protein that provides tolerance from exposure to an herbicide applied at levels that are lethal to a wild type of said plant cell. Such tolerance is especially useful not only as an advantageous trait in such plants but is also useful in a selection step in the methods of the invention. In aspects of the invention the agent of such herbicide is a glyphosate, dicamba, or glufosinate compound.

Yet other aspects of the invention provide transgenic plants which are homozygous for the recombinant DNA and transgenic seed of the invention from corn, soybean, cotton, canola, alfalfa, wheat or rice plants.

In other important embodiments for practice of various aspects of the invention, the plants of this invention can be further enhanced with stacked traits, e.g., a crop having an enhanced agronomic trait resulting from expression of DNA disclosed herein, in combination with herbicide, disease, and/or pest resistance traits.

This invention also provides methods for manufacturing non-natural, transgenic seed that can be used to produce a crop of transgenic plants with an enhanced trait resulting from expression of stably-integrated, recombinant DNA for expressing a protein having at least one domain of amino acids in a sequence that exceeds the Pfam gathering cutoff for amino acid sequence alignment with a protein domain family identified by a Pfam name in the group of Pfam names identified in Table 11. More specifically the method comprises (a) screening a population of plants for an enhanced trait and a recombinant DNA, where individual plants in the population can exhibit the trait at a level less than, essentially the same as or greater than the level that the trait is exhibited in control plants which do not express the recombinant DNA, (b) selecting from the population one or more plants that exhibit the trait at a level greater than the level that said trait is exhibited in control plants, (c) verifying that the recombinant DNA is stably integrated in said selected plants, (d) analyzing tissue of a selected plant to determine the production of a protein having the function of a protein encoded by nucleotides in a sequence of one of SEQ ID NO:1-193; and (e) collecting seed from a selected plant. In one aspect of the invention the plants in the population further comprise DNA expressing a protein that provides tolerance to exposure to an herbicide applied at levels that are lethal to wild type plant cells and the selecting is effected by treating the population with the herbicide, e.g. a glyphosate, dicamba, or glufosinate compound. In another aspect of the invention the plants are selected by identifying plants with the enhanced trait. The methods are especially useful for manufacturing corn, soybean, cotton, alfalfa, wheat or rice seed.

Another aspect of the invention provides a method of producing hybrid corn seed comprising acquiring hybrid corn seed from a herbicide tolerant corn plant which also has stably-integrated, recombinant DNA comprising a promoter that is (a) functional in plant cells and (b) is operably linked to DNA that encodes a protein having at least one domain of amino acids in a sequence that exceeds the Pfam gathering cutoff for amino acid sequence alignment with a protein domain family identified by a Pfam name in the group of Pfam names identified in Table 11. The methods further comprise producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA; selecting corn plants which are homozygous and hemizygous for said recombinant DNA by treating with an herbicide; collecting seed from herbicide-treated-surviving corn plants and planting said seed to produce further progeny corn plants; repeating the selecting and collecting steps at least once to produce an inbred corn line; and crossing the inbred corn line with a second corn line to produce hybrid seed.

Another aspect of the invention provides a method of selecting a plant comprising plant cells of the invention by using an immunoreactive antibody to detect the presence of protein expressed by recombinant DNA in seed or plant tissue. Yet another aspect of the invention provides anti-counterfeit milled seed having, as an indication of origin, a plant cell of this invention.

Still other aspects of this invention relate to transgenic plants with enhanced water use efficiency or enhanced nitrogen use efficiency. For instance, this invention provides methods of growing a corn, cotton or soybean crop without irrigation water comprising planting seed having plant cells of the invention which are selected for enhanced water use efficiency. Alternatively methods comprise applying reduced irrigation water, e.g. providing up to 300 millimeters of ground water during the production of a corn crop. This invention also provides methods of growing a corn, cotton or soybean crop without added nitrogen fertilizer comprising planting seed having plant cells of the invention which are selected for enhanced nitrogen use efficiency.

### BRIEF DESCRIPTION OF FIGURES

FIG. 1-4 illustrate plasmid maps.

### DETAILED DESCRIPTION OF THE INVENTION

In the attached sequence listing:

SEQ ID NO:1-193 are nucleotide sequences of the coding strand of DNA for “genes” used in the recombinant DNA imparting an enhanced trait in plant cells, i.e. each represents a coding sequence for a protein;

SEQ ID NO:194-386 are amino acid sequences of the cognate protein of the “genes” with nucleotide coding sequence 1-193;

SEQ ID NO: 387-12580 are amino acid sequences of homologous proteins;

SEQ ID NO: 12581-12601 are nucleotide sequences of the elements in base plasmid vectors

SEQ ID NO: 12602 is a consensus amino acid sequence.

SEQ ID NO: 12603 is a nucleotide sequence of a base plasmid vector useful for corn transformation; and

SEQ ID NO: 12604 is a nucleotide sequence of a base plasmid vector useful for soybean transformation.

SEQ ID NO: 12605 is a nucleotide sequence of a base plasmid vector useful for cotton transformation.

SEQ ID NO: 12606 is the nucleotide sequence of plasmid PMON17730.

SEQ ID NO: 12607 is the nucleotide sequence of PHE0010424_PMON17730.

SEQ ID NO: 12608 is the consensus sequence of SEQ ID NO: 371 and 11 homologs

As used herein, a “transgenic plant” means a plant whose genome has been altered by the incorporation of exogenous DNA, e.g., by transformation as described herein. The term “transgenic plant” is used to refer to the plant produced from an original transformation event, or progeny from later generations or crosses of a plant so transformed, so long as the progeny contains the exogenous genetic material in its genome. “Exogenous DNA” means DNA, e.g., recombinant DNA, originating from or constructed outside of the plant including natural or artificial DNA derived from the host “transformed” organism of a different organism.

As used herein, “recombinant DNA” means DNA which has been a genetically engineered or constructed outside of a cell, including DNA containing naturally occurring DNA or cDNA, or synthetic DNA.

As used herein, a “functional portion” of DNA is that part which comprises an encoding region for a protein segment that is sufficient to provide the desired enhanced agronomic trait in plants transformed with the DNA activity. Where expression of protein is desired, a functional portion will generally comprise the entire coding region for the protein, although certain deletions, truncations, rearrangements and the like of the protein may also maintain, or in some cases improve, the desired activity. One skilled in the art is aware of methods to screen for such desired modifications and such functional portion of the protein is considered within the scope of the present invention.

As used herein, “consensus sequence” means an artificial, amino acid sequence of conserved parts of the proteins encoded by homologous genes, e.g., as determined by a CLUSTALW alignment of amino acid sequence of homolog proteins.

As used herein, “homolog” means a protein in a group of proteins that perform the same biological function, e.g., provide an enhanced agronomic trait in transgenic plants of this invention. Homologs are expressed by homologous genes which are genes that encode proteins with the same or similar biological function. Homologous genes may be generated by the event of speciation (see ortholog) or by the event of genetic duplication (see paralog). Orthologs refer to a set of homologous genes in different species that evolved from a common ancestral gene by specification. Normally, orthologs retain the same function in the course of evolution; and paralogs refer to a set of homologous genes in the same species that have diverged from each other as a consequence of genetic duplication. Thus, homologous genes can be from the same or a different organism. Homologous DNA includes naturally occurring and synthetic variants. For instance, degeneracy of the genetic code provides the possibility to substitute at least one base of the protein encoding sequence of a gene with a different base without causing the amino acid sequence of the polypeptide produced from the gene to be changed. Hence, a polynucleotide useful in the present invention may have any base sequence that has been changed from SEQ ID NO:1 through SEQ ID NO: 193 by substitution in accordance with degeneracy of the genetic code. Homologs are proteins which, when optimally aligned, has at least 60% identity (say at least 70% or 80% or 90% identity) over the full length of a protein identified herein, or a higher percent identity especially over a shorter functional part of the protein, e.g., 70% to 80 or 90% amino acid identity over a window of comparison comprising a functional part of the protein imparting the enhanced agronomic trait. Homologs include proteins with an amino acid sequence that has at least 90% identity to a consensus amino acid sequence of proteins and homologs disclosed herein.

Homologs can be identified by comparison of amino acid sequence, e.g., manually or by using known homology-based search algorithms such as those commonly known and referred to as BLAST, FASTA, and Smith-Waterman. A local sequence alignment program, e.g., BLAST, can be used to search a database of sequences to find similar sequences, and the summary Expectation value (E-value) used to measure the sequence base similarity. As a protein hit with the best E-value for a particular organism may not necessarily be an ortholog or the only ortholog, a reciprocal query is used in the present invention to filter hit sequences with significant E-values for ortholog identification. The reciprocal query entails search of the significant hits against a database of amino acid sequences from the base organism that are similar to the sequence of the query protein. A hit is a likely ortholog, when the reciprocal query's best hit is the query protein itself or a protein encoded by a duplicated gene after speciation. A further aspect of the invention comprises functional homolog proteins which differ in one or more amino acids from those of disclosed protein as the result of conservative amino acid substitutions, e.g., substitutions are among: acidic (negatively charged) amino acids such as aspartic acid and glutamic acid; basic (positively charged) amino acids such as arginine, histidine, and lysine; neutral polar amino acids such as glycine, serine, threonine, cysteine, tyrosine, asparagine, and glutamine; neutral nonpolar (hydrophobic) amino acids such as alanine, leucine, isoleucine, valine, proline, phenylalanine, tryptophan, and methionine; amino acids having aliphatic side chains such as glycine, alanine, valine, leucine, and isoleucine; amino acids having aliphatic-hydroxyl side chains such as serine and threonine; amino acids having amide-containing side chains such as asparagine and glutamine; amino acids having aromatic side chains such as phenylalanine, tyrosine, and tryptophan; amino acids having basic side chains such as lysine, arginine, and histidine; amino acids having sulfur-containing side chains such as cysteine and methionine; naturally conservative amino acids such as valine-leucine, valine-isoleucine, phenylalanine-tyrosine, lysine-arginine, alanine-valine, aspartic acid-glutamic acid, and asparagine-glutamine. A further aspect of the homologs encoded by DNA useful in the transgenic plants of the invention are those proteins which differ from a disclosed protein as the result of deletion or insertion of one or more amino acids in a native sequence.

As used herein, “transcription factor gene” refers to a gene that encodes a protein that binds to regulatory regions and is involved in control gene expression. Therefore, as used herein, a target gene refers to a gene whose expression is controlled by a transcription factor gene.

As used herein, “percent identity” means the extent to which two optimally aligned DNA or protein segments are invariant throughout a window of alignment of components, e.g., nucleotide sequence or amino acid sequence. An “identity fraction” for aligned segments of a test sequence and a reference sequence is the number of identical components which are shared by sequences of the two aligned segments divided by the total number of sequence components in the reference segment over a window of alignment which is the smaller of the full test sequence or the full reference sequence. “Percent identity” (“% identity”) is the identity fraction times 100.

As used herein “Pfam” refers to a large collection of multiple sequence alignments and hidden Markov models covering many common protein families, e.g. Pfam version 19.0 (December 2005) contains alignments and models for 8183 protein families and is based on the Swissprot 47.0 and SP-TrEMBL 30.0 protein sequence databases. See S. R. Eddy, “Profile Hidden Markov Models”, Bioinformatics 14:755-763, 1998. Pfam is currently maintained and updated by a Pfam Consortium. The alignments represent some evolutionary conserved structure that has implications for the protein's function. Profile hidden Markov models (profile HMMs) built from the Pfam alignments are useful for automatically recognizing that a new protein belongs to an existing protein family even if the homology by alignment appears to be low. Once one DNA is identified as encoding a protein which imparts an enhanced trait when expressed in transgenic plants, other DNA encoding proteins in the same protein family are identified by querying the amino acid sequence of protein encoded by candidate DNA against the Hidden Markov Model which characterizes the Pfam domain using HMMER software, a current version of which is provided in the appended computer listing. Candidate proteins meeting the gathering cutoff for the alignment of a particular Pfam are in the protein family and have cognate DNA that is useful in constructing recombinant DNA for the use in the plant cells of this invention. Hidden Markov Model databases for use with HMMER software in identifying DNA expressing protein in a common Pfam for recombinant DNA in the plant cells of this invention are also included in the appended computer listing. The HMMER software and Pfam databases are version 19.0 and were used to identify known domains in the proteins corresponding to amino acid sequence of SEQ ID NO: 194 through SEQ ID NO: 386. All DNA encoding proteins that have scores higher than the gathering cutoff disclosed in Table 11 by Pfam analysis disclosed herein can be used in recombinant DNA of the plant cells of this invention, e.g. for selecting transgenic plants having enhanced agronomic traits. The relevant Pfams for use in this invention, as more specifically disclosed below, are FAD_binding_{—}4, MtN3_slv, Homeobox, FAD_binding_{—}6, RWP-RK, PMEI, FAD_binding_{—}7, RRM_{—}1, Transaldolase, RNA_pol_L, WD40, U-box, Cyclin_N, Skp1, Redoxin, DZC, PBP, TPP_enzyme_M, CBFD_NFYB_HMF, TPP_enzyme_N, PFK, Caleosin, Iso_dh, Ribosomal_L18p, Metallophos, zf-A20, Ras, BBE, NAF, PLDc, DUF1242, Pkinase, C2, p450, Pyridoxal_deC, FBD, UPF0005, HEAT_PBS, GST_N, PEP-utilizers, Alpha-amylase, Amino_oxidase, SRF-TF, Phi_{—}1, Malic_M, Tryp_alpha_amyl, GSHPx, Miro, HSF_DNA-bind, DNA_photolyase, Sina, CTP_transf_{—}2, Abhydrolase_{—}3, Chal_sti_synt_C, ACP_syn_III_C, ADH_zinc_N, CSD, Globin, GATase_{—}2, Amidohydro_{—}1, HLH, HALZ, Amidohydro_{—}3, Lactamase_B, HSP20, DAO, DUF296, AT_hook, A WPM-19, Dimerisation, Suc_Fer-like, Methyltransf_{—}2, Aminotran_{—}3, PHD, MMR_HSR1, Aldo_ket_red, zf-AN1, malic, Fasciclin, UPF0057, DUF221, Pkinase_Tyr, DnaJ, Cofilin_ADF, Orn_Arg_deC_N, Skp1_POZ, Asn_synthase, K-box, LRR_{—}2, Ribosomal_L12, Ammonium_transp, Ribosomal_L14, KOW, DUF1336, DS, Aa_trans, CcmH, peroxidase, Aldedh, PEP-utilizers_C, ADH_N, UIM, NAD_binding_{—}1, zf-C3HC4, Spermine_synth, AUX_IAA, LIM, Anti-silence, X8, Citrate_synt, 14-3-3, RMMBL, efhand, NPH3, CAF1, ICL, FAE1_CUT1_RppA, Orn_DAP_Arg_deC, PPDK_N, Myb_DNA-binding, AP2, F-box, and APS_kinase

As used herein, “promoter” means regulatory DNA for initializing transcription. A “plant promoter” is a promoter capable of initiating transcription in plant cells whether or not its origin is a plant cell, e.g., is it well known that viral promoters are functional in plants. Thus, plant promoters include promoter DNA obtained from plants, plant viruses, and bacteria such as Agrobacterium and Rhizobium bacteria. Examples of promoters under developmental control include promoters that preferentially initiate transcription in certain tissues, such as leaves, roots, or seeds. Such promoters are referred to as “tissue preferred”. Promoters which initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions.

As used herein, “operably linked” means the association of two or more DNA fragments in a DNA construct so that the function of one, e.g., protein-encoding DNA, is affected by the other, e.g., a promoter.

As used herein, “expression” means the process that includes transcription of DNA to produce RNA and translation of the cognate protein encoded by the DNA and RNA.

As used herein, a “control plant” means a plant that does not contain the recombinant DNA that confers an enhanced agronomic trait. A control plant is used to compare against a transgenic plant, to identify an enhanced agronomic trait in the transgenic plant. A suitable control plant may be a non-transgenic plant of the parental line used to generate a transgenic plant. A control plant may in some cases be a transgenic plant line that comprises an empty vector or marker gene, but does not contain the recombinant DNA.

As used herein, an “agronomic trait” means a characteristic of a plant, which includes, but are not limited to, plant morphology, physiology, growth and development, yield, nutritional enhancement, disease or pest resistance, or environmental or chemical tolerance. In the plants of this invention the expression of identified recombinant DNA confers an agronomically important trait, e.g., increased yield. An “enhanced agronomic trait” refers to a measurable improvement in an agronomic trait including, but not limited to, yield increase, including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also affected by efficiency of germination (including germination in stressed conditions), growth rate (including growth rate in stressed conditions), ear number, seed number per ear, seed size, composition of seed (starch, oil, protein) and characteristics of seed fill.

Increased yield of a transgenic plant of the present invention can be measured in a number of ways, including test weight, seed number per plant, seed weight, seed number per unit area (i.e. seeds, or weight of seeds, per acre), bushels per acre, tones per acre, tons per acre, kilo per hectare. For example, maize yield may be measured as production of shelled corn kernels per unit of production area, e.g., in bushels per acre or metric tons per hectare, often reported on a moisture adjusted basis, e.g., at 15.5% moisture. Increased yield may result from enhanced utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having enhanced growth and development, and ultimately increased yield, as the result of modified expression of plant growth regulators or modification of cell cycle or photosynthesis pathways.

Also of interest is the generation of transgenic plants that demonstrate enhanced yield with respect to a seed component that may or may not correspond to an increase in overall plant yield. Such properties include enhancements in seed oil, seed molecules such as tocopherol, protein and starch, or oil particular oil components as may be manifest by an alteration in the ratios of seed components.

A subset of the nucleic molecules of this invention includes fragments of the disclosed recombinant DNA consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the group consisting of SEQ ID NO:1 through SEQ ID NO:193, and find use, for example as probes and primers for detection of the polynucleotides of the present invention.

In some embodiments of the invention a constitutively active mutant is constructed to achieve the desired effect. SEQ ID NO: 3-6 encodes only the kinase domain of a calcium dependent protein kinase (CDPK). CDPK1 has a domain structure similar to other calcium-dependant protein kinase in which the protein kinase domain is separated from four efhand domains by 42 amino acid “spacer” region. Calcium-dependent protein kinases are thought to be activated by a calcium-induced conformational change that results in movement of an autoinhibitory domain away form the protein kinase active site (Yokokura et al., 1995). Thus, consitutively active proteins can be made by over expressing the protein kinase domain alone.

In other embodiments of the invention a chimeric gene is constructed between homologous genes from different species to obtain a protein with certain characteristics superior to either native protein, e.g., enhanced stability and favorable enzymatic kinetics. Exemplary chimeric DNA molecules provided by the present invention are set forth as SEQ ID NO: 1 and 2 that encode a Arabidopsis-Corn chimeric pyruvate orthophosphate dikinase (PPDK).

In yet other embodiments of the invention, a codon optimized gene is synthesized to achieve a desirable expression level. Synthetic DNA molecules can be designed by a variety of methods, such as, methods known in the art that are based upon substituting the codon(s) of a first polynucleotide to create an equivalent, or even an improved, second-generation artificial polynucleotide, where this new artificial polynucleotide is useful for enhanced expression in transgenic plants. The design aspect often employs a codon usage table. The table is produced by compiling the frequency of occurrence of codons in a collection of coding sequences isolated from a plant, plant type, family or genus. Other design aspects include reducing the occurrence of polyadenylation signals, intron splice sites, or long AT or GC stretches of sequence (U.S. Pat. No. 5,500,365). Full length coding sequences or fragments thereof can be made of artificial DNA using methods known to those skilled in the art. Such exemplary synthetic DNA molecules provided by the present invention are set forth as SEQ ID NO: 38.

DNA constructs are assembled using methods well known to persons of ordinary skill in the art and typically comprise a promoter operably linked to DNA, the expression of which provides the enhanced agronomic trait. Other construct components may include additional regulatory elements, such as 5′ introns for enhancing transcription, 3′ untranslated regions (such as polyadenylation signals and sites), DNA for transit or signal peptides.

In accordance with the current invention, constitutive promoters are active under most environmental conditions and states of development or cell differentiation. These promoters are likely to provide expression of the polynucleotide sequence at many stages of plant development and in a majority of tissues. A variety of constitutive promoters are known in the art. Examples of constitutive promoters that are active in plant cells include but are not limited to the nopaline synthase (NOS) promoters; the cauliflower mosaic virus (CaMV) 19S and 35S promoters (U.S. Pat. No. 5,858,642); the figwort mosaic virus promoter (P-FMV, U.S. Pat. No. 6,051,753); actin promoters, such as the rice actin promoter (P-Os.Act1, U.S. Pat. No. 5,641,876).

Furthermore, the promoters may be altered to contain one or more “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 (see U.S. Pat. No. 5,641,876), rice actin 2 genes and the maize heat shock protein 70 gene intron (U.S. Pat. No. 5,593,874). Examples of other enhancers that can be used in accordance with the invention include elements from the CaMV 35S promoter, octopine synthase genes, the maize alcohol dehydrogenase gene, the maize shrunken 1 gene and promoters from non-plant eukaryotes.

Tissue-specific promoters cause transcription or enhanced transcription of a polynucleotide sequence in specific cells or tissues at specific times during plant development, such as in vegetative or reproductive tissues. Examples of tissue-specific promoters under developmental control include promoters that initiate transcription primarily in certain tissues, such as vegetative tissues, e.g., roots, leaves or stems, or reproductive tissues, such as fruit, ovules, seeds, pollen, pistils, flowers, or any embryonic tissue, or any combination thereof. Reproductive tissue specific promoters may be, e.g., ovule-specific, embryo-specific, endosperm-specific, integument-specific, pollen-specific, petal-specific, sepal-specific, or some combination thereof. Tissue specific promoter(s) will also include promoters that can cause transcription, or enhanced transcription in a desired plant tissue at a desired plant developmental stage. An example of such a promoter includes, but is not limited to, a seedling or an early seedling specific promoter. One skilled in the art will recognize that a tissue-specific promoter may drive expression of operably linked polynucleotide molecules in tissues other than the target tissue. Thus, as used herein, a tissue-specific promoter is one that drives expression preferentially not only in the target tissue, but may also lead to some expression in other tissues as well.

In one embodiment of this invention, preferential expression in plant green tissues is desired. Promoters of interest for such uses include those from genes such as maize aldolase gene FDA (U.S. patent application publication No. 20040216189), aldolase and pyruvate orthophosphate dikinase (PPDK) (Taniguchi et al. (2000) Plant Cell Physiol. 41(1):42-48).

In another embodiment of this invention, preferential expression in plant root tissue is desired. An exemplary promoter of interest for such uses is derived from Corn Nicotianamine Synthase gene (U.S. patent application publication No. 20030131377).

In yet another embodiment of this invention, preferential expression in plant phloem tissue is desired. An exemplary promoter of interest for such use is the rice tungro bacilliform virus (RTBV) promoter (U.S. Pat. No. 5,824,857).

In practicing this invention, an inducible promoter may also be used to ectopically express the structural gene in the recombinant DNA construct. The inducible promoter may cause conditional expression of a polynucleotide sequence under the influence of changing environmental conditions or developmental conditions. For example, such promoters may cause expression of the polynucleotide sequence at certain temperatures or temperature ranges, or in specific stage(s) of plant development such as in early germination or late maturation stage(s) of a plant. Examples of inducible promoters include, but are not limited to, the light-inducible promoter from the small subunit of ribulose-1,5-bis-phosphate carboxylase (ssRUBISCO) (Fischhoff et al. (1992) Plant Mol Biol. 20:81-93); the drought-inducible promoter of maize (Busk et al., Plant J. 11:1285-1295, 1997), the cold, drought, and high salt inducible promoter from potato (Kirch, Plant Mol. Biol. 33:897-909, 1997), and many cold inducible promoters known in the art; for example rd29a and cor15a promoters from Arabidopsis (Genbank ID: D13044 and U01377), blt101 and blt4.8 from barley (Genbank ID: AJ310994 and U63993), wcs120 from wheat (Genbank ID: AF031235), mlip15 from corn (Genbank ID: D26563) and bn115 from Brassica (Genbank ID: U01377).

In some aspects of the invention, sufficient expression in plant seed tissues is desired to effect improvements in seed composition. Exemplary promoters for use for seed composition modification include promoters from seed genes such as napin (U.S. Pat. No. 5,420,034), maize L3 oleosin (U.S. Pat. No. 6,433,252), zein Z27 (Russell et al. (1997) Transgenic Res. 6(2):157-166), glutelinl (Russell (1997) supra), peroxiredoxin antioxidant (Per1) (Stacy et al. (1996) Plant Mol Biol. 31(6):1205-1216), and globulin 1 (Belanger et al (1991) Genetics 129:863-872).

Recombinant DNA constructs prepared in accordance with the invention will also generally include a 3′ element that typically contains a polyadenylation signal and site. Well-known 3′ elements include those from Agrobacterium tumefaciens genes such as nos 3′, tml 3′, tmr 3′, tms 3′, ocs 3′, tr7 3′, e.g., disclosed in U.S. Pat. No. 6,090,627, incorporated herein by reference; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene a rice lactate dehydrogenase gene and a rice beta-tubulin gene, all of which are disclosed in U.S. published patent application 2002/0192813 A1, incorporated herein by reference; and the pea (Pisum sativum) ribulose biphosphate carboxylase gene (rbs 3′), and 3′ elements from the genes within the host plant.

Constructs and vectors may also include a transit peptide for targeting of a gene target to a plant organelle, particularly to a chloroplast, leucoplast or other plastid organelle. For descriptions of the use of chloroplast transit peptides see U.S. Pat. No. 5,188,642 and U.S. Pat. No. 5,728,925, incorporated herein by reference. For description of the transit peptide region of an Arabidopsis EPSPS gene useful in the present invention, see Klee, H. J. et al., (MGG (1987) 210:437-442).

The recombinant DNA construct may include other elements. For example, the construct may contain DNA segments that provide replication function and antibiotic selection in bacterial cells. For example, the construct may contain an E. coli origin of replication such as ori322 or a broad host range origin of replication such as oriV, oriRi or oriColE.

The construct may also comprise a selectable marker such as an Ec-ntpII-Tn5 that encodes a neomycin phosphotransferase II gene obtained from Tn5 conferring resistance to a neomycin and kanamysin, Spc/Str that encodes for Tn7 aminoglycoside adenyltransferase (aadA) conferring resistance to spectinomycin or streptomycin, or a gentamicin (Gm, Gent) or one of many known selectable marker gene.

The vector or construct may also include a screenable marker and other elements as appropriate for selection of plant or bacterial cells having DNA constructs of the invention. DNA constructs are designed with suitable selectable markers that can confer antibiotic or herbicide tolerance to the cell. The antibiotic tolerance polynucleotide sequences include, but are not limited to, polynucleotide sequences encoding for proteins involved in tolerance to kanamycin, neomycin, hygromycin, and other antibiotics known in the art. An antibiotic tolerance gene in such a vector may be replaced by herbicide tolerance gene encoding for 5-enolpyruvylshikimate-3-phosphate synthase (EPSPS, described in U.S. Pat. Nos. 5,627,061, and 5,633,435; Padgette et al., Herbicide Resistant Crops, Lewis Publishers, 53-85, 1996; and in Penaloza-Vazquez, et al., Plant Cell Reports 14:482-487, 1995) and aroA (U.S. Pat. No. 5,094,945) for glyphosate tolerance, bromoxynil nitrilase (Bxn) for Bromoxynil tolerance (U.S. Pat. No. 4,810,648), phytoene desaturase (crtI (Misawa et al., Plant J. 4:833-840, 1993; and Misawa et al., Plant J. 6:481-489, 1994) for tolerance to norflurazon, acetohydroxyacid synthase (AHAS, Sathasiivan et al., Nucl. Acids Res. 18:2188-2193, 1990). Herbicides for which transgenic plant tolerance has been demonstrated and for which the method of the present invention can be applied include, but are not limited to: glyphosate, sulfonylureas, imidazolinones, bromoxynil, delapon, cyclohezanedione, protoporphyrionogen oxidase inhibitors, and isoxaslutole herbicides.

Other examples of selectable markers, screenable markers and other elements are well known in the art and may be readily used in the present invention. Those skilled in the art should refer to the following for details (for selectable markers, see Potrykus et al., Mol. Gen. Genet. 199:183-188, 1985; Hinchee et al., Bio. Techno. 6:915-922, 1988; Stalker et al., J. Biol. Chem. 263:6310-6314, 1988; European Patent Application 154,204; Thillet et al., J. Biol. Chem. 263:12500-12508, 1988; for screenable markers see, Jefferson, Plant Mol. Biol, Rep. 5: 387-405, 1987; Jefferson et al., EMBO J. 6: 3901-3907, 1987; Sutcliffe et al., Proc. Natl. Acad. Sci. U.S.A. 75: 3737-3741, 1978; Ow et al., Science 234: 856-859, 1986; Ikatu et al., Bio. Technol. 8: 241-242, 1990; and for other elements see, European Patent Application Publication Number 0218571; Koziel et al., Plant Mol. Biol. 32: 393-405; 1996).

The plants of this invention can be further enhanced with stacked traits, e.g., a crop having an enhanced agronomic trait resulting from expression of DNA disclosed herein, in combination with herbicide, disease, and/or pest resistance traits. The recombinant DNA is provided in plant cells derived from corn lines that maintain resistance to a virus such as the Mal de Rio Cuarto virus or a fungus such as the Puccina sorghi fungus or both, which are common plant diseases in Argentina. For example, genes of the current invention can be stacked with other traits of agronomic interest, such as a trait providing herbicide resistance, or insect resistance, such as using a gene from Bacillus thuringiensis to provide resistance against lepidopteran, coliopteran, homopteran, hemiopteran, and other insects. Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent Application publication 2003/0083480 A1 also for imparting glyphosate tolerance; dicamba monooxygenase disclosed in U.S. Patent Application publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J. 4:833-840 and Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for impartinig pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260, all of said U.S. patents and patent application Publications are incorporated herein by reference. Molecules and methods for imparting insect/nematode/virus resistance is disclosed in U.S. Pat. Nos. 5,250,515; 5,880,275; 6,506,599; 5,986,175 and U.S. Patent Application Publication 2003/0150017 A1, all of which are incorporated herein by reference.

In particular embodiments, the inventors contemplate the use of antibodies, either monoclonal or polyclonal which bind to the proteins disclosed herein. Means for preparing and characterizing antibodies are well known in the art (See, e.g., Antibodies: A Laboratory Manual, Cold Spring Harbor Laboratory, 1988; incorporated herein by reference). The methods for generating monoclonal antibodies (mAbs) generally begin along the same lines as those for preparing polyclonal antibodies. Briefly, a polyclonal antibody is prepared by immunizing an animal with an immunogenic composition in accordance with the present invention and collecting antisera from that immunized animal. A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include using glutaraldehyde, m-maleimidobencoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified antifungal protein, polypeptide or peptide. The immunizing composition is administered in a manner effective to stimulate antibody producing cells. Rodents such as mice and rats are preferred animals, however, the use of rabbit, sheep, or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas).

Any one of a number of myeloma cells may be used, as are known to those of skill in the art (Goding, 1986, pp. 65-66; Campbell, 1984, pp. 75-83). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the MOMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Spend virus have been described (Kohler and Milstein, 1975; 1976), and those using polyethylene glycol (PEG), such as 37% (v/v) PEG, (Gefter et al., 1977). The use of electrically induced fusion methods is also appropriate (Goding, 1986, pp. 71-74).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azasenne blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn) and U.S. Pat. No. 6,153,812 (wheat) and Agrobacterium-mediated transformation is described in U.S. Pat. No. 5,159,135 (cotton); U.S. Pat. No. 5,824,877 (soybean); U.S. Pat. No. 5,591,616 (corn); and U.S. Pat. No. 6,384,301 (soybean), and in US Patent Application Publication 2004/0244075, all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, e.g., to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695, both incorporated herein by reference.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, e.g., various media and recipient target cells, transformation of immature embryos and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plants line for screening of plants having an enhanced agronomic trait. In addition to direct transformation of a plant with a recombinant DNA, transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced agronomic trait, e.g., enhanced yield, can be crossed with transgenic plant line having other recombinant DNA that confers another trait, e.g., herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA Progeny plants carrying DNA for both parental traits can be crossed back into the female parent line multiple times, e.g., usually 6 to 8 generations, to produce a progeny plant with substantially the same genotype as one original transgenic parental line but for the recombinant DNA of the other transgenic parental line.

In the practice of transformation DNA is typically introduced into only a small percentage of target cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV) and gentamycin (aac3 and aacC4) or resistance to herbicides such as glufosinate (bar or pat) and glyphosate (aroA or EPSPS). Examples of such selectable are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Cells that survive exposure to the selective agent, or cells that have been scored positive in a screening assay, may be cultured in regeneration media and allowed to mature into plants. Developing plantlets can be transferred to plant growth mix, and hardened off, e.g., in an environmentally controlled chamber at about 85% relative humidity, 600 ppm CO₂, and 25-250 microeinsteins m⁻²s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, e.g., self-pollination is commonly used with transgenic corn. The regenerated transformed plant or its progeny seed or plants can be tested for expression of the recombinant DNA and screened for the presence of enhanced agronomic trait.

Transgenic plant seed provided by this invention are grown to generate transgenic plants having an enhanced trait as compared to a control plant. Such seed for plants with enhanced agronomic trait is identified by screening transformed plants or progeny seed for enhanced trait. For efficiency a screening program is designed to evaluate multiple transgenic plants (events) comprising the recombinant DNA, e.g., multiple plants from 2 to 20 or more transgenic events.

Transgenic plants grown from transgenic seed provided herein demonstrate enhanced agronomic traits that contribute to increased yield or other trait that provides increased plant value, including, for example, enhanced seed quality. Of particular interest are plants having enhanced yield resulting from enhanced plant growth and development, stress tolerance, enhanced seed development, higher light response, enhanced flower development, or enhanced carbon and/or nitrogen metabolism.

Table 1 provides a list of protein encoding DNA (“genes”) that are useful as recombinant DNA for production of transgenic plants with enhanced agronomic trait, the elements of Table 1 are described by reference to:

“NUC SEQ ID NO” which is a SEQ ID NO for a DNA sequence in the Sequence Listing.
“PEP SEQ ID NO” which is a SEQ ID NO for an amino acid sequence in the Sequence Listing.
GENE ID″ which is an arbitrary name for the recombinant DNA.
“Base Vector” which is a reference to the identifying number in Table 5 of base vectors used for transformation of the recombinant DNA. Construction of plant transformation constructs is illustrated in Example 1.
“annotation” refers to a description of the top hit protein obtained from an amino acid sequence query of each PEP SEQ ID NO to GenBank database of the National Center for Biotechnology Information (NCBI). Identifier is the GenBank ID number for the informative BLAST hit with −F T.

TABLE 1

| NUC | PEP | | | annotation | | | |
|---|---|---|---|---|---|---|---|
| SEQ | SEQ | Base | | | % | | |
| ID NO | ID NO | vector | GENE ID | e-value | identity | identifier | description |
| 1 | 194 | 1 | PHE0003351_PMON81242 | 0 | 98 | 168586 | gb\|AAA33498.1\|pyruvate, orthophosphate |
| | | | | | | | dikinase |
| 2 | 195 | 7 | PHE0003351_PMON83625 | 0 | 98 | 168586 | gb\|AAA33498.1\|pyruvate, orthophosphate |
| | | | | | | | dikinase |
| 3 | 196 | 1 | PHE0000207_PMON77878 | 1.00E−144 | 96 | 34907990 | ref\|NP_915342.1\|putative |
| | | | | | | | calcium-dependent protein |
| | | | | | | | kinase [Oryza sativa |
| | | | | | | | (japonica cultivar-group)] |
| 4 | 197 | 1 | PHE0000208_PMON77879 | 1.00E−143 | 94 | 50919297 | ref\|XP_470045.1\|putative |
| | | | | | | | calmodulin-domain protein |
| | | | | | | | kinase [Oryza sativa |
| | | | | | | | (japonica cultivar-group)] |
| 5 | 198 | 1 | PHE0000209_PMON77891 | 1.00E−135 | 89 | 53850561 | gb\|AAU95457.1\|At5g12180 |
| | | | | | | | [Arabidopsis thaliana] |
| | | | | | | | dbj\|BAB10036.1\|calcium- |
| | | | | | | | dependent protein kinase |
| 6 | 199 | 1 | PHE0000210_PMON77880 | 1.00E−137 | 89 | 26452430 | dbj\|BAC43300.1\|putative |
| | | | | | | | calcium-dependent protein |
| | | | | | | | kinase [Arabidopsis |
| | | | | | | | thaliana] |
| 7 | 200 | 8 | PHE0001329_PMON92878 | 0 | 100 | 34903780 | dbj\|BAB92151.1\|putative |
| | | | | | | | CBL-interacting protein |
| | | | | | | | kinase 2 [Oryza sativa |
| | | | | | | | (japonica |
| 8 | 201 | 1 | PHE0001425_PMON79162 | 1.00E−154 | 100 | 51979679 | ref\|XP_507586.1\|PREDICTED |
| | | | | | | | P0524F03.33 gene |
| | | | | | | | product [Oryza sativa |
| | | | | | | | (japonica cultivar-group)] |
| | | | | | | | ref\|XP_482612.1\|putative |
| | | | | | | | CCR4-NOT transcription |
| | | | | | | | complex, subunit 7 |
| 9 | 202 | 8 | PHE0001573_PMON92870 | 0 | 78 | 984262 | emb\|CAA58052.1\|asparragine |
| | | | | | | | synthetase [Zea mays] |
| 10 | 203 | 12 | PHE0001664_PMON99280 | 0 | 100 | 34906358 | sp\|Q9LDE6\|CKX1_ORYSA |
| | | | | | | | Probable cytokinin |
| | | | | | | | dehydrogenase precursor |
| | | | | | | | (Cytokinin oxidase) (CKO) |
| 11 | 204 | 1 | PHE0001674_PMON79194 | 5.00E−12 | 50 | 15223390 | ref\|NP_171645.1\|myb |
| | | | | | | | family transcription factor |
| | | | | | | | [Arabidopsis thaliana] |
| 12 | 205 | 10 | PHE0002026_PMON96489 | 0 | 87 | 32488298 | emb\|CAE03364.1\|OSJNBb0065L13.7 |
| | | | | | | | [Oryza sativa |
| | | | | | | | (japonica cultivar-group)] |
| 13 | 206 | 8 | PHE0002108_PMON92821 | 2.00E−31 | 100 | 10176234 | dbj\|BAB07329.1\|cold-shock |
| | | | | | | | protein [Bacillus halodurans |
| | | | | | | | C-125] |
| 14 | 207 | 8 | PHE0002109_PMON93856 | 6.00E−33 | 100 | 41324401 | emb\|CAF18741.1\|COLD- |
| | | | | | | | SHOCK PROTEIN CSPA |
| | | | | | | | [Corynebacterium |
| | | | | | | | glutamicum ATCC 13032] |
| 15 | 208 | 8 | PHE0002508_PMON92607 | 2.00E−79 | 72 | 50509850 | dbj\|BAD32022.1\|putative |
| | | | | | | | transcription factor [Oryza |
| | | | | | | | sativa |
| 16 | 209 | 1 | PHE0002650_PMON81832 | 1.00E−132 | 100 | 9964296 | gb\|AAG09919.1\|MADS |
| | | | | | | | box protein 2 [Zea mays] |
| 17 | 210 | 2 | PHE0002989_PMON95630 | 1.00E−117 | 100 | 7271044 | emb\|CAB80652.1\|small |
| | | | | | | | GTP-binding protein-like |
| | | | | | | | [Arabidopsis thaliana] |
| 18 | 211 | 6 | PHE0003290_PMON95107 | 4.00E−29 | 34 | 7269078 | emb\|CAB79187.1\|hypothetical |
| | | | | | | | protein [Arabidopsis |
| | | | | | | | thaliana] |
| 19 | 212 | 6 | PHE0003300_PMON95106 | 7.00E−81 | 54 | 50908933 | ref\|XP_465955.1\|putative |
| | | | | | | | nodulin 3 [Oryza sativa |
| | | | | | | | (japonica cultivar-group)] |
| 20 | 213 | 6 | PHE0003303_PMON95080 | 2.00E−96 | 69 | 38347194 | emb\|CAD37109.2\|OSJNBa0024J22.22 |
| | | | | | | | [Oryza sativa |
| | | | | | | | (japonica cultivar-group)] |
| 21 | 214 | 8 | PHE0003389_PMON94682 | 0 | 65 | 52076827 | dbj\|BAD45770.1\|putative |
| | | | | | | | Cyt-P450 monooxygenase |
| | | | | | | | [Oryza sativa (japonica |
| | | | | | | | cultivar-group)] |
| 22 | 215 | 8 | PHE0003614_PMON95111 | 0 | 94 | 32309578 | gb\|AAP79441.1\|glutamate |
| | | | | | | | decarboxylase [Oryza sativa |
| | | | | | | | (japonica cultivar-group)] |
| 23 | 216 | 8 | PHE0003684_PMON92807 | 1.00E−72 | 68 | 34906004 | dbj\|BAB63676.1\|induced |
| | | | | | | | protein MgI1 [Oryza sativa |
| | | | | | | | (japonica cultivar-group)] |
| 24 | 217 | 9 | PHE0003684_PMON93378 | 1.00E−72 | 68 | 34906004 | dbj\|BAB63676.1\|induced |
| | | | | | | | protein MgI1 [Oryza sativa |
| | | | | | | | (japonica cultivar-group)] |
| 25 | 218 | 8 | PHE0003853_PMON92602 | 1.00E−179 | 98 | 62320210 | ref\|NP_195478.2\|cyclin |
| | | | | | | | family protein [Arabidopsis |
| | | | | | | | thaliana] gb\|AAS49095.1\| |
| | | | | | | | At4g37630 [Arabidopsis |
| | | | | | | | thaliana] |
| 26 | 219 | 11 | PHE0003903_PMON98271 | 0 | 99 | 19851522 | gb\|AAL99744.1\|pyruvate |
| | | | | | | | decarboxylase [Zea mays] |
| 27 | 220 | 11 | PHE0003905_PMON99283 | 0 | 92 | 11995457 | gb\|AAG43027.1\|aldehyde |
| | | | | | | | dehydrogenase [Oryza |
| | | | | | | | sativa] |
| 28 | 221 | 11 | PHE0003907_PMON98066 | 5.00E−87 | 86 | 50906015 | ref\|XP_464496.1\|ribosomal |
| | | | | | | | protein L12-like protein |
| | | | | | | | [Oryza sativa (japonica |
| | | | | | | | cultivar-group)] |
| 29 | 222 | 11 | PHE0003908_PMON98064 | 0 | 84 | 51535811 | dbj\|BAD37896.1\|ARG1- |
| | | | | | | | like protein [Oryza sativa |
| | | | | | | | (japonica cultivar-group)] |
| 30 | 223 | 6 | PHE0003960_PMON95079 | 1.00E−156 | 87 | 50905641 | ref\|XP_464309.1\|putative |
| | | | | | | | choline-phosphate |
| | | | | | | | cytidylyltransferase [Oryza |
| | | | | | | | sativa (japonica cultivar- |
| | | | | | | | group)] |
| 31 | 224 | 5 | PHE0003967_PMON95088 | 1.00E−102 | 83 | 55168334 | gb\|AAV44199.1\|dehydroascorbate |
| | | | | | | | reductase [Oryza |
| | | | | | | | sativa (japonica cultivar- |
| | | | | | | | group)] |
| 32 | 225 | 10 | PHE0003985_PMON96457 | 1.00E−30 | 58 | 55770043 | ref\|XP_550011.1\|hypothetical |
| | | | | | | | protein [Oryza sativa |
| | | | | | | | (japonica cultivar-group)] |
| 33 | 226 | 10 | PHE0003987_PMON96134 | 5.00E−41 | 74 | 50919885 | ref\|XP_470303.1\|hypothetical |
| | | | | | | | protein [Oryza sativa |
| | | | | | | | (japonica cultivar-group)] |
| 34 | 227 | 10 | PHE0004001_PMON96453 | 4.00E−22 | 66 | 51978970 | ref\|XP_507362.1\|PREDICTED |
| | | | | | | | OSJNBa0077F02.127 |
| | | | | | | | gene product [Oryza sativa |
| | | | | | | | (japonica cultivar-group)] |
| 35 | 228 | 8 | PHE0004023_PMON92446 | 1.00E−132 | 88 | 12651665 | gb\|AAA20093.2\|Alfin-1 |
| | | | | | | | [Medicago sativa] |
| | | | | | | | pir\|\|T09646 probable zinc |
| | | | | | | | finger protein - alfalfa |
| | | | | | | | (fragment) |
| 36 | 229 | 4 | PHE0004026_PMON93885 | 0 | 100 | 21592703 | gb\|AAM64652.1\|LAX1/ |
| | | | | | | | AUX1-like permease |
| | | | | | | | [Arabidopsis thaliana] |
| 37 | 230 | 4 | PHE0004027_PMON93860 | 0 | 100 | 7269873 | emb\|CAB79732.1\|cytokinin |
| | | | | | | | oxidase-like protein |
| | | | | | | | [Arabidopsis thaliana] |
| 38 | 231 | 15 | PHE0004028_PMON94697 | 0 | 100 | 216765 | dbj\|BAA14344.1\|sucrose |
| | | | | | | | phosphorylase |
| | | | | | | | [Leuconostoc |
| | | | | | | | mesenteroides] |
| 12607 | 231 | n/a | PHE0010424_PMON17730 | 0 | 100 | 216765 | dbj\|BAA14344.1\|sucrose |
| | | | | | | | phosphorylase |
| | | | | | | | [Leuconostoc |
| | | | | | | | mesenteroides] |
| 39 | 232 | 8 | PHE0004034_PMON92631 | 0 | 100 | 6520233 | dbj\|BAA87958.1\|CW14 |
| | | | | | | | [Arabidopsis thaliana] |
| 40 | 233 | 8 | PHE0004039_PMON92634 | 1.00E−178 | 65 | 26452061 | ref\|NP_191207.2\|myosin |
| | | | | | | | heavy chain-related |
| | | | | | | | [Arabidopsis thaliana] |
| 41 | 234 | 8 | PHE0004047_PMON92619 | 4.00E−79 | 74 | 62087121 | dbj\|BAD91881.1\|transcription |
| | | | | | | | factor lim1 [Eucalyptus |
| | | | | | | | camaldulensis] |
| 42 | 235 | 14 | PHE0004047_PMON93388 | 4.00E−79 | 74 | 62087121 | dbj\|BAD91881.1\|transcription |
| | | | | | | | factor lim1 [Eucalyptus |
| | | | | | | | camaldulensis] |
| 43 | 236 | 8 | PHE0004068_PMON93663 | 3.00E−94 | 100 | 15293293 | ref\|NP_563710.1\|AWPM- |
| | | | | | | | 19-like membrane family |
| | | | | | | | protein [Arabidopsis |
| | | | | | | | thaliana] |
| 44 | 237 | 8 | PHE0004071_PMON93311 | 1.00E−130 | 100 | 21358850 | ref\|NP_568751.1\| |
| | | | | | | | polyadenylate-binding |
| | | | | | | | protein, putative/PABP, |
| | | | | | | | putative [Arabidopsis |
| | | | | | | | thaliana] |
| 45 | 238 | 8 | PHE0004072_PMON93654 | 0 | 100 | 23297397 | ref\|NP_192188.2\|GTP- |
| | | | | | | | binding family protein |
| | | | | | | | [Arabidopsis thaliana] |
| 46 | 239 | 14 | PHE0004072_PMON93669 | 0 | 100 | 23297397 | ref\|NP_192188.2\|GTP- |
| | | | | | | | binding family protein |
| | | | | | | | [Arabidopsis thaliana] |
| 47 | 240 | 8 | PHE0004074_PMON94164 | 0 | 100 | 9759255 | ref\|NP_196133.3\| |
| | | | | | | | transcription elongation |
| | | | | | | | factor-related [Arabidopsis |
| | | | | | | | thaliana] |
| 48 | 241 | 8 | PHE0004075_PMON92851 | 1.00E−132 | 100 | 11994587 | ref\|NP_566493.1\|nodulin |
| | | | | | | | MtN3 family protein |
| | | | | | | | [Arabidopsis thaliana] |
| 49 | 242 | 8 | PHE0004080_PMON93321 | 1.00E−143 | 99 | 16173 | emb\|CAA42168.1\|L- |
| | | | | | | | ascorbate peroxidase |
| | | | | | | | [Arabidopsis thaliana] |
| 50 | 243 | 14 | PHE0004084_PMON95141 | 0 | 100 | 7267537 | emb\|CAB78019.1\|putative |
| | | | | | | | phi-1-like phosphate- |
| | | | | | | | induced protein |
| | | | | | | | [Arabidopsis thaliana] |
| | | | | | | | gb\|AAM18526.1\|cell cycle- |
| | | | | | | | related protein [Arabidopsis |
| | | | | | | | thaliana] |
| 51 | 244 | 8 | PHE0004093_PMON93332 | 0 | 100 | 12744973 | gb\|AAK06866.1\|putative |
| | | | | | | | ATPase [Arabidopsis |
| | | | | | | | thaliana] ref\|NP_173536.1\| |
| | | | | | | | O-methyltransferase, |
| | | | | | | | putative [Arabidopsis |
| | | | | | | | thaliana] |
| 52 | 245 | 14 | PHE0004093_PMON94155 | 0 | 100 | 12744973 | gb\|AAK06866.1\|putative |
| | | | | | | | ATPase [Arabidopsis |
| | | | | | | | thaliana] ref\|NP_173536.1\| |
| | | | | | | | O-methyltransferase, |
| | | | | | | | putative [Arabidopsis |
| | | | | | | | thaliana] |
| 53 | 246 | 8 | PHE0004139_PMON92898 | 2.00E−88 | 100 | 21554099 | ref\|NP_568761.1\|expressed |
| | | | | | | | protein [Arabidopsis |
| | | | | | | | thaliana] |
| 54 | 247 | 8 | PHE0004144_PMON93842 | 1.00E−78 | 100 | 21555039 | ref\|NP_565390.1\|actin- |
| | | | | | | | depolymerizing factor 5 |
| | | | | | | | (ADF5) [Arabidopsis |
| | | | | | | | thaliana] |
| 55 | 248 | 8 | PHE0004148_PMON92574 | 0 | 100 | 48768596 | ref\|ZP_00272945.1\|COG0538: |
| | | | | | | | Isocitrate |
| | | | | | | | dehydrogenases [Ralstonia |
| | | | | | | | metallidurans CH34] |
| 56 | 249 | 8 | PHE0004149_PMON92471 | 1.00E−148 | 99 | 31096331 | ref\|NP_441003.1\| |
| | | | | | | | phycocyanin alpha |
| | | | | | | | phycocyanobilin lyase; |
| | | | | | | | CpcE [Synechocystis sp. |
| | | | | | | | PCC 6803] |
| 57 | 250 | 14 | PHE0004149_PMON93899 | 1.00E−148 | 99 | 31096331 | ref\|NP_441003.1\| |
| | | | | | | | phycocyanin alpha |
| | | | | | | | phycocyanobilin lyase; |
| | | | | | | | CpcE [Synechocystis sp. |
| | | | | | | | PCC 6803] |
| 58 | 251 | 15 | PHE0004152_PMON93672 | 3.00E−85 | 60 | 8978267 | ref\|NP_199781.1\|DNA- |
| | | | | | | | binding protein-related |
| | | | | | | | [Arabidopsis thaliana] |
| 59 | 252 | 8 | PHE0004155_PMON92626 | 0 | 100 | 22136876 | ref\|NP_200010.1\|sorbitol |
| | | | | | | | dehydrogenase, putative/ |
| | | | | | | | L-iditol 2-dehydrogenase, |
| | | | | | | | putative [Arabidopsis |
| | | | | | | | thaliana] |
| 60 | 253 | 8 | PHE0004156_PMON92623 | 0 | 98 | 12322729 | ref\|NP_187478.1\| |
| | | | | | | | phototropic-responsive |
| | | | | | | | protein, putative |
| | | | | | | | [Arabidopsis thaliana] |
| 61 | 254 | 8 | PHE0004162_PMON92481 | 3.00E−77 | 57 | 7269806 | emb\|CAB79666.1\|phytochrome- |
| | | | | | | | associated protein |
| | | | | | | | PAP2 [Arabidopsis |
| | | | | | | | thaliana] |
| 62 | 255 | 8 | PHE0004164_PMON92465 | 4.00E−67 | 100 | 21537028 | ref\|NP_198423.1\|glycosyl |
| | | | | | | | hydrolase family protein 17 |
| | | | | | | | [Arabidopsis thaliana] |
| 63 | 256 | 8 | PHE0004166_PMON93801 | 6.00E−09 | 100 | 13374861 | emb\|CAC34495.1\|putative |
| | | | | | | | strictosidine synthase-like |
| | | | | | | | [Arabidopsis thaliana] |
| 64 | 257 | 8 | PHE0004167_PMON93333 | 1.00E−176 | 100 | 28827764 | ref\|NP_569050.1\| |
| | | | | | | | adenylylsulfate kinase, |
| | | | | | | | putative [Arabidopsis |
| | | | | | | | thaliana] |
| 65 | 258 | 8 | PHE0004168_PMON93855 | 0 | 100 | 18176302 | ref\|NP_199253.1\|FAD- |
| | | | | | | | binding domain-containing |
| | | | | | | | protein [Arabidopsis |
| | | | | | | | thaliana] |
| 66 | 259 | 8 | PHE0004169_PMON92568 | 0 | 100 | 5080826 | gb\|AAD39335.1\|Putative |
| | | | | | | | Aldo/keto reductase |
| | | | | | | | [Arabidopsis thaliana] |
| 67 | 260 | 8 | PHE0004184_PMON92565 | 0 | 100 | 7270846 | emb\|CAB80527.1\|multiubiquitin |
| | | | | | | | chain binding protein |
| | | | | | | | (MBP1) [Arabidopsis |
| | | | | | | | thaliana] |
| 68 | 261 | 8 | PHE0004185_PMON92802 | 0 | 100 | 28460683 | ref\|NP_182075.1\| |
| | | | | | | | cytochrome P450, putative |
| | | | | | | | [Arabidopsis thaliana] |
| 69 | 262 | 8 | PHE0004188_PMON92803 | 0 | 100 | 20465485 | ref\|NP_200218.1\|heat |
| | | | | | | | shock transcription factor |
| | | | | | | | family protein [Arabidopsis |
| | | | | | | | thaliana] |
| 70 | 263 | 8 | PHE0004190_PMON92801 | 1.00E−167 | 98 | 7267277 | ref\|NP_192426.1\|basic |
| | | | | | | | helix-loop-helix (bHLH) |
| | | | | | | | family protein [Arabidopsis |
| | | | | | | | thaliana] |
| 71 | 264 | 8 | PHE0004208_PMON92834 | 1.00E−83 | 55 | 21555865 | gb\|AAS09998.1\|MYB |
| | | | | | | | transcription factor |
| | | | | | | | [Arabidopsis thaliana] |
| 72 | 265 | 8 | PHE0004215_PMON92827 | 2.00E−55 | 65 | 7320708 | ref\|NP_195750.1\| |
| | | | | | | | phosphatidylethanolamine- |
| | | | | | | | binding family protein |
| | | | | | | | [Arabidopsis thaliana] |
| 73 | 266 | 8 | PHE0004223_PMON92840 | 0 | 100 | 6523058 | ref\|NP_190239.1\|fasciclin- |
| | | | | | | | like arabinogalactan family |
| | | | | | | | protein [Arabidopsis |
| | | | | | | | thaliana] |
| 74 | 267 | 8 | PHE0004225_PMON94167 | 0 | 99 | 1421730 | gb\|AAC49371.1\|RF2 |
| | | | | | | | gb\|AAG43988.1\|T |
| | | | | | | | cytoplasm male sterility |
| | | | | | | | restorer factor 2 [Zea mays] |
| 75 | 268 | 10 | PHE0004226_PMON95114 | 0 | 100 | 53793208 | dbj\|BAD54414.1\|aldehyde |
| | | | | | | | dehydrogenase ALDH2b |
| | | | | | | | [Oryza sativa (japonica |
| | | | | | | | cultivar-group)] |
| 76 | 269 | 8 | PHE0004227_PMON92605 | 5.00E−26 | 100 | 21314334 | gb\|AAM46894.1\|early |
| | | | | | | | drought induced protein |
| | | | | | | | [Oryza sativa (indica |
| | | | | | | | cultivar-group)] |
| 77 | 270 | 8 | PHE0004229_PMON92867 | 1.00E−24 | 100 | 6320482 | ref\|NP_010562.1\|Small |
| | | | | | | | plasma membrane protein |
| | | | | | | | related to a family of plant |
| | | | | | | | polypeptides that are |
| | | | | | | | overexpressed under high |
| | | | | | | | salt concentration or low |
| | | | | | | | temperature, not essential |
| | | | | | | | for viability, deletion causes |
| | | | | | | | hyperpolarization of the |
| | | | | | | | plasma membrane potential; |
| | | | | | | | Pmp3p [Saccharomyces |
| | | | | | | | cerevisiae] |
| 78 | 271 | 8 | PHE0004233_PMON92843 | 0 | 100 | 19310749 | ref\|NP_188922.1\|heat |
| | | | | | | | shock transcription factor |
| | | | | | | | family protein [Arabidopsis |
| | | | | | | | thaliana] |
| 79 | 272 | 13 | PHE0004237_PMON93673 | 9.00E−85 | 100 | 16338 | emb\|CAA45039.1\|heat |
| | | | | | | | shock protein 17.6-II |
| | | | | | | | [Arabidopsis thaliana] |
| 80 | 273 | 8 | PHE0004243_PMON92621 | 3.00E−72 | 82 | 30409461 | dbj\|BAC76332.1\|HAP3 |
| | | | | | | | [Oryza sativa (japonica |
| | | | | | | | cultivar-group)] |
| 81 | 274 | 8 | PHE0004244_PMON92858 | 1.00E−159 | 96 | 15321716 | gb\|AAK95562.1\|leafy |
| | | | | | | | cotyledon1 [Zea mays] |
| 82 | 275 | 8 | PHE0004245_PMON93813 | 1.00E−131 | 100 | 50509850 | dbj\|BAD32022.1\|putative |
| | | | | | | | transcription factor [Oryza |
| | | | | | | | sativa (japonica cultivar- |
| | | | | | | | group)] |
| 83 | 276 | 8 | PHE0004248_PMON94672 | 1.00E−98 | 100 | 34907184 | ref\|NP_914939.1\|putative |
| | | | | | | | CCAAT-binding |
| | | | | | | | transcription factor subunit |
| | | | | | | | A(CBF-A) [Oryza sativa |
| 84 | 277 | 8 | PHE0004249_PMON95137 | 1.00E−48 | 100 | 12642910 | ref\|NP_850005.1\|expressed |
| | | | | | | | protein [Arabidopsis |
| | | | | | | | thaliana] |
| 85 | 278 | 8 | PHE0004250_PMON92881 | 5.00E−78 | 100 | 30409463 | dbj\|BAC76333.1\|HAP3 |
| | | | | | | | [Oryza sativa (japonica |
| | | | | | | | cultivar-group)] |
| 86 | 279 | 8 | PHE0004252_PMON92606 | 1.00E−173 | 100 | 18481620 | gb\|AAL73485.1\|repressor |
| | | | | | | | protein [Oryza sativa] |
| 87 | 280 | 8 | PHE0004253_PMON92874 | 1.00E−143 | 100 | 18481626 | gb\|AAL73488.1\|repressor |
| | | | | | | | protein [Zea mays] |
| 88 | 281 | 14 | PHE0004258_PMON93385 | 0 | 100 | 1871189 | gb\|AAB63549.1\|putative |
| | | | | | | | protein kinase [Arabidopsis |
| | | | | | | | thaliana] |
| 89 | 282 | 8 | PHE0004258_PMON93806 | 0 | 100 | 1871189 | gb\|AAB63549.1\|putative |
| | | | | | | | protein kinase [Arabidopsis |
| | | | | | | | thaliana] |
| 90 | 283 | 14 | PHE0004259_PMON93384 | 0 | 100 | 9755654 | ref\|NP_197112.1\|expressed |
| | | | | | | | protein [Arabidopsis |
| | | | | | | | thaliana] |
| 91 | 284 | 8 | PHE0004260_PMON92854 | 1.00E−48 | 100 | 12642910 | ref\|NP_850005.1\|expressed |
| | | | | | | | protein [Arabidopsis |
| | | | | | | | thaliana] |
| 92 | 285 | 14 | PHE0004261_PMON93389 | 1.00E−170 | 100 | 7270230 | ref\|NP_195009.1\|protein |
| | | | | | | | kinase, putative |
| | | | | | | | [Arabidopsis thaliana] |
| 93 | 286 | 8 | PHE0004261_PMON93655 | 1.00E−170 | 100 | 7270230 | ref\|NP_195009.1\|protein |
| | | | | | | | kinase, putative |
| | | | | | | | [Arabidopsis thaliana] |
| 94 | 287 | 8 | PHE0004262_PMON92862 | 0 | 100 | 42570809 | ref\|NP_973478.1\|protein |
| | | | | | | | kinase, putative |
| | | | | | | | [Arabidopsis thaliana] |
| 95 | 288 | 14 | PHE0004262_PMON93360 | 0 | 100 | 42570809 | ref\|NP_973478.1\|protein |
| | | | | | | | kinase, putative |
| | | | | | | | [Arabidopsis thaliana] |
| 96 | 289 | 8 | PHE0004264_PMON92845 | 3.00E−95 | 100 | 21554624 | ref\|NP_201267.1\| |
| | | | | | | | invertase/pectin |
| | | | | | | | methylesterase inhibitor |
| | | | | | | | family protein [Arabidopsis |
| | | | | | | | thaliana] |
| 97 | 290 | 14 | PHE0004264_PMON93354 | 3.00E−95 | 100 | 21554624 | ref\|NP_201267.1\| |
| | | | | | | | invertase/pectin |
| | | | | | | | methylesterase inhibitor |
| | | | | | | | family protein [Arabidopsis |
| | | | | | | | thaliana] |
| 98 | 291 | 8 | PHE0004265_PMON92873 | 0 | 100 | 642305 | ref\|NP_013662.1\| |
| | | | | | | | Hypothetical ORF; |
| | | | | | | | Yml050wp [Saccharomyces |
| | | | | | | | cerevisiae] |
| 99 | 292 | 14 | PHE0004265_PMON93807 | 0 | 100 | 642305 | ref\|NP_013662.1\| |
| | | | | | | | Hypothetical ORF; |
| | | | | | | | Yml050wp [Saccharomyces |
| | | | | | | | cerevisiae] |
| 100 | 293 | 8 | PHE0004266_PMON92877 | 0 | 99 | 23506085 | ref\|NP_567548.1\|pseudo- |
| | | | | | | | response regulator 2 |
| | | | | | | | (APRR2) (TOC2) |
| | | | | | | | [Arabidopsis thaliana] |
| 101 | 294 | 8 | PHE0004284_PMON93857 | 0 | 99 | 18399375 | ref\|NP_566402.1\|U-box |
| | | | | | | | domain-containing protein |
| | | | | | | | [Arabidopsis thaliana] |
| 102 | 295 | 10 | PHE0004285_PMON95136 | 1.00E−161 | 96 | 37542675 | gb\|AAL47207.1\|HAP3-like |
| | | | | | | | transcriptional-activator |
| | | | | | | | [Oryza sativa (indica |
| | | | | | | | cultivar-group)] |
| 103 | 296 | 8 | PHE0004286_PMON93666 | 0 | 99 | 255220 | gb\|AAB23208.1\|isocitrate |
| | | | | | | | lyase, threo-D S-isocitrate |
| | | | | | | | glyoxylate-lyase, IL {EC |
| | | | | | | | 4.1.3.1} [Brassica napus, |
| | | | | | | | seedlings, Peptide, 576 aa] |
| 104 | 297 | 8 | PHE0004287_PMON93344 | 0 | 88 | 50937953 | ref\|XP_478504.1\|putative |
| | | | | | | | isocitrate lyase [Oryza |
| | | | | | | | sativa (japonica cultivar- |
| | | | | | | | group)] |
| 105 | 298 | 2 | PHE0004307_PMON94102 | 1.00E−105 | 62 | 38345397 | emb\|CAE03088.2\|OSJNBa0017B10.3 |
| | | | | | | | [Oryza sativa |
| | | | | | | | (japonica cultivar-group)] |
| 106 | 299 | 14 | PHE0004314_PMON93397 | 9.00E−52 | 54 | 55740645 | gb\|AAV63915.1\|hypothetical |
| | | | | | | | protein At4g03965 |
| | | | | | | | [Arabidopsis thaliana] |
| 107 | 300 | 8 | PHE0004321_PMON93811 | 1.00E−128 | 100 | 18655355 | sp\|O48646\|GPX4_ARATH |
| | | | | | | | Probable phospholipid |
| | | | | | | | hydroperoxide glutathione |
| | | | | | | | peroxidase, mitochondrial |
| | | | | | | | precursor (PHGPx) |
| | | | | | | | (AtGPX1) |
| 108 | 301 | 14 | PHE0004321_PMON93834 | 1.00E−128 | 100 | 18655355 | ref\|NP_192897.2\| |
| | | | | | | | glutathione peroxidase, |
| | | | | | | | putative [Arabidopsis |
| | | | | | | | thaliana] |
| 109 | 302 | 8 | PHE0004325_PMON93818 | 5.00E−78 | 89 | 50906887 | ref\|XP_464932.1\|cytochrome |
| | | | | | | | c biogenesis protein-like |
| | | | | | | | [Oryza sativa (japonica |
| | | | | | | | cultivar-group)] |
| 110 | 303 | 8 | PHE0004335_PMON93850 | 0 | 100 | 28393953 | gb\|AAO42384.1\|putative |
| | | | | | | | major intrinsic protein |
| | | | | | | | [Arabidopsis thaliana] |
| 111 | 304 | 8 | PHE0004336_PMON93858 | 1.00E−146 | 69 | 51964952 | ref\|XP_482812.1\|major |
| | | | | | | | intrinsic protein-like [Oryza |
| | | | | | | | sativa (japonica cultivar- |
| | | | | | | | group)] |
| 112 | 305 | 4 | PHE0004337_PMON93886 | 0 | 62 | 50943587 | ref\|XP_481321.1\|unknown |
| | | | | | | | protein [Oryza sativa |
| | | | | | | | (japonica cultivar-group)] |
| 113 | 306 | 8 | PHE0004348_PMON93810 | 1.00E−32 | 100 | 15644431 | ref\|NP_229483.1\|cold shock |
| | | | | | | | protein [Thermotoga |
| | | | | | | | maritima MSB8] |
| 114 | 307 | 8 | PHE0004349_PMON93812 | 8.00E−33 | 100 | 15644617 | ref\|NP_229670.1\|cold shock |
| | | | | | | | protein [Thermotoga |
| | | | | | | | maritima MSB8] |
| 115 | 308 | 8 | PHE0004350_PMON93826 | 3.00E−31 | 100 | 20808157 | ref\|NP_623328.1\|Cold |
| | | | | | | | shock proteins |
| | | | | | | | [Thermoanaerobacter |
| | | | | | | | tengcongensis MB4] |
| 116 | 309 | 8 | PHE0004351_PMON93821 | 7.00E−32 | 100 | 56419891 | ref\|YP_147209.1\|cold shock |
| | | | | | | | protein [Geobacillus |
| | | | | | | | kaustophilus HTA426] |
| 117 | 310 | 8 | PHE0004352_PMON93824 | 1.00E−27 | 88 | 49611845 | ref\|YP_050486.1\|cold |
| | | | | | | | shock protein [Erwinia |
| | | | | | | | carotovora subsp. |
| | | | | | | | atroseptica SCRI1043] |
| 118 | 311 | 8 | PHE0004383_PMON93816 | 1.00E−34 | 98 | 50899510 | ref\|XP_450543.1\|unknown |
| | | | | | | | protein [Oryza sativa |
| | | | | | | | (japonica cultivar-group)] |
| 119 | 312 | 8 | PHE0004393_PMON94192 | 8.00E−95 | 100 | 42572939 | ref\|NP_974566.1\|calcineurin |
| | | | | | | | B-like protein 1 (CBL1) |
| | | | | | | | [Arabidopsis thaliana] |
| 120 | 313 | 8 | PHE0004395_PMON94145 | 0 | 100 | 30690488 | ref\|NP_849501.1\|phospholipase |
| | | | | | | | D delta/PLD delta |
| | | | | | | | (PLDDELTA) [Arabidopsis |
| | | | | | | | thaliana] |
| 121 | 314 | 8 | PHE0004396_PMON94137 | 0 | 100 | 7270422 | emb\|CAB80188.1\|arginine |
| | | | | | | | decarboxylase SPE2 |
| | | | | | | | [Arabidopsis thaliana] |
| 122 | 315 | 8 | PHE0004417_PMON94190 | 1.00E−170 | 100 | 1230677 | gb\|AAC17191.1\| |
| | | | | | | | spermidine synthase |
| | | | | | | | [Saccharomyces cerevisiae] |
| 123 | 316 | 8 | PHE0004418_PMON94368 | 0 | 100 | 798930 | sp\|P50264\|FMS1_YEAST |
| | | | | | | | Polyamine oxidase FMS1 |
| | | | | | | | (Fenpropimorph resistance |
| | | | | | | | multicopy suppressor 1) |
| 124 | 317 | 8 | PHE0004419_PMON95100 | 0 | 66 | 21281139 | ref\|NP_567276.1\| |
| | | | | | | | amidohydrolase family |
| | | | | | | | protein [Arabidopsis |
| | | | | | | | thaliana] |
| 125 | 318 | 10 | PHE0004421_PMON95120 | 2.00E−53 | 78 | 33321848 | gb\|AAQ06658.1\|apetala2 |
| | | | | | | | domain-containing CBF1- |
| | | | | | | | like protein [Oryza sativa] |
| 126 | 319 | 10 | PHE0004422_PMON95123 | 3.00E−51 | 80 | 25991254 | gb\|AAN76804.1\|DREB-like |
| | | | | | | | protein [Zea mays] |
| 127 | 320 | 8 | PHE0004425_PMON94428 | 7.00E−37 | 98 | 11762134 | gb\|AAG40345.1\|AT5g17460 |
| | | | | | | | [Arabidopsis thaliana] |
| 128 | 321 | 8 | PHE0004431_PMON94398 | 1.00E−159 | 99 | 557818 | ref\|NP_012214.1\|Pho85p |
| | | | | | | | cyclin of the Pho80p |
| | | | | | | | subfamily, forms a |
| | | | | | | | functional kinase complex |
| | | | | | | | with Pho85p which |
| | | | | | | | phosphorylates Mmr1p and |
| | | | | | | | is regulated by Pho81p; |
| | | | | | | | involved in glycogen |
| | | | | | | | metabolism, expression is |
| | | | | | | | cell-cycle regulated; Pcl7p |
| | | | | | | | [Saccharomyces cerevisiae] |
| 129 | 322 | 8 | PHE0004432_PMON94112 | 0 | 100 | 15156338 | ref\|NP_354295.1\| |
| | | | | | | | hypothetical protein |
| | | | | | | | AGR_C_2368 |
| | | | | | | | [Agrobacterium |
| | | | | | | | tumefaciens str. C58] |
| 130 | 323 | 8 | PHE0004472_PMON94115 | 1.00E−128 | 100 | 16323494 | ref\|NP_187978.1\|seven in |
| | | | | | | | absentia (SINA) family |
| | | | | | | | protein [Arabidopsis |
| | | | | | | | thaliana] |
| 131 | 324 | 14 | PHE0004472_PMON94126 | 1.00E−128 | 100 | 16323494 | ref\|NP_187978.1\|seven in |
| | | | | | | | absentia (SINA) family |
| | | | | | | | protein [Arabidopsis |
| | | | | | | | thaliana] |
| 132 | 325 | 14 | PHE0004488_PMON95609 | 1.00E−123 | 100 | 21554344 | ref\|NP_198627.1\|ASF1- |
| | | | | | | | like anti-silencing family |
| | | | | | | | protein [Arabidopsis |
| | | | | | | | thaliana] |
| 133 | 326 | 14 | PHE0004491_PMON95628 | 3.00E−12 | 45 | 14916641 | dbj\|BAB19648.1\| |
| | | | | | | | preprophytosulfokine |
| | | | | | | | [Oryza sativa] |
| 134 | 327 | 14 | PHE0004492_PMON95614 | 0 | 100 | 22331730 | ref\|NP_190653.2\|phototropic- |
| | | | | | | | responsive NPH3 family |
| | | | | | | | protein [Arabidopsis |
| | | | | | | | thaliana] |
| 135 | 328 | 10 | PHE0004545_PMON95117 | 1.00E−106 | 100 | 28973235 | ref\|NP_173200.1\| |
| | | | | | | | ribosomal protein L14 |
| | | | | | | | family protein [Arabidopsis |
| | | | | | | | thaliana] |
| 136 | 329 | 8 | PHE0004574_PMON94433 | 0 | 100 | 16329404 | ref\|NP_440132.1\|transaldolase |
| | | | | | | | [Synechocystis sp. PCC |
| | | | | | | | 6803] |
| 137 | 330 | 14 | PHE0004606_PMON95627 | 0 | 100 | 130709 | pir\|\|S29317 phosphoprotein |
| | | | | | | | phosphatase (EC 3.1.3.16) 1 - |
| | | | | | | | maize gb\|AAA33545.1\| |
| | | | | | | | protein phosphatase-1 |
| 138 | 331 | 8 | PHE0004620_PMON94189 | 1.00E−101 | 57 | 56421275 | ref\|YP_148593.1\|6- |
| | | | | | | | phosphofructokinase |
| | | | | | | | (phosphofructokinase) |
| | | | | | | | (phosphohexokinase) |
| | | | | | | | [Geobacillus kaustophilus |
| | | | | | | | HTA426] |
| 139 | 332 | 14 | PHE0004620_PMON94442 | 1.00E−101 | 57 | 56421275 | ref\|YP_148593.1\|6- |
| | | | | | | | phosphofructokinase |
| | | | | | | | (phosphofructokinase) |
| | | | | | | | (phosphohexokinase) |
| | | | | | | | [Geobacillus kaustophilus |
| | | | | | | | HTA426] |
| 140 | 333 | 14 | PHE0004622_PMON95621 | 0 | 100 | 10177836 | ref\|NP_974942.1\|F-box |
| | | | | | | | family protein [Arabidopsis |
| | | | | | | | thaliana] |
| 141 | 334 | 8 | PHE0004626_PMON95101 | 0 | 88 | 50942161 | ref\|XP_480608.1\|putative |
| | | | | | | | gamma-aminobutyrate |
| | | | | | | | transaminase subunit |
| | | | | | | | precursor isozyme 3 [Oryza |
| | | | | | | | sativa (japonica cultivar- |
| | | | | | | | group)] |
| 142 | 335 | 8 | PHE0004630_PMON94367 | 0 | 100 | 7270516 | emb\|CAB80281.1\|NAD+ |
| | | | | | | | dependent isocitrate |
| | | | | | | | dehydrogenase-like protein |
| | | | | | | | [Arabidopsis thaliana] |
| 143 | 336 | 3 | PHE0004634_PMON94385 | 1.00E−102 | 100 | 61656127 | ref\|NP_176491.1\|AP2 |
| | | | | | | | domain-containing |
| | | | | | | | transcription factor, putative |
| | | | | | | | [Arabidopsis thaliana] |
| 144 | 337 | 2 | PHE0004640_PMON95066 | 0 | 73 | 34913436 | ref\|NP_918065.1\|putative |
| | | | | | | | fatty acid condensing |
| | | | | | | | enzyme CUT1 [Oryza |
| | | | | | | | sativa (japonica cultivar- |
| | | | | | | | group)] |
| 145 | 338 | 8 | PHE0004645_PMON94655 | 1.00E−136 | 100 | 18411867 | ref\|NP_565174.1\|14-3-3 |
| | | | | | | | protein GF14 pi (GRF13) |
| | | | | | | | [Arabidopsis thaliana] |
| 146 | 339 | 14 | PHE0004645_PMON94685 | 1.00E−136 | 100 | 18411867 | ref\|NP_565174.1\|14-3-3 |
| | | | | | | | protein GF14 pi (GRF13) |
| | | | | | | | [Arabidopsis thaliana] |
| 147 | 340 | 8 | PHE0004647_PMON94651 | 1.00E−117 | 100 | 21554066 | pir\|\|T02447 hypothetical |
| | | | | | | | protein At2g46000 |
| | | | | | | | Arabidopsis thaliana |
| 148 | 341 | 14 | PHE0004647_PMON94688 | 1.00E−117 | 100 | 21554066 | gb\|AAM63147.1\|unknown |
| | | | | | | | [Arabidopsis thaliana] |
| 149 | 342 | 14 | PHE0004650_PMON94686 | 1.00E−112 | 100 | 67633514 | gb\|AAY78681.1\|putative E3 |
| | | | | | | | ubiquitin ligase SCF |
| | | | | | | | complex subunit |
| | | | | | | | SKP1/ASK1 [Arabidopsis |
| | | | | | | | thaliana] |
| 150 | 343 | 8 | PHE0004652_PMON94657 | 1.00E−138 | 100 | 38603872 | dbj\|BAD43212.1\|putative |
| | | | | | | | glutamate/aspartate-binding |
| | | | | | | | peptide [Arabidopsis |
| | | | | | | | thaliana] |
| 151 | 344 | 14 | PHE0004652_PMON94687 | 1.00E−138 | 100 | 38603872 | dbj\|BAD43212.1\|putative |
| | | | | | | | glutamate/aspartate-binding |
| | | | | | | | peptide [Arabidopsis |
| | | | | | | | thaliana] |
| 152 | 345 | 8 | PHE0004687_PMON94669 | 7.00E−61 | 91 | 21592528 | ref\|NP_568396.1\|ring-box |
| | | | | | | | protein-related [Arabidopsis |
| | | | | | | | thaliana] |
| 153 | 346 | 10 | PHE0004689_PMON95131 | 0 | 100 | 7268004 | emb\|CAB78344.1\|serine/threonine- |
| | | | | | | | specific protein |
| | | | | | | | kinase MHK [Arabidopsis |
| | | | | | | | thaliana] |
| 154 | 347 | 10 | PHE0004691_PMON95129 | 0 | 100 | 51978966 | emb\|CAB61629.1\| |
| | | | | | | | spermidine synthase 1 |
| | | | | | | | [Oryza sativa] |
| 155 | 348 | 14 | PHE0004719_PMON94698 | 1.00E−147 | 100 | 28416631 | ref\|NP_564556.1\|zinc |
| | | | | | | | finger (C3HC4-type RING |
| | | | | | | | finger) family protein |
| | | | | | | | [Arabidopsis thaliana] |
| 156 | 349 | 8 | PHE0004719_PMON95089 | 1.00E−147 | 100 | 28416631 | ref\|NP_564556.1\|zinc |
| | | | | | | | finger (C3HC4-type RING |
| | | | | | | | finger) family protein |
| | | | | | | | [Arabidopsis thaliana] |
| 157 | 350 | 8 | PHE0004734_PMON94667 | 1.00E−87 | 100 | 5080771 | ref\|NP_172848.1\| |
| | | | | | | | eukaryotic translation |
| | | | | | | | initiation factor 5A-1/eIF- |
| | | | | | | | 5A 1 [Arabidopsis thaliana] |
| 158 | 351 | 10 | PHE0004735_PMON95116 | 9.00E−88 | 100 | 21592652 | ref\|NP_177100.1\| |
| | | | | | | | eukaryotic translation |
| | | | | | | | initiation factor 5A, putative/ |
| | | | | | | | eIF-5A, putative |
| | | | | | | | [Arabidopsis thaliana] |
| 159 | 352 | 8 | PHE0004739_PMON95110 | 1.00E−109 | 100 | 6562282 | emb\|CAB62652.1\|rac-like |
| | | | | | | | GTP binding protein |
| | | | | | | | Arac11 [Arabidopsis |
| | | | | | | | thaliana] |
| 160 | 353 | 8 | PHE0004753_PMON95105 | 0 | 100 | 6684442 | ref\|NP_178062.1\| |
| | | | | | | | succinate-semialdehyde |
| | | | | | | | dehydrogenase (SSADH1) |
| | | | | | | | [Arabidopsis thaliana] |
| 161 | 354 | 8 | PHE0004759_PMON95109 | 0 | 100 | 29824301 | ref\|NP_849582.1\|expressed |
| | | | | | | | protein [Arabidopsis |
| | | | | | | | thaliana] |
| 162 | 355 | 10 | PHE0004770_PMON95122 | 1.00E−32 | 92 | 51038072 | gb\|AAT93875.1\|unknown |
| | | | | | | | protein [Oryza sativa |
| | | | | | | | (japonica cultivar-group)] |
| 163 | 356 | 10 | PHE0004772_PMON95132 | 6.00E−36 | 33 | 9758946 | ref\|NP_200265.1\| |
| | | | | | | | expressed protein |
| | | | | | | | [Arabidopsis thaliana] |
| 164 | 357 | 10 | PHE0004774_PMON95147 | 6.00E−52 | 66 | 50909195 | ref\|XP_466086.1\|putative |
| | | | | | | | multiple stress-responsive |
| | | | | | | | zinc-finger protein [Oryza |
| | | | | | | | sativa (japonica cultivar- |
| | | | | | | | group)] |
| 165 | 358 | 10 | PHE0004777_PMON95118 | 2.00E−64 | 100 | 26452894 | ref\|NP_180514.1\|DNA- |
| | | | | | | | directed RNA polymerase |
| | | | | | | | I(A) and III(C) 14 kDa |
| | | | | | | | subunit (RPAC14) |
| | | | | | | | [Arabidopsis thaliana] |
| 166 | 359 | 14 | PHE0004785_PMON95057 | 1.00E−145 | 84 | 34484312 | sp\|Q6UNT2\|RL5_CUCSA |
| | | | | | | | 60S ribosomal protein L5 |
| 167 | 360 | 10 | PHE0004786_PMON95604 | 0 | 100 | 7267537 | ref\|NP_192634.1\| |
| | | | | | | | phosphate-responsive |
| | | | | | | | protein, putative (EXO) |
| | | | | | | | [Arabidopsis thaliana] |
| 168 | 361 | 8 | PHE0004788_PMON95092 | 0 | 84 | 31126776 | ref\|XP_506910.1\| |
| | | | | | | | PREDICTED |
| | | | | | | | OSJNBa0057G07.4 gene |
| | | | | | | | product [Oryza sativa |
| | | | | | | | (japonica cultivar-group)] |
| 169 | 362 | 10 | PHE0004799_PMON95602 | 0 | 99 | 9843858 | emb\|CAC03739.1\|flavin |
| | | | | | | | containing polyamine |
| | | | | | | | oxidase [Zea mays] |
| 170 | 363 | 10 | PHE0004841_PMON95636 | 0 | 100 | 50909767 | ref\|XP_466372.1\|cryptochrome |
| | | | | | | | 1a [Oryza sativa |
| | | | | | | | (japonica cultivar-group)] |
| 171 | 364 | 10 | PHE0004844_PMON95637 | 3.00E−53 | 100 | 62734659 | gb\|AAX96768.1\|expressed |
| | | | | | | | protein [Oryza sativa |
| | | | | | | | (japonica cultivar-group)] |
| 172 | 365 | 14 | PHE0004854_PMON95611 | 1.00E−163 | 100 | 21592743 | ref\|NP_199265.1\|ribose 5- |
| | | | | | | | phosphate isomerase-related |
| | | | | | | | [Arabidopsis thaliana] |
| 173 | 366 | 10 | PHE0004862_PMON95601 | 5.00E−56 | 100 | 34902924 | dbj\|BAB07982.1\|FPF1 |
| | | | | | | | protein-like [Oryza sativa |
| | | | | | | | (japonica cultivar-group)] |
| 174 | 367 | 10 | PHE0004888_PMON95603 | 0 | 100 | 32405610 | ref\|XP_323418.1\|hypothetical |
| | | | | | | | protein [Neurospora |
| | | | | | | | crassa] |
| 175 | 368 | n/a | At1g21790.1 | 1.00E−168 | 100 | 21593249 | ref\|NP_564152.1\|expressed |
| | | | | | | | protein [Arabidopsis |
| | | | | | | | thaliana] |
| 176 | 369 | n/a | ERD4 | 0 | 100 | 17104683 | ref\|NP_564354.1\|early- |
| | | | | | | | responsive to dehydration |
| | | | | | | | stress protein (ERD4) |
| | | | | | | | [Arabidopsis thaliana] |
| 177 | 370 | n/a | At1g78070.2 | 0 | 100 | 42572153 | ref\|NP_974167.1\|WD-40 |
| | | | | | | | repeat family protein |
| | | | | | | | [Arabidopsis thaliana] |
| 178 | 371 | n/a | At1g78070.1 | 1.00E−128 | 100 | 18411805 | ref\|NP_565168.1\|WD-40 |
| | | | | | | | repeat family protein |
| | | | | | | | [Arabidopsis thaliana] |
| 179 | 372 | n/a | At3g47340.1 | 0 | 100 | 5541701 | ref\|NP_190318.1\| |
| | | | | | | | asparagine synthetase 1 |
| | | | | | | | [glutamine-hydrolyzing]/ |
| | | | | | | | glutamine-dependent |
| | | | | | | | asparagine synthetase 1 |
| | | | | | | | (ASN1) [Arabidopsis |
| | | | | | | | thaliana] |
| 180 | 373 | n/a | At3g47340.3 | 0 | 100 | 30692853 | ref\|NP_850664.1\|asparagine |
| | | | | | | | synthetase 1 [glutamine- |
| | | | | | | | hydrolyzing]/glutamine- |
| | | | | | | | dependent asparagine |
| | | | | | | | synthetase 1 (ASN1) |
| | | | | | | | [Arabidopsis thaliana] |
| 181 | 374 | n/a | At3g47340.2 | 0 | 100 | 30692849 | ref\|NP_850663.1\|asparagine |
| | | | | | | | synthetase 1 [glutamine- |
| | | | | | | | hydrolyzing]/glutamine- |
| | | | | | | | dependent asparagine |
| | | | | | | | synthetase 1 (ASN1) |
| | | | | | | | [Arabidopsis thaliana] |
| 182 | 375 | n/a | At5g13170.1 | 1.00E−163 | 100 | 9955561 | ref\|NP_196821.1\|nodulin |
| | | | | | | | MtN3 family protein |
| | | | | | | | [Arabidopsis thaliana] |
| 183 | 376 | n/a | At2g19900.1 | 0 | 100 | 28059162 | ref\|NP_179580.1\|malate |
| | | | | | | | oxidoreductase, putative |
| | | | | | | | [Arabidopsis thaliana] |
| 184 | 377 | n/a | At5g09480.1 | 8.00E−80 | 100 | 9955535 | ref\|NP_196510.1\| |
| | | | | | | | hydroxyproline-rich |
| | | | | | | | glycoprotein family protein |
| | | | | | | | [Arabidopsis thaliana] |
| 185 | 378 | n/a | At5g09530.1 | 0 | 100 | 7671436 | ref\|NP_196515.1\| |
| | | | | | | | hydroxyproline-rich |
| | | | | | | | glycoprotein family protein |
| | | | | | | | [Arabidopsis thaliana] |
| 186 | 379 | n/a | At2g42790.1 | 0 | 100 | 21700853 | ref\|NP_181807.1\|citrate |
| | | | | | | | synthase, glyoxysomal, |
| | | | | | | | putative [Arabidopsis |
| | | | | | | | thaliana] |
| 187 | 380 | n/a | At3g56200.1 | 0 | 100 | 7572918 | ref\|NP_191179.1\|amino |
| | | | | | | | acid transporter family |
| | | | | | | | protein [Arabidopsis |
| | | | | | | | thaliana] |
| 188 | 381 | n/a | At5g01520.1 | 1.00E−141 | 100 | 7327811 | ref\|NP_195772.1\|zinc |
| | | | | | | | finger (C3HC4-type RING |
| | | | | | | | finger) family protein |
| | | | | | | | [Arabidopsis thaliana] |
| 189 | 382 | n/a | At5g01520.2 | 2.00E−97 | 100 | 7327811 | ref\|NP_195772.1\|zinc |
| | | | | | | | finger (C3HC4-type RING |
| | | | | | | | finger) family protein |
| | | | | | | | [Arabidopsis thaliana] |
| 190 | 383 | n/a | At5g66780.1 | 2.00E−66 | 100 | 9758128 | d ref\|NP_201479.1\| |
| | | | | | | | expressed protein |
| | | | | | | | [Arabidopsis thaliana] |
| 191 | 384 | n/a | At5g59320.1 | 1.00E−61 | 100 | 24417292 | ref\|NP_568905.1\|lipid |
| | | | | | | | transfer protein 3 (LTP3) |
| | | | | | | | [Arabidopsis thaliana] |
| 192 | 385 | n/a | AtHB7 | 1.00E−151 | 100 | 20259175 | gb\|AAM14303.1\|putative |
| | | | | | | | homeodomain transcription |
| | | | | | | | factor protein ATHB-7 |
| | | | | | | | [Arabidopsis thaliana] |
| 193 | 386 | n/a | RD20 | 1.00E−136 | 100 | 20465881 | ref\|NP_180896.1\|calcium- |
| | | | | | | | binding RD20 protein |
| | | | | | | | (RD20) [Arabidopsis |
| | | | | | | | thaliana] |

Screening Methods for Transgenic Plants with Enhanced Agronomic Trait

Many transgenic events which survive to fertile transgenic plants that produce seeds and progeny plants will not exhibit an enhanced agronomic trait. Screening is necessary to identify the transgenic plant of this invention. Transgenic plants having enhanced agronomic traits are identified from populations of plants transformed as described herein by evaluating the trait in a variety of assays to detect an enhanced agronomic trait. These assays also may take many forms, including but not limited to, analyses to detect changes in the chemical composition, biomass, physiological properties, morphology of the plant. Changes in chemical compositions such as nutritional composition of grain can be detected by analysis of the seed composition and content of protein, free amino acids, oil, free fatty acids, starch or tocopherols. Changes in biomass characteristics can be made on greenhouse or field grown plants and can include plant height, stem diameter, root and shoot dry weights; and, for corn plants, ear length and diameter. Changes in physiological properties can be identified by evaluating responses to stress conditions, e.g., assays using imposed stress conditions such as water deficit, nitrogen deficiency, cold growing conditions, pathogen or insect attack or light deficiency, or increased plant density. Changes in morphology can be measured by visual observation of tendency of a transformed plant with an enhanced agronomic trait to also appear to be a normal plant as compared to changes toward bushy, taller, thicker, narrower leaves, striped leaves, knotted trait, chlorosis, albino, anthocyanin production, or altered tassels, ears or roots. Other screening properties include days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality.

Although preferred seeds for transgenic plants with enhanced agronomic traits of this invention are corn and soybean plants, other seeds are for cotton, canola, wheat, sunflower, sorghum, alfalfa, barley, millet, rice, tobacco, fruit and vegetable crops, and turfgrass

### Example 1

### Plant Expression Constructs

This example illustrates the construction of plasmids for transferring recombinant DNA into plant cells which can be regenerated into transgenic plants of this invention.

Primers for PCR amplification of protein coding nucleotides of recombinant DNA are designed at or near the start and stop codons of the coding sequence, in order to eliminate most of the 5′ and 3′ untranslated regions. Each recombinant DNA coding for a protein identified in Table 1 is amplified by PCR prior to insertion into the insertion site of one of the base vectors as referenced in Table 5.

A. Corn Transformation Constructs

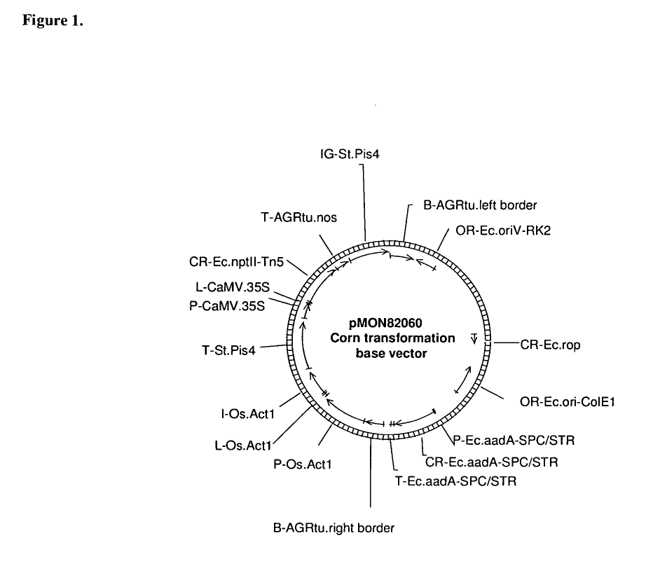

With reference to Table 2 and FIG. 1, pMON82060 illustrates the elements of base vector 1 for corn transformation. Other base vectors for corn transformation were also constructed by replacing the gene of interest plant expression cassette elements of base vector 1, i.e. the promoter, leader, intron and terminator elements, with the elements listed in Table 5 to provide base vectors 2-12 for corn transformation. Each of the protein encoding DNA as identified in Table 1 is placed in the gene of interest plant expression cassette before the termination sequence in each of the base vector 1-12.

TABLE 2
pMON82060

| | | | Coordinates |
|---|---|---|---|
| | | | of SEQ ID |
| function | name | annotation | NO: 12603 |
| Agro | B-AGRtu.right border | Agro right border sequence, essential for | 5235-5591 |
| transformation | | transfer of T-DNA. | |
| Gene of | P-Os.Act1 | Promoter from the rice actin gene act1. | 5609-7009 |
| interest plant | L-Os.Act1 | Leader (first exon) from the rice actin 1 | |
| expression | | gene. | |
| cassette | I-Os.Act1 | First intron and flanking UTR exon | |
| | | sequences from the rice actin 1 gene | |
| | T-St.Pis4 | The 3′ non-translated region of the | 7084-8026 |
| | | potato proteinase inhibitor II gene which | |
| | | functions to direct polyadenylation of the | |
| | | mRNA | |
| Plant | P-CaMV.35S | CaMV 35S promoter | 8075-8398 |
| selectable | L-CaMV.35S | 5′ UTR from the 35S RNA of CaMV | |
| marker | CR-Ec.nptII-Tn5 | nptII selectable marker that confers | 8432-9226 |
| expression | | resistance to neomycin and kanamycin | |
| cassette | T-AGRtu.nos | A 3′ non-translated region of the | 9255-9507 |
| | | nopaline synthase gene of | |
| | | Agrobacterium tumefaciens Ti plasmid | |
| | | which functions to direct | |
| | | polyadenylation of the mRNA. . | |
| Agro | B-AGRtu.left border | Agro left border sequence, essential for | 39-480 |
| transformation | | transfer of T-DNA. | |
| Maintenance | OR-Ec.oriV-RK2 | The vegetative origin of replication from | 567-963 |
| in E. coli | | plasmid RK2. | |
| | CR-Ec.rop | Coding region for repressor of primer | 2472-2663 |
| | | from the ColE1 plasmid. Expression of | |
| | | this gene product interferes with primer | |
| | | binding at the origin of replication, | |
| | | keeping plasmid copy number low. | |
| | OR-Ec.ori-ColE1 | The minimal origin of replication from | 3091-3679 |
| | | the E. coli plasmid ColE1. | |
| | P-Ec.aadA-SPC/STR | promoter for Tn7 adenylyltransferase | 4210-4251 |
| | | (AAD(3″)) | |
| | CR-Ec.aadA- | Coding region for Tn7 | 4252-5040 |
| | SPC/STR | adenylyltransferase (AAD(3″)) | |
| | | conferring spectinomycin and | |
| | | streptomycin resistance. | |
| | T-Ec.aadA-SPC/STR | 3′ UTR from the Tn7 adenylyltransferase | 5041-5098 |
| | | (AAD(3″)) gene of E. coli. | |

Elements of a corn transformation plasmid, pMON17730, for expressing a Leuconostoc mesenteroides sucrose phosphorylase are illustrated in Table 3. This construct was assembled using the technology known in the art.

TABLE 3
pMON17730

| | | | Coordinates |
|---|---|---|---|
| | | | of SEQ ID |
| function | name | annotation | NO: 12606 |
| Agro | B-AGRtu.right | Agro right border sequence, essential | 4862-5218 |
| transformation | border | for transfer of T-DNA. | |
| Gene of | P-Zm.Brittle2 | Promoter from thecorn brittle 2 gene | 5276-6375 |
| interest plant | L-Zm.Brittle2 | 5′ untranslated region from the corn | 6385-7857 |
| expression | | brittel 2 gene. | 7870-8079 |
| cassette | L-Ta.Lhcb1 | wheat CAB leader | |
| | I-Os.Act1 | First intron and flanking UTR exon | |
| | | sequences from the rice actin 1 gene | |
| | CR-Lm.spl1 | PHE0004028_PMON17730 SPL | |
| | | coding region | |
| | T-Ta.Hsp17 | The 3′ non-translated region of the | |
| | | wheat low molecular weight heat | |
| | | shock protein gene | |
| Plant | P-CaMV.35S | CaMV 35S promoter | 8226-8518 |
| selectable | CR-Ec.nptII-Tn5 | nptII selectable marker that confers | 8583-9377 |
| marker | | resistance to neomycin and | |
| expression | | kanamycin | |
| cassette | T-AGRtu.nos | A 3′ non-translated region of the | 9409-9661 |
| | | nopaline synthase gene of | |
| | | Agrobacterium tumefaciens Ti | |
| | | plasmid which functions to direct | |
| | | polyadenylation of the mRNA. . | |
| Agro | B-AGRtu.left | Agro left border sequence, essential | 10003-10026 |
| transformation | border | for transfer of T-DNA. | |
| Maintenance | OR-Ec.oriV- | The vegetative origin of replication | 194-590 |
| in E. coli | RK2 | from plasmid RK2. | |
| | CR-Ec.rop | Coding region for repressor of | 2099-2290 |
| | | primer from the ColE1 plasmid. | |
| | | Expression of this gene product | |
| | | interferes with primer binding at the | |
| | | origin of replication, keeping | |
| | | plasmid copy number low. | |
| | OR-Ec.ori- | The minimal origin of replication | 2718-3306 |
| | ColE1 | from the E. coli plasmid ColE1. | |
| | P-Ec.aadA- | promoter for Tn7 | 3837-3878 |
| | SPC/STR | adenylyltransferase (AAD(3″)) | |
| | CR-Ec.aadA- | Coding region for Tn7 | 3879-4667 |
| | SPC/STR | adenylyltransferase (AAD(3″)) | |
| | | conferring spectinomycin and | |
| | | streptomycin resistance. | |
| | T-Ec.aadA- | 3′ UTR from the Tn7 | 4668-4725 |
| | SPC/STR | adenylyltransferase (AAD(3″)) gene | |
| | | of E. coli. | |

B. Soybean Transformation Constructs

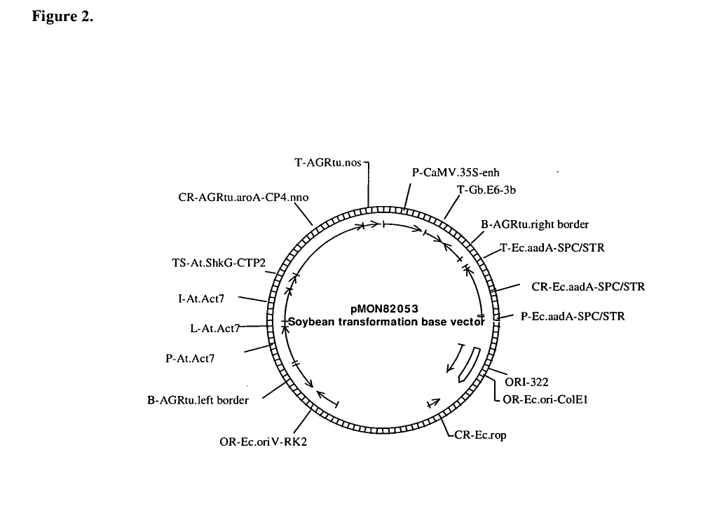

Plasmids for use in transformation of soybean are also prepared. Elements of an exemplary common expression vector plasmid pMON82053 are shown in Table 4 and FIG. 2. Other base vectors for soybean transformation were also constructed by replacing the gene of interest plant expression cassette elements of base vector 13, i.e. the promoter, leader, intron and terminator elements, with the elements listed in Table 5 to provide base vectors 13-15 for soybean transformation. Each of the protein encoding DNA as identified in Table 1 is placed in the gene of interest plant expression cassette before the termination sequence in each of the base vector 13-15.

TABLE 4
pMON82053

| | | | Coordinates |
|---|---|---|---|
| | | | of SEQ ID |
| function | name | annotation | NO: 12604 |
| Agro | B-AGRtu.left border | Agro left border | 6144-6585 |
| transforamtion | | sequence, essential for | |
| | | transfer of T-DNA. | |
| Plant | P-At.Act7 | Promoter from the | 6624-7861 |
| selectable | | arabidopsis actin 7 gene | |
| marker | L-At.Act7 | 5′UTR of Arabidopsis | |
| expression | | Act7 gene | |
| cassette | I-At.Act7 | Intron from the | |
| | | Arabidopsis actin7 gene | |
| | TS-At.ShkG-CTP2 | Transit peptide region of | 7864-8091 |
| | | Arabidopsis EPSPS | |
| | CR-AGRtu.aroA- | Synthetic CP4 coding | 8092-9459 |
| | CP4.nno_At | region with dicot | |
| | | preferred codon usage. | |
| | T-AGRtu.nos | A 3′ non-translated region | 9466-9718 |
| | | of the nopaline synthase | |
| | | gene of Agrobacterium tumefaciens | |
| | | Ti plasmid which functions | |
| | | to direct polyadenylation | |
| | | of the mRNA. | |
| Gene of | P-CaMV.35S-enh | Promoter for 35S RNA | 1-613 |
| interest | | from CaMV containing a | |
| expression | | duplication of the −90 | |
| cassette | | to −350 region. | |
| | T-Gb.E6-3b | 3′ untranslated region | 688-1002 |
| | | from the fiber protein E6 | |
| | | gene of sea-island cotton; | |
| Agro | B-AGRtu.right border | Agro right border | 1033-1389 |
| transformation | | sequence, essential for | |
| | | transfer of T-DNA. | |
| Maintenance | OR-Ec.oriV-RK2 | The vegetative origin of | 5661-6057 |
| in E. coli | | replication from plasmid | |
| | | RK2. | |
| | CR-Ec.rop | Coding region for | 3961-4152 |
| | | repressor of primer from | |
| | | the ColE1 plasmid. | |
| | | Expression of this gene | |
| | | product interferes with | |
| | | primer binding at the | |
| | | origin of replication, | |
| | | keeping plasmid copy | |
| | | number low. | |
| | OR-Ec.ori-ColE1 | The minimal origin of | 2945-3533 |
| | | replication from the | |
| | | E. coli plasmid ColE1. | |
| | P-Ec.aadA-SPC/STR | romoter for Tn7 | 2373-2414 |
| | | adenylyltransferase | |
| | | (AAD(3″)) | |
| | CR-Ec.aadA- | Coding region for Tn7 | 1584-2372 |
| | SPC/STR | adenylyltransferase | |
| | | (AAD(3″)) conferring | |
| | | spectinomycin and | |
| | | streptomycin resistance. | |
| | T-Ec.aadA-SPC/STR | 3′ UTR from the Tn7 | 1526-1583 |
| | | adenylyltransferase | |
| | | (AAD(3″)) gene of | |
| | | E. coli. | |

TABLE 5
Compositions of expression cassettes for gene of interest in
plant transformation base vectors

| | | SEQ | | SEQ | | SEQ | | SEQ |
|---|---|---|---|---|---|---|---|---|
| | | ID | | ID | | ID | | ID |
| | promoter | NO | leader | NO | intron | NO | terminator | NO |
| Base | | | | | | | | |
| vector | | | | | | | | |
| for corn | | | | | | | | |
| 1 | P-Os.Act1 | 12581 | L-Os.Act1 | 12592 | I-Os.Act1 | 12596 | T-St.Pis4 | 12598 |
| 2 | P-Hv.Per1 | 12582 | L-Hv.Per1 | 12593 | I-Zm.DnaK | 12597 | T-St.Pis4 | 12598 |
| 3 | P-Zm.RAB17 | 12591 | NONE | / | I-Zm.DnaK | 12597 | T-St.Pis4 | 12598 |
| 4 | P-Zm.NAS2 | 12584 | L-Zm.NAS2 | 12595 | I-Zm.DnaK | 12597 | T-St.Pis4 | 12598 |
| 5 | P-Zm.PPDK | 12585 | L-Zm.PPDK | 12588 | I-Zm.DnaK | 12597 | T-St.Pis4 | 12598 |
| 6 | P-Os.GT1 | 12586 | NONE | / | I-Zm.DnaK | 12597 | T-St.Pis4 | 12598 |
| 7 | P-Zm.PPDK | 12587 | L-Zm.PPDK | 12588 | I-Zm.DnaK | 12597 | T-St.Pis4 | 12600 |
| 8 | P-Os.Act1 | 12581 | L-Os.Act1 | 12592 | I-Os.Act1 | 12597 | T-St.Pis4 | 12598 |
| 9 | P-Zm.PPDK | 12587 | L-Zm.PPDK | 12588 | I-Zm.DnaK | 12597 | T-St.Pis4 | 12600 |
| 10 | P-Os.Act1 | 12581 | L-Os.Act1 | 12592 | I-Os.Act1 | 12596 | T-St.Pis4 | 12598 |
| 11 | P-Zm.SzeinC1 | 12589 | L- | 12601 | I-Zm.DnaK | 12597 | T-St.Pis4 | 12598 |
| | | | Zm.SzeinC1 | | | | | |
| 12 | P-Zm.NAS2 | 12584 | L-Zm.NAS2 | 12595 | I-Zm.DnaK | 12597 | T-St.Pis4 | 12598 |
| Base | | | | | | | | |
| vector | | | | | | | | |
| for | | | | | | | | |
| Soybean | | | | | | | | |
| 13 | P-CaMV.35S- | 12590 | NONE | / | NONE | / | T-Gb.E6 | 12599 |
| | enh | | | | | | | |
| 14 | P-CaMV.35S- | 12590 | NONE | / | NONE | / | T-Gb.E6 | 12599 |
| | enh | | | | | | | |
| 15 | P-Gm.Sphas 1 | 12583 | L- | 12594 | NONE | / | T-Gb.E6 | 12599 |
| | | | Gm.Sphas1 | | | | | |

DNA constructs with some recombinant DNA of interest, e.g., SEQ ID NO: 72, also contain a chloroplast transit peptide adjacent to the recombinant DNA.

C. Cotton Transformation Vector

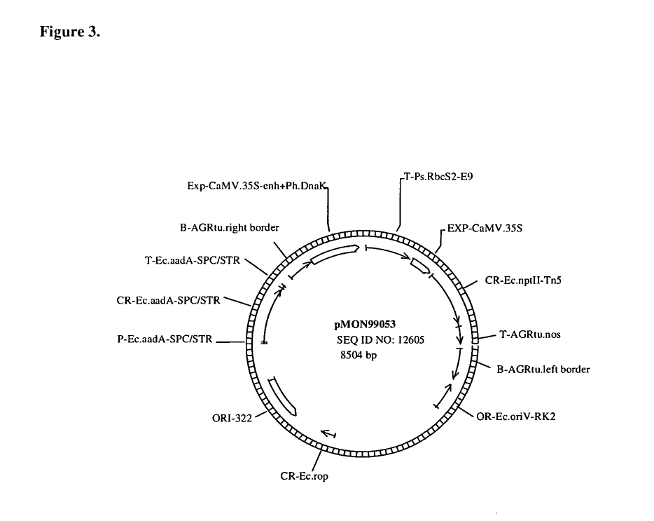

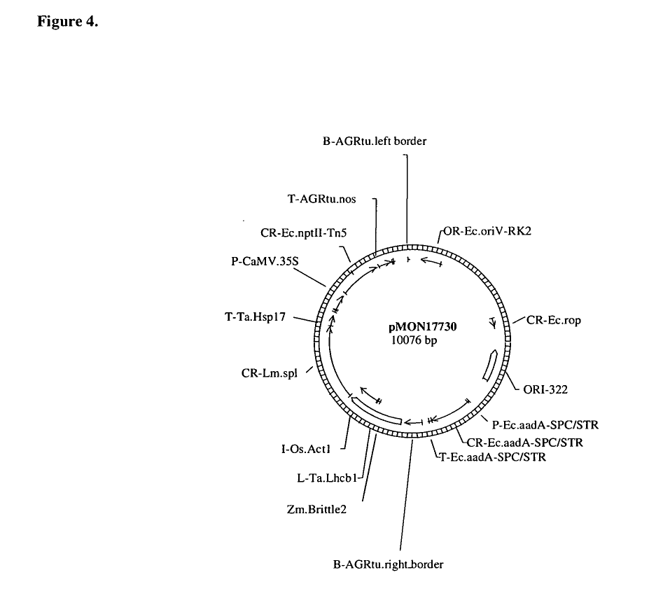

Plasmids for use in transformation of cotton are also prepared. Elements of an exemplary common expression vector plasmid pMON99053 are shown in Table 6 below and FIG. 3. Primers for PCR amplification of protein coding nucleotides of recombinant DNA are designed at or near the start and stop codons of the coding sequence, in order to eliminate most of the 5′ and 3′ untranslated regions. Each recombinant DNA coding for a protein identified in Table 1 is amplified by PCR prior to insertion into the insertion site within the gene of interest expression cassette of pMON99053

TABLE 6

| | | | Coordinates |
|---|---|---|---|
| | | | of SEQ ID |
| function | name | annotation | NO: 12606 |
| Agro | B-AGRtu.right border | Agro right border sequence, | 11364-11720 |
| transforamtion | | essential for transfer of T-DNA. | |
| Gene of interest | Exp-CaMV.35S- | Enhanced version of the 35S RNA | 7794-8497 |
| expression | enh+ph.DnaK | promoter from CaMV plus the | |
| cassette | | petunia hsp70 5′ untranslated | |
| | | region | |
| | T-Ps.RbcS2-E9 | The 3′ non-translated region of the | 67-699 |
| | | pea RbcS2 gene which functions | |
| | | to direct polyadenylation of the | |
| | | mRNA. | |
| Plant selectable | Exp-CaMV.35S | Promoter from the rice actin 1 | 730-1053 |
| marker | | gene | |
| expression | CR-Ec.nptII-Tn5 | first exon of the rice actin 1 gene | 1087-1881 |
| cassette | T-AGRtu.nos | A 3′ non-translated region of the | 1913-2165 |
| | | nopaline synthase gene of | |
| | | Agrobacterium tumefaciens Ti | |
| | | plasmid which functions to direct | |
| | | polyadenylation of the mRNA. | |
| Agro | B-AGRtu.left border | Agro left border sequence, | 2211-2652 |
| transformation | | essential for transfer of T-DNA. | |
| Maintenance in | OR-Ec.oriV-RK2 | The vegetative origin of | 2739-3135 |
| E. coli | | replication from plasmid RK2. | |
| | CR-Ec.rop | Coding region for repressor of | 4644-4835 |
| | | primer from the ColE1 plasmid. | |
| | | Expression of this gene product | |
| | | interferes with primer binding at | |
| | | the origin of replication, keeping | |
| | | plasmid copy number low. | |
| | OR-Ec.ori-ColE1 | The minimal origin of replication | 5263-5851 |
| | | from the E. coli plasmid ColE1. | |
| | P-Ec.aadA-SPC/STR | romoter for Tn7 | 6382-6423 |
| | | adenylyltransferase (AAD(3″)) | |
| | CR-Ec.aadA-SPC/STR | Coding region for Tn7 | 6424-7212 |
| | | adenylyltransferase (AAD(3″)) | |
| | | conferring spectinomycin and | |
| | | streptomycin resistance. | |
| | T-Ec.aadA-SPC/STR | 3′ UTR from the Tn7 | 7213-7270 |
| | | adenylyltransferase (AAD(3″)) | |
| | | gene of E. coli. | |

### Example 2

### Corn Plant Transformation

This example illustrates the production and identification of transgenic corn cells in seed of transgenic corn plants having an enhanced agronomic trait, i.e. enhanced nitrogen use efficiency, increased yield, enhanced water use efficiency, enhanced tolerance to cold and/or improved seed compositions as compared to control plants. Transgenic corn cells are prepared with recombinant DNA expressing each of the protein encoding DNAs listed in Table 1 by Agrobacterium-mediated transformation using the corn transformation vectors 1-12 prepared as disclosed in Example 1. Corn transformation is effected using methods disclosed in U.S. Patent Application Publication 2004/0344075 A1 where corn embryos are inoculated and co-cultured with the Agrobacterium tumefaciens strain ABI and the corn transformation vector. To regenerate transgenic corn plants the transgenic callus resulting from transformation is placed on media to initiate shoot development in plantlets which are transferred to potting soil for initial growth in a growth chamber followed by a mist bench before transplanting to pots where plants are grown to maturity. The plants are self fertilized and seed is harvested for screening as seed, seedlings or progeny R2 plants or hybrids, e.g., for yield trials in the screens indicated above.

Many transgenic events which survive to fertile transgenic plants that produce seeds and progeny plants do not exhibit an enhanced agronomic trait. The transgenic plants and seeds having the transgenic cells of this invention which have recombinant DNA imparting the enhanced agronomic traits are identified by screening for nitrogen use efficiency, yield, water use efficiency, cold tolerance and improved seed composition.

### Example 3

### Soybean Plant Transformation

This example illustrates the production and identification of transgenic soybean cells in seed of transgenic soybean plants having an enhanced agronomic trait, i.e. enhanced nitrogen use efficiency, increased yield, enhanced water use efficiency, enhanced tolerance to cold and/or improved seed compositions as compared to control plants. Transgenic soybean cells are prepared with recombinant DNA expressing each of the protein encoding DNAs listed in Table 1 by Agrobacterium-mediated transformation using the soybean transformation vectors 13-15 prepared as disclosed in Example 1. Soybean transformation is effected using methods disclosed in U.S. Pat. No. 6,384,301 where soybean meristem explants are wounded then inoculated and co-cultured with the soybean transformation vector, then transferred to selection media for 6-8 weeks to allow selection and growth of transgenic shoots.

The transformation is repeated for each of the protein encoding DNAs identified in Table 1 in one of the base vectors 13-15.

Transgenic shoots producing roots are transferred to the greenhouse and potted in soil. Many transgenic events which survive to fertile transgenic plants that produce seeds and progeny plants do not exhibit an enhanced agronomic trait. The transgenic plants and seeds having the transgenic cells of this invention which have recombinant DNA imparting the enhanced agronomic traits are identified by screening for nitrogen use efficiency, yield, water use efficiency, cold tolerance and improved seed composition.

### Example 4

### Cotton Transgenic Plants with Enhanced Agronomic Traits

Cotton transformation is performed as generally described in WO0036911 and in U.S. Pat. No. 5,846,797. Transgenic cotton plants containing the recombinant DNA having a sequence of SEQ ID NO: 1 through SEQ ID NO: 193 are obtained by transforming with the cotton transformation vector identified in Example 1.

Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA. The specified culture conditions are growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions, i.e. irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential, increased biomass, enhanced leaf extension rates, and improved fiber parameters.

Cotton plants with the transgenic cells by this invention are identified from among the transgenic cotton plants by agronomic trait screening as having increased yield and enhanced water use efficiency.

### Example 5

### Homolog Identification

This example illustrates the identification of homologs of proteins encoded by the DNA identified in Table 1 which is used to provide transgenic seed and plants having enhanced agronomic traits. From the sequence of the homologs, homologous DNA sequence can be identified for preparing additional transgenic seeds and plants of this invention with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism.

The All Protein Database was queried using amino acid sequences provided herein as SEQ ID NO: 194 through SEQ ID NO: 386 using NCBI “blastp” program with E-value cutoff of 1 e-8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided herein as SEQ ID NO: 194 through SEQ ID NO: 386 using NCBI “blastp” program with E-value cutoff of 1e-4. Up to 1000 top hits were kept. A BLAST searchable database was constructed based on these hits, and is referred to as “SubDB”. SubDB was queried with each sequence in the Hit List using NCBI “blastp” program with E-value cutoff of 1e-8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. Homologs from a large number of distinct organisms were identified and are reported by amino acid sequences of SEQ ID NO: 387 through SEQ ID NO: 12580. These relationships of proteins of SEQ ID NO: 194 through 386 and homologs of SEQ ID NO: 387 through 12580 is identified in Table 7. The source organism for each homolog is found in the Sequence Listing.

TABLE 7

| SEQ ID NO: | homolog SEQ ID NOs | | | | | | | | | | | |
|---|---|---|---|---|---|---|---|---|---|---|---|---|
| 196: | 3549 | 1976 | 8970 | 12287 | 11799 | 758 | 6083 | 9821 | 8256 | 7610 | 7869 | 4091 |
| | 1111 | 1113 | 8630 | 7054 | 10917 | 3094 | 6712 | 9080 | 2702 | 2718 | 1130 | 1131 |
| | 5382 | 6582 | 559 | 2169 | 1134 | 1132 | 1139 | 2295 | 11615 | 8090 | 2133 | 5063 |
| | 5000 | 10336 | 12279 | 3828 | 7214 | 1485 | 2156 | 2232 | 2229 | 2242 | 2209 | 2203 |
| | 2177 | 2207 | 2160 | 2151 | 11166 | 3220 | | | | | | |
| 197: | 3549 | 1976 | 4850 | 8970 | 12287 | 11799 | 758 | 6083 | 9821 | 4946 | 11935 | 8256 |
| | 7610 | 7869 | 1841 | 9456 | 4091 | 1113 | 1111 | 8630 | 7054 | 7880 | 6876 | 6237 |
| | 6712 | 9080 | 2702 | 2718 | 1130 | 1131 | 5382 | 6582 | 559 | 2169 | 1134 | 1132 |
| | 1139 | 2295 | 11615 | 8090 | 5063 | 5000 | 10336 | 12279 | 3828 | 7214 | 1485 | 2156 |
| | 2232 | 2229 | 2242 | 2209 | 2203 | 2207 | 2177 | 2160 | 2151 | 7622 | 1377 | 6970 |
| | 6143 | | | | | | | | | | | |
| 198: | 3549 | 1976 | 2210 | 6154 | 1028 | 1769 | 758 | 12325 | 9821 | 2973 | 4946 | 11935 |
| | 8256 | 7610 | 5387 | 5384 | 5361 | 10434 | 8983 | 5051 | 4091 | 2766 | 6248 | 1113 |
| | 1111 | 8630 | 9080 | 2702 | 2718 | 1131 | 1130 | 5382 | 7052 | 6582 | 1134 | 1132 |
| | 1139 | 11615 | 2295 | 8090 | 6572 | 4803 | 1970 | 8113 | 3883 | 9565 | 1707 | 517 |
| | 12372 | 11514 | 5441 | 5421 | 3828 | 7214 | 1485 | 1097 | | | | |
| 199: | 3549 | 4850 | 2210 | 8970 | 12287 | 2360 | 11500 | 11799 | 6912 | 1028 | 6154 | 758 |
| | 5783 | 6083 | 9552 | 12325 | 9821 | 2973 | 4946 | 8256 | 7610 | 5387 | 5361 | 5384 |
| | 5300 | 10434 | 8983 | 5051 | 1111 | 1113 | 8630 | 9080 | 2702 | 2718 | 1130 | 1131 |
| | 5382 | 7052 | 6582 | 559 | 2169 | 1134 | 1132 | 1139 | 8090 | 6572 | 11350 | 7138 |
| | 1730 | 10762 | 11345 | 527 | 8679 | 5063 | 5000 | 2879 | 517 | 7986 | 12372 | 11514 |
| | 10336 | 6955 | 12279 | 5441 | 5421 | 3828 | 7214 | 1485 | 2229 | 2207 | 2242 | 2232 |
| | 2209 | 2203 | 2156 | 2160 | 2151 | 5328 | 8248 | | | | | |
| 200: | 11500 | 5617 | 8150 | 3321 | 2181 | 4364 | 1769 | 1028 | 5122 | 11328 | 6042 | 2711 |
| | 1760 | 4874 | 4098 | 1914 | 11853 | 7334 | 6504 | 10624 | 2638 | 11705 | 7913 | 12171 |
| | 12198 | 10430 | 12189 | 12219 | 10404 | 10432 | 10408 | 6957 | 8282 | 6184 | 11935 | 580 |
| | 10470 | 1940 | 11039 | 8629 | 1096 | 742 | 12505 | 5801 | 11671 | 4006 | 12473 | 6778 |
| | 2607 | 10849 | 6279 | 7500 | 2657 | 5584 | 8059 | 2622 | 2043 | 3269 | 10363 | 6186 |
| | 9631 | 9243 | 11098 | 1168 | 6690 | 8584 | 10577 | 687 | 2977 | 9804 | 9337 | 6306 |
| | 9118 | 4356 | 10225 | 9740 | 6652 | 5251 | 12514 | 7463 | 706 | 3048 | 3780 | 1925 |
| | 11765 | 9803 | 10824 | 3004 | 5275 | 8642 | 1664 | 12173 | 4049 | 2031 | 11681 | 8980 |
| | 2339 | 9172 | 11955 | 10576 | 9333 | 10482 | 813 | 5656 | 4628 | 10843 | 2352 | 5484 |
| | 2856 | 4313 | 2877 | 1633 | 11143 | 6066 | 7722 | 7746 | 10941 | 11741 | 2941 | 2745 |
| | 11364 | 7638 | 7884 | 1328 | 5606 | 6580 | 11262 | 7483 | 8156 | 412 | 453 | 7288 |
| | 6842 | 1286 | 7896 | 9734 | 6570 | 10595 | 8863 | 1246 | 7112 | 12464 | 1373 | 3779 |
| | 2705 | 5044 | 4017 | 5712 | 4619 | 3539 | 1029 | 1610 | 5976 | 4964 | 11724 | 9037 |
| | 8989 | 1126 | 4073 | 395 | 10344 | 5428 | 4845 | 1611 | 10484 | 4496 | 3517 | 3418 |
| | 10294 | 2427 | 3442 | 9747 | 5534 | 9571 | 1125 | 9720 | 9319 | 12346 | 3417 | 1588 |
| | 2779 | 4611 | 5312 | 10179 | 6867 | 3049 | 3051 | 9900 | 1265 | 9463 | 4576 | 764 |
| | 6024 | 432 | 8921 | 11379 | 2141 | 1755 | 9498 | 7395 | 8179 | 7462 | 7279 | 8729 |
| | 9676 | 11351 | 1758 | 10907 | 4995 | 1205 | 608 | 12100 | 8331 | 8341 | 10326 | 6852 |
| | 11947 | 6597 | 2475 | 6407 | 8077 | 10788 | 11815 | 5269 | 489 | 9317 | 5574 | 11240 |
| | 11821 | 11485 | 2868 | 9753 | 676 | 11223 | 1924 | 8045 | 1689 | 12035 | 11980 | 5906 |
| | 7805 | 6728 | 5177 | 1711 | 1715 | 5050 | 1601 | 11242 | 1010 | 11286 | 7814 | 7152 |
| | 3730 | 5888 | 615 | 11078 | 9681 | 2883 | 8522 | 8210 | 4450 | 11632 | 7573 | 6031 |
| | 2713 | 3861 | 9480 | 5307 | 7874 | 2048 | 5136 | 8625 | 2168 | 4580 | 10634 | 5772 |
| | 5082 | 8731 | 2678 | 9311 | 10561 | 7803 | 4408 | 6227 | 12026 | 11234 | 7247 | 5578 |
| | 9683 | 3999 | 2953 | 2193 | 3370 | 11542 | 10711 | 6403 | 4207 | 11251 | 8447 | 6805 |
| | 727 | 951 | 737 | 9090 | 1828 | 1928 | 2277 | 986 | 739 | 7044 | 10025 | 7409 |
| | 9449 | 944 | 8427 | 10911 | 3965 | 1299 | 5294 | 6332 | 5145 | 9418 | 6150 | 9008 |
| | 1004 | 3831 | 5157 | 6968 | 11922 | 7392 | 9855 | 5061 | 5448 | 6857 | 2354 | 2879 |
| | 620 | 7986 | 10208 | 4520 | 9003 | 8015 | 525 | 8013 | 11884 | 10726 | 12493 | 9260 |
| | 8508 | 5693 | 1450 | 4258 | | | | | | | | |
| 201: | 7470 | 10842 | 5790 | 6772 | 1530 | 9966 | 9973 | 10368 | 655 | 4677 | 4157 | 1015 |
| | 9967 | 9732 | 1621 | 1702 | 2553 | 11599 | 9342 | 3724 | 6613 | 4462 | 2681 | 4577 |
| | 3827 | 8039 | 2557 | 8538 | 9605 | 12321 | 3228 | 2139 | 9255 | 11428 | 3022 | 5404 |
| | 9564 | 12166 | 8047 | 11255 | 11888 | 1492 | 5870 | 4250 | 554 | 12481 | 8585 | 5674 |
| | 2062 | 2021 | 6718 | 2810 | 4015 | 12306 | 8941 | 3135 | 7850 | 7009 | 4247 | 5760 |
| | 643 | 2512 | 2422 | 8709 | 5661 | 2437 | 11487 | 8706 | 3703 | 6811 | 5006 | 6000 |
| | 2290 | 11973 | 8426 | 2912 | 6498 | 10642 | 8257 | 5362 | 1189 | 996 | 1740 | 10904 |
| | 5778 | 4372 | 12095 | 1616 | 9708 | 1598 | 4525 | 7513 | 1934 | 10939 | 9044 | 7273 |
| | 6105 | 6950 | 12122 | 5936 | 2802 | 3711 | 8640 | 6644 | 9842 | 6994 | 2587 | 7510 |
| | 8609 | 1877 | 5408 | 8009 | 9943 | 8475 | 4333 | 8476 | 2651 | 5379 | 11144 | |
| 202: | 7477 | 1676 | 4448 | 2400 | 6045 | 6940 | 8526 | 9923 | 11995 | 8913 | 10513 | 634 |
| | 7969 | 11746 | 6446 | 4371 | 1018 | 4026 | 10874 | 11604 | 5505 | 9219 | 4140 | 11205 |
| | 12025 | 3605 | 1669 | 1987 | 2822 | 2279 | 10124 | 11930 | 4546 | 3504 | 1950 | 7696 |
| | 1604 | 4492 | 710 | 11737 | 3171 | 8574 | 11646 | 9030 | 765 | | | |
| 203: | 9581 | 1789 | 9205 | 10127 | 507 | 7859 | 5085 | 10794 | 2201 | 5072 | 1384 | 7541 |
| | 12225 | 5253 | 4000 | 8561 | 1469 | 3834 | 12504 | 9837 | 7137 | 4670 | 9143 | 1972 |
| | 230 | 11807 | 7457 | 3867 | 12503 | 9644 | 10286 | 686 | 3416 | 8708 | 913 | 9391 |
| | 9343 | 1949 | 971 | 11938 | 12315 | 7511 | 9076 | 8346 | 3455 | 1790 | 6685 | 11054 |
| | 10989 | 4775 | 9544 | 2197 | 3225 | 1198 | 7996 | 9715 | 6751 | 11217 | 3189 | 10361 |
| | 3589 | 2768 | 4753 | 393 | 7426 | 9423 | 2744 | 1339 | 10139 | 2332 | 8771 | 3079 |
| | 4312 | 7098 | 11256 | 1681 | 642 | 411 | 5179 | 11964 | 5793 | 8376 | 2386 | 9500 |
| | 2401 | 5669 | 10501 | 1939 | 11311 | 4977 | 7401 | 8266 | 12472 | 480 | 10947 | 12116 |
| | 539 | 7591 | 1020 | 1493 | 9017 | 2513 | 3100 | 4405 | 5679 | 3373 | 3795 | 10805 |
| | 11445 | 10653 | 5898 | 5556 | 12139 | 12448 | 8448 | 5245 | 12533 | 10039 | 1324 | 2498 |
| | 9955 | 6104 | 5516 | | | | | | | | | |
| 204: | 3474 | 7088 | 4085 | 10331 | 6972 | 7065 | 2023 | 10909 | 5915 | 5913 | 6491 | 5970 |
| | 6936 | 5920 | 5919 | 5966 | 5944 | 5738 | 5968 | 6663 | 1233 | 5947 | 2258 | 10694 |
| | 9592 | 4692 | 12344 | 11227 | 6753 | 8618 | | | | | | |
| 205: | 9144 | 6127 | 6445 | 4401 | 3645 | 9756 | 5274 | 8302 | 1548 | 9875 | 9979 | 1922 |
| | 1941 | 9100 | 7274 | 12121 | 11051 | 11528 | 9523 | 7830 | 3543 | 6760 | 1979 | 3997 |
| | 9779 | 9635 | 4955 | 1818 | 946 | 5201 | 12580 | 8270 | 10531 | 415 | 4910 | 1802 |
| | 2256 | 2979 | 7899 | 3139 | 3777 | 10332 | 10536 | 4842 | 8280 | 9000 | 1327 | 10950 |
| | 8576 | 513 | 4263 | 6884 | 8684 | 3877 | 7243 | 7262 | 6420 | 1424 | 2680 | 10546 |
| | 9965 | 11711 | 6656 | 1164 | 1160 | 5248 | 4812 | 11605 | 3598 | 8386 | 12446 | 3922 |
| | 10305 | 467 | 5963 | 948 | 11998 | 4655 | 4064 | 446 | 6112 | 6111 | 4689 | 3743 |
| | 449 | 1123 | 11231 | 11434 | 2456 | 471 | 11629 | 6249 | 2152 | 2171 | 6494 | 8636 |
| | 11953 | 5487 | 7844 | 6164 | 11566 | 1495 | 4623 | 6920 | 3447 | 3181 | 3153 | 1081 |
| | 11890 | 3476 | 1127 | 1195 | 1192 | 12349 | 3600 | 11090 | 5377 | 8022 | 7160 | 11091 |
| | 10643 | 7586 | 12247 | 6202 | 6217 | 4617 | 2237 | 2380 | 6219 | 1756 | 7456 | 925 |
| | 3237 | | | | | | | | | | | |
| 206: | 5804 | 12016 | 10678 | 10712 | 10735 | 7448 | 9024 | 10738 | 10708 | 12014 | 10638 | 7423 |
| | 7421 | 7417 | 309 | 10586 | 10603 | 10589 | 10584 | 7444 | 10644 | 7446 | 5047 | 10645 |
| | 10646 | 12280 | 207 | 9710 | 2096 | 11839 | 9709 | 1612 | 8993 | 10037 | 6780 | 11613 |
| | 9034 | 306 | 307 | 2004 | 11103 | 8166 | 6931 | 7311 | 6922 | 8933 | 10494 | 3783 |
| | 308 | 11857 | 12034 | 3781 | 916 | 6666 | 9745 | 9140 | 6285 | 12449 | 10356 | 9452 |
| | 4275 | 12246 | 9728 | 9405 | 2987 | 7223 | 2067 | 3934 | 8138 | 11430 | 9052 | 12318 |
| | 6252 | 410 | 2407 | 6792 | 3564 | 2073 | 4786 | 11326 | 9877 | 3397 | 310 | 11058 |
| | 9105 | 8474 | 12047 | 6860 | 7715 | 860 | 8446 | 4050 | 6973 | 6725 | 9408 | 4088 |
| | 3842 | 1902 | 4332 | 2342 | 1701 | 10402 | 11870 | 4672 | 3986 | 10725 | 12181 | 1973 |
| | 3950 | 9992 | 4578 | 10224 | 862 | 7045 | 11785 | 4789 | 5465 | 8088 | 3553 | 10189 |
| | 9964 | 2793 | 6677 | 10001 | 3375 | 4200 | 10391 | 1361 | 1234 | 10741 | 10641 | 10683 |
| | 11712 | 10743 | 10575 | 10581 | 4747 | | | | | | | |
| 207: | 12016 | 7448 | 12014 | 7423 | 309 | 5047 | 9710 | 2096 | 11839 | 1612 | 8993 | 10037 |
| | 6780 | 9034 | 307 | 306 | 2004 | 6931 | 6922 | 8933 | 10494 | 308 | 11857 | 3781 |
| | 916 | 9745 | 6666 | 9140 | 6285 | 12449 | 9452 | 8035 | 10356 | 11492 | 12021 | 4443 |
| | 10064 | 8344 | 2067 | 3934 | 4275 | 8138 | 12246 | 9405 | 2987 | 9728 | 11430 | 9052 |
| | 12318 | 6252 | 7223 | 410 | 6792 | 3564 | 2073 | 4786 | 9877 | 3397 | 310 | 11058 |
| | 8474 | 9105 | 11870 | 12047 | 4672 | 3986 | 6860 | 7715 | 860 | 2342 | 8446 | 1701 |
| | 4050 | 10402 | 6973 | 9408 | 4088 | 3842 | 4332 | 6725 | 1902 | 10725 | 12181 | 6779 |
| | 1973 | 2823 | 9849 | 10154 | 862 | 7045 | 11785 | 4789 | 5465 | 10224 | 8088 | 4578 |
| | 10189 | 3553 | 2793 | 6677 | 10001 | 3375 | 4200 | 10391 | 3950 | 9992 | 1361 | 1468 |
| | 9964 | 7410 | 2176 | 10741 | 11712 | 10743 | 10575 | 10581 | 206 | | | |
| 208: | 8564 | 10720 | 7580 | 12251 | 9922 | 5975 | 8617 | 4257 | 645 | 3210 | 4615 | 8228 |
| | 747 | 1408 | 10412 | 3357 | 4397 | 7547 | 10137 | 3018 | 7289 | 11413 | 1687 | 2058 |
| | 4738 | 1274 | 12252 | 8769 | 6626 | 4708 | 275 | 11442 | 2843 | 10230 | 6198 | 10814 |
| | 2304 | 9207 | | | | | | | | | | |
| 209: | 9386 | 8213 | 8184 | 6094 | 8240 | 8242 | 8209 | 8211 | 5327 | 9254 | 10652 | 9428 |
| | 11965 | 11812 | 11814 | 9275 | 9274 | 6208 | 8173 | 7971 | 9276 | 9278 | 9280 | 9297 |
| | 9253 | 8100 | 9330 | 9303 | 9305 | 4986 | 4730 | 10770 | 11755 | 3994 | 5070 | 7569 |
| | 5734 | 3989 | 3985 | 9531 | 9214 | 9429 | 9365 | 11108 | 6372 | 5373 | 2117 | 3351 |
| | 12521 | 4075 | 1896 | 3535 | 10982 | 4340 | 2371 | 858 | 3813 | 10602 | 5493 | 5548 |
| | 10627 | 5552 | 2460 | 4278 | 1787 | 3297 | 2964 | 2965 | 2962 | 3630 | 9434 | 3625 |
| | 4592 | 10087 | 8272 | 3870 | 4415 | 8484 | 5940 | 10629 | 10623 | 10636 | 10174 | 10667 |
| | 5553 | 10670 | 10671 | 2562 | 2568 | 8456 | 5226 | 5200 | 11493 | 7169 | 9374 | 7962 |
| | 11722 | 5462 | 2866 | 10150 | 10170 | 10153 | 4425 | 1856 | 4727 | 9772 | 6514 | 2550 |
| | 9367 | 4482 | 9458 | 9455 | 2869 | 2162 | 9300 | 9302 | 10632 | 3616 | | |
| 210: | 2857 | 3612 | 6601 | 1183 | 1181 | 1182 | 6604 | 1159 | 10118 | 10806 | 11819 | 11745 |
| | 6639 | 11715 | 7049 | 10888 | 10024 | 7122 | 8076 | 8876 | 8903 | 1266 | 10535 | 624 |
| | 7532 | 4011 | 5266 | 6168 | 6326 | 11178 | 2641 | 2461 | 6646 | 8758 | 7990 | 9318 |
| | 8505 | 7393 | 2727 | 6008 | 3940 | 9115 | 5137 | 9096 | 1148 | 1363 | 10193 | 9377 |
| | 9250 | 5445 | 11200 | 11273 | 11276 | | | | | | | |
| 211: | 11176 | 8570 | 11245 | 10274 | 6081 | 7181 | 6450 | 4624 | 9320 | 6129 | 984 | 7196 |
| | 7388 | 2804 | 542 | 11805 | | | | | | | | |
| 212: | 2393 | 3407 | 11789 | 8124 | 2340 | 3714 | 1395 | 1433 | 12303 | 375 | 2814 | 6364 |
| | 9438 | 3292 | 12390 | 2984 | 6746 | 9695 | 675 | 2101 | 3618 | 12081 | 6128 | 1892 |
| | 3448 | 9864 | 6152 | 2844 | 7381 | 4291 | 4973 | 5447 | 10140 | 11877 | 8566 | 7624 |
| | 6472 | 10665 | 2089 | 9925 | 938 | 8536 | 6156 | 10608 | 11433 | 5967 | 1511 | 11974 |
| | 12573 | 4734 | 11501 | 5076 | 12428 | 8275 | 2769 | 4402 | 11854 | | | |
| 213: | 4784 | 5997 | 2399 | 6338 | 3933 | 4092 | 10151 | 2740 | 10610 | | | |
| 214: | 10855 | 2954 | 6766 | 2958 | 8910 | 12101 | 6783 | 3620 | 7658 | 7785 | 3180 | 9266 |
| | 9246 | 9247 | 1792 | 8649 | 5777 | 10173 | 10178 | 3461 | 9046 | 5810 | 5806 | 1226 |
| | 3287 | 12557 | 8375 | 12235 | 8403 | 8384 | 7414 | 5429 | 4396 | 6501 | 8433 | 7094 |
| | 8413 | 7920 | 5588 | 9853 | 6890 | 6483 | 9273 | 9841 | 683 | 9313 | 6871 | 6899 |
| | 6877 | 2491 | 4890 | 9129 | 5744 | 9572 | 11085 | 12037 | 11048 | 12113 | 613 | 9424 |
| | 6574 | 12066 | 7504 | 5863 | 8409 | 4273 | 10572 | 5923 | 1895 | 1893 | 9040 | 3665 |
| | 5481 | 7755 | 8408 | 924 | 1454 | 12140 | 8378 | 5510 | 5509 | 5513 | 3124 | 3103 |
| | 11911 | 4141 | 2082 | 2247 | 4630 | 8299 | 6667 | 702 | 8975 | 6801 | 745 | 741 |
| | 779 | 770 | 772 | 744 | 771 | 11549 | 719 | 7117 | 5565 | 11875 | | |
| 215: | 11919 | 9154 | 5594 | 10308 | 2827 | 2830 | 3408 | 3403 | 2471 | 5367 | 1120 | 5371 |
| | 5081 | 4880 | 10931 | 7367 | 6883 | 11808 | 6136 | 2549 | 11638 | 6868 | 8315 | 3118 |
| | 10508 | 10877 | 650 | 5616 | 4115 | 3026 | 3028 | 9516 | 785 | 9083 | 7596 | 8108 |
| | 4176 | 6525 | 5765 | 3802 | 1806 | 8081 | 7208 | 8893 | 12007 | 8654 | 9048 | 9072 |
| | 8575 | 8423 | 6300 | 6409 | 4165 | 6095 | 9477 | 2485 | 10112 | 5117 | 2278 | 2281 |
| | 2264 | 2284 | 6055 | 2348 | 4251 | 8187 | 10826 | 9660 | 9216 | 2777 | 4403 | 7239 |
| | 2643 | 782 | 2262 | 8111 | 1799 | 781 | 2696 | 8265 | 821 | 6575 | 9029 | 6259 |
| | 5907 | 2153 | 9132 | 1008 | 9697 | 11658 | 5996 | 6135 | 3512 | | | |
| 216: | 1063 | 9995 | 9748 | 8083 | 4921 | 10081 | 2976 | 7153 | 8380 | 1072 | 2845 | 2124 |
| | 5604 | 2742 | | | | | | | | | | |
| 217: | 1063 | 9995 | 9748 | 8083 | 4921 | 10081 | 2976 | 7153 | 8380 | 1072 | 2845 | 2124 |
| | 5604 | 2742 | | | | | | | | | | |
| 218: | 10265 | 3604 | 11692 | 2087 | 2100 | 2084 | 4972 | 8627 | 4940 | 10555 | 4941 | 652 |
| | 1430 | 11778 | 7581 | 915 | 1478 | 8934 | 1244 | 9538 | 6106 | 9540 | 6923 | 5854 |
| | 6892 | 9462 | 3486 | 10996 | 12018 | 9346 | 3284 | 6742 | 8247 | | | |
| 219: | 5171 | 3451 | 10952 | 6452 | 5333 | 11383 | 12420 | 9816 | 9099 | 11249 | 528 | 11871 |
| | 11060 | 8935 | 3521 | 3063 | 10253 | 9510 | 10954 | 6303 | 6941 | 523 | 904 | 5364 |
| | 4534 | 1993 | 9623 | 3245 | 12506 | 8843 | 10612 | 7200 | 2319 | 7201 | 1746 | 9164 |
| | 1043 | | | | | | | | | | | |
| 220: | 6376 | 1316 | 5391 | 12526 | 7194 | 2996 | 3154 | 10569 | 11756 | 11824 | 3924 | 9004 |
| | 5150 | 5993 | 10023 | 5309 | 10233 | 5582 | 9183 | 5649 | 2780 | 11917 | 6719 | 11145 |
| | 10056 | 2516 | 1372 | 5622 | 7269 | 2665 | 1402 | 5885 | 7636 | 6193 | 3223 | 2719 |
| | 6657 | 1867 | 7660 | 12334 | 9360 | 5492 | 2710 | 2076 | 8465 | 7571 | 11887 | 2033 |
| | 8847 | 3260 | 10323 | 11018 | 7553 | 6905 | 5747 | 10773 | 5018 | 9023 | 9420 | 9484 |
| | 9512 | 8291 | 2650 | 4553 | 2233 | 4983 | 7834 | 11916 | 8565 | 4123 | 1090 | 3981 |
| | 610 | 2885 | 5427 | 3349 | 649 | 9974 | 10523 | 10337 | 5840 | 8815 | 6996 | 11041 |
| | 1321 | 11532 | 11331 | 9757 | 6755 | 2327 | 2730 | 5199 | 5280 | 11943 | 3656 | 6297 |
| | 4570 | 2983 | 6557 | 12145 | 2376 | 7618 | 6924 | 9049 | 10975 | 8678 | 12452 | 7263 |
| | 2204 | 3741 | 7210 | 7502 | 4325 | 11408 | 1350 | 6089 | 2892 | 8054 | 8643 | 2501 |
| | 1647 | 11693 | 6378 | 1729 | 6966 | 8734 | 9027 | 8827 | 5647 | 9075 | 7286 | 659 |
| | 4113 | 6496 | 4454 | 11650 | 4378 | 2224 | 2687 | 1763 | 830 | 3255 | 5001 | 3830 |
| | 6495 | 3121 | 1757 | 7740 | 8530 | 2770 | 1866 | 459 | 2049 | 4814 | 12517 | 2408 |
| | 8583 | 6850 | 7550 | 5545 | 2042 | 3709 | 5474 | 11062 | 4761 | 10345 | 7778 | 1449 |
| | 1562 | 8901 | 4943 | 10916 | 11403 | 6820 | 3167 | 1997 | 7484 | 9833 | 12022 | 8573 |
| | 5100 | 5639 | 7158 | 8791 | 9723 | 4484 | 10282 | 1334 | 11312 | 11317 | 4294 | 9400 |
| | 4982 | 7125 | 2655 | 10854 | 9131 | 992 | 5153 | 2528 | 12519 | 12187 | 6818 | 799 |
| | 861 | 11120 | 11361 | 6634 | 12230 | 10852 | 8817 | 3105 | 9513 | 8235 | | |
| 221: | 6205 | 11358 | 3072 | 2888 | 2907 | 6203 | 2800 | 7221 | 4750 | 3627 | 12485 | 2816 |
| | 10896 | 4463 | 3774 | 8273 | 5002 | 4122 | 8581 | 8364 | 3273 | 6044 | 6503 | 6451 |
| | 6887 | 4226 | 5120 | 9987 | 679 | 12019 | 1695 | 939 | 9726 | 8964 | 2326 | 6178 |
| | 6080 | 8551 | 12220 | 926 | 10271 | 3458 | 983 | 6773 | 5354 | 551 | 12326 | 1673 |
| | 10474 | 7111 | 503 | 3261 | 7427 | 7498 | 5710 | 9522 | 12089 | 8842 | 8147 | 8799 |
| | 9369 | 2355 | 6063 | 3582 | 3537 | 3557 | 1618 | 2519 | 10121 | 9781 | 10031 | 1438 |
| | 4529 | 11657 | 7069 | 3979 | 1260 | 8752 | 9515 | 1762 | 10093 | 875 | 12460 | 12052 |
| | 9166 | 7493 | 12523 | 10742 | 10451 | 8622 | 8931 | 10210 | 7668 | 3177 | 3657 | 6276 |
| | 625 | 6423 | | | | | | | | | | |
| 222: | 9766 | 2574 | 8653 | 12518 | 6881 | 10011 | 1281 | 4435 | 3555 | 696 | 5489 | 10478 |
| | 6961 | 6001 | 1591 | 1453 | 10635 | 2267 | 6727 | 12366 | 4551 | 1889 | 1367 | 1388 |
| | 9264 | 8099 | 5016 | 1033 | 4094 | 12546 | 7145 | 6511 | 1331 | 1524 | 3894 | 1943 |
| | 8569 | 11313 | 5235 | | | | | | | | | |
| 223: | 12210 | 2632 | 5689 | 5995 | 9108 | 6848 | 3162 | 5357 | 9825 | 6099 | 9769 | 11406 |
| | 12011 | 4089 | 11037 | 2154 | 7634 | 2930 | 6937 | | | | | |
| 224: | 11851 | 9599 | 392 | 3514 | 5363 | 9918 | 7949 | 12550 | 981 | 8255 | 3499 | 2997 |
| | 9043 | 6076 | 2056 | 2922 | 11064 | 11131 | 9209 | 5316 | 10222 | 11118 | 1947 | 4743 |
| 225: | 12336 | 2351 | 3767 | 1826 | | | | | | | | |
| 226: | 9174 | 12242 | 516 | 9436 | 5692 | 6101 | 8462 | 9960 | 3910 | | | |
| 227: | 882 | 7014 | 8781 | 8246 | 10705 | 2703 | 8520 | 6497 | 7900 | 6599 | 3575 | 3216 |
| 228: | 2359 | 5356 | 6318 | 6123 | 588 | 7908 | 6312 | 4748 | 9929 | 6824 | 6509 | |
| 229: | 9744 | 8168 | 1420 | 6853 | 7687 | 2503 | 7653 | 5252 | 787 | 6057 | 2759 | 8114 |
| | 9054 | 8122 | 8127 | 4410 | 5238 | 4675 | 7892 | 11484 | 12365 | 11744 | 3437 | 6705 |
| | 3241 | 11187 | | | | | | | | | | |
| 230: | 9581 | 9205 | 10794 | 5085 | 2201 | 1384 | 4000 | 8605 | 4670 | 1972 | 10286 | 11576 |
| | 686 | 913 | 2768 | 203 | 393 | 4753 | 1339 | 2332 | 8771 | 9423 | 7426 | 2744 |
| | 3079 | 10139 | 4312 | 11256 | 7098 | 642 | 1681 | 411 | 11964 | 5179 | 1324 | 9955 |
| | 6104 | 5516 | 9644 | 11807 | 7457 | | | | | | | |
| 231: | 12356 | 4958 | 6943 | 8532 | 8516 | 9081 | 4754 | 8450 | 8451 | 10677 | 4939 | 12575 |
| | 11787 | 7205 | 4213 | 972 | 3291 | 9604 | 11517 | 7192 | 10860 | 5598 | 12538 | 4035 |
| | 11116 | 695 | 7007 | 479 | 4154 | 10733 | | | | | | |
| 232: | 641 | 10835 | 7416 | 7705 | 8597 | 5506 | 5365 | 2998 | 4911 | 1710 | 4507 | 7519 |
| | 1965 | | | | | | | | | | | |
| 233: | 7211 | 12486 | 7508 | 11321 | 5086 | 11818 | 8707 | 9321 | 1682 | 4612 | 3885 | 10374 |
| | 3698 | 2956 | 2709 | 2789 | 9060 | 3654 | 4690 | 9089 | 7726 | 3369 | 8385 | 2927 |
| | 2192 | 5052 | 11202 | 11758 | 10190 | 5874 | 8038 | 8631 | 537 | 10655 | 4768 | 2120 |
| | 3687 | 4281 | 11320 | 6521 | 4769 | 7545 | 7786 | 7407 | 12108 | 9206 | 12454 | 2147 |
| | 7282 | 12432 | 3610 | 8128 | 5956 | 3069 | | | | | | |
| 234: | 9373 | 9421 | 11561 | 11557 | 12294 | 10301 | 9284 | 6616 | 1308 | 6809 | 3915 | 11093 |
| | 3919 | 11088 | 11597 | 11298 | 11592 | 6281 | 3917 | 11137 | 1726 | 11130 | 1230 | 3689 |
| | 4740 | 3725 | 11047 | 2975 | 6172 | 1216 | 3544 | 4142 | 7375 | 746 | 11962 | 6474 |
| | 12427 | | | | | | | | | | | |
| 235: | 9373 | 9421 | 11561 | 11557 | 12294 | 10301 | 9284 | 6616 | 1308 | 6809 | 3915 | 11093 |
| | 3919 | 11088 | 11597 | 11298 | 11592 | 6281 | 3917 | 11137 | 1726 | 11130 | 1230 | 3689 |
| | 4740 | 3725 | 11047 | 2975 | 6172 | 1216 | 3544 | 4142 | 7375 | 746 | 11962 | 6474 |
| | 12427 | | | | | | | | | | | |
| 236: | 444 | 10758 | 1559 | 12502 | 889 | 9874 | 9788 | 7310 | 12020 | 6831 | 7980 | 10109 |
| | 5949 | 6731 | 11689 | 7825 | 3697 | 1264 | 4393 | 548 | 2268 | 1773 | 3208 | 1147 |
| | 4029 | 9056 | 1141 | 7469 | 5188 | 10443 | 7314 | 1452 | 1744 | 5383 | | |
| 237: | 5650 | 5881 | 10697 | 3343 | 2506 | 6706 | 9195 | 3119 | 609 | 11113 | 12263 | 12264 |
| | 9501 | 8410 | 8925 | 3221 | 7983 | 7956 | 933 | 2361 | 8269 | 9921 | 6336 | 10563 |
| | 632 | 12541 | 10155 | 10751 | 9511 | 7976 | 6351 | 5482 | 10797 | 4571 | 1776 | 12112 |
| | 7190 | 1900 | 9324 | 6339 | 7001 | 2317 | 9820 | 7015 | 6384 | 4917 | 11822 | 4227 |
| | 11377 | 6229 | 10949 | 11498 | 1448 | 8172 | 10908 | 7776 | 6183 | | | |
| 238: | 3651 | 11823 | 2950 | 1915 | 5176 | 4381 | 8742 | 6316 | 9780 | 3427 | 8319 | 899 |
| | 4829 | 11372 | 12232 | 6415 | 3788 | 1658 | 9838 | 11020 | 8918 | 7485 | 10102 | 8428 |
| | 1054 | 2552 | 11363 | 12489 | 9487 | 10566 | 9535 | 11344 | 4210 | 1739 | 5067 | 8368 |
| | 9789 | 7897 | 2937 | 10388 | 8859 | 10675 | 3146 | 1783 | 2989 | 3471 | 4847 | 919 |
| | 918 | 5832 | 1172 | 2121 | 5023 | 806 | 11459 | 12478 | 12285 | 11359 | 2683 | 11412 |
| | 12180 | 11214 | 5716 | 7022 | 8289 | 6594 | 7858 | 11270 | 1848 | 12273 | 9776 | 6464 |
| | 1578 | 4239 | 7235 | 5329 | 9074 | 3608 | 6048 | 1812 | 3310 | 7872 | 5540 | 8662 |
| | 4796 | 790 | 2336 | 6532 | 8866 | 6741 | 7383 | 5683 | 4201 | 1638 | 1583 | 6819 |
| | 11937 | 2788 | 11593 | 12298 | 6125 | 6977 | 1956 | 8141 | 7002 | 1569 | 11618 | 3937 |
| | 5648 | 10925 | 10480 | 9137 | 6221 | 2366 | 6277 | 10503 | 5161 | 12302 | 5628 | 4791 |
| 239: | 3651 | 11823 | 2950 | 1915 | 5176 | 4381 | 8742 | 6316 | 9780 | 3427 | 8319 | 899 |
| | 4829 | 11372 | 12232 | 6415 | 3788 | 1658 | 9838 | 11020 | 8918 | 7485 | 10102 | 8428 |
| | 1054 | 2552 | 11363 | 12489 | 9487 | 10566 | 9535 | 11344 | 4210 | 1739 | 5067 | 8368 |
| | 9789 | 7897 | 2937 | 10388 | 8859 | 10675 | 3146 | 1783 | 2989 | 3471 | 4847 | 919 |
| | 918 | 5832 | 1172 | 2121 | 5023 | 806 | 11459 | 12478 | 12285 | 11359 | 2683 | 11412 |
| | 12180 | 11214 | 5716 | 7022 | 8289 | 6594 | 7858 | 11270 | 1848 | 12273 | 9776 | 6464 |
| | 1578 | 4239 | 7235 | 5329 | 9074 | 3608 | 6048 | 1812 | 3310 | 7872 | 5540 | 8662 |
| | 4796 | 790 | 2336 | 6532 | 8866 | 6741 | 7383 | 5683 | 4201 | 1638 | 1583 | 6819 |
| | 11937 | 2788 | 11593 | 12298 | 6125 | 6977 | 1956 | 8141 | 7002 | 1569 | 11618 | 3937 |
| | 5648 | 10925 | 10480 | 9137 | 6221 | 2366 | 6277 | 10503 | 5161 | 12302 | 5628 | 4791 |
| 240: | 5298 | 3673 | 6171 | 5229 | 8230 | 6271 | 9427 | 1356 | 10882 | 11852 | 10687 | 6088 |
| | 10076 | 9830 | 10597 | 6373 | 3987 | 10322 | | | | | | |
| 241: | 2393 | 3407 | 11789 | 11391 | 11346 | 5568 | 689 | 9121 | 3768 | 6558 | 5447 | 1870 |
| | 7849 | 2504 | 8733 | 10066 | 994 | 11743 | 980 | 4909 | 7933 | 8486 | 8369 | 5152 |
| | 1705 | 6156 | 10608 | | | | | | | | | |
| 242: | 3431 | 5395 | 4346 | 8330 | 8327 | 8702 | 7787 | 5265 | 8943 | 12561 | 4536 | 11625 |
| | 4411 | 1035 | 11796 | 6078 | 2720 | 4449 | 10010 | 3057 | 9876 | 3536 | 5603 | 11727 |
| | 5025 | 698 | 9899 | 6457 | 10804 | 3454 | 2741 | 11343 | 11668 | 12537 | 9198 | 9194 |
| | 6906 | 11749 | 2886 | 4118 | 11050 | 3125 | 3104 | 8238 | 7647 | 11157 | 11552 | 5735 |
| | 3190 | 1224 | 2010 | 10669 | 3186 | 12278 | 10534 | 9546 | 10088 | 3888 | 1521 | 10626 |
| | 10413 | 11620 | 12324 | 1406 | 12498 | 3067 | 7386 | 6359 | 10120 | 6004 | 2803 | 9290 |
| | 11141 | 854 | 2391 | 6032 | 10433 | 12371 | 11636 | 11795 | 6713 | 8567 | 10754 | 717 |
| | 2465 | 9545 | 9886 | 6990 | 4012 | 8324 | 3742 | 1053 | 8586 | 8683 | 10073 | 12149 |
| | 7481 | 2755 | 2646 | 6082 | 8956 | 4440 | 4579 | 4447 | 6886 | 4268 | 561 | 11512 |
| | 3439 | 1568 | 8328 | 1091 | 7948 | 5861 | 726 | 582 | 11893 | 2118 | 12271 | 6845 |
| | 6843 | 6847 | 2068 | 9119 | 2022 | 8587 | 12175 | 8754 | 6777 | 497 | 9325 | 6872 |
| | 7531 | 11335 | 2928 | 9885 | 5358 | 1963 | 6109 | 7533 | 11337 | 11444 | 9889 | 6179 |
| | 1632 | 6874 | 3342 | 12072 | 12199 | 11476 | 9892 | 6224 | 6254 | 5355 | 11338 | 7625 |
| | 10426 | 10428 | 11395 | 1315 | 11066 | 11063 | 11076 | 5351 | 1440 | 11336 | 11316 | 3538 |
| | 8749 | 9778 | 5154 | 9356 | 835 | 831 | 2589 | 8503 | 10727 | 2891 | 8958 | 11046 |
| | 6790 | 9818 | 9094 | 9828 | 5374 | 8610 | 7368 | | | | | |
| 244: | 9283 | 4282 | 1754 | 571 | 9388 | 10252 | 4060 | 4063 | 10254 | 9392 | 10256 | 10251 |
| | 8049 | 10250 | 10237 | 10240 | 8942 | 10188 | 9163 | 9457 | 9412 | 4148 | 2795 | 2440 |
| | 10370 | 3577 | 434 | 12383 | 7841 | 4935 | 4928 | 4931 | 4933 | 4908 | 701 | 7640 |
| | 2602 | 8966 | 4824 | 5455 | 4822 | 4823 | 5454 | 9020 | 8692 | 9551 | 6293 | 8301 |
| | 3456 | 1197 | 12421 | 3247 | 6475 | 7319 | 9389 | 569 | 6169 | 1497 | 1499 | 9460 |
| | 2309 | 600 | 2172 | 2178 | 6997 | 2668 | 8309 | 2821 | 4458 | 2940 | 9832 | 5380 |
| | 6918 | 732 | 9375 | 9376 | 9294 | 2109 | 12129 | 8117 | 12379 | 4965 | 10550 | 7218 |
| | 12182 | 11653 | 9067 | 4197 | 9956 | 4905 | 3379 | 8543 | 5663 | 10834 | 8946 | 3814 |
| | 4643 | 1788 | 6732 | 9857 | 4189 | 10740 | 6347 | 1240 | 5417 | 6399 | 1781 | 1782 |
| | 3068 | 3398 | 10951 | 3402 | 4557 | 3406 | 1307 | 4161 | 11960 | 8337 | 8307 | 7812 |
| | 2486 | 2510 | 1431 | 6430 | 894 | 5282 | 12422 | 6029 | 10938 | 10935 | 10936 | 2671 |
| | 4260 | 10891 | 7914 | 5646 | 9237 | 11207 | 11111 | 4869 | 10685 | 6067 | 4596 | 12455 |
| | 4572 | 7777 | 2072 | 1364 | 3840 | 4879 | 1193 | 7010 | 1667 | 2748 | 4809 | 1850 |
| | 8067 | 8922 | 9994 | 5724 | 12160 | 6849 | 3432 | 11503 | 11999 | 2619 | 11453 | 5074 |
| | 12050 | 3193 | 3298 | 5344 | 3303 | 3301 | 3300 | 3362 | 3283 | 3251 | 3211 | 9607 |
| | 3277 | 4512 | 10924 | 8703 | 8838 | 4726 | 6467 | 2527 | 962 | 5203 | 4589 | 9809 |
| | 9806 | 9805 | 9812 | 12423 | 12314 | 3674 | 3728 | 3676 | 4145 | 7690 | 4132 | 3000 |
| | 3002 | 2009 | 2415 | 3348 | 2543 | 1825 | 3368 | 1753 | 11838 | 6270 | 11163 | 4736 |
| | 11353 | 3911 | 10235 | 10232 | 9385 | 10283 | 10275 | 10280 | 10279 | 1285 | 1293 | 1279 |
| | 1312 | 1291 | 1089 | 2708 | 1407 | 10065 | 9873 | 12041 | 9138 | 11097 | 4104 | 405 |
| | 10732 | 3843 | 10734 | 9232 | 1527 | 5586 | 4841 | | | | | |
| 245: | 9283 | 4282 | 1754 | 571 | 9388 | 10252 | 4060 | 4063 | 10254 | 9392 | 10256 | 10251 |
| | 8049 | 10250 | 10237 | 10240 | 8942 | 10188 | 9163 | 9457 | 9412 | 4148 | 2795 | 2440 |
| | 10370 | 3577 | 434 | 12383 | 7841 | 4935 | 4928 | 4931 | 4933 | 4908 | 701 | 7640 |
| | 2602 | 8966 | 4824 | 5455 | 4822 | 4823 | 5454 | 9020 | 8692 | 9551 | 6293 | 8301 |
| | 3456 | 1197 | 12421 | 3247 | 6475 | 7319 | 9389 | 569 | 6169 | 1497 | 1499 | 9460 |
| | 2309 | 600 | 2172 | 2178 | 6997 | 2668 | 8309 | 2821 | 4458 | 2940 | 9832 | 5380 |
| | 6918 | 732 | 9375 | 9376 | 9294 | 2109 | 12129 | 8117 | 12379 | 4965 | 10550 | 7218 |
| | 12182 | 11653 | 9067 | 4197 | 9956 | 4905 | 3379 | 8543 | 5663 | 10834 | 8946 | 3814 |
| | 4643 | 1788 | 6732 | 9857 | 4189 | 10740 | 6347 | 1240 | 5417 | 6399 | 1781 | 1782 |
| | 3068 | 3398 | 10951 | 3402 | 4557 | 3406 | 1307 | 4161 | 11960 | 8337 | 8307 | 7812 |
| | 2486 | 2510 | 1431 | 6430 | 894 | 5282 | 12422 | 6029 | 10938 | 10935 | 10936 | 2671 |
| | 4260 | 10891 | 7914 | 5646 | 9237 | 11207 | 11111 | 4869 | 10685 | 6067 | 4596 | 12455 |
| | 4572 | 7777 | 2072 | 1364 | 3840 | 4879 | 1193 | 7010 | 1667 | 2748 | 4809 | 1850 |
| | 8067 | 8922 | 9994 | 5724 | 12160 | 6849 | 3432 | 11503 | 11999 | 2619 | 11453 | 5074 |
| | 12050 | 3193 | 3298 | 5344 | 3303 | 3301 | 3300 | 3362 | 3283 | 3251 | 3211 | 9607 |
| | 3277 | 4512 | 10924 | 8703 | 8838 | 4726 | 6467 | 2527 | 962 | 5203 | 4589 | 9809 |
| | 9806 | 9805 | 9812 | 12423 | 12314 | 3674 | 3728 | 3676 | 4145 | 7690 | 4132 | 3000 |
| | 3002 | 2009 | 2415 | 3348 | 2543 | 1825 | 3368 | 1753 | 11838 | 6270 | 11163 | 4736 |
| | 11353 | 3911 | 10235 | 10232 | 9385 | 10283 | 10275 | 10280 | 10279 | 1285 | 1293 | 1279 |
| | 1312 | 1291 | 1089 | 2708 | 1407 | 10065 | 9873 | 12041 | 9138 | 11097 | 4104 | 405 |
| | 10732 | 3843 | 10734 | 9232 | 1527 | 5586 | 4841 | | | | | |
| 246: | 6942 | 11388 | 3778 | 2672 | 12375 | 7074 | 1179 | 12458 | 1855 | 12015 | 9980 | 10716 |
| | 10918 | 11770 | 10255 | 581 | 10006 | 4467 | 10169 | 7675 | 1250 | 5319 | 9827 | 496 |
| | 1732 | 8778 | | | | | | | | | | |
| 247: | 9530 | 12217 | 826 | 5267 | 2006 | 547 | 7365 | 1816 | 10509 | 6784 | 2509 | 7339 |
| | 3841 | 5471 | 11850 | 11519 | 2806 | 7443 | 8018 | 11751 | 1529 | 4077 | 12088 | 6149 |
| | 1489 | 868 | 7473 | 2466 | 614 | 1176 | 6038 | 2195 | 1557 | 8182 | 3984 | |
| 248: | 2809 | 1347 | 5519 | 6353 | 8932 | 7422 | 7557 | 4683 | 11147 | 10637 | 9404 | 11768 |
| | 910 | 8870 | 6324 | 5045 | 4945 | 9453 | 7164 | 4152 | 3322 | 10499 | 7328 | 8994 |
| | 11460 | 9634 | 9064 | 9047 | 10906 | 11904 | 749 | 5014 | 9906 | 9939 | 9963 | 2364 |
| | 6617 | 1635 | 4234 | 3462 | 12043 | 10075 | 3459 | 10489 | 8606 | 1999 | 4508 | 10028 |
| | 5341 | 6180 | 9971 | 7215 | 3422 | 5091 | 9652 | 10601 | 8028 | 11427 | 6678 | 4365 |
| | 7082 | 1022 | 3209 | 4357 | 9159 | 7321 | 12077 | 11967 | 680 | 7943 | 661 | 11246 |
| | 10622 | 7676 | 818 | 3364 | 9160 | 6187 | 8531 | 4604 | 6381 | 4859 | 2298 | 10753 |
| | 12368 | 9200 | 5155 | 2175 | 8058 | 11106 | 1639 | 5299 | 1905 | 10681 | 10937 | 1738 |
| | 3580 | 7344 | 8320 | 8466 | 5687 | 5293 | 8844 | 5030 | 1572 | 1735 | 543 | 1105 |
| | 562 | 3483 | 9147 | 12109 | 11282 | 7291 | 10666 | 11009 | 2523 | 12408 | 12128 | 12406 |
| | 2535 | 10560 | 12212 | 10101 | 11610 | 5148 | 8687 | 5945 | 11651 | 12286 | 5942 | 12291 |
| | 11624 | 11675 | 12078 | 4588 | 1480 | 1513 | 4665 | 759 | 5868 | 705 | 4662 | 1458 |
| | 4585 | 4555 | 5193 | 754 | 756 | 5855 | 4641 | 4563 | 1455 | 761 | 5867 | 5859 |
| | 5756 | 1520 | 4608 | 1437 | 5787 | 1459 | 671 | 1515 | 5784 | 5824 | 5864 | 5902 |
| | 5791 | 678 | 5169 | 5797 | 5166 | 807 | 808 | 5897 | 5173 | 793 | 5839 | 5899 |
| | 5761 | 700 | 5752 | 5748 | 848 | 845 | 792 | 786 | 815 | 673 | 853 | 1429 |
| | 812 | 850 | 849 | 817 | 1432 | 5172 | 5009 | 5062 | 5040 | 4868 | 5060 | 5066 |
| | 5032 | 5034 | 5008 | 5092 | 9098 | 5069 | 5717 | 7327 | 3898 | 5904 | 8185 | 10968 |
| | 12327 | 12382 | 12376 | 12380 | 12358 | 12355 | 4920 | 5826 | 4918 | 9879 | 6009 | 6638 |
| | 4899 | 6614 | 6005 | 9882 | 9880 | 9915 | 6609 | 6007 | 9959 | 4923 | 9936 | 9912 |
| | 5794 | 9935 | 9908 | 6637 | 9941 | 9962 | 9957 | 9970 | 6610 | 9996 | 9993 | 9991 |
| | 9998 | 4974 | 5857 | 5931 | 5830 | 5865 | 5007 | 5903 | 5852 | 5833 | 5862 | 5202 |
| | 5093 | 4978 | 5098 | 5096 | 4892 | 6002 | 5934 | 5788 | 5751 | 5785 | 5981 | 5985 |
| | 5822 | 5958 | 5227 | 6671 | 6643 | 6645 | 6640 | 1393 | 12542 | 1691 | 10538 | 9575 |
| | 2875 | 1909 | 6944 | 2273 | 7752 | 7749 | 7747 | 7774 | 7150 | 7754 | 7753 | 7720 |
| | 7717 | 7154 | 7723 | 6454 | 9178 | 1910 | 9184 | 12431 | 11386 | 11005 | 3701 | 2612 |
| | 991 | 4598 | 6761 | 458 | 1261 | 11303 | 11387 | 5560 | | | | |
| 249: | 11466 | 10817 | 8971 | 4262 | 10839 | 329 | 8600 | 5335 | 896 | 791 | 1352 | 7322 |
| | 10270 | 7741 | 9035 | 6714 | 10000 | 8201 | 3746 | 5174 | 4398 | 7207 | 587 | 4853 |
| | 4851 | 2261 | 11405 | 10793 | 3440 | 3463 | 7505 | 1693 | 2532 | 2862 | 2859 | 5792 |
| | 2825 | 11560 | 7637 | 7633 | 7614 | 2464 | 11222 | 1778 | 11268 | 11462 | 2158 | 10647 |
| | 1146 | 7316 | 6100 | 12545 | 1698 | 4358 | 879 | 1727 | 12205 | 7170 | 6117 | 10722 |
| | 12516 | 7294 | 12005 | 11165 | 1857 | 12042 | 6879 | 3473 | 4353 | 2798 | 10096 | 2424 |
| | 7595 | 9288 | 3396 | 4644 | 7051 | 6749 | 4932 | 1417 | 11847 | 8676 | 2675 | 3149 |
| | 4493 | 8041 | 2231 | 10890 | 2301 | 3232 | 3614 | 9139 | 3530 | 7139 | 7836 | 3718 |
| | 12250 | 881 | 2373 | 4126 | 9707 | 5779 | 9630 | 2905 | 11956 | 10260 | 8176 | 8370 |
| | 1834 | 1301 | 5330 | 2572 | 10162 | 3878 | 8872 | 7166 | 12030 | 9648 | 2689 | 10149 |
| | 10973 | 9109 | 4471 | 11920 | 685 | 6584 | 10680 | 5472 | 5665 | 3253 | 2889 | 12470 |
| | 8726 | 6034 | 6623 | 4199 | 4362 | 12405 | 6411 | 10429 | 12497 | 9570 | 1214 | 1030 |
| | 617 | 829 | 10859 | 11229 | 1603 | 3932 | 5522 | 1255 | 12084 | 10987 | 900 | 11188 |
| | 2018 | 616 | 10866 | 6138 | 1003 | 7084 | 10459 | 1656 | 9217 | 4190 | 10206 | 4395 |
| | 2310 | 3027 | 3199 | 8196 | 12409 | 1333 | 1930 | 7140 | 8939 | 3081 | 5939 | 1360 |
| | 6958 | 10063 | 5908 | 2324 | 5587 | 735 | 10365 | 8940 | 1173 | 7035 | 8162 | 10945 |
| | 7665 | 8546 | 4249 | 8439 | 4095 | 10156 | 10177 | 10176 | 931 | 2377 | 8634 | 420 |
| | 2894 | 3098 | 4069 | 5285 | 1311 | 9272 | 6242 | 7611 | 3944 | 3947 | 5722 | 5719 |
| | 5695 | 5720 | 5753 | 5727 | 5731 | 12515 | 9999 | 6710 | 9394 | 9395 | 4022 | 4024 |
| | 10165 | 3990 | 10801 | 3995 | 10163 | 1553 | 3993 | 11441 | 10846 | 11136 | 7843 | 10491 |
| | 4725 | 11489 | 4723 | 10823 | 10498 | 10615 | 626 | 11339 | 10409 | 11480 | 1805 | 7639 |
| | 10281 | 10034 | 8405 | 11038 | 3492 | 8645 | 5037 | 12001 | 5800 | 7175 | 4668 | 9467 |
| | 3156 | 11075 | 2995 | 9347 | 7905 | 1423 | 7870 | 7748 | 2283 | 9865 | 11865 | 5561 |
| | 8231 | 8097 | 6110 | 8686 | 7829 | 8871 | 5715 | 1804 | 5644 | 4423 | 2259 | 11914 |
| | 11250 | 8485 | 9933 | 716 | 6323 | 6799 | 3509 | 12565 | 7764 | 5263 | 9011 | 6246 |
| | 4504 | 2358 | 8877 | 2182 | 11148 | 10158 | 5262 | 5258 | 4660 | 3899 | 6021 | 10821 |
| | 10978 | 1801 | 8831 | 1936 | 6826 | 9977 | 3546 | 5982 | 3472 | 8460 | 3460 | 12528 |
| | 5242 | 8527 | 8657 | 8552 | 7744 | 6286 | 2066 | 9201 | 506 | 6016 | 5456 | 399 |
| | 481 | 11107 | 9066 | 4390 | 6748 | 11573 | 5819 | 5182 | 9176 | 11244 | 8029 | 3399 |
| | 7013 | 10161 | 10468 | 11469 | 5983 | 9301 | 5544 | 7268 | 5412 | 8121 | 3720 | 9353 |
| | 2299 | 6358 | 3224 | 11486 | 2423 | 1330 | 12174 | 3550 | 4953 | 2783 | 11429 | 8828 |
| | 8602 | 1300 | 9104 | 7287 | 8658 | 1986 | 5677 | 8336 | 12262 | 3449 | 4980 | 9514 |
| | 7606 | 2388 | 11771 | 10820 | 1780 | 11551 | 2392 | 10995 | 9489 | 2737 | 8454 | 10493 |
| | 975 | 1824 | 7713 | 12036 | 7172 | 8395 | 6062 | 4409 | 8222 | 5608 | 5031 | 12363 |
| | 11455 | 2661 | 2189 | 5632 | 6561 | 3215 | 11858 | 2652 | 9866 | 8620 | 11816 | 2701 |
| | 10674 | 568 | 11663 | 11649 | 9706 | 12317 | 12525 | 2916 | 8590 | 7129 | 9310 | 11073 |
| | 7694 | 1472 | 5767 | 7178 | 8963 | 4159 | 3109 | 8186 | 2200 | 5114 | 9659 | 6461 |
| | 8688 | 6519 | 5873 | 668 | 11777 | 10392 | 4625 | 10853 | 6210 | 3001 | 4310 | 1966 |
| | 1885 | 7538 | 5702 | 8513 | 9937 | 10901 | 11642 | 2005 | 4671 | 9435 | 3667 | 4480 |
| | 5740 | 6785 | 4971 | 10956 | 5657 | 2890 | 7670 | 8813 | 6102 | 8483 | 8452 | 11464 |
| | 10848 | 11438 | 10851 | 10769 | 10766 | 10764 | 10763 | 6869 | 8373 | 1564 | 5524 | 10079 |
| | 11491 | 11440 | 10713 | 7635 | 7631 | 5372 | 5348 | 9233 | 9231 | 9221 | 9240 | 9235 |
| | 9220 | 3029 | 10761 | 9983 | 814 | 7040 | 12560 | 7016 | 8158 | 8759 | 8762 | 8163 |
| | 7037 | 7822 | 8767 | 7066 | 7061 | 10679 | 11467 | | | | | |
| 250: | 11466 | 10817 | 8971 | 4262 | 10839 | 329 | 8600 | 5335 | 896 | 791 | 1352 | 7322 |
| | 10270 | 7741 | 9035 | 6714 | 10000 | 8201 | 3746 | 5174 | 4398 | 7207 | 587 | 4853 |
| | 4851 | 2261 | 11405 | 10793 | 3440 | 3463 | 7505 | 1693 | 2532 | 2862 | 2859 | 5792 |
| | 2825 | 11560 | 7637 | 7633 | 7614 | 2464 | 11222 | 1778 | 11268 | 11462 | 2158 | 10647 |
| | 1146 | 7316 | 6100 | 12545 | 1698 | 4358 | 879 | 1727 | 12205 | 7170 | 6117 | 10722 |
| | 12516 | 7294 | 12005 | 11165 | 1857 | 12042 | 6879 | 3473 | 4353 | 2798 | 10096 | 2424 |
| | 7595 | 9288 | 3396 | 4644 | 7051 | 6749 | 4932 | 1417 | 11847 | 8676 | 2675 | 3149 |
| | 4493 | 8041 | 2231 | 10890 | 2301 | 3232 | 3614 | 9139 | 3530 | 7139 | 7836 | 3718 |
| | 12250 | 881 | 2373 | 4126 | 9707 | 5779 | 9630 | 2905 | 11956 | 10260 | 8176 | 8370 |
| | 1834 | 1301 | 5330 | 2572 | 10162 | 3878 | 8872 | 7166 | 12030 | 9648 | 2689 | 10149 |
| | 10973 | 9109 | 4471 | 11920 | 685 | 6584 | 10680 | 5472 | 5665 | 3253 | 2889 | 12470 |
| | 8726 | 6034 | 6623 | 4199 | 4362 | 12405 | 6411 | 10429 | 12497 | 9570 | 1214 | 1030 |
| | 617 | 829 | 10859 | 11229 | 1603 | 3932 | 5522 | 1255 | 12084 | 10987 | 900 | 11188 |
| | 2018 | 616 | 10866 | 6138 | 1003 | 7084 | 10459 | 1656 | 9217 | 4190 | 10206 | 4395 |
| | 2310 | 3027 | 3199 | 8196 | 12409 | 1333 | 1930 | 7140 | 8939 | 3081 | 5939 | 1360 |
| | 6958 | 10063 | 5908 | 2324 | 5587 | 735 | 10365 | 8940 | 1173 | 7035 | 8162 | 10945 |
| | 7665 | 8546 | 4249 | 8439 | 4095 | 10156 | 10177 | 10176 | 931 | 2377 | 8634 | 420 |
| | 2894 | 3098 | 4069 | 5285 | 1311 | 9272 | 6242 | 7611 | 3944 | 3947 | 5722 | 5719 |
| | 5695 | 5720 | 5753 | 5727 | 5731 | 12515 | 9999 | 6710 | 9394 | 9395 | 4022 | 4024 |
| | 10165 | 3990 | 10801 | 3995 | 10163 | 1553 | 3993 | 11441 | 10846 | 11136 | 7843 | 10491 |
| | 4725 | 11489 | 4723 | 10823 | 10498 | 10615 | 626 | 11339 | 10409 | 11480 | 1805 | 7639 |
| | 10281 | 10034 | 8405 | 11038 | 3492 | 8645 | 5037 | 12001 | 5800 | 7175 | 4668 | 9467 |
| | 3156 | 11075 | 2995 | 9347 | 7905 | 1423 | 7870 | 7748 | 2283 | 9865 | 11865 | 5561 |
| | 8231 | 8097 | 6110 | 8686 | 7829 | 8871 | 5715 | 1804 | 5644 | 4423 | 2259 | 11914 |
| | 11250 | 8485 | 9933 | 716 | 6323 | 6799 | 3509 | 12565 | 7764 | 5263 | 9011 | 6246 |
| | 4504 | 2358 | 8877 | 2182 | 11148 | 10158 | 5262 | 5258 | 4660 | 3899 | 6021 | 10821 |
| | 10978 | 1801 | 8831 | 1936 | 6826 | 9977 | 3546 | 5982 | 3472 | 8460 | 3460 | 12528 |
| | 5242 | 8527 | 8657 | 8552 | 7744 | 6286 | 2066 | 9201 | 506 | 6016 | 5456 | 399 |
| | 481 | 11107 | 9066 | 4390 | 6748 | 11573 | 5819 | 5182 | 9176 | 11244 | 8029 | 3399 |
| | 7013 | 10161 | 10468 | 11469 | 5983 | 9301 | 5544 | 7268 | 5412 | 8121 | 3720 | 9353 |
| | 2299 | 6358 | 3224 | 11486 | 2423 | 1330 | 12174 | 3550 | 4953 | 2783 | 11429 | 8828 |
| | 8602 | 1300 | 9104 | 7287 | 8658 | 1986 | 5677 | 8336 | 12262 | 3449 | 4980 | 9514 |
| | 7606 | 2388 | 11771 | 10820 | 1780 | 11551 | 2392 | 10995 | 9489 | 2737 | 8454 | 10493 |
| | 975 | 1824 | 7713 | 12036 | 7172 | 8395 | 6062 | 4409 | 8222 | 5608 | 5031 | 12363 |
| | 11455 | 2661 | 2189 | 5632 | 6561 | 3215 | 11858 | 2652 | 9866 | 8620 | 11816 | 2701 |
| | 10674 | 568 | 11663 | 11649 | 9706 | 12317 | 12525 | 2916 | 8590 | 7129 | 9310 | 11073 |
| | 7694 | 1472 | 5767 | 7178 | 8963 | 4159 | 3109 | 8186 | 2200 | 5114 | 9659 | 6461 |
| | 8688 | 6519 | 5873 | 668 | 11777 | 10392 | 4625 | 10853 | 6210 | 3001 | 4310 | 1966 |
| | 1885 | 7538 | 5702 | 8513 | 9937 | 10901 | 11642 | 2005 | 4671 | 9435 | 3667 | 4480 |
| | 5740 | 6785 | 4971 | 10956 | 5657 | 2890 | 7670 | 8813 | 6102 | 8483 | 8452 | 11464 |
| | 10848 | 11438 | 10851 | 10769 | 10766 | 10764 | 10763 | 6869 | 8373 | 1564 | 5524 | 10079 |
| | 11491 | 11440 | 10713 | 7635 | 7631 | 5372 | 5348 | 9233 | 9231 | 9221 | 9240 | 9235 |
| | 9220 | 3029 | 10761 | 9983 | 814 | 7040 | 12560 | 7016 | 8158 | 8759 | 8762 | 8163 |
| | 7037 | 7822 | 8767 | 7066 | 7061 | 10679 | 11467 | | | | | |
| 251: | 7566 | 9095 | 9909 | 985 | 3712 | 4673 | 10400 | 6894 | 5106 | 4420 | 10664 | |
| 252: | 1273 | 6354 | 10628 | 10067 | 5950 | 6721 | 684 | 10319 | 709 | 8095 | 11478 | 8177 |
| | 9858 | 2059 | 488 | 468 | 466 | 443 | 6770 | 8271 | 8157 | 9002 | 4162 | 7370 |
| | 1098 | 10796 | 8991 | 10090 | 3419 | 1137 | 3784 | 4900 | 1074 | 6158 | 9679 | 1086 |
| | 7420 | 7419 | 11662 | 4061 | 7548 | 11926 | 460 | 10362 | 8812 | 6107 | 10946 | 6408 |
| | 8006 | 2847 | 1989 | 3661 | 2787 | 1619 | 1169 | 7204 | 7238 | 10070 | 8193 | 12501 |
| | 5485 | 1845 | 2794 | 7224 | 9406 | 3749 | 12378 | 7011 | 3438 | 11084 | 5896 | 11474 |
| | 11425 | 546 | 7789 | 4010 | 6448 | 7795 | 7357 | 11260 | 11210 | 8046 | 7430 | 5071 |
| | 10524 | 9742 | 3252 | 9006 | 10057 | 646 | 7991 | 5217 | 3450 | 5973 | 6730 | 3980 |
| | 4722 | 10407 | 12354 | 911 | 10043 | 9379 | 9461 | 9509 | 6855 | 11475 | 4699 | 4707 |
| | 3903 | 7938 | 9028 | 10396 | 7951 | 1494 | 6633 | 5811 | 1535 | 1538 | 1708 | 5130 |
| | 11886 | 5347 | 10359 | 6327 | 7431 | 11504 | 7564 | 4929 | 6272 | 10295 | 1505 | 3845 |
| | 4840 | 5433 | 8232 | 12488 | 4783 | 9665 | 667 | 1675 | 7251 | 12058 | 1860 | 10394 |
| | 8850 | 8383 | 714 | 6900 | 2682 | 4992 | 12142 | 10423 | 878 | 6382 | 4981 | 6573 |
| | 9261 | 426 | 2584 | 2775 | 2459 | 8550 | 6512 | 3410 | 10959 | 3484 | 8923 | 9711 |
| | 619 | 9357 | 3314 | 7516 | 5046 | 10986 | 11915 | 4893 | 6348 | 6418 | 3650 | 5909 |
| | 6647 | 12436 | 7700 | 1565 | 10495 | 10526 | 10511 | 10870 | 9093 | 4720 | 2493 | 9031 |
| | 11617 | 9494 | 11310 | 11940 | 4618 | 11774 | 692 | 1411 | 9722 | 4468 | 12568 | 11407 |
| | 7852 | 5755 | 8339 | 8261 | 1024 | 8260 | 11252 | 8668 | 12554 | 11172 | 2126 | 3350 |
| | 8274 | 2633 | 2347 | 867 | 9039 | 11029 | 518 | 11209 | 8727 | 10724 | 3847 | 2316 |
| | 1653 | 909 | 1651 | 2630 | 6035 | 567 | 11952 | 6533 | 2167 | 6980 | 2903 | 5645 |
| | 2985 | 1419 | 2272 | 8096 | 2028 | 6291 | 11208 | 11025 | 11024 | 5181 | 7856 | 8183 |
| | 7234 | 7750 | 3519 | 10320 | 10862 | 1964 | 3457 | 3453 | 5882 | 5849 | 8580 | 4942 |
| | 3849 | 5260 | 6222 | 9848 | 5523 | 8905 | 11374 | 436 | 465 | 464 | 1140 | 11443 |
| | 10042 | 3806 | 9553 | 10185 | 7826 | 10304 | 8012 | 5024 | 2583 | 2439 | 8659 | 11296 |
| | 4457 | 3826 | 3825 | 2123 | 6153 | 2580 | 3823 | 11235 | 5180 | 1488 | 1490 | 5211 |
| | 2474 | 1881 | 12311 | 1354 | 9614 | 7907 | 6374 | 3347 | 10926 | 1920 | 6334 | 5431 |
| | 8595 | 7641 | 5799 | 11247 | 7348 | 12051 | 5914 | 3330 | 8845 | 6621 | 4241 | 11797 |
| | 2199 | 12467 | 478 | 1427 | 11654 | 1136 | 8093 | 577 | 6132 | 2150 | 5407 | 11992 |
| | 8519 | 5378 | 6745 | 2449 | 11259 | 1376 | 10055 | 7661 | 4076 | 8854 | 8603 | 10662 |
| | 6195 | 12075 | 11323 | 11304 | 7916 | 8839 | 1138 | 7326 | 8295 | 12332 | 9482 | 11477 |
| | 484 | 7028 | 7408 | 7379 | 5325 | 4461 | 9084 | 5254 | 12236 | 5518 | 9609 | 442 |
| | 8120 | 4033 | 10357 | 514 | 6417 | 3763 | 2952 | 10085 | 1346 | 11907 | 10086 | 11849 |
| | 11941 | 2826 | 10100 | 5164 | 4319 | 7506 | 2567 | 5959 | 2764 | 12070 | 2700 | 7978 |
| | 3044 | 3064 | 1422 | 715 | 3359 | 9532 | 441 | 440 | 1862 | 740 | 718 | 8115 |
| | 3061 | 534 | 3160 | 10696 | 10298 | | | | | | | |
| 253: | 5927 | 3822 | 5709 | 5332 | 11543 | 10238 | 4587 | 4298 | | | | |
| 254: | 3005 | 4566 | 3638 | 3636 | 4949 | 5593 | 4950 | 4952 | 6386 | 6624 | 4307 | 3140 |
| | 6319 | 7213 | 7928 | 8481 | 10333 | 12107 | 5205 | 4749 | 1984 | 5730 | 3405 | 11861 |
| | 3819 | 11697 | 3214 | 6534 | 9110 | 11404 | 7447 | 7451 | 2747 | | | |
| 255: | 6851 | 10963 | 4533 | 8701 | 12352 | 4018 | | | | | | |
| 256: | 1398 | | | | | | | | | | | |
| 257: | 4311 | 9437 | 6301 | 9698 | 8305 | 4430 | 1916 | 3966 | 2446 | 8223 | 12071 | 7173 |
| | 11279 | 2994 | 12463 | 6181 | 12347 | 6140 | 9285 | 6201 | 11237 | 4813 | 11876 | 1006 |
| | 7131 | 9122 | 9924 | 9082 | 6064 | 8234 | 4545 | 11494 | 6466 | 1475 | 6806 | 9092 |
| | 6515 | 10540 | 856 | 2077 | 6207 | 8044 | 6949 | 8542 | 10988 | 7589 | 12288 | 4280 |
| | 9331 | 1713 | 1349 | 9061 | 4855 | 4694 | 10186 | 10818 | 12322 | 777 | 803 | 4460 |
| | 8010 | 8775 | 12388 | 6586 | 9554 | 5768 | 4599 | 10157 | 570 | 5597 | 4451 | 607 |
| | 1039 | 7554 | 2538 | 11891 | 3123 | 5515 | 536 | 4495 | 5739 | 3594 | 9870 | 4947 |
| | 9673 | 4798 | 1280 | 4136 | 2322 | 11007 | 6288 | 4646 | 6933 | 1844 | | |
| 258: | 9859 | 12104 | 8809 | 8807 | 6357 | 6356 | 1789 | 10837 | 2642 | 3264 | 9441 | 8984 |
| | 8089 | 4196 | 9932 | 3047 | 6863 | 7859 | 9791 | 10592 | 12563 | 993 | 9958 | 7654 |
| | 4937 | 4540 | 8667 | 1650 | 1296 | 7541 | 6815 | 1649 | 1469 | 8561 | 3834 | 11908 |
| | 12504 | 8493 | 9837 | 7137 | 7837 | 9143 | 3913 | 8926 | 669 | 602 | 397 | 11747 |
| | 10928 | 3857 | 2947 | 3905 | 7688 | 6370 | 7161 | 1745 | 7945 | 2746 | 10541 | 4925 |
| | 5430 | 5845 | 1383 | 1484 | 11461 | 7123 | 11294 | 2690 | 9638 | 7340 | 1898 | 10844 |
| | 4970 | 7629 | 12350 | 6619 | 7029 | 654 | 4815 | 12384 | 9127 | 11340 | 2498 | 1184 |
| | 3150 | 11558 | 12295 | | | | | | | | | |
| 259: | 8388 | 3722 | 7698 | 3015 | 10098 | 10548 | 508 | 4289 | 7734 | 7728 | 8287 | 7796 |
| | 7582 | 8616 | 10704 | 7030 | 4169 | 7702 | 7706 | 7760 | 5133 | 7788 | 7765 | 7767 |
| | 9315 | 6998 | 6984 | 7008 | 7005 | 7058 | 7114 | 7092 | 7063 | 7091 | 7096 | 7067 |
| | 7000 | 7012 | 10040 | 1961 | 1397 | 9439 | 8663 | 4664 | 8071 | 7086 | 6419 | 7987 |
| | 12490 | 1679 | 1657 | 4331 | 1983 | 6707 | 3391 | 7497 | 7380 | 7845 | 7666 | 9658 |
| | 7955 | 1477 | 11341 | 8188 | 12012 | 775 | 12065 | 8672 | 1245 | 4446 | 3055 | 11555 |
| | 8112 | 886 | 8711 | 8285 | 8283 | 7104 | 1258 | 3726 | 5057 | 9344 | 1937 | 12566 |
| | 8716 | 11643 | 5989 | 6696 | 2375 | 2637 | 11384 | 8563 | 3794 | 3791 | 3013 | 2235 |
| | 893 | 12177 | 1467 | 895 | 8779 | 4771 | 6545 | 757 | 2136 | 2135 | 2138 | 10125 |
| | 10542 | 4351 | 10760 | 8345 | 6952 | 3663 | 2436 | 10787 | 2205 | 4770 | 5789 | 1191 |
| | 7588 | 4737 | 1645 | 3194 | 1643 | 5301 | 9568 | 4597 | 606 | 8738 | 8740 | 688 |
| | 1447 | 11990 | 725 | 11030 | 7724 | 11012 | 7085 | 1201 | 4153 | 2707 | 1236 | 10243 |
| | 3571 | 4602 | 2754 | 10867 | 4661 | 5479 | 11835 | 7842 | 6726 | 2808 | 5829 | 8796 |
| | 4175 | 4871 | 6440 | 8878 | 2385 | 11553 | 3306 | 4444 | 1014 | 7794 | 8496 | 6687 |
| | 4238 | 425 | 8393 | 5926 | 6037 | 11564 | 937 | 6397 | 11272 | 11274 | 6981 | 597 |
| | 4128 | 8492 | 4235 | 4252 | 3066 | 1793 | 10958 | 11713 | 9976 | 10957 | 4236 | 8721 |
| | 5036 | 6827 | 1967 | 3424 | 2431 | 10983 | 9952 | 5142 | 2900 | 8262 | 12459 | 2776 |
| | 2253 | 12260 | 3016 | 5741 | 11451 | 1820 | 2515 | 3609 | 11133 | 2251 | 7893 | 12254 |
| | 5141 | 12377 | 5004 | 12351 | 5115 | 1696 | 5979 | 3793 | 3263 | 11081 | 6921 | 2030 |
| | 11957 | 4960 | 10571 | 7486 | 5011 | 8357 | 6335 | 10863 | 7264 | 10012 | 2344 | 7350 |
| | 5370 | 1724 | 5528 | 8203 | 2297 | 10903 | 8202 | 6704 | 3288 | 3690 | 8804 | 5418 |
| | 2130 | 1392 | 522 | 9793 | 1466 | 7038 | 12237 | 4120 | 8407 | 12161 | 3787 | 8131 |
| | 6605 | 5883 | 1830 | 8928 | 10864 | 6480 | 7542 | 11726 | 3185 | 6635 | 3525 | 5209 |
| | 5234 | 2592 | 5776 | 10562 | 11330 | 9613 | 11397 | 8358 | 9161 | 3218 | 9729 | 6444 |
| | 7598 | 7957 | 4667 | 4732 | 3764 | 11232 | 7992 | 7758 | 7742 | 7792 | 8316 | 8321 |
| | 8313 | 8318 | 8898 | 8314 | 8896 | 7036 | 7708 | 2838 | 2837 | 9204 | 9196 | 9165 |
| | 9169 | 9167 | 3629 | 7679 | 12494 | 7034 | 7730 | 7736 | 7901 | 2749 | 5013 | 9348 |
| | 10267 | 9407 | 4173 | 7059 | 7060 | 8764 | 5946 | 3386 | 3233 | 4464 | 7886 | 9574 |
| | 5105 | 5163 | 3187 | 3949 | 10398 | 3963 | 6241 | 6243 | 5232 | 5231 | 723 | 4071 |
| | 9692 | 10318 | 1592 | 1640 | 1593 | 6043 | 6028 | 2626 | 6027 | 8571 | 6486 | 6490 |
| | 8490 | 9869 | 6434 | 3326 | 7917 | 4490 | 10856 | 2497 | 8664 | 3138 | 6108 | 12215 |
| | 12152 | 5483 | 1775 | 5517 | 2378 | 8613 | 4639 | 6675 | 9175 | 3415 | 4345 | 7783 |
| | 4417 | 11725 | 2331 | 636 | 5449 | 2434 | 6189 | 4360 | 4452 | 3601 | 1706 | 2313 |
| | 5292 | 3390 | 5676 | 5698 | 6810 | 1021 | 1242 | 844 | 4121 | 6897 | 9485 | 11171 |
| | 5444 | 6735 | 1209 | 10132 | 10389 | 3113 | 10103 | 11622 | 11619 | 5681 | 5315 | 8607 |
| | 1700 | 11450 | 1379 | 6862 | 6993 | 9444 | 4352 | 6954 | 9727 | 10997 | 9454 | 9474 |
| | 9450 | 9445 | 2470 | 8722 | 7620 | 12274 | 9335 | 7283 | 920 | 6069 | 9134 | 7032 |
| | 8458 | 8960 | 7699 | 7097 | 4729 | 2280 | 4472 | 6660 | | | | |
| 260: | 4214 | 10052 | 2812 | 5162 | 6750 | 9903 | 2712 | 3501 | 7527 | 5168 | 5346 | 10564 |
| | 1609 | 10272 | 4848 | 8554 | 7685 | 7584 | 7875 | 4883 | 12569 | 2098 | 9005 | 8016 |
| | 1217 | 4832 | 4511 | 10354 | 4039 | 4267 | 11757 | 666 | 4526 | 4827 | 12579 | 2173 |
| | 8776 | 3900 | 2116 | 6377 | 3983 | 9759 | 955 | 5583 | 4721 | 3576 | 1498 | 8723 |
| | 6768 | 8718 | 3644 | 2075 | 5987 | 7882 | 5225 | 3196 | 7716 | 495 | 6935 | 9761 |
| | 3085 | 5953 | 9503 | 8755 | 5029 | 10373 | 10700 | 9192 | 7081 | 3865 | 7894 | |
| 261: | 485 | 7151 | 1320 | 1290 | 451 | 4232 | 7155 | 450 | 2079 | 447 | 12101 | 9224 |
| | 6447 | 8360 | 4519 | 6844 | 8050 | 1655 | 8361 | 5543 | 2398 | 10378 | 6673 | 9961 |
| | 998 | 3436 | 9533 | 8649 | 10960 | 11580 | 9946 | 3430 | 6890 | 6483 | 7347 | 3268 |
| | 5572 | 3753 | 7695 | 5744 | 8043 | 8832 | 7891 | 6441 | 3622 | 9579 | 3776 | 3752 |
| | 3773 | 6487 | 10375 | 8408 | 7209 | 1630 | 9190 | 3033 | 7177 | 1534 | 5860 | 2083 |
| | 8378 | 2654 | 5222 | 6744 | 2982 | 505 | 690 | 7195 | 6667 | 702 | 4348 | 5933 |
| | 4349 | 12102 | 12119 | 10347 | 8591 | 12096 | 4828 | 10352 | 10351 | 6392 | 771 | 770 |
| | 744 | 772 | 779 | 741 | 745 | 6274 | 719 | 4243 | 7117 | 5591 | 12243 | |
| 262: | 8290 | 9642 | 1843 | 11732 | 12475 | 1766 | 8069 | 520 | 6505 | 2170 | 9021 | 10836 |
| | 7854 | 11836 | 4028 | 3732 | | | | | | | | |
| 263: | 11809 | 8254 | 6051 | 11522 | 760 | 6489 | 10701 | | | | | |
| 264: | 8907 | 8133 | 10594 | 3921 | 5803 | 9945 | 10922 | 7645 | | | | |
| 265: | 4066 | 1661 | 3518 | 11779 | 11827 | 2479 | 11987 | 1102 | 9997 | 4456 | 4994 | 1897 |
| | 6649 | 6269 | 2684 | 3489 | 6608 | 10414 | 7293 | 502 | 967 | 6655 | 9887 | 2074 |
| | 4887 | 2305 | 7607 | 1852 | 1582 | 11458 | 4253 | 11609 | 11608 | 7459 | 6103 | 1624 |
| | 1305 | 10639 | 10069 | 4293 | 10871 | 3496 | 10261 | 7784 | 10568 | 6526 | 10840 | 3542 |
| | 7396 | 12120 | 5547 | 7108 | 1586 | 6694 | 4687 | 8748 | 2430 | 2105 | 541 | 1325 |
| | 2500 | 4205 | 9610 | 10752 | 2166 | 6371 | 10316 | 10321 | 4329 | 12333 | 5381 | 2921 |
| | 811 | 12135 | 10383 | 1428 | 3930 | 10207 | 6670 | | | | | |
| 266: | 2090 | 9566 | 9464 | 2088 | 2091 | 2106 | 10582 | 5288 | 7559 | 7950 | 2246 | 2729 |
| | 2621 | 12256 | 11470 | 6622 | 780 | 7206 | 11763 | 3939 | 2108 | 9703 | 9655 | 9674 |
| | 3145 | 9678 | | | | | | | | | | |
| 267: | 2306 | 2969 | 1292 | 5197 | 12526 | 12222 | 2996 | 3154 | 11518 | 9179 | 3130 | 11756 |
| | 11824 | 3924 | 9004 | 5150 | 1597 | 4858 | 5733 | 8995 | 10095 | 6969 | 5582 | 2383 |
| | 9888 | 6686 | 5932 | 3811 | 12143 | 5699 | 9088 | 9087 | 8207 | 1596 | 9901 | 6137 |
| | 1254 | 2780 | 4194 | 9091 | 8401 | 9488 | 1969 | 658 | 11948 | 4979 | 8425 | 9465 |
| | 789 | 7551 | 7265 | 10056 | 2516 | 11201 | 5622 | 7269 | 10981 | 10587 | 2719 | 6657 |
| | 10054 | 9953 | 8025 | 2252 | 4474 | 5694 | 9696 | 10809 | 10346 | 3591 | 12540 | 2472 |
| | 8500 | 7526 | 6542 | 8794 | 6428 | 3260 | 3853 | 834 | 11437 | 7553 | 2104 | 1174 |
| | 4070 | 10284 | 11846 | 6905 | 3318 | 3112 | 7937 | 11417 | 1512 | 7587 | 8381 | 8471 |
| | 6595 | 1879 | 5084 | 3895 | 4878 | 7737 | 7879 | 4626 | 4820 | 3745 | 8954 | 4765 |
| | 734 | 7376 | 2762 | 4983 | 4164 | 11186 | 7226 | 9505 | 6206 | 2370 | 7873 | 7227 |
| | 1335 | 2656 | 12316 | 9150 | 10736 | 5160 | 12224 | 3384 | 4212 | 1038 | 1090 | 7813 |
| | 2000 | 11019 | 8633 | 9202 | 12547 | 610 | 1151 | 477 | 11842 | 4459 | 10523 | 846 |
| | 6976 | 6177 | 3478 | 7601 | 7819 | 12396 | 3429 | 9472 | 1952 | 9073 | 6996 | 9361 |
| | 9298 | 8548 | 3686 | 3959 | 8332 | 8435 | 11759 | 6603 | 3632 | 9256 | 7646 | 4433 |
| | 10471 | 8992 | 2187 | 1321 | 8167 | 1369 | 12178 | 6756 | 431 | 11621 | 3129 | 9398 |
| | 7887 | 6330 | 1777 | 4570 | 7576 | 5170 | 3648 | 2127 | 1482 | 4993 | 4083 | 1890 |
| | 9636 | 10445 | 10415 | 2376 | 3523 | 6676 | 8678 | 6924 | 7263 | 9813 | 1641 | 8070 |
| | 4031 | 1716 | 9502 | 8473 | 3741 | 4330 | 11966 | 7877 | 10022 | 12534 | 9351 | 5643 |
| | 9443 | 9022 | 4325 | 11408 | 1742 | 9580 | 2032 | 2312 | 12130 | 6053 | 12465 | 5399 |
| | 9807 | 2943 | 5566 | 5880 | 4392 | 1847 | 2346 | 4013 | 4682 | 6974 | 5503 | 1470 |
| | 2501 | 1647 | 5895 | 11762 | 11022 | 7003 | 3169 | 11760 | 7954 | 3040 | 4710 | 3312 |
| | 3856 | 11269 | 10512 | 8997 | 10381 | 1594 | 11874 | 3858 | 5763 | 2945 | 2389 | 1287 |
| | 3666 | 12296 | 11449 | 6966 | 8724 | 3091 | 1104 | 1526 | 4106 | 2426 | 7312 | 5089 |
| | 4558 | 1362 | 784 | 7286 | 6804 | 2692 | 9155 | 638 | 4087 | 7165 | 4778 | 6496 |
| | 6596 | 11650 | 4378 | 2529 | 10338 | 3020 | 2704 | 2613 | 10136 | 2968 | 1289 | 2913 |
| | 12208 | 12213 | 2687 | 1763 | 1605 | 3255 | 5001 | 10446 | 3830 | 622 | 4919 | 7514 |
| | 6495 | 8862 | 3508 | 1757 | 6913 | 2770 | 456 | 682 | 12248 | 6264 | 10756 | 7128 |
| | 2597 | 11198 | 8880 | 1229 | 11102 | 2417 | 665 | 9103 | 6909 | 2972 | 4374 | 8782 |
| | 12517 | 10310 | 9656 | 8637 | 959 | 10314 | 2408 | 8583 | 12484 | 5284 | 8004 | 7865 |
| | 11095 | 5879 | 10104 | 8003 | 8292 | 6166 | 5477 | 12443 | 8881 | 7245 | 6369 | 4831 |
| | 5113 | 2644 | 1519 | 7906 | 7797 | 6820 | 3167 | 8998 | 1997 | 268 | 10780 | 11194 |
| | 2311 | 6443 | 12293 | 9910 | 9913 | 11253 | 2438 | 3383 | 11083 | 1199 | 5020 | 8646 |
| | 5389 | 5140 | 825 | 4294 | 9400 | 9402 | 9536 | 4982 | 4149 | 8342 | 524 | 10019 |
| | 6757 | 1894 | 7267 | 7062 | 6209 | 2655 | 10289 | 6225 | 423 | 6999 | 2829 | 4645 |
| | 408 | 9131 | 10854 | 7186 | 9600 | 3389 | 3166 | 4041 | 9764 | 8477 | 11226 | 2528 |
| | 12519 | 987 | 1542 | 11389 | 1514 | 6818 | 6026 | 4183 | 3426 | 9065 | 8073 | 7662 |
| | 521 | 1525 | 6163 | 8690 | 2566 | 11120 | 6634 | 12230 | 3498 | 10852 | 11230 | 8317 |
| | 9286 | 4782 | 2861 | 5592 | | | | | | | | |
| 268: | 2306 | 2969 | 631 | 4857 | 1313 | 7109 | 1316 | 1358 | 6436 | 6376 | 1292 | 5197 |
| | 12526 | 12222 | 2996 | 3154 | 7194 | 11518 | 9179 | 4127 | 11305 | 10569 | 10567 | 3130 |
| | 11756 | 11824 | 3924 | 9004 | 5636 | 5150 | 1597 | 4858 | 3748 | 898 | 7330 | 7336 |
| | 5733 | 1840 | 8995 | 10095 | 6969 | 10023 | 5215 | 5309 | 5582 | 10233 | 2383 | 9888 |
| | 6686 | 5932 | 9183 | 3811 | 12143 | 5699 | 9088 | 9087 | 8207 | 1596 | 6137 | 9901 |
| | 2799 | 8411 | 9354 | 6560 | 3176 | 10832 | 2488 | 9926 | 11193 | 11195 | 5123 | 1254 |
| | 2780 | 4194 | 12044 | 1537 | 4188 | 9091 | 1969 | 8401 | 9488 | 658 | 11948 | 4979 |
| | 8425 | 9465 | 4172 | 1215 | 5187 | 11001 | 6054 | 396 | 6093 | 5210 | 11954 | 789 |
| | 7551 | 7265 | 10056 | 2516 | 11201 | 1372 | 5622 | 7269 | 10981 | 1402 | 10587 | 10020 |
| | 6657 | 11034 | 11032 | 10054 | 9953 | 8025 | 2252 | 4474 | 5694 | 9696 | 5691 | 10809 |
| | 8977 | 10346 | 3591 | 9180 | 7391 | 7394 | 5492 | 12540 | 2472 | 2710 | 2686 | 2076 |
| | 2660 | 8500 | 8465 | 7571 | 11887 | 416 | 7526 | 6542 | 8794 | 8847 | 6428 | 3260 |
| | 3853 | 11018 | 10379 | 10323 | 834 | 11437 | 7553 | 4070 | 2104 | 1174 | 10284 | 11846 |
| | 388 | 1135 | 9188 | 4081 | 504 | 11872 | 1116 | 6905 | 3318 | 3112 | 11306 | 7937 |
| | 11417 | 7202 | 929 | 9811 | 7587 | 1512 | 8381 | 8471 | 6595 | 1879 | 5084 | 8348 |
| | 9653 | 4878 | 3895 | 7737 | 7879 | 4820 | 4626 | 3745 | 8954 | 4765 | 5018 | 734 |
| | 9023 | 11647 | 10134 | 7376 | 2762 | 4553 | 4983 | 4164 | 2233 | 11186 | 7226 | 9505 |
| | 1463 | 8789 | 7834 | 2370 | 6206 | 4286 | 11883 | 2350 | 1779 | 7873 | 7227 | 1335 |
| | 8565 | 2293 | 12316 | 2656 | 10736 | 9150 | 5160 | 4096 | 1749 | 1878 | 1023 | 10744 |
| | 12224 | 3384 | 4212 | 4181 | 1038 | 968 | 5850 | 4844 | 1090 | 7813 | 2000 | 11019 |
| | 8633 | 1077 | 11086 | 5295 | 3092 | 9202 | 12547 | 5178 | 1585 | 5771 | 2349 | 2911 |
| | 610 | 1151 | 477 | 2885 | 651 | 9730 | 4897 | 9257 | 1434 | 10783 | 3065 | 3735 |
| | 8911 | 3349 | 847 | 4678 | 4283 | 11842 | 2667 | 11788 | 10003 | 7487 | 5930 | 5684 |
| | 11740 | 7691 | 6817 | 5962 | 1052 | 10745 | 439 | 4459 | 10523 | 846 | 6976 | 6177 |
| | 3478 | 7601 | 7819 | 1952 | 12396 | 3429 | 9472 | 4315 | 10929 | 10731 | 1117 | 7823 |
| | 12261 | 8415 | 9073 | 6996 | 9361 | 8548 | 9298 | 3686 | 10143 | 8957 | 3959 | 8332 |
| | 8435 | 10657 | 2315 | 11759 | 6603 | 3632 | 9256 | 7646 | 4433 | 1683 | 10471 | 8992 |
| | 2187 | 1321 | 8167 | 9757 | 1369 | 12178 | 6756 | 431 | 6755 | 7219 | 11621 | 3129 |
| | 2327 | 9398 | 7887 | 6330 | 2730 | 1777 | 4570 | 11848 | 7576 | 5170 | 3715 | 5103 |
| | 5411 | 11655 | 2924 | 10812 | 3648 | 2127 | 1482 | 4922 | 8103 | 12323 | 9555 | 9526 |
| | 12027 | 4993 | 4083 | 1890 | 3046 | 4303 | 9846 | 12410 | 1069 | 9636 | 10445 | 10415 |
| | 2376 | 3523 | 7618 | 6676 | 11793 | 8678 | 6924 | 9049 | 10975 | 7263 | 9813 | 1641 |
| | 8070 | 4031 | 1716 | 3030 | 6543 | 10257 | 12281 | 9502 | 8473 | 5890 | 6492 | 3741 |
| | 4330 | 11966 | 7877 | 10022 | 12534 | 5643 | 9351 | 6220 | 9022 | 9443 | 8593 | 9086 |
| | 8252 | 3053 | 637 | 11399 | 3927 | 3956 | 6740 | 12074 | 3207 | 6653 | 4325 | 11408 |
| | 1742 | 2842 | 4629 | 8906 | 5476 | 6247 | 2037 | 7968 | 12118 | 9580 | 2032 | 12130 |
| | 2312 | 6053 | 12465 | 5399 | 9807 | 3325 | 9881 | 11280 | 8190 | 3467 | 8160 | 8985 |
| | 6516 | 7967 | 3272 | 2943 | 5566 | 1284 | 389 | 5614 | 9307 | 5191 | 10111 | 6835 |
| | 3477 | 1982 | 5273 | 12440 | 2901 | 5880 | 1847 | 4392 | 2346 | 4013 | 11723 | 1891 |
| | 4191 | 7522 | 4412 | 9130 | 4218 | 4682 | 5503 | 6974 | 1470 | 4766 | 8026 | 1555 |
| | 3869 | 2323 | 8023 | 630 | 7300 | 3951 | 8763 | 8948 | 2501 | 1647 | 5895 | 11762 |
| | 5216 | 7003 | 11022 | 3169 | 11760 | 7954 | 3040 | 10199 | 4710 | 11126 | 10147 | 8857 |
| | 5621 | 5668 | 11780 | 11939 | 5425 | 3312 | 3856 | 11269 | 8997 | 10512 | 1594 | 11874 |
| | 10381 | 3858 | 5763 | 2945 | 2389 | 1287 | 3666 | 11449 | 12296 | 2318 | 10463 | 1558 |
| | 448 | 3760 | 11055 | 1343 | 7993 | 2443 | 10324 | 3916 | 5526 | 9528 | 10453 | 2276 |
| | 7298 | 12136 | 11969 | 2482 | 6966 | 8724 | 3091 | 8734 | 8951 | 1104 | 1526 | 2426 |
| | 4106 | 5647 | 2321 | 7312 | 5089 | 1362 | 4558 | 784 | 10556 | 7286 | 2692 | 6804 |
| | 9155 | 10921 | 10659 | 638 | 659 | 4087 | 7165 | 4778 | 7233 | 6020 | 7284 | 10698 |
| | 6496 | 6596 | 11650 | 4378 | 2529 | 10338 | 3020 | 2704 | 2613 | 10136 | 1375 | 1948 |
| | 1712 | 2968 | 1289 | 2913 | 2224 | 11190 | 3021 | 12208 | 12213 | 2687 | 12495 | 1763 |
| | 1605 | 3255 | 830 | 8955 | 5001 | 10446 | 3830 | 622 | 4919 | 6716 | 9314 | 7514 |
| | 6495 | 8862 | 3508 | 8463 | 1757 | 6913 | 2770 | 456 | 459 | 4180 | 682 | 6264 |
| | 10756 | 12248 | 2597 | 11198 | 7128 | 8880 | 1229 | 11102 | 2417 | 665 | 3855 | 10502 |
| | 9103 | 6909 | 2972 | 4374 | 8782 | 12517 | 6215 | 10310 | 9656 | 8637 | 959 | 10314 |
| | 6073 | 550 | 6945 | 9390 | 11644 | 3958 | 9252 | 2408 | 8583 | 12484 | 5284 | 9146 |
| | 353 | 4888 | 8004 | 7865 | 6919 | 3790 | 3191 | 5474 | 11095 | 5879 | 8003 | 10104 |
| | 9560 | 7254 | 3313 | 6166 | 8292 | 5477 | 12443 | 8881 | 7245 | 4818 | 6523 | 601 |
| | 7467 | 5655 | 7904 | 2616 | 7385 | 11356 | 10441 | 4896 | 10757 | 5707 | 2099 | 10943 |
| | 3339 | 10559 | 10300 | 6401 | 2699 | 12163 | 6765 | 7800 | 6015 | 796 | 9475 | 5508 |
| | 1048 | 6449 | 4831 | 6369 | 5113 | 2644 | 1519 | 1551 | 1516 | 7906 | 3948 | 3970 |
| | 1241 | 8048 | 7797 | 6820 | 3167 | 8998 | 1997 | 10780 | 11194 | 2311 | 6443 | 7250 |
| | 5239 | 4290 | 8558 | 2604 | 1457 | 9596 | 9792 | 7405 | 4277 | 12381 | 5426 | 10573 |
| | 11978 | 7424 | 2328 | 2813 | 8478 | 10887 | 10427 | 2478 | 267 | 4782 | 8317 | 9286 |
| | 2861 | 5592 | 4717 | 1627 | 11355 | 4384 | 1626 | 12203 | 1628 | 1634 | 3012 | 5310 |
| | 10932 | 1938 | 1631 | 9170 | 11150 | 9904 | 12196 | 5943 | 7142 | 474 | 9062 | 10827 |
| | 2811 | 11127 | 11368 | 5339 | 9304 | 6956 | 10334 | 4001 | 6413 | 5342 | 7798 | 3896 |
| | 11894 | 7780 | 6114 | 3142 | 5535 | 11149 | 6833 | 3647 | 4112 | 12293 | 9910 | 9913 |
| | 11253 | 11312 | 2438 | 3383 | 11537 | 12138 | 2772 | 11083 | 1199 | 5020 | 10690 | 8646 |
| | 5389 | 5140 | 825 | 8086 | 4294 | 9402 | 9400 | 9536 | 4982 | 4149 | 8342 | 524 |
| | 10019 | 6757 | 1894 | 7267 | 7062 | 6209 | 2655 | 10289 | 6225 | 423 | 6999 | 2829 |
| | 4645 | 408 | 6995 | 8495 | 12394 | 9131 | 10854 | 7186 | 9600 | 3389 | 3166 | 5667 |
| | 7275 | 4542 | 6618 | 519 | 12086 | 1366 | 4041 | 9764 | 8477 | 7372 | 1002 | 9763 |
| | 11446 | 11226 | 2528 | 12519 | 987 | 1542 | 11389 | 1514 | 6818 | 6026 | 4183 | 9065 |
| | 3426 | 8073 | 7662 | 521 | 1525 | 6163 | 8690 | 2566 | 11120 | 6634 | 11361 | 9685 |
| | 12230 | 3498 | 10852 | 11230 | 8817 | 11471 | 3901 | | | | | |
| 269: | 10246 | 7020 | 10686 | 1680 | 2796 | 1248 | 4594 | 2505 | 2649 | 8814 | 4168 | 8374 |
| | 6389 | 751 | 11074 | 5159 | 11572 | 9199 | 8170 | 11830 | 3320 | 1694 | 6988 | 4377 |
| | 1158 | 2140 | 6808 | 1540 | 816 | 2611 | 2241 | 4706 | 12106 | 2495 | 7793 | 6342 |
| | 12477 | 3412 | 7006 | 12049 | 10544 | 4477 | 12125 | 7475 | 4354 | 2846 | 7156 | 6363 |
| | 2055 | 7558 | 5296 | 12398 | 1019 | 2781 | 7458 | 6926 | 7121 | 11829 | 3754 | 9602 |
| | 3038 | 2174 | 11439 | 11516 | 8441 | 3201 | 538 | 4055 | 8783 | 11982 | 11239 | 4860 |
| | 6050 | 7851 | 6664 | 4339 | 8596 | 1835 | 4062 | 8288 | 1678 | 5580 | 3810 | 8594 |
| | 7041 | 6577 | 1960 | | | | | | | | | |
| 270: | 4062 | 1835 | 12226 | | | | | | | | | |
| 271: | 8290 | 9642 | 1843 | 11732 | 12475 | 1766 | 8069 | 520 | 1037 | 6505 | 5910 | 11968 |
| | 2170 | 9735 | 9733 | 3296 | 9919 | 9736 | 6388 | 4211 | 10836 | 7854 | 6903 | 4895 |
| | 4891 | 2824 | 11297 | 6396 | 5660 | 3159 | 4028 | 3732 | | | | |
| 272: | 8063 | 5732 | 3188 | 12369 | 8106 | 5080 | 8559 | 5286 | 9212 | 6803 | 750 | 4584 |
| | 1070 | 6395 | 12048 | 6368 | 4780 | 6720 | 3285 | 7270 | 5175 | 10059 | 3031 | 9890 |
| | 4441 | 8785 | 8432 | 7543 | 8947 | 2382 | 9338 | 9916 | 2726 | 5706 | 4904 | 1112 |
| | 8534 | 12046 | 5615 | 4488 | 804 | 3480 | 1954 | 12218 | 6267 | 11927 | 6258 | 8125 |
| | 8126 | 5527 | 6214 | 11902 | 1822 | 1837 | 6240 | 7878 | 12091 | 8556 | 9969 | 9381 |
| | 11837 | 7476 | 8665 | 452 | 3702 | 12328 | 10209 | 2368 | 7672 | 6087 | 4676 | 4229 |
| | 7434 | 3076 | 10466 | 6404 | 841 | 4861 | 4575 | 2872 | 9917 | 8967 | 9749 | 12115 |
| | 10692 | 5473 | 7256 | 1873 | 2198 | 1336 | 3931 | 5798 | 5808 | 8673 | 3377 | 510 |
| | 3158 | 3925 | 8389 | 12141 | 9014 | 11932 | 3569 | 10651 | 2208 | 8990 | 3345 | 906 |
| | 12209 | 1050 | 7346 | 9893 | 1587 | 3161 | 10825 | 3649 | 1918 | 6767 | 501 | 4795 |
| | 10221 | 5149 | 6439 | 2784 | 10214 | 1584 | 3120 | 12202 | 5678 | 428 | 10578 | 7612 |
| | 4274 | 6191 | 4074 | 6190 | 7590 | 10144 | 3043 | 5323 | 3640 | 5250 | 1128 | 8414 |
| | 9490 | 9112 | 9758 | | | | | | | | | |
| 273: | 8564 | 10720 | 7580 | 12251 | 9922 | 5975 | 9534 | 9393 | 11158 | 3613 | 2357 | 8444 |
| | 2119 | 7602 | 3108 | 2579 | 8365 | 9547 | 11895 | 2245 | 12158 | 11802 | 1274 | 6626 |
| | 8769 | 278 | 2843 | 10230 | 6198 | 10814 | 2304 | 9207 | 9542 | 6236 | 10051 | 10027 |
| 274: | 8564 | 10720 | 7580 | 12251 | 9922 | 9576 | 4259 | 7808 | 6453 | 6362 | 10533 | 12131 |
| | 4838 | 10436 | 9019 | 7182 | 4738 | 7025 | 1326 | 7147 | 8769 | 6626 | 4708 | 11348 |
| | 8116 | 2843 | 10230 | 6198 | 10814 | 2304 | 9207 | 9975 | | | | |
| 275: | 8564 | 10720 | 7580 | 12251 | 9922 | 5975 | 8617 | 4257 | 645 | 295 | 9393 | 9534 |
| | 11158 | 3613 | 6982 | 2357 | 9576 | 4259 | 7808 | 8296 | 3210 | 4615 | 747 | 8228 |
| | 8444 | 6520 | 1408 | 1212 | 2119 | 12131 | 4397 | 10412 | 3357 | 10137 | 7547 | 3018 |
| | 7602 | 1687 | 7289 | 2579 | 11413 | 4889 | 3108 | 2245 | 2058 | 1274 | 1304 | 1326 |
| | 12252 | 9409 | 6626 | 8769 | 4708 | 11442 | 5397 | 12002 | 8139 | 5642 | 11431 | 8792 |
| | 387 | 7509 | 208 | 2269 | 11533 | 6769 | 2676 | 1510 | 921 | 3668 | 8728 | 2843 |
| | 10230 | 6198 | 10814 | 2304 | 9207 | | | | | | | |
| 276: | 8564 | 10720 | 7580 | 12251 | 9922 | 5975 | 6953 | 3210 | 8228 | 4615 | 1408 | 747 |
| | 8444 | 2119 | 6520 | 12131 | 1212 | 8085 | 4738 | 11349 | 8769 | 6626 | 4708 | 2149 |
| | 6060 | 1642 | 2843 | 10230 | 6198 | 10814 | 2304 | 9207 | | | | |
| 278: | 8564 | 10720 | 7580 | 12251 | 9922 | 5975 | 8365 | 9547 | 11895 | 12158 | 4163 | 4240 |
| | 4160 | 5925 | 3184 | 8350 | 7490 | 11199 | 6290 | 9672 | 12433 | 3775 | 6965 | 6236 |
| | 2843 | 10230 | 6198 | 10814 | 2304 | 9207 | | | | | | |
| 279: | 11873 | 12447 | 2706 | 9271 | 8545 | 8588 | 8589 | 1577 | 1575 | 1576 | 1556 | 1574 |
| | 1554 | 12551 | 2725 | 3886 | 4500 | 6611 | 2287 | 8846 | 8469 | 10838 | 672 | 10223 |
| | 7975 | 8434 | 3868 | 4882 | 10126 | 11563 | 3642 | 9352 | 11652 | 8174 | 11905 | 9191 |
| | 8406 | 4808 | 4695 | 731 | 743 | 11308 | 557 | 8768 | 8766 | 7331 | 8826 | 8798 |
| | 8805 | 8823 | 8802 | 8770 | 8810 | 7337 | 10227 | 12576 | 6424 | 8751 | 2625 | 2329 |
| | 11117 | 5782 | 5613 | 12126 | 3234 | 11981 | 7912 | 4328 | 8756 | 10460 | 11394 | 11912 |
| | 11140 | 11794 | 5736 | 7997 | 8001 | 8833 | 8858 | 8229 | 11559 | 4865 | 2620 | 4139 |
| | 10306 | 12509 | 7191 | 6463 | 3414 | 7888 | 9622 | 2636 | 10442 | 1509 | 8263 | 5935 |
| | 4620 | 8920 | 7281 | 978 | 12244 | 8303 | 4679 | 10588 | 12456 | 8675 | 1462 | 10865 |
| | 2027 | 9686 | 1337 | 7315 | 2477 | 4322 | 2786 | 12487 | 3434 | 8685 | 1110 | 10015 |
| | 12292 | 1614 | 10953 | 5654 | 1401 | 5132 | 4731 | 4059 | 10385 | 8101 | 1092 | 2544 |
| | 3727 | 8068 | 9657 | 11690 | 9843 | 902 | 11114 | 11508 | 9248 | 5844 | 4093 | 4193 |
| | 7973 | 7272 | 9752 | 3395 | 7480 | 12062 | 7735 | 7260 | 5272 | 5194 | 2631 | 1302 |
| | 11584 | 6056 | 11710 | 6971 | 11324 | 10325 | 2534 | 3684 | 9590 | 5816 | 1644 | 7616 |
| | 5600 | 5596 | 8889 | 8884 | 7853 | 7024 | 1648 | 8367 | 5246 | 4913 | 12176 | 704 |
| | 10123 | 9378 | 1836 | 11691 | 1654 | 9950 | 2628 | 11218 | 5390 | 4684 | 8860 | 5562 |
| | 4654 | 5424 | 4056 | 4054 | 4649 | 5575 | 5573 | 4685 | 4681 | 794 | 1741 | 10798 |
| | 10795 | 10815 | 3025 | | | | | | | | | |
| 280: | 8944 | 10309 | 11523 | 5409 | 9218 | 5496 | 3060 | 12127 | 10717 | 1563 | 11079 | 5075 |
| | 5446 | 7162 | 6394 | 475 | 2895 | 11855 | | | | | | |
| 281: | 8713 | 1390 | 12290 | 11594 | 6366 | 5219 | 9822 | 472 | 990 | 1864 | 5322 | 3734 |
| | 5671 | 8402 | 1798 | 5624 | 8175 | 8143 | 2112 | 2007 | 9228 | 8075 | 8074 | 9689 |
| | 1977 | 6691 | 4246 | 11342 | 9339 | 12570 | 3946 | 1709 | 3643 | 7404 | 4008 | 822 |
| | 2906 | 9396 | 6308 | 7762 | 8245 | 5247 | 8501 | 5398 | 9724 | 10792 | 9537 | 8154 |
| | 1704 | 3799 | 12444 | 7567 | 4631 | 10236 | 4053 | 10234 | 12524 | 11204 | 12520 | |
| 282: | 8713 | 1390 | 12290 | 11594 | 6366 | 5219 | 9822 | 472 | 990 | 1864 | 5322 | 3734 |
| | 5671 | 8402 | 1798 | 5624 | 8175 | 8143 | 2112 | 2007 | 9228 | 8075 | 8074 | 9689 |
| | 1977 | 6691 | 4246 | 11342 | 9339 | 12570 | 3946 | 1709 | 3643 | 7404 | 4008 | 822 |
| | 2906 | 9396 | 6308 | 7762 | 8245 | 5247 | 8501 | 5398 | 9724 | 10792 | 9537 | 8154 |
| | 1704 | 3799 | 12444 | 7567 | 4631 | 10236 | 4053 | 10234 | 12524 | 11204 | 12520 | |
| 283: | 5129 | 579 | 7910 | 6041 | 4924 | 9938 | 8648 | 1959 | 5116 | 11840 | 10410 | 6723 |
| | 2627 | 2374 | 8349 | 5198 | 4261 | 7308 | 12451 | 4744 | 8824 | 5423 | 9208 | 7703 |
| | 8379 | 7382 | 4873 | 7258 | 887 | 12265 | 9643 | 1685 | 7499 | 8488 | 3329 | 7902 |
| | 9795 | 3563 | 11706 | 486 | 9611 | 5352 | 8424 | 2190 | 6175 | 6882 | 6684 | 10772 |
| | 7323 | 5838 | 5796 | 12157 | 5042 | 12530 | 8929 | 2774 | 7524 | 3019 | 6134 | 9705 |
| | 11596 | 12028 | 5028 | 8919 | 6435 | 10672 | 5108 | 3737 | 6630 | 1204 | 10355 | 3914 |
| | 934 | 8102 | 11080 | 3227 | 5595 | 4999 | 11933 | 10068 | 493 | 10775 | 6715 | 4025 |
| | 748 | 3338 | 2537 | 4002 | 9897 | 12249 | 4601 | 2307 | 2406 | 12399 | 3839 | 11641 |
| | 7811 | 855 | 11881 | 4418 | 874 | 11400 | 3526 | 2517 | 5240 | 7534 | 9327 | 9479 |
| | 6916 | 4651 | 2448 | 4764 | 7115 | 7359 | 4745 | 2218 | 2445 | 5550 | 11288 | 4565 |
| | 10376 | 10600 | 10339 | 6052 | 5107 | 10317 | 2243 | 9128 | 4595 | 3864 | 2303 | 9770 |
| | 9539 | 9743 | 11803 | 7677 | 11096 | 8297 | 1154 | 3485 | 10991 | 876 | 4100 | 7296 |
| | 4016 | 2065 | 7236 | 12548 | 10273 | 12097 | 2595 | 7217 | 1666 | 8670 | 10349 | 1774 |
| | 2476 | 7363 | 4741 | 6796 | 6565 | 11292 | 1849 | 4373 | 3435 | 7230 | 3341 | 2541 |
| | 1821 | 8498 | 5396 | 3198 | 11538 | 3685 | 8651 | 10348 | 7686 | 3494 | 2601 | 3152 |
| | 2923 | 7678 | 10044 | 12272 | 8979 | 8978 | 9671 | 457 | 3653 | 1629 | 4117 | 4614 |
| 284: | 6668 | 3235 | 3818 | 3280 | 4758 | 4297 | 9603 | 12233 | 6255 | 277 | 11983 | 5049 |
| | 1677 | 10510 | 5376 | 4876 | 7709 | 2606 | 7855 | 5817 | 5502 | 2752 | 8873 | 7831 |
| | 12155 | 4505 | 3421 | 8647 | 2936 | 8837 | 1861 | 10226 | 3621 | | | |
| 285: | 1976 | 5617 | 4364 | 10244 | 11750 | 7026 | 10598 | 10444 | 12198 | 6957 | 11935 | 1931 |
| | 721 | 957 | 10530 | 5964 | 3926 | 2863 | 5386 | 3808 | 8614 | 2702 | 6345 | 10476 |
| | 5312 | 4110 | 6414 | 9914 | 11598 | 7824 | 5318 | 417 | 12305 | 7779 | 5448 | 12493 |
| | 4038 | | | | | | | | | | | |
| 286: | 1976 | 5617 | 4364 | 10244 | 11750 | 7026 | 10598 | 10444 | 12198 | 6957 | 11935 | 1931 |
| | 721 | 957 | 10530 | 5964 | 3926 | 2863 | 5386 | 3808 | 8614 | 2702 | 6345 | 10476 |
| | 5312 | 4110 | 6414 | 9914 | 11598 | 7824 | 5318 | 417 | 12305 | 7779 | 5448 | 12493 |
| | 4038 | | | | | | | | | | | |
| 287: | 4532 | 4537 | 4535 | 4321 | 5690 | 3863 | 4040 | 5167 | 9368 | 6992 | 1071 | 1871 |
| | 3307 | 1504 | 9777 | 11731 | 3677 | 930 | 7604 | 4057 | 11684 | 4610 | 4616 | 4007 |
| | 2093 | 1382 | 8118 | 1103 | 4486 | 5467 | 5393 | 4515 | 4521 | 4523 | 4494 | 4498 |
| | 4491 | 4501 | 4517 | 2428 | 10002 | 7721 | | | | | | |
| 288: | 4532 | 4537 | 4535 | 4321 | 5690 | 3863 | 4040 | 5167 | 9368 | 6992 | 1071 | 1871 |
| | 3307 | 1504 | 9777 | 11731 | 3677 | 930 | 7604 | 4057 | 11684 | 4610 | 4616 | 4007 |
| | 2093 | 1382 | 8118 | 1103 | 4486 | 5467 | 5393 | 4515 | 4521 | 4523 | 4494 | 4498 |
| | 4491 | 4501 | 4517 | 2428 | 10002 | 7721 | | | | | | |
| 289: | 5602 | 4469 | 5419 | 5432 | 4637 | 10619 | 12255 | 11347 | 1232 | 1294 | 10091 | 12571 |
| | 3340 | 7341 | 10614 | 3641 | 10384 | 2591 | 10195 | 6615 | 3382 | | | |
| 290: | 5602 | 4469 | 5419 | 5432 | 4637 | 10619 | 12255 | 11347 | 1232 | 1294 | 10091 | 12571 |
| | 3340 | 7341 | 10614 | 3641 | 10384 | 2591 | 10195 | 6615 | 3382 | | | |
| 291: | 956 | 4465 | 4807 | 7960 | 12053 | | | | | | | |
| 292: | 956 | 4465 | 4807 | 7960 | 12053 | | | | | | | |
| 293: | 9995 | 10229 | 5629 | 4497 | 5478 | 1295 | 6535 | 7277 | 5314 | 5311 | 2845 | 2893 |
| | 1590 | 3892 | 5403 | 9930 | 12093 | | | | | | | |
| 294: | 5961 | 4836 | 7630 | 4296 | 5666 | 7068 | 3635 | 8611 | 3090 | 1533 | 3559 | 10613 |
| | 6581 | 2421 | 6383 | 11801 | | | | | | | | |
| 295: | 8564 | 10036 | 10720 | 7580 | 12251 | 9922 | 5975 | 3210 | 4615 | 747 | 8228 | 10137 |
| | 4889 | 11413 | 1687 | 7547 | 3357 | 10412 | 4397 | 4738 | 1304 | 8769 | 6626 | 4708 |
| | 921 | 2843 | 10230 | 6198 | 10814 | 2304 | 9207 | | | | | |
| 296: | 10164 | 12268 | 7193 | 12156 | 12154 | 9486 | 8455 | 12552 | 5871 | 11173 | 2548 | 2542 |
| | 5633 | 3141 | 3766 | 590 | 1082 | 3992 | 8267 | 6299 | 778 | 2533 | 1059 | 4146 |
| | 7163 | 5256 | 11156 | 11648 | 11529 | 5921 | 8825 | 6682 | 6157 | 8241 | 1259 | 10579 |
| | 11293 | 3172 | 3890 | 3244 | 1660 | 5461 | 9077 | 10417 | 11782 | 9241 | 4647 | 6085 |
| | 5533 | 10786 | 11283 | 8864 | 7613 | 10145 | 3023 | 2254 | 11899 | 9693 | 4998 | 2249 |
| | 6410 | 5641 | 7093 | 4052 | 4549 | 1355 | 3042 | 7495 | 9483 | 1005 | 6235 | 11042 |
| | 9349 | 7772 | 11454 | 7077 | 2216 | 6433 | 7119 | 629 | 6578 | 12361 | 12367 | 12339 |
| | 12304 | 1095 | 5688 | 9382 | 10369 | 3991 | 7244 | 3978 | 8632 | 6688 | 297 | 4237 |
| | 8212 | 10490 | 8019 | 10723 | 6507 | 7628 | 10789 | 3579 | 7364 | 8000 | 1445 | 663 |
| | 12144 | 9990 | 11984 | 1945 | 8030 | 3293 | 8031 | 2228 | 3137 | 5015 | 11472 | |
| 297: | 10164 | 12268 | 7193 | 9486 | 8455 | 12552 | 5871 | 11173 | 2548 | 2542 | 5633 | 3141 |
| | 3766 | 590 | 1082 | 3992 | 8267 | 6299 | 778 | 2533 | 1059 | 4146 | 7163 | 5256 |
| | 11156 | 11648 | 11529 | 5921 | 8825 | 6157 | 8241 | 1259 | 10579 | 11293 | 3172 | 3890 |
| | 3244 | 1660 | 5461 | 9077 | 10417 | 11782 | 9241 | 4647 | 6085 | 5533 | 10786 | 11283 |
| | 8864 | 7613 | 10145 | 3023 | 2254 | 11899 | 9693 | 4998 | 2249 | 6410 | 296 | 5015 |
| | 11472 | 9171 | 5641 | 7093 | 4052 | 1355 | 4549 | 3042 | 7495 | 9483 | 6433 | 7119 |
| | 6578 | 12361 | 12367 | 12339 | 12304 | 1095 | 5688 | 9382 | 10369 | 3991 | 7244 | 3978 |
| | 8632 | 6688 | 8000 | 663 | 12144 | 9990 | 11984 | 1945 | 8030 | 8031 | 3293 | 2228 |
| | 3137 | | | | | | | | | | | |
| 298: | 6538 | 1032 | 1988 | 11733 | 10816 | 4334 | 10053 | 7132 | 8765 | | | |
| 299: | 9629 | 10933 | 11170 | 4429 | 11119 | 10276 | 2041 | 7360 | 11044 | 10557 | | |
| 300: | 8084 | 5326 | 12114 | 2801 | 859 | 5498 | 5501 | 5500 | 4097 | 2853 | 11026 | 1093 |
| | 6864 | 7572 | 1322 | 1998 | 9312 | 3195 | 3212 | 9141 | 6500 | 7593 | 2695 | 5994 |
| | 11381 | 2046 | 12450 | 1175 | 11496 | 10580 | 3308 | 10507 | 1359 | 766 | 482 | 7144 |
| | 3680 | 12207 | 5610 | 4785 | 5099 | 837 | 3691 | 11589 | 4538 | 9550 | 8239 | 10350 |
| | 4306 | 6253 | 8310 | 6929 | 7570 | 10029 | 6839 | 11665 | 12183 | 8699 | 10366 | 1752 |
| | 4109 | 9684 | 10416 | 12239 | 2071 | 10593 | 11167 | 4792 | 8276 | 4032 | 1858 | 10979 |
| | 11380 | 1446 | 7503 | 10114 | 1439 | 8461 | 3367 | 8284 | 8579 | 10447 | 5422 | 6013 |
| | 11212 | 11985 | 10505 | 10684 | 1958 | 5243 | 5224 | 3420 | 5244 | 2666 | 3503 | 10830 |
| | 7361 | 11291 | 3084 | 4658 | 1222 | 7043 | 833 | 2929 | 9358 | 7216 | 3236 | 4806 |
| | 1737 | 1662 | 2206 | 10693 | 4204 | 2008 | 12168 | 9731 | 3859 | 8398 | 6959 | 5618 |
| | 6121 | 10749 | 3974 | 1725 | 3083 | 9145 | 10401 | 3583 | 3584 | 3534 | 5237 | 11267 |
| | 5317 | 1076 | 5918 | 5308 | 8214 | 7478 | 11067 | 7070 | 703 | 1013 | 6708 | 857 |
| | 3213 | 6390 | 10358 | 1129 | 3328 | 11224 | 1067 | 2452 | 12064 | 7929 | 2677 | 12496 |
| | 2669 | 11302 | 5579 | 9844 | 11950 | 6986 | 5851 | 9106 | 12319 | 3231 | 10187 | 11369 |
| | 3962 | 2664 | 708 | 10537 | 8869 | 11611 | 10873 | 7664 | 9942 | 2756 | 11666 | 605 |
| | 2372 | 2185 | 4657 | 12151 | 917 | 8091 | 1353 | 2942 | 12000 | 401 | 10695 | 9397 |
| | 3623 | 4907 | 4103 | 2739 | 3833 | 8806 | 9107 | 5443 | 3374 | 578 | 2520 | 6380 |
| | 9491 | 10688 | 783 | 3548 | 403 | 5055 | 8515 | 11236 | 7936 | 2002 | 8293 | 5277 |
| | 1426 | 11734 | 9896 | 12360 | 3552 | 11263 | 4613 | 2881 | 873 | 969 | 7729 | 11390 |
| | 10130 | | | | | | | | | | | |
| 301: | 8084 | 5326 | 12114 | 2801 | 859 | 5498 | 5501 | 5500 | 4097 | 2853 | 11026 | 1093 |
| | 6864 | 7572 | 1322 | 1998 | 9312 | 3195 | 3212 | 9141 | 6500 | 7593 | 2695 | 5994 |
| | 11381 | 2046 | 12450 | 1175 | 11496 | 10580 | 3308 | 10507 | 1359 | 766 | 482 | 7144 |
| | 3680 | 12207 | 5610 | 4785 | 5099 | 837 | 3691 | 11589 | 4538 | 9550 | 8239 | 10350 |
| | 4306 | 6253 | 8310 | 6929 | 7570 | 10029 | 6839 | 11665 | 12183 | 8699 | 10366 | 1752 |
| | 4109 | 9684 | 10416 | 12239 | 2071 | 10593 | 11167 | 4792 | 8276 | 4032 | 1858 | 10979 |
| | 11380 | 1446 | 7503 | 10114 | 1439 | 8461 | 3367 | 8284 | 8579 | 10447 | 5422 | 6013 |
| | 11212 | 11985 | 10505 | 10684 | 1958 | 5243 | 5224 | 3420 | 5244 | 2666 | 3503 | 10830 |
| | 7361 | 11291 | 3084 | 4658 | 1222 | 7043 | 833 | 2929 | 9358 | 7216 | 3236 | 4806 |
| | 1737 | 1662 | 2206 | 10693 | 4204 | 2008 | 12168 | 9731 | 3859 | 8398 | 6959 | 5618 |
| | 6121 | 10749 | 3974 | 1725 | 3083 | 9145 | 10401 | 3583 | 3584 | 3534 | 5237 | 11267 |
| | 5317 | 1076 | 5918 | 5308 | 8214 | 7478 | 11067 | 7070 | 703 | 1013 | 6708 | 857 |
| | 3213 | 6390 | 10358 | 1129 | 3328 | 11224 | 1067 | 2452 | 12064 | 7929 | 2677 | 12496 |
| | 2669 | 11302 | 5579 | 9844 | 11950 | 6986 | 5851 | 9106 | 12319 | 3231 | 10187 | 11369 |
| | 3962 | 2664 | 708 | 10537 | 8869 | 11611 | 10873 | 7664 | 9942 | 2756 | 11666 | 605 |
| | 2372 | 2185 | 4657 | 12151 | 917 | 8091 | 1353 | 2942 | 12000 | 401 | 10695 | 9397 |
| | 3623 | 4907 | 4103 | 2739 | 3833 | 8806 | 9107 | 5443 | 3374 | 578 | 2520 | 6380 |
| | 9491 | 10688 | 783 | 3548 | 403 | 5055 | 8515 | 11236 | 7936 | 2002 | 8293 | 5277 |
| | 1426 | 11734 | 9896 | 12360 | 3552 | 11263 | 4613 | 2881 | 873 | 969 | 7729 | 11390 |
| | 10130 | | | | | | | | | | | |
| 302: | 7930 | 8544 | 10850 | 11315 | 1622 | 713 | 7552 | 12402 | 12054 | 2832 | 1378 | 7648 |
| | 5146 | 2634 | 9070 | 7958 | 4361 | 8568 | 5530 | 3222 | 7766 | 9447 | 7141 | 3358 |
| | 2648 | 3815 | 12564 | 7471 | 949 | 6651 | 11701 | 3772 | 8852 | 4184 | 7621 | 7652 |
| | 9840 | 9038 | | | | | | | | | | |
| 303: | 1087 | 3127 | 12531 | 11944 | 8119 | 5400 | 10714 | 2653 | 9713 | 6738 | 7171 | 7017 |
| | 3271 | 304 | 3126 | 11365 | 6566 | 6841 | 4270 | 4954 | 5831 | 3493 | 3736 | |
| 304: | 9927 | 8180 | 9829 | 3960 | 3127 | 2849 | 1133 | 11944 | 7171 | 4954 | 3126 | |
| 305: | 4622 | 10899 | 8164 | 3782 | 5729 | 798 | | | | | | |
| 306: | 5804 | 12016 | 7448 | 10738 | 10708 | 12014 | 10638 | 7423 | 7421 | 7417 | 309 | 10586 |
| | 7444 | 7446 | 5047 | 10645 | 10646 | 12280 | 207 | 2096 | 11839 | 7072 | 9709 | 1612 |
| | 8993 | 10037 | 6780 | 11613 | 9034 | 2004 | 8166 | 6931 | 7311 | 6922 | 8933 | 10494 |
| | 3783 | 308 | 12034 | 3781 | 9745 | 916 | 6666 | 9140 | 6285 | 9452 | 8035 | 12449 |
| | 10356 | 10064 | 4275 | 2067 | 3934 | 11430 | 12246 | 9405 | 2987 | 9052 | 12318 | 6252 |
| | 11326 | 8138 | 3397 | 9877 | 310 | 11058 | 8474 | 9105 | 11870 | 12047 | 3986 | 6725 |
| | 1902 | 4332 | 4672 | 9408 | 4088 | 3842 | 2342 | 1701 | 10402 | 12181 | 8446 | 4050 |
| | 6973 | 6860 | 7715 | 860 | 10725 | 4578 | 862 | 7045 | 11785 | 4789 | 5465 | 10224 |
| | 8088 | 10189 | 3553 | 9992 | 9964 | 3950 | 2793 | 6677 | 10001 | 3375 | 4200 | 10391 |
| | 10676 | 11712 | 10682 | 4747 | 206 | 307 | | | | | | |
| 307: | 12016 | 7448 | 9024 | 10738 | 10708 | 12014 | 7423 | 7421 | 7417 | 309 | 10586 | 7444 |
| | 7446 | 5047 | 10646 | 12280 | 207 | 2096 | 11839 | 9709 | 1612 | 8993 | 9034 | 2004 |
| | 8166 | 6931 | 7311 | 6922 | 3783 | 308 | 12034 | 3781 | 916 | 9745 | 9140 | 6285 |
| | 12449 | 9452 | 8035 | 10356 | 4443 | 10064 | 8138 | 2067 | 3934 | 4275 | 11430 | 9052 |
| | 12318 | 6252 | 12246 | 9405 | 2987 | 9728 | 11326 | 3397 | 9877 | 8474 | 310 | 11058 |
| | 9105 | 6860 | 7715 | 860 | 2342 | 1701 | 10402 | 8446 | 4050 | 6973 | 3986 | 11870 |
| | 12047 | 4332 | 6725 | 1902 | 9408 | 4088 | 3842 | 4672 | 10725 | 12181 | 1973 | 10224 |
| | 3950 | 4578 | 862 | 7045 | 11785 | 4789 | 5465 | 8088 | 9992 | 3553 | 10189 | 9964 |
| | 2793 | 6677 | 10001 | 3375 | 4200 | 10391 | 4747 | 206 | 306 | | | |
| 308: | 5804 | 12016 | 10678 | 10712 | 10735 | 7448 | 10738 | 10708 | 12014 | 10638 | 7423 | 7421 |
| | 7417 | 309 | 10586 | 10589 | 10603 | 10584 | 7444 | 10644 | 7446 | 5047 | 10645 | 10646 |
| | 12280 | 207 | 9710 | 2096 | 11839 | 9709 | 1612 | 8993 | 10037 | 8354 | 6780 | 11613 |
| | 9034 | 306 | 307 | 2004 | 11103 | 8166 | 6931 | 7311 | 6922 | 8933 | 3783 | 11857 |
| | 12034 | 3781 | 6666 | 9745 | 12449 | 10356 | 4275 | 12246 | 9405 | 2987 | 2067 | 3934 |
| | 8138 | 11430 | 9052 | 12318 | 6252 | 7223 | 9728 | 410 | 6792 | 9877 | 3397 | 310 |
| | 11058 | 9105 | 8474 | 8446 | 4050 | 6973 | 6860 | 7715 | 860 | 4332 | 6725 | 1902 |
| | 2342 | 1701 | 10402 | 12181 | 9408 | 4088 | 3842 | 10725 | 11870 | 12047 | 3986 | 4672 |
| | 6779 | 3950 | 862 | 7045 | 11785 | 4789 | 5465 | 10224 | 8088 | 3553 | 9992 | 10189 |
| | 9964 | 4578 | 2793 | 6677 | 10001 | 3375 | 4200 | 10391 | 11712 | 4747 | 206 | |
| 309: | 5804 | 12016 | 10678 | 10712 | 10735 | 7448 | 10738 | 10708 | 12014 | 10638 | 7423 | 7421 |
| | 7417 | 10586 | 10603 | 10589 | 10584 | 7444 | 10644 | 7446 | 5047 | 10645 | 10646 | 12280 |
| | 207 | 9710 | 2096 | 11839 | 1612 | 8993 | 10037 | 6780 | 11613 | 9034 | 306 | 307 |
| | 2004 | 8166 | 6931 | 7311 | 6922 | 8933 | 10494 | 3783 | 308 | 12034 | 3781 | 9745 |
| | 12449 | 10356 | 4275 | 2067 | 3934 | 9728 | 12246 | 9405 | 2987 | 8138 | 3564 | 2073 |
| | 4786 | 9877 | 310 | 11058 | 8474 | 9105 | 6779 | 9408 | 4088 | 3842 | 4672 | 6725 |
| | 1902 | 6860 | 7715 | 860 | 2342 | 8446 | 1701 | 4050 | 10402 | 6973 | 4332 | 11870 |
| | 12047 | 3986 | 10725 | 10224 | 862 | 7045 | 11785 | 4789 | 5465 | 8088 | 3553 | 10189 |
| | 9992 | 3950 | 4578 | 1468 | 10676 | 11712 | 10682 | 4747 | 206 | | | |
| 310: | 5804 | 2397 | 12016 | 7448 | 10738 | 10708 | 12014 | 10638 | 7423 | 7421 | 7417 | 309 |
| | 7444 | 7446 | 10645 | 12280 | 207 | 9710 | 3151 | 3170 | 2096 | 11839 | 9709 | 1612 |
| | 8993 | 10037 | 8354 | 6780 | 8499 | 11613 | 1088 | 9034 | 306 | 307 | 2004 | 8166 |
| | 888 | 7311 | 3783 | 308 | 12034 | 7362 | 6666 | 8344 | 7302 | 1234 | 10676 | 10741 |
| | 10660 | 10641 | 10683 | 11712 | 10743 | 10575 | 10581 | 10682 | 4747 | 206 | 8474 | 9105 |
| 311: | 10915 | 10878 | 1068 | 12529 | 8032 | 413 | 5270 | 2499 | 8936 | 5781 | 4938 | 5276 |
| | 1252 | 892 | 4567 | | | | | | | | | |
| 312: | 8987 | 9785 | 7079 | 7212 | 7143 | 1975 | 6697 | 2771 | 8204 | 12195 | 8537 | 585 |
| | 2367 | 8528 | 2778 | 10971 | 5848 | 3923 | 2481 | 10194 | 10765 | 6901 | | |
| 313: | 9794 | 11709 | 6934 | 6911 | 3465 | 11447 | 8784 | 2016 | 8641 | 5611 | 8517 | 8671 |
| | 3003 | 7544 | 6479 | 6231 | 563 | 8829 | 10089 | 2790 | 628 | 627 | 9649 | 1297 |
| | 11694 | 5577 | 8598 | 9675 | 10148 | 4363 | 3836 | 4733 | 1188 | 3242 | 5221 | 11035 |
| | 2914 | 8480 | 11016 | 4957 | 7466 | 6072 | 7775 | 10813 | 3443 | 7004 | 1031 | 12329 |
| | 2384 | 8400 | 3441 | 6113 | 1400 | 10790 | 3796 | 7441 | 1736 | 6771 | 5928 | 2870 |
| | 5451 | 8104 | 10128 | 10965 | 2791 | 9293 | 11843 | 11570 | 8042 | 1481 | 6529 | 11667 |
| | 866 | 7292 | 6602 | | | | | | | | | |
| 314: | 12162 | 11631 | 3039 | 3335 | 1817 | 6144 | 11507 | 11509 | 10668 | 9931 | 7389 | 11289 |
| | 3607 | 1332 | 7818 | 7488 | 11660 | 11565 | 11685 | 11567 | 11688 | 10617 | 5147 | 2492 |
| | 1968 | 10962 | 3770 | 7759 | 8253 | 7833 | 2978 | 10138 | 9767 | 3688 | 11601 | 11695 |
| | 4434 | 10033 | 407 | 4317 | 10372 | 540 | 8736 | 5135 | 6797 | 470 | 12257 | 7890 |
| | 2547 | 9902 | 8793 | 1833 | 11410 | 5625 | 3866 | 10200 | 2909 | 10621 | 5905 | 11153 |
| | 769 | 6607 | 7608 | 11014 | 10403 | 1714 | 1985 | 8277 | 11535 | 11659 | 11637 | 12345 |
| | 12301 | 11505 | 11530 | 11656 | 11525 | 11606 | 12307 | 11634 | 12340 | | | |
| 315: | 712 | 11134 | 11581 | 8135 | 891 | 5866 | 7918 | 1721 | 10277 | 4963 | 7671 | 9567 |
| | 512 | 10630 | 6458 | 3683 | 4951 | 8419 | 4724 | 5955 | 3114 | 1441 | 4439 | 8965 |
| | 11628 | 9774 | 5184 | 10737 | 9308 | 6612 | 10159 | 5721 | 6759 | 12507 | 2586 | 7299 |
| | 10335 | 9387 | 5504 | 6758 | 9577 | 5043 | 9868 | 6733 | 9951 | 5843 | 11373 | 4090 |
| | 3481 | 2186 | 10450 | 10709 | 11672 | 6568 | 11882 | 5745 | 9469 | 9268 | 11804 | 10475 |
| | 11196 | 6964 | 2129 | 10514 | 7931 | 8436 | 1370 | 7659 | 6865 | 1238 | 3006 | 635 |
| | 7240 | 5350 | 6661 | 12111 | 9988 | 1853 | 1109 | 2594 | 12193 | 12188 | 1329 | 10264 |
| | 11716 | 4872 | 347 | 4739 | 7259 | 3909 | 3596 | 6541 | 2688 | 2146 | 5708 | 10807 |
| | 10781 | 10784 | 10777 | 9045 | 8986 | 8786 | 9839 | 11901 | 1929 | 1933 | 1935 | 10650 |
| | 10654 | 7773 | 7801 | 7804 | 1143 | 2054 | 6917 | 1476 | 10477 | 4712 | 1561 | 10094 |
| | 1078 | 1684 | 10485 | 2080 | 10545 | 2225 | 11028 | 8886 | 9355 | 2815 | 2957 | 3147 |
| | 7080 | 5087 | 2577 | 11228 | 10228 | 4716 | 4719 | 8219 | 1623 | 5563 | 11698 | 4688 |
| | 6802 | 7656 | 7655 | 11357 | 11544 | 11574 | 12191 | 9823 | 707 | 4304 | 11582 | 11993 |
| | 11988 | 11991 | 12562 | 11182 | 11164 | 11162 | 10876 | 11184 | 2858 | 1541 | 9124 | 7770 |
| | 11817 | 2155 | 12468 | 8830 | 10980 | 5410 | 8443 | 6061 | 2736 | 11463 | 8780 | 9125 |
| | 11997 | 935 | 8250 | 9251 | 4203 | 9291 | 6598 | 4948 | 6065 | 2047 | 10171 | 7719 |
| | 6540 | 3804 | 2670 | 3982 | 11540 | 11059 | 3392 | 10297 | 6763 | 9898 | 2286 | 6482 |
| | 1045 | 7087 | 8619 | 7714 | 3117 | 7324 | 7301 | 7454 | 9151 | 10810 | 2165 | 1994 |
| | 11179 | 11370 | 8371 | 7116 | 7222 | 9215 | | | | | | |
| 316: | 10181 | 5990 | 406 | 6910 | 5922 | 9771 | 8885 | 1859 | 574 | 2867 | 532 | 1868 |
| | 6925 | 10421 | 3134 | 12068 | 5705 | 2337 | 12241 | 9985 | 3554 | 11752 | 5564 | 2432 |
| | 2463 | 1750 | 7099 | 9628 | 6813 | 6873 | 1600 | 1443 | 7309 | 12216 | 6438 | 10892 |
| 317: | 2001 | 9411 | 1075 | 7903 | 8973 | 6228 | 11216 | 3706 | 9101 | 10886 | 2855 | 12572 |
| | 10961 | 7464 | 2904 | 10516 | 4510 | 8333 | 8057 | 11958 | 4305 | 6046 | 1672 | 5230 |
| | 11536 | 3762 | 2038 | 11435 | 2899 | 6559 | 5359 | 494 | 6709 | 9645 | 9316 | 8626 |
| | 4344 | 943 | 5569 | 2274 | 12370 | 3372 | 2986 | 9817 | 2723 | 8355 | 5821 | 6502 |
| | 9481 | 6030 | 1829 | 7862 | 8577 | 2818 | 10521 | 2507 | 1904 | 11531 | 2831 | 9309 |
| | 8347 | 3705 | 6837 | 1942 | 529 | 3692 | 884 | 6075 | 11053 | 5261 | 1823 | 5521 |
| | 5620 | 9741 | 5458 | 4209 | 10496 | 3133 | 10883 | 10748 | 11524 | 8416 | 6456 | 9739 |
| | 10729 | 3716 | 1869 | 3943 | 10259 | 989 | 1566 | 9414 | 5570 | 3969 | 2061 | 12023 |
| | 4834 | 7329 | 1599 | 12133 | 1298 | 997 | 3452 | 3409 | 7876 | 500 | 6317 | 10340 |
| | 9615 | 5619 | 2025 | 11396 | 5304 | 1410 | 5125 | 11354 | 7934 | 11109 | 10205 | 11465 |
| | 10315 | 7718 | 3708 | 12353 | 7369 | 4198 | 11754 | 9213 | 720 | 2565 | 4404 | 8816 |
| | 2763 | 9750 | 3487 | 2107 | 2416 | 5876 | 1811 | 10553 | 2898 | 1699 | 11813 | 9078 |
| | 839 | 6693 | 3694 | 2275 | 8945 | 6825 | 429 | 10377 | 3278 | 10269 | 9851 | 4308 |
| | 3366 | 5469 | 4158 | 11942 | 10539 | 11281 | 4989 | 10437 | 11448 | 11023 | 4266 | 1399 |
| | 5475 | 1474 | 3089 | 8937 | 752 | 8129 | 9624 | 603 | 6938 | 4302 | 6071 | 5887 |
| | 8953 | 9754 | 11068 | 6539 | 11602 | 11856 | 11773 | | | | | |
| 318: | 1808 | 1810 | 1809 | 1815 | 8197 | 7925 | 3733 | 9562 | 9559 | 8735 | 9493 | 11825 |
| | 7039 | 2571 | 4406 | 1717 | 4426 | 11479 | 5470 | 5490 | 5468 | 12179 | 12073 | 9263 |
| | 1882 | 6962 | 8737 | 5554 | 454 | 6331 | 9197 | 7474 | | | | |
| 319: | 1608 | 435 | 1810 | 1809 | 1808 | 8197 | 9562 | 9559 | 8735 | 9493 | 4045 | 7039 |
| | 2571 | 4406 | 1717 | 4426 | 11479 | 5470 | 5490 | 5468 | 6962 | 8737 | 5554 | 10895 |
| | 10799 | 10893 | 10845 | 10872 | 10869 | 10868 | 1277 | 1036 | 7857 | | | |
| 320: | 11284 | 3835 | 1257 | 7050 | 4894 | 995 | 10631 | 1387 | 3315 | 5206 | 2848 | 2458 |
| | 5893 | | | | | | | | | | | |
| 321: | 1992 | 7276 | 11661 | 3904 | 3713 | 8363 | 12462 | 7790 | 8181 | 11322 | 1115 | 2876 |
| | 4777 | 11426 | 5331 | 3812 | 11811 | 10448 | 730 | 1688 | 10292 | 8788 | 10266 | 7189 |
| | 7105 | 5338 | 7555 | 6641 | 12172 | 10702 | 3961 | 2554 | 10739 | 3771 | 10525 | 11295 |
| | 10175 | 3346 | 9422 | 3617 | 1340 | 3652 | 8834 | 1785 | 10107 | 6885 | 9797 | 2092 |
| | 7460 | 8036 | 1518 | 4343 | 9541 | 10966 | 8739 | 7343 | 7970 | 1357 | 7692 | |
| 322: | 7998 | 11945 | 3324 | 2518 | 2605 | 12395 | 12415 | 6120 | 4228 | 1728 | 6481 | 9287 |
| | 8237 | 7203 | 4544 | 11071 | 6485 | 7597 | 3710 | 4975 | 7397 | 5640 | 5151 | 10017 |
| | 11392 | 8033 | 6793 | 5938 | 12275 | 2260 | 11004 | 9055 | 4389 | 391 | 2053 | 9786 |
| | 5557 | 7782 | 1275 | | | | | | | | | |
| 323: | 2163 | 12492 | 12513 | 2433 | 12362 | 5815 | 6948 | 1690 | 4436 | 11925 | 10565 | 3671 |
| | 12009 | 5590 | 2338 | | | | | | | | | |
| 324: | 2163 | 12492 | 12513 | 2433 | 12362 | 5815 | 6948 | 1690 | 4436 | 11925 | 10565 | 3671 |
| | 12009 | 5590 | 2338 | | | | | | | | | |
| 325: | 5223 | 7733 | 3595 | 6743 | 8171 | 1884 | 4936 | 7342 | 544 | 11333 | 4021 | 10203 |
| | 1099 | 788 | 10465 | 1185 | 6579 | 12206 | 11278 | 12277 | 7727 | 3862 | 11362 | 6398 |
| | 11319 | 5259 | 1270 | 11110 | 3045 | 511 | 3792 | 5551 | | | | |
| 326: | 4483 | 7102 | 12574 | 11511 | 5281 | | | | | | | |
| 327: | 5332 | 5927 | 4548 | 5709 | 11543 | 8482 | 3822 | 5041 | 9863 | 838 | 4020 | 11868 |
| | 5102 | 9911 | 4642 | 10599 | 1012 | 6488 | 4691 | 9525 | 1062 | 7489 | 6816 | 2714 |
| | 8445 | | | | | | | | | | | |
| 328: | 11721 | 5605 | 2086 | 4366 | 1269 | 3758 | 7461 | 3682 | 4125 | 10030 | 11534 | 7537 |
| | 8615 | 11301 | 4300 | 9716 | 7707 | 7358 | 12567 | 10049 | 7574 | 2467 | 8459 | 7101 |
| | 2732 | 4569 | 7261 | 7972 | 12098 | 982 | 5952 | 3290 | 953 | 12385 | 11696 | 9296 |
| | 5713 | 11495 | 9981 | 6322 | 6795 | 8719 | 8387 | 6978 | 9584 | 7285 | 810 | 8024 |
| | 596 | 10008 | 7197 | 4265 | 3603 | 4391 | 9425 | 12253 | 390 | 9350 | 6547 | 3679 |
| | 7413 | 11860 | 8874 | 476 | 4966 | 11483 | 10611 | 5073 | 8720 | 6724 | 8818 | 4326 |
| | 8743 | | | | | | | | | | | |
| 329: | 2805 | 11466 | 10817 | 12063 | 4835 | 12061 | 4262 | 10839 | 12227 | 4562 | 4636 | 4607 |
| | 4586 | 4609 | 3893 | 791 | 12164 | 1352 | 7322 | 5134 | 2955 | 2878 | 5825 | 10270 |
| | 10747 | 6822 | 6782 | 10822 | 7599 | 8525 | 8453 | 2918 | 1122 | 11630 | 4524 | 9768 |
| | 6459 | 5336 | 11488 | 10000 | 1807 | 9627 | 2250 | 5726 | 2230 | 3561 | 10425 | 1547 |
| | 3423 | 6893 | 7821 | 7482 | 4962 | 1100 | 1663 | 10881 | 6320 | 8875 | 1487 | 3299 |
| | 6493 | 7820 | 973 | 4224 | 6789 | 7146 | 11806 | 10590 | 8431 | 11300 | 1913 | 9775 |
| | 7743 | 2113 | 3428 | 5019 | 11401 | 5529 | 1903 | 7592 | 3655 | 8165 | 11845 | 5638 |
| | 6858 | 11376 | 7403 | 824 | 7304 | 8472 | 3295 | 8136 | 7078 | 2556 | 2691 | 4804 |
| | 4179 | 11583 | 11575 | 9102 | 11213 | 3074 | 11867 | 3761 | 9059 | 4156 | 6311 | 10457 |
| | 12201 | 3400 | 662 | 7056 | 8999 | 9787 | 1318 | 7425 | 1235 | 9162 | 462 | 11826 |
| | 2887 | 1846 | 8604 | 871 | 9612 | 9641 | 8005 | 6947 | 8418 | 6650 | 8835 | 2217 |
| | 1607 | 587 | 4853 | 4851 | 2261 | 11405 | 5131 | 3440 | 3463 | 8753 | 7505 | 1693 |
| | 2532 | 7018 | 7816 | 2862 | 2859 | 5792 | 2825 | 11560 | 2464 | 7947 | 11222 | 8549 |
| | 250 | 249 | 1118 | 1532 | 12578 | 6821 | 1665 | 5514 | 5491 | 5464 | 5495 | 5457 |
| | 5437 | 5435 | 4797 | 3327 | 2871 | 1883 | 1886 | 8888 | 3837 | 11290 | 3378 | 9236 |
| | 3257 | 8982 | 7124 | 9026 | 11772 | 12359 | 12060 | 9068 | 9120 | 5737 | 9856 | 6932 |
| | 5948 | 7673 | 4368 | 9281 | 2134 | 3824 | 10371 | 2540 | 4223 | 2694 | 10633 | 2639 |
| | 11261 | 9978 | 5068 | 7848 | 774 | 7835 | 5894 | 6079 | 11670 | 8244 | 10750 | 10141 |
| | 2381 | 9058 | 6257 | 6588 | 3394 | 6012 | 1778 | 11268 | 11462 | 8660 | 2158 | 12387 |
| | 10746 | 9187 | 4432 | 7406 | 10647 | 1146 | 7316 | 6100 | 1698 | 4358 | 879 | 1727 |
| | 12205 | 7170 | 12545 | 6117 | 10722 | 12516 | 7294 | 12005 | 11165 | 1857 | 12042 | 6879 |
| | 3473 | 4353 | 2798 | 10096 | 2424 | 7595 | 9288 | 3396 | 4644 | 7051 | 6749 | 4932 |
| | 1417 | 11847 | 8676 | 2675 | 3149 | 4493 | 8041 | 2231 | 10890 | 2301 | 3232 | 9139 |
| | 3530 | 7139 | 7836 | 3718 | 12250 | 881 | 2373 | 3614 | 4126 | 9707 | 5779 | 9630 |
| | 2905 | 11956 | 10260 | 8176 | 8370 | 1834 | 1301 | 5330 | 2572 | 10162 | 3878 | 8872 |
| | 7166 | 12030 | 9648 | 2689 | 10149 | 10973 | 9109 | 4471 | 11920 | 685 | 6584 | 2239 |
| | 10680 | 5472 | 5665 | 3253 | 2889 | 12470 | 8726 | 4199 | 6034 | 6623 | 4362 | 12405 |
| | 6411 | 10429 | 12497 | 9570 | 1214 | 1030 | 617 | 829 | 10859 | 11229 | 1603 | 3932 |
| | 1255 | 12084 | 10987 | 900 | 11188 | 2018 | 616 | 10866 | 6138 | 5522 | 1003 | 7084 |
| | 10459 | 1656 | 9217 | 4190 | 10206 | 4395 | 11017 | 2310 | 3027 | 3199 | 8196 | 12409 |
| | 1333 | 1930 | 7140 | 8939 | 3081 | 5939 | 1360 | 6958 | 10063 | 5908 | 2324 | 5587 |
| | 735 | 1173 | 7035 | 8162 | 10945 | 7665 | 8546 | 4249 | 8439 | 4095 | 10156 | 10177 |
| | 10176 | 931 | 2302 | 2377 | 8634 | 420 | 11000 | 2894 | 3098 | 4069 | 5285 | 1311 |
| | 9272 | 6242 | 7611 | 3944 | 3947 | 5722 | 5719 | 5695 | 5720 | 5753 | 5727 | 5731 |
| | 12515 | 9999 | 6710 | 9394 | 9395 | 4022 | 4024 | 10165 | 3990 | 10801 | 3995 | 10163 |
| | 1553 | 3993 | 11441 | 10846 | 11136 | 7843 | 10491 | 4725 | 11489 | 4723 | 10823 | 5021 |
| | 10914 | 3017 | 7338 | 10498 | 7898 | 10615 | 3506 | 626 | 4799 | 10409 | 11339 | 1805 |
| | 7639 | 10281 | 10034 | 8405 | 11038 | 3492 | 8645 | 12001 | 5800 | 7175 | 4668 | 11480 |
| | 9467 | 3156 | 11075 | 2995 | 7905 | 1423 | 5037 | 7870 | 7748 | 2283 | 9865 | 11865 |
| | 5561 | 8231 | 8097 | 6110 | 8686 | 7829 | 8871 | 5715 | 1804 | 5644 | 4423 | 2259 |
| | 11914 | 11250 | 8485 | 9933 | 716 | 6323 | 6799 | 3509 | 12565 | 7764 | 5263 | 9011 |
| | 6246 | 4504 | 2358 | 8877 | 2182 | 11148 | 10158 | 5262 | 5258 | 4660 | 3899 | 6021 |
| | 10821 | 10978 | 1801 | 8831 | 1936 | 6826 | 9977 | 3546 | 5982 | 3472 | 8460 | 3460 |
| | 12528 | 5242 | 9347 | 8527 | 8657 | 8552 | 7744 | 6286 | 2066 | 9201 | 506 | 6016 |
| | 5456 | 399 | 481 | 11107 | 9066 | 4390 | 6748 | 11573 | 5819 | 5182 | 9176 | 11244 |
| | 8029 | 3399 | 7013 | 10161 | 10468 | 11469 | 5983 | 9301 | 5544 | 7268 | 5412 | 8121 |
| | 3720 | 9353 | 2299 | 6358 | 3224 | 11486 | 2423 | 1330 | 12174 | 3550 | 4953 | 2783 |
| | 11429 | 8828 | 8602 | 1300 | 9104 | 7287 | 8658 | 1986 | 5677 | 8336 | 12262 | 3449 |
| | 4980 | 9514 | 7606 | 2388 | 11771 | 10820 | 1780 | 11551 | 2392 | 10995 | 9489 | 2737 |
| | 8454 | 10493 | 975 | 1824 | 7713 | 12036 | 7172 | 8395 | 6062 | 4409 | 8222 | 5608 |
| | 5031 | 12363 | 11455 | 2661 | 2189 | 5632 | 6561 | 3215 | 11858 | 2652 | 9866 | 8620 |
| | 11816 | 2701 | 10674 | 568 | 11663 | 11649 | 9706 | 12317 | 12525 | 2916 | 8590 | 7129 |
| | 9310 | 11073 | 7694 | 1472 | 5767 | 7178 | 8963 | 4159 | 3109 | 8186 | 2200 | 8688 |
| | 5114 | 9659 | 6461 | 6519 | 5873 | 668 | 11777 | 10392 | 4625 | 10853 | 6210 | 3001 |
| | 4310 | 1966 | 1885 | 7538 | 5702 | 8513 | 9937 | 10901 | 11642 | 2005 | 4671 | 9435 |
| | 3667 | 4480 | 5740 | 6785 | 4971 | 10956 | 8452 | 11464 | 10848 | 11438 | 10851 | 10769 |
| | 10766 | 10764 | 10763 | 3757 | 4574 | 11739 | 6869 | 8373 | 8666 | 10481 | 9606 | 12245 |
| | 3756 | 3197 | 10861 | 6465 | 3593 | 6775 | 5984 | 599 | 7355 | 6537 | 7494 | 10728 |
| | 12397 | 9341 | 2226 | 6185 | 10343 | 1288 | 4414 | 6983 | 8412 | 11161 | 11579 | 7999 |
| | 8479 | 4489 | 1371 | 4138 | 5186 | 5969 | 6473 | 8915 | 8236 | 2240 | 2531 | 11898 |
| | 4870 | 5512 | 1564 | 12165 | 5524 | 8233 | 5143 | 5631 | 5607 | 4034 | 2935 | 2934 |
| | 12500 | 5110 | 8078 | 5324 | 762 | 823 | 10079 | 11491 | 8938 | 11159 | 11132 | 6131 |
| | 11124 | 11129 | 11122 | 11440 | 6280 | 2750 | 8169 | 7635 | 7631 | 5372 | 5348 | 9233 |
| | 9231 | 9221 | 9240 | 9235 | 9220 | 2122 | 3029 | 7040 | 12560 | 7016 | 8158 | 8759 |
| | 8762 | 8163 | 7037 | 7822 | 8767 | 7066 | 7061 | 11467 | 942 | 6314 | 7384 | |
| 330: | 12032 | 12416 | 9702 | 8311 | 9871 | 9085 | 3041 | 8757 | 2659 | 10930 | 4428 | 11833 |
| | 11792 | 681 | 10543 | 7911 | 9410 | 8467 | 8008 | 9867 | 1772 | 8996 | 7033 | 11906 |
| | 10828 | 7889 | 12234 | 1084 | 3276 | 8149 | 11266 | 8572 | 1344 | 7106 | 7107 | 4195 |
| | 4547 | 11422 | 9561 | 4650 | 4648 | 2144 | 2142 | 2148 | 4680 | 4656 | 4653 | 633 |
| | 5402 | 409 | 6305 | 10014 | 6859 | 12364 | 7575 | 5289 | 3907 | 7452 | 3669 | 11548 |
| | 2333 | 4742 | 6830 | 2981 | | | | | | | | |
| 331: | 10160 | 10640 | 8343 | 4802 | 1528 | 8856 | 515 | 677 | 952 | 7450 | 6251 | 4755 |
| | 8449 | 10473 | 7390 | 6576 | 11718 | 2615 | 7103 | 10211 | 10977 | 4800 | 4314 | 7867 |
| | 9639 | 7932 | 11248 | 3355 | 7617 | 2525 | 10518 | 5743 | 1589 | 8208 | 9668 | 5774 |
| | 5974 | 5814 | 2961 | 9399 | 11092 | 5321 | 5264 | 1080 | 6145 | 7689 | 3942 | 7442 |
| | 2483 | 11748 | 12331 | 12214 | 5980 | 5480 | 7320 | 5440 | 10776 | 4781 | 9666 | 3971 |
| | 7979 | 3468 | 8146 | 4220 | 4413 | 3920 | 5416 | 8390 | 1957 | 5313 | 2735 | 11318 |
| | 4437 | 9259 | 8429 | 11015 | 7585 | 7435 | 4475 | 12123 | 4830 | 7053 | 7352 | 3938 |
| | 7110 | 6593 | 10656 | 966 | 3928 | 10099 | 12555 | 8109 | 11578 | 870 | 5664 | 6989 |
| | 1416 | 12103 | 11181 | 5195 | 3488 | 8294 | 9944 | 970 | 7199 | 2188 | 8851 | 11185 |
| | 6333 | 10583 | 8715 | 828 | 11105 | 2300 | 1272 | 4605 | 6204 | 7241 | 4961 | 932 |
| | 1479 | 3941 | 6155 | 6654 | 2411 | 10311 | 3132 | 10779 | 6695 | 9113 | 4382 | 4370 |
| | 3238 | 9765 | 5394 | 7378 | 8693 | 4023 | 3953 | 8976 | 2263 | 7064 | 10047 | 461 |
| | 9468 | 1101 | 4079 | 4728 | 5158 | 8968 | 12479 | 5828 | 4916 | 9417 | 3032 | 2685 |
| | 9018 | 10135 | 7048 | 897 | 10841 | 6006 | 3968 | 7120 | 6124 | 3102 | 7921 | 2064 |
| | 9239 | 11783 | 2698 | 7415 | 5823 | 5635 | 11864 | 4640 | 593 | 4046 | 5343 | 6406 |
| | 2792 | 8092 | 11513 | 1919 | 11764 | 10026 | 8691 | 2679 | 7600 | 10399 | 2289 | 5567 |
| | 9801 | 2125 | 6284 | 9433 | 2993 | 427 | 11366 | 3581 | 3097 | 4014 | 12532 | 10913 |
| | 8020 | 4751 | 4316 | 6734 | 1442 | 674 | 6025 | 5541 | 2179 | 7168 | 12170 | 805 |
| | 3080 | 12469 | 11468 | 11287 | 5842 | 3282 | 9440 | 4309 | 12511 | 12508 | 2063 | 10517 |
| | 6891 | 9594 | 11087 | 4606 | 8366 | 12400 | 7712 | 4288 | 6764 | | | |
| 332: | 10160 | 10640 | 8343 | 4802 | 1528 | 8856 | 515 | 677 | 952 | 7450 | 6251 | 4755 |
| | 8449 | 10473 | 7390 | 6576 | 11718 | 2615 | 7103 | 10211 | 10977 | 4800 | 4314 | 7867 |
| | 9639 | 7932 | 11248 | 3355 | 7617 | 2525 | 10518 | 5743 | 1589 | 8208 | 9668 | 5774 |
| | 5974 | 5814 | 2961 | 9399 | 11092 | 5321 | 5264 | 1080 | 6145 | 7689 | 3942 | 7442 |
| | 2483 | 11748 | 12331 | 12214 | 5980 | 5480 | 7320 | 5440 | 10776 | 4781 | 9666 | 3971 |
| | 7979 | 3468 | 8146 | 4220 | 4413 | 3920 | 5416 | 8390 | 1957 | 5313 | 2735 | 11318 |
| | 4437 | 9259 | 8429 | 11015 | 7585 | 7435 | 4475 | 12123 | 4830 | 7053 | 7352 | 3938 |
| | 7110 | 6593 | 10656 | 966 | 3928 | 10099 | 12555 | 8109 | 11578 | 870 | 5664 | 6989 |
| | 1416 | 12103 | 11181 | 5195 | 3488 | 8294 | 9944 | 970 | 7199 | 2188 | 8851 | 11185 |
| | 6333 | 10583 | 8715 | 828 | 11105 | 2300 | 1272 | 4605 | 6204 | 7241 | 4961 | 932 |
| | 1479 | 3941 | 6155 | 6654 | 2411 | 10311 | 3132 | 10779 | 6695 | 9113 | 4382 | 4370 |
| | 3238 | 9765 | 5394 | 7378 | 8693 | 4023 | 3953 | 8976 | 2263 | 7064 | 10047 | 461 |
| | 9468 | 1101 | 4079 | 4728 | 5158 | 8968 | 12479 | 5828 | 4916 | 9417 | 3032 | 2685 |
| | 9018 | 10135 | 7048 | 897 | 10841 | 6006 | 3968 | 7120 | 6124 | 3102 | 7921 | 2064 |
| | 9239 | 11783 | 2698 | 7415 | 5823 | 5635 | 11864 | 4640 | 593 | 4046 | 5343 | 6406 |
| | 2792 | 8092 | 11513 | 1919 | 11764 | 10026 | 8691 | 2679 | 7600 | 10399 | 2289 | 5567 |
| | 9801 | 2125 | 6284 | 9433 | 2993 | 427 | 11366 | 3581 | 3097 | 4014 | 12532 | 10913 |
| | 8020 | 4751 | 4316 | 6734 | 1442 | 674 | 6025 | 5541 | 2179 | 7168 | 12170 | 805 |
| | 3080 | 12469 | 11468 | 11287 | 5842 | 3282 | 9440 | 4309 | 12511 | 12508 | 2063 | 10517 |
| | 6891 | 9594 | 11087 | 4606 | 8366 | 12400 | 7712 | 4288 | 6764 | | | |
| 333: | 1001 | 4906 | 8800 | 3344 | 2015 | 2020 | 4318 | 7710 | 10721 | 2563 | 3717 | 1228 |
| | 4603 | 9718 | 1317 | 2819 | 8797 | 6896 | 9688 | 6275 | 6022 | 6023 | 6010 | 1908 |
| | 12491 | 3275 | 4299 | 8601 | 4383 | 7942 | 5268 | 10515 | 3747 | 558 | 7924 | 8917 |
| | 7861 | 7946 | 3744 | 1396 | 12348 | 7923 | 10606 | 2285 | 5758 | 7667 | 5463 | 5546 |
| | 7922 | 5549 | 12374 | 2282 | 4005 | 11378 | 11112 | 7984 | 8811 | 12299 | 4713 | 3333 |
| | 5889 | 7864 | 7940 | 1761 | 11398 | 3821 | 2238 | 4004 | 12159 | 9680 | 8705 | 9007 |
| | 3972 | 10492 | 1765 | 8840 | 7860 | 1405 | 6159 | 11243 | 3817 | 1502 | 4711 | 9800 |
| | 3945 | 8082 | 664 | 4003 | 6437 | 7684 | 4102 | 3973 | | | | |
| 334: | 1523 | 2933 | 4956 | 6234 | 6928 | 5998 | 10172 | 7536 | 3631 | 3520 | 11142 | 5900 |
| | 1996 | 8969 | 9226 | 7768 | 10920 | 10831 | 3174 | 10390 | 6829 | 6832 | 8422 | 3388 |
| | 1617 | 5714 | 4375 | 10999 | 8464 | 2255 | 5214 | 4427 | 1955 | 4279 | 11633 | 594 |
| | 3168 | 10884 | 6049 | 6631 | 6194 | 6223 | 5917 | 1368 | 1418 | 1351 | 5670 | 5697 |
| | 1491 | 7354 | 7180 | 7371 | 6553 | 6011 | 928 | 10117 | 12297 | 3664 | 5929 | 4043 |
| | 10992 | 1144 | 3217 | 7021 | 5189 | 4788 | 7603 | 5872 | 1107 | 10072 | 6527 | 5109 |
| | 4881 | 9852 | 4108 | 7739 | 6628 | 8123 | 1613 | 4323 | 7377 | 1697 | 9620 | 11880 |
| | 7781 | 3078 | 12259 | 3551 | 12426 | 10058 | 8744 | 1061 | 5971 | 584 | 4219 | 12527 |
| | 954 | 11627 | 2114 | 1581 | 4135 | 3267 | 8356 | 9499 | 5531 | 7817 | 653 | 5065 |
| | 6344 | 8377 | 12512 | 10041 | 10082 | 9401 | 6854 | 3502 | 5742 | 5954 | 4072 | 6097 |
| | 3479 | 7398 | 11510 | 2184 | 11738 | 9872 | 6915 | 6429 | 1743 | 9416 | 9586 | 5241 |
| | 9345 | 2234 | 6173 | 3646 | 4997 | 10420 | 10452 | 10367 | 5827 | 8145 | 11490 | 8669 |
| | 4217 | 10730 | 2926 | 9796 | 6632 | 5532 | 4988 | 10910 | 8890 | 9229 | 10771 | 12439 |
| | 6960 | 5406 | 1309 | 7523 | 7626 | 4222 | 6737 | 8760 | 1394 | 2420 | 1149 | 7297 |
| | 8017 | 7952 | 5653 | 1150 | 738 | 4811 | 6665 | 8704 | 6846 | 5297 | 11123 | 11135 |
| | 11027 | 11963 | 4709 | 11761 | 3739 | 2835 | 10287 | 6587 | 9001 | 1595 | 1283 | 9529 |
| | 8087 | 10291 | 8801 | 8105 | 7255 | 2212 | 8972 | 5764 | 10625 | 3266 | 1263 | 4582 |
| | 1874 | 10424 | 10061 | 6256 | 2767 | 6798 | 11099 | 7366 | 6400 | 2490 | 890 | 1425 |
| | 9601 | 2202 | 4027 | 2609 | 8582 | 2990 | 3014 | 11219 | 545 | 1065 | 2693 | 3848 |
| | 11452 | 768 | 7539 | 6889 | 5039 | 3505 | 11776 | 4037 | 10554 | 2960 | 5723 | 8927 |
| | 2765 | 6091 | 11885 | 2884 | 8635 | 1971 | 800 | 7345 | 6787 | 9790 | 8855 | 2143 |
| | 10288 | 4846 | 12471 | 3707 | 4833 | 11065 | 8698 | 10819 | 2897 | 1946 | 1486 | 3365 |
| | 4763 | 2908 | 8774 | 3723 | 9111 | 8259 | 8421 | 8420 | 1073 | 1243 | 1671 | 2441 |
| | 5303 | 941 | 11089 | 1314 | 10431 | 4320 | 598 | 6379 | 7019 | 10113 | 10060 | 4886 |
| | 8623 | 840 | 8916 | 8438 | | | | | | | | |
| 335: | 2809 | 1347 | 3070 | 5519 | 6353 | 4683 | 10278 | 7995 | 11147 | 4915 | 4817 | 7113 |
| | 11420 | 1517 | 6416 | 864 | 2738 | 9453 | 8524 | 6360 | 1213 | 5208 | 9694 | 8994 |
| | 10342 | 4151 | 9222 | 923 | 12313 | 3570 | 12419 | 12544 | 5536 | 8592 | 12258 | 1951 |
| | 2817 | 1282 | 3183 | 945 | 10906 | 11904 | 5014 | 9906 | 9939 | 9963 | 1814 | 12017 |
| | 11152 | 11151 | 10075 | 10440 | 8950 | 4508 | 1999 | 4019 | 10028 | 5341 | 5555 | 6544 |
| | 4470 | 5111 | 2582 | 5095 | 8529 | 9270 | 10532 | 2603 | 6266 | 5183 | 10616 | 12434 |
| | 8392 | 9934 | 8028 | 11427 | 6678 | 10220 | 9984 | 10875 | 11154 | 2840 | 1899 | 1022 |
| | 10969 | 3615 | 6536 | 4357 | 11961 | 3073 | 9159 | 4192 | 8746 | 6211 | 7321 | 6531 |
| | 7520 | 7943 | 11246 | 661 | 8912 | 7676 | 3024 | 8161 | 6508 | 3996 | 5054 | 10071 |
| | 12039 | 2298 | 9149 | 9126 | 9123 | 9751 | 9200 | 7546 | 2175 | 9419 | 9670 | 3977 |
| | 8562 | 7711 | 4388 | 11106 | 10681 | 1738 | 3580 | 7344 | 5299 | 8466 | 1572 | 1735 |
| | 543 | 562 | 9152 | 9147 | 2211 | 2353 | 11077 | 8007 | 12109 | 11282 | 8761 | 1917 |
| | 10666 | 2558 | 2560 | 9258 | 6729 | 10691 | 10570 | 8130 | 10658 | 11009 | 1060 | 2523 |
| | 12128 | 11669 | 1851 | 3077 | 10021 | 8747 | 3062 | 7751 | 8521 | 8185 | 10968 | 7149 |
| | 4920 | 5826 | 9879 | 6009 | 4918 | 6638 | 6614 | 4899 | 6609 | 9959 | 9996 | 6005 |
| | 9882 | 6007 | 9936 | 9970 | 9880 | 9915 | 9993 | 4923 | 5794 | 9941 | 6610 | 9912 |
| | 9935 | 9962 | 9991 | 6637 | 9908 | 9957 | 9998 | 4974 | 5931 | 5857 | 5830 | 5852 |
| | 5865 | 5903 | 5833 | 5862 | 5007 | 11889 | 5093 | 5096 | 5098 | 4978 | 4892 | 6002 |
| | 5934 | 5788 | 5751 | 5785 | 5958 | 5985 | 5981 | 5822 | 6640 | 6671 | 6643 | 6645 |
| | 5227 | 12542 | 2875 | 1909 | 2273 | 10016 | 12407 | 9117 | 9178 | 9210 | 9182 | 9211 |
| | 1910 | 9186 | 9184 | 9148 | 12431 | 8650 | 2612 | 6985 | 991 | 8849 | 3562 | 3545 |
| | 10129 | 11329 | 3522 | 3568 | 3590 | 422 | 3516 | 8470 | 3541 | 3587 | 3515 | 3566 |
| | 1261 | 11303 | 11387 | | | | | | | | | |
| 336: | 1608 | 435 | 1809 | 1808 | 1819 | 1810 | 1815 | 9824 | 7252 | 398 | 2455 | 9562 |
| | 9559 | 9493 | 5003 | 10119 | 12283 | 9948 | 9836 | 733 | 7039 | 4426 | 11479 | 5470 |
| | 5490 | 5468 | 8773 | 3533 | 7619 | 7474 | 9197 | 6331 | 8737 | 6962 | 6914 | 5554 |
| | 10872 | 10869 | 10868 | 10799 | 10895 | 10845 | 10893 | 3095 | 11702 | 12076 | | |
| 337: | 6292 | 6296 | 2925 | 9234 | 4245 | 4030 | 8504 | 3729 | 1278 | 1276 | 2356 | 11921 |
| | 6564 | 9267 | 7681 | 6571 | 6315 | 4473 | 7249 | 6569 | 1545 | 1549 | 8359 | 8695 |
| | 6567 | 8697 | 1550 | 6294 | 5078 | 11515 | 1009 | 9626 | 6313 | 8442 | | |
| 338: | 9269 | 4225 | 5257 | 2039 | 4327 | 4244 | 3256 | 9569 | 11115 | 2874 | 10078 | 11414 |
| | 2797 | 1571 | 3740 | 5525 | 4757 | 2614 | 4167 | 2496 | 7866 | 1249 | 5672 | 2395 |
| | 4801 | 11979 | 7313 | 3495 | 4528 | 7535 | 5017 | 5728 | 2539 | 3381 | 9306 | 1978 |
| | 4272 | 8853 | 5847 | 7159 | 9691 | 3906 | 6563 | 1506 | | | | |
| 339: | 9269 | 4225 | 5257 | 2039 | 4327 | 4244 | 3256 | 9569 | 11115 | 2874 | 10078 | 11414 |
| | 2797 | 1571 | 3740 | 5525 | 4757 | 2614 | 4167 | 2496 | 7866 | 1249 | 5672 | 2395 |
| | 4801 | 11979 | 7313 | 3495 | 4528 | 7535 | 5017 | 5728 | 2539 | 3381 | 9306 | 1978 |
| | 4272 | 8853 | 5847 | 7159 | 9691 | 3906 | 6563 | 1506 | | | | |
| 340: | 9784 | 5190 | 4849 | 6165 | 8502 | 4479 | 10406 | 10268 | 5977 | | | |
| 341: | 9784 | 5190 | 4849 | 6165 | 8502 | 4479 | 10406 | 10268 | 5977 | | | |
| 342: | 647 | 2051 | 8014 | 3393 | 10620 | 7556 | 1770 | 6506 | 9947 | 5875 | 5960 | 5682 |
| | 2115 | 7965 | 1026 | 4342 | 9448 | 12338 | 2530 | 2052 | 2057 | 9802 | 4635 | 6263 |
| | 6250 | 6265 | 648 | 7349 | 7090 | 4593 | 1421 | 11800 | 7799 | 10710 | 6823 | 12510 |
| | 6930 | 3206 | 10032 | | | | | | | | | |
| 343: | 6115 | 8689 | 3274 | 2618 | 8107 | 4944 | 6200 | 8541 | 802 | 2608 | 8397 | 6422 |
| | 7563 | 3305 | 10923 | 2865 | 2164 | 4187 | 4295 | 4903 | 10152 | 6888 | 2980 | 11640 |
| | 4652 | 592 | 4424 | 7353 | 9920 | 9891 | 11011 | 11040 | 1231 | 8681 | 10778 | 1227 |
| | 8900 | 4843 | 9156 | 2011 | 1415 | 8510 | 869 | 3034 | 3801 | 11645 | 10469 | 10547 |
| | 455 | 693 | 2502 | 3309 | 12029 | 12320 | 1471 | 7941 | 1465 | 11436 | 4387 | 1876 |
| | 7757 | 4591 | 2992 | 12535 | 5012 | 1066 | 5511 | 7220 | 6287 | 9982 | 639 | 5884 |
| 344: | 6115 | 8689 | 3274 | 2618 | 8107 | 4944 | 6200 | 8541 | 802 | 2608 | 8397 | 6422 |
| | 7563 | 3305 | 10923 | 2865 | 2164 | 4187 | 4295 | 4903 | 10152 | 6888 | 2980 | 11640 |
| | 4652 | 592 | 4424 | 7353 | 9920 | 9891 | 11011 | 11040 | 1231 | 8681 | 10778 | 1227 |
| | 8900 | 4843 | 9156 | 2011 | 1415 | 8510 | 869 | 3034 | 3801 | 11645 | 10469 | 10547 |
| | 455 | 693 | 2502 | 3309 | 12029 | 12320 | 1471 | 7941 | 1465 | 11436 | 4387 | 1876 |
| | 7757 | 4591 | 2992 | 12535 | 5012 | 1066 | 5511 | 7220 | 6287 | 9982 | 639 | 5884 |
| 345: | 6421 | 3565 | 10219 | 3371 | 7840 | 1723 | 6161 | 8243 | 1865 | 1827 | 11869 | 623 |
| | 11423 | | | | | | | | | | | |
| 346: | 4514 | 1219 | 10858 | 6672 | 3259 | 7266 | 4215 | 8062 | 4171 | 9227 | 9633 | 8002 |
| | 12033 | 8080 | 11367 | 8440 | 6700 | 1911 | 6590 | 9230 | 12194 | 6068 | 7562 | 11521 |
| | 11587 | 4044 | 9133 | 6736 | 1178 | 4773 | 9782 | 5388 | 3122 | 8821 | 6226 | 3789 |
| | 3527 | 8952 | 6047 | 7411 | 5290 | 6426 | 6216 | 10419 | 4816 | 7615 | 2761 | 9079 |
| | 11600 | 4502 | 9850 | 8065 | 6902 | 1436 | 5686 | 10898 | 8152 | 8155 | 4230 | 5104 |
| | 1659 | 7568 | 8560 | 5585 | 5128 | 6679 | 9332 | 9329 | 2095 | 6346 | 6659 | 5701 |
| | 4884 | 9654 | 8034 | 10115 | 8557 | 9587 | 3182 | 7231 | 9717 | 11375 | 6070 | 2404 |
| | 1767 | 7075 | 7455 | 2457 | 5255 | 7959 | 6350 | 5750 | 1414 | 903 | 2341 | 7578 |
| | 10703 | 927 | 2248 | 12132 | 863 | 9883 | 618 | 3678 | 12480 | 8396 | 7847 | 9558 |
| | 11682 | 2469 | 7373 | 10386 | | | | | | | | |
| 347: | 712 | 11134 | 11581 | 8135 | 891 | 5866 | 7918 | 1721 | 10277 | 4963 | 7671 | 9567 |
| | 512 | 10630 | 6458 | 3683 | 8419 | 4724 | 5955 | 3114 | 1441 | 4439 | 11628 | 5184 |
| | 10737 | 9308 | 6612 | 10159 | 5721 | 6759 | 12507 | 7299 | 10335 | 5504 | 6758 | 9577 |
| | 5043 | 4852 | 9868 | 1887 | 6733 | 9951 | 5843 | 11373 | 4090 | 3481 | 10450 | 10709 |
| | 11672 | 6568 | 11882 | 3797 | 5745 | 9469 | 9268 | 11804 | 10475 | 11196 | 6964 | 2129 |
| | 10514 | 1370 | 7659 | 6865 | 3006 | 7240 | 6661 | 9988 | 1853 | 1109 | 2594 | 12193 |
| | 12188 | 1329 | 10264 | 9045 | 8986 | 1929 | 1933 | 1935 | 10650 | 10654 | 7773 | 7801 |
| | 7804 | 10477 | 8219 | 11698 | 7655 | 11574 | 11544 | 11582 | 12562 | 11164 | 10876 | 11182 |
| | 11162 | 11184 | 2858 | 7770 | 11817 | 2155 | 8830 | 5410 | 2736 | 6061 | 6540 | 2047 |
| | 6065 | 10171 | 3804 | 3392 | 10297 | 3117 | 315 | 8052 | 9815 | 7301 | 7324 | 7454 |
| | 9151 | 2165 | 1994 | 11179 | 11370 | 7116 | 7222 | 9215 | | | | |
| 348: | 2559 | 10307 | 12499 | 8094 | 9328 | 612 | 11307 | 6139 | 3619 | 3556 | 2873 | 5711 |
| | 1391 | 1177 | 10048 | 10133 | | | | | | | | |
| 349: | 2559 | 10307 | 12499 | 8094 | 9328 | 612 | 11307 | 6139 | 3619 | 3556 | 2873 | 5711 |
| | 1391 | 1177 | 10048 | 10133 | | | | | | | | |
| 350: | 6170 | 10387 | 820 | 4182 | 9177 | 11720 | 535 | 1444 | 1262 | 10488 | 10083 | 6092 |
| | 11526 | 11371 | 6812 | 8205 | 9677 | 11325 | 10718 | 2183 | 531 | 3846 | 7492 | 6927 |
| | 3466 | 4554 | 2717 | 6059 | 7927 | 9446 | 9471 | 9470 | 9476 | 9478 | 9497 | 9473 |
| | 4854 | 7761 | 2526 | 10504 | 7846 | 1389 | 4350 | 1888 | 4581 | 4583 | 4560 | 4564 |
| | 1751 | 8487 | 8489 | 11499 | 9701 | 5856 | 8745 | 2880 | 1803 | 1156 | 3107 | 8710 |
| | 3704 | 3010 | 2932 | 6861 | 2743 | 4442 | 4774 | 3639 | 1620 | | | |
| 351: | 6170 | 10387 | 820 | 4182 | 9177 | 11720 | 535 | 1444 | 1262 | 10488 | 10083 | 6092 |
| | 11526 | 11371 | 6812 | 8205 | 9677 | 11325 | 10718 | 2183 | 531 | 3846 | 7492 | 6927 |
| | 3466 | 4554 | 2717 | 7927 | 9446 | 9471 | 9470 | 9476 | 9497 | 9478 | 9473 | 4854 |
| | 7761 | 2526 | 7846 | 10504 | 1389 | 4350 | 1888 | 4560 | 4583 | 4581 | 4564 | 1751 |
| | 8489 | 8491 | 8487 | 11499 | 9701 | 5497 | 1579 | 5856 | 8745 | 6856 | 6470 | 11959 |
| | 10131 | 827 | 11101 | 1374 | 5291 | 940 | 6870 | 10411 | 4058 | 11708 | 1247 | 2414 |
| | 4133 | 2045 | 6039 | 6478 | 9442 | 3602 | 2111 | 438 | 1381 | 10648 | 6711 | 3008 |
| | 11169 | 8555 | 7810 | 1872 | 10183 | 6946 | 2555 | 12266 | 6834 | 5375 | 7465 | 11021 |
| | 2915 | 644 | 9326 | 3230 | 9185 | 11781 | 6084 | 7725 | 8511 | 12308 | 7518 | 6096 |
| | 8053 | 10529 | 4914 | 11994 | 2145 | 11924 | 1386 | 1880 | 7453 | 1927 | 8819 | 1341 |
| | 2473 | 11742 | 8710 | 3107 | 3704 | 10948 | 9928 | 3010 | 2932 | 6861 | 2743 | 4442 |
| | 4774 | 3639 | 3165 | | | | | | | | | |
| 352: | 2857 | 10800 | 6701 | 7881 | 9041 | 3658 | 3547 | 9249 | 12438 | 6098 | 7560 | 8334 |
| | 2860 | 10885 | 3337 | 6907 | 11049 | 8959 | 5754 | 5088 | 12031 | 10458 | 6468 | 3887 |
| | 3163 | 9860 | 9548 | 487 | 8326 | 1271 | 5539 | 9371 | 9340 | 11769 | 1153 | 11238 |
| | 11180 | 10497 | 10116 | 1083 | 7915 | 11006 | 5436 | 5438 | 5434 | 1342 | 3075 | 9664 |
| | 9905 | 8539 | 2635 | 2390 | 1771 | 7095 | 11918 | 3829 | 6122 | 9096 | 11562 | 7609 |
| | 773 | 12429 | 11502 | 1670 | 1652 | 9013 | 9009 | 9012 | 5992 | 1460 | 3254 | 5360 |
| | 3832 | 8714 | 4271 | 11931 | 11928 | 10964 | 8362 | 12312 | 7680 | 8286 | 9295 | 4400 |
| | 11687 | 9265 | 8142 | 8140 | | | | | | | | |
| 353: | 1313 | 5197 | 12526 | 7194 | 4127 | 10569 | 10567 | 3130 | 11756 | 3924 | 11824 | 5150 |
| | 1597 | 7330 | 7336 | 5733 | 1840 | 8995 | 10023 | 5309 | 9183 | 3811 | 2799 | 3176 |
| | 1007 | 2780 | 10768 | 11917 | 12044 | 4188 | 1537 | 9091 | 4172 | 1215 | 11954 | 5210 |
| | 11001 | 6054 | 396 | 6093 | 789 | 7265 | 10056 | 5622 | 7269 | 2665 | 1409 | 1402 |
| | 5885 | 10020 | 10587 | 2719 | 6657 | 10054 | 6174 | 9953 | 8025 | 2252 | 4474 | 1348 |
| | 5691 | 2034 | 9180 | 3591 | 10519 | 5492 | 4422 | 2710 | 2076 | 8465 | 12482 | 7571 |
| | 11887 | 6428 | 3260 | 11018 | 10323 | 834 | 11437 | 5486 | 604 | 7553 | 388 | 11872 |
| | 9188 | 4081 | 6905 | 11306 | 7202 | 8348 | 5018 | 9023 | 9484 | 9512 | 4553 | 11385 |
| | 2233 | 1463 | 8789 | 7834 | 11916 | 4286 | 2350 | 11883 | 8565 | 1749 | 1023 | 10744 |
| | 4181 | 6476 | 5850 | 1090 | 11086 | 5178 | 2911 | 2349 | 5771 | 610 | 2885 | 5427 |
| | 3735 | 3349 | 649 | 847 | 11740 | 439 | 4459 | 10523 | 846 | 4315 | 7823 | 8415 |
| | 6975 | 9073 | 10143 | 8957 | 2315 | 2292 | 6603 | 1321 | 8167 | 11331 | 9757 | 6755 |
| | 2327 | 2730 | 11943 | 11848 | 5411 | 5103 | 10812 | 8103 | 4922 | 9555 | 12323 | 9526 |
| | 3046 | 4303 | 9846 | 9636 | 10415 | 2376 | 7618 | 9049 | 11863 | 12452 | 9813 | 8070 |
| | 10257 | 3030 | 4031 | 4065 | 1716 | 12281 | 6543 | 5890 | 6492 | 3741 | 8608 | 6220 |
| | 3053 | 9086 | 637 | 6653 | 8593 | 11408 | 12357 | 6247 | 4629 | 2842 | 5476 | 10303 |
| | 2032 | 8190 | 1284 | 12440 | 9307 | 3477 | 2901 | 4407 | 5191 | 10111 | 4218 | 4412 |
| | 11723 | 7627 | 1891 | 7522 | 2323 | 8023 | 4766 | 8763 | 3951 | 8643 | 2501 | 5759 |
| | 1647 | 1794 | 11762 | 5216 | 5233 | 8857 | 11939 | 11126 | 5668 | 5621 | 11780 | 2443 |
| | 5526 | 448 | 7298 | 6966 | 8734 | 1104 | 5647 | 2321 | 12229 | 10556 | 1362 | 7312 |
| | 5089 | 4558 | 784 | 7286 | 2999 | 10921 | 10659 | 6804 | 659 | 6020 | 7233 | 6496 |
| | 4454 | 10299 | 11650 | 6596 | 4378 | 2529 | 10338 | 1375 | 573 | 2224 | 12213 | 1763 |
| | 1605 | 830 | 5001 | 10446 | 3830 | 4701 | 776 | 9314 | 6716 | 7514 | 3121 | 8862 |
| | 3508 | 8463 | 1757 | 6913 | 8530 | 5097 | 7740 | 2770 | 459 | 4180 | 2049 | 3855 |
| | 9103 | 6909 | 2972 | 4374 | 2939 | 12517 | 6215 | 6073 | 6945 | 11644 | 2408 | 8583 |
| | 12484 | 6919 | 5474 | 3709 | 3313 | 12163 | 3116 | 9475 | 8417 | 10673 | 6369 | 4831 |
| | 1516 | 1241 | 7797 | 6820 | 8998 | 1997 | 1768 | 2328 | 7424 | 11978 | 8750 | 7780 |
| | 11894 | 3142 | 12577 | 12293 | 11312 | 2772 | 11537 | 10690 | 1718 | 8646 | 8086 | 4294 |
| | 9402 | 9400 | 4982 | 4149 | 8342 | 12024 | 2655 | 10289 | 6995 | 8495 | 7186 | 9136 |
| | 9600 | 4718 | 3389 | 4542 | 519 | 4041 | 992 | 7372 | 1002 | 11226 | 2528 | 12519 |
| | 1542 | 11389 | 1514 | 6818 | 3426 | 4183 | 9065 | 7662 | 8073 | 521 | 1525 | 6163 |
| | 861 | 8690 | 2566 | 11361 | 9685 | 12230 | 8817 | 4888 | | | | |
| 354: | 5626 | 2966 | 4663 | 4805 | 5637 | 8787 | 12445 | 11680 | | | | |
| 355: | 9746 | 2454 | 2820 | 6126 | 9527 | 11327 | 4206 | 1124 | | | | |
| 356: | 3803 | 2159 | 11265 | 10262 | 1208 | 2265 | 6548 | 4559 | 9949 | 8892 | 1253 | 5869 |
| | 6212 | 6017 | 11175 | 3155 | 5749 | 2919 | 12006 | 2161 | 7594 | 10558 | 12270 | |
| 357: | 11897 | 12310 | 11976 | 5165 | 7935 | 565 | 11277 | 908 | 12483 | 7089 | 11639 | |
| 358: | 7374 | 4134 | 5499 | 4481 | 11052 | 4793 | 5466 | 6747 | 3954 | 6213 | 640 | 4867 |
| | 12055 | 11571 | 3592 | 1079 | 5345 | 10097 | 11174 | 7895 | | | | |
| 359: | 10455 | 9279 | 2524 | 4394 | 10985 | 4178 | 3240 | 6391 | 10552 | 3567 | 2029 | 1042 |
| | 1797 | 11402 | 7449 | 8268 | 2462 | 865 | 5988 | 3908 | 1404 | 11416 | 5916 | 7278 |
| | 7325 | 12549 | 7885 | 7883 | 6455 | 11569 | 6302 | 12413 | 9181 | 1962 | 2013 | 3143 |
| | 8399 | 10462 | 7807 | 7806 | 3136 | 3131 | 11003 | 1251 | 7387 | 2412 | 10649 | 10077 |
| | 4839 | 7683 | 6752 | 8353 | 3902 | 11971 | 9700 | 7909 | 1974 | 1145 | 11946 | 8178 |
| | 8457 | | | | | | | | | | | |
| 360: | 243 | 8836 | 3558 | 6739 | 5558 | 11923 | 11784 | 11730 | 10397 | 3528 | 7134 | 8772 |
| | 7228 | 2453 | 8340 | 7839 | 1256 | 10038 | 12184 | | | | | |
| 361: | 10213 | 10215 | 12148 | 10197 | 1692 | 10198 | 10202 | 12341 | 3385 | 2836 | 6484 | 11717 |
| | 10618 | 3411 | 10927 | 6460 | 1921 | 10241 | 1907 | 12389 | 7827 | 9025 | 5807 | 6681 |
| | 12391 | 12411 | 6550 | 974 | 5415 | 9578 | 5213 | 4987 | 4080 | 12522 | 2369 | 12124 |
| | 4767 | 6352 | 7944 | 10217 | 10216 | 3413 | 9071 | 3511 | 11013 | 10218 | 7981 | 8298 |
| | 9862 | 2132 | 530 | 3844 | 6662 | 6625 | 4550 | 6341 | 1722 | 7027 | 9323 | 5094 |
| | 3611 | 2266 | 4530 | 7763 | 7290 | 11753 | 2081 | 4242 | 11360 | 1239 | 2673 | 4531 |
| | 8677 | 4930 | 2570 | 7994 | 6309 | 4476 | 9282 | 6794 | 3851 | 3634 | 3637 | 10242 |
| | 10245 | | | | | | | | | | | |
| 362: | 922 | 10181 | 10184 | 6562 | 3258 | 6528 | 6629 | 1180 | 9451 | 406 | 6910 | 5922 |
| | 8885 | 9557 | 3599 | 7579 | 7047 | 2882 | 10405 | 3975 | 532 | 1868 | 2867 | 574 |
| | 1859 | 6925 | 6880 | 950 | 10421 | 6722 | 1464 | 1500 | 5038 | 12068 | 3134 | 12241 |
| | 9985 | 5705 | 3554 | 11752 | 2337 | 1748 | 4287 | 7838 | 8011 | 5651 | 5941 | 9051 |
| | 5564 | 4621 | 7863 | 2536 | 5059 | 5773 | 5837 | 12335 | 6680 | 7099 | 7118 | 10998 |
| | 2432 | 2463 | 1750 | 9628 | 6813 | 3628 | 9033 | 5836 | 10449 | 5420 | 5207 | 3387 |
| | 7412 | 3524 | 394 | 11949 | 8547 | 4036 | 6873 | 1600 | 1443 | 5349 | 4233 | 9783 |
| | 7318 | 2864 | 11057 | 1323 | 3052 | 10007 | 2599 | 6329 | 9359 | 7436 | 7657 | 11879 |
| | 1025 | 12197 | 9682 | 10707 | 9968 | 4445 | 801 | 8656 | 6142 | 2948 | 7184 | 4255 |
| | 10302 | 10364 | 12386 | 7309 | 12216 | 6438 | 2050 | 3050 | 2920 | 5965 | 11072 | 9831 |
| | 3854 | 5662 | 12003 | 3011 | 2728 | 9738 | 1017 | 8134 | 526 | | | |
| 363: | 6176 | 8639 | 1152 | 1522 | 8352 | 6375 | 10201 | 5139 | 8072 | 2308 | 4150 | 3445 |
| | 7428 | 2024 | 4633 | 4866 | 2581 | 12337 | 12476 | 10035 | 8153 | 11036 | 3036 | 7174 |
| | 5414 | 3759 | 9595 | 1795 | 7561 | 5236 | 11547 | 1813 | 1163 | 4779 | 3695 | 4760 |
| | 6556 | 8661 | 11299 | 11986 | 8914 | 10050 | 11070 | 4068 | 809 | 4568 | 3229 | 10263 |
| | 4659 | 10993 | 8382 | 1047 | 2931 | 9895 | 4478 | 1570 | 7237 | 4269 | 3838 | 1016 |
| | 4137 | 11183 | 12276 | 11482 | 9854 | 11264 | 3433 | 7046 | 8216 | 3513 | 9383 | 8148 |
| | 1602 | 6058 | 2215 | 1842 | 5999 | 10212 | 8258 | 6591 | 4686 | 9520 | 8497 | 6648 |
| | 3088 | 6786 | 5986 | 4715 | 7335 | 6510 | 1839 | 5658 | 2103 | 11352 | 7577 | 4634 |
| | 7828 | 9426 | 11791 | 11970 | 560 | 12269 | 10454 | 3529 | 7253 | 5891 | 880 | 4009 |
| | 2760 | 7540 | 9015 | 8638 | 3356 | 5775 | 10574 | 1338 | 12117 | 3249 | 7644 | 9762 |
| | 3751 | 7042 | 3672 | 4202 | 5589 | 6019 | 9835 | 7229 | 7693 | 3086 | 10296 | 9608 |
| | 11707 | 2588 | 6425 | 9954 | 7071 | 10204 | 7525 | 6469 | 3192 | 5805 | 6230 | 4385 |
| | 2180 | 6991 | 11841 | 9662 | 5353 | 2773 | 10791 | 3059 | 6304 | 7745 | 5886 | 6518 |
| | 1536 | 1863 | 1385 | 5022 | 6003 | 8904 | 7257 | 9755 | 11607 | 11221 | 6807 | 4696 |
| | 8329 | 9798 | 1221 | | | | | | | | | |
| 364: | 4714 | 6552 | 6036 | 9637 | 11177 | 7157 | 2425 | 8841 | 4455 | 8468 | 3444 | 8404 |
| | 3936 | 763 | 10942 | 1668 | 885 | 1786 | 10802 | 7271 | 8055 | 4969 | 11623 | 3054 |
| | 11828 | 2575 | 5685 | 5924 | 5460 | 4819 | 9989 | 11834 | 10380 | 5520 | 9589 | 3334 |
| | 4912 | 9894 | 7974 | 8700 | 12342 | | | | | | | |
| 365: | 4543 | 9647 | 3507 | 1211 | 3246 | 10549 | 2019 | 2094 | 4177 | 4338 | 9238 | 5770 |
| | 4772 | 6343 | 4048 | 12231 | 10905 | 12008 | 4926 | 12442 | 2270 | 5780 | 11506 | 7565 |
| | 1094 | 12147 | 1451 | 473 | 3294 | 11314 | 9173 | 2035 | 2593 | 12094 | 5494 | 5488 |
| | 3087 | 2026 | 8206 | 9878 | 4705 | 10472 | 4746 | 6642 | 5937 | 8790 | 11896 | 2244 |
| | 5026 | 5835 | 7953 | 11936 | 10110 | 7133 | 3785 | | | | | |
| 366: | 10341 | 5696 | 6298 | 2325 | 2938 | 8027 | 1580 | 7429 | 589 | 12057 | 10353 | 3976 |
| | 3860 | 1646 | 9225 | 842 | 5818 | 4573 | 1901 | 8803 | | | | |
| 367: | 4208 | 3058 | 2394 | 8507 | 2917 | 6904 | 1345 | 10919 | 4336 | 1202 | 7187 | 1114 |
| | 3353 | 3660 | 2387 | 8437 | 9496 | 9413 | 11082 | 8079 | 12040 | 2841 | 4934 | 12221 |
| | 8040 | 4875 | 7674 | 11844 | 10774 | 6033 | 11664 | 5283 | 5192 | 1085 | 3967 | 2564 |
| | 3007 | 11334 | 11393 | 9940 | 872 | 2508 | 12169 | 3721 | 6130 | 1306 | 5401 | 10879 |
| | 883 | 9814 | 5802 | 10180 | 6895 | 7491 | 3952 | 4386 | 8159 | 1106 | 5271 | 6278 |
| | 4124 | 11612 | 6546 | 556 | 9556 | 5366 | 11798 | 6295 | 10902 | 3531 | 3572 | 3574 |
| | 7418 | 3765 | 2343 | 7356 | 6349 | 9808 | 3624 | 9583 | 6431 | 3128 | 8098 | 3871 |
| | 2494 | 1319 | 2271 | 4787 | 1838 | 1456 | 5064 | 6878 | 9972 | 8908 | 6774 | 3809 |
| | 8730 | 11094 | 670 | 1496 | 5212 | 3588 | 9459 | 8518 | 6963 | 10018 | 7791 | 8264 |
| | 2418 | 6442 | 11211 | 1636 | 6014 | 5278 | 4155 | 11100 | 10528 | 11481 | 10313 | 7126 |
| | 8887 | 2600 | 11160 | 3425 | 5846 | 4898 | 4527 | 6393 | 12267 | 1747 | 10239 | 10330 |
| | 3670 | 10329 | 10327 | 11203 | 10715 | 8883 | 10249 | 1046 | 11155 | 5228 | 2070 | 2128 |
| | 7076 | 6412 | 6875 | 5853 | 8795 | 564 | 9262 | 2640 | 5083 | 1875 | 6898 | 6951 |
| | 12373 | 7127 | | | | | | | | | | |
| 368: | 6754 | 10585 | 6689 | 9826 | 964 | 4693 | 8822 | 6462 | 691 | 2410 | 9712 | 9884 |
| | 3219 | 7232 | 12539 | 1991 | 11008 | 9322 | 8725 | | | | | |
| 369: | 1461 | 7305 | 10596 | 7966 | 8323 | 9036 | 4856 | 433 | 2663 | 8335 | 8509 | 11775 |
| | 10231 | 5820 | 8056 | 12556 | 5630 | 11866 | 9292 | 2220 | 509 | 12153 | 7303 | 12393 |
| | 7926 | | | | | | | | | | | |
| 370: | 1200 | 12300 | 576 | 5306 | 8372 | 2320 | 7732 | 6788 | 3633 | 10013 | 657 | 1403 |
| | 10074 | 7663 | 6585 | 8051 | 2569 | 2722 | 8325 | 1121 | 11616 | 4248 | 6146 | 4276 |
| | 2647 | 5766 | 9625 | 10767 | 6273 | 4186 | 6188 | 3988 | 4877 | 3998 | 8151 | 6018 |
| | 2040 | 595 | | | | | | | | | | |
| 371: | 1200 | 12300 | 6283 | 5306 | 1573 | 8372 | 9432 | 1142 | 533 | 2320 | 7732 | 370 |
| | 1267 | 595 | 3852 | | | | | | | | | |
| 372: | 7477 | 1676 | 4448 | 2400 | 6045 | 6940 | 9923 | 11995 | 8913 | 10513 | 634 | 7969 |
| | 11746 | 6446 | 4371 | 1018 | 4026 | 10874 | 11604 | 5505 | 9219 | 4140 | 6427 | 12025 |
| | 1669 | 3605 | 5027 | 10847 | 11128 | 1483 | 9362 | 2044 | 12343 | 3560 | 430 | 11220 |
| | 12146 | 10479 | 7769 | 4704 | 5385 | 12424 | 3286 | 1435 | 12461 | 10990 | 7985 | 5652 |
| | 6086 | 12067 | 2785 | 6367 | 3470 | 2598 | 10591 | 4756 | 2959 | 10900 | 11878 | 12536 |
| | 5795 | 12559 | 5450 | 8391 | 10046 | 7073 | 3157 | 2834 | 8144 | 2236 | 2782 | 1926 |
| | 9617 | 1987 | 2822 | 2279 | 2196 | 10312 | 5369 | 10124 | 11903 | 12289 | 4170 | 12092 |
| | 8540 | 2362 | 12010 | 5204 | 9431 | 6620 | 3500 | 9585 | 7989 | 1508 | 4885 | 877 |
| | 6119 | 3807 | 2585 | 12330 | 4380 | 3820 | 5302 | 3056 | 6337 | 2060 | 3096 | 202 |
| | 4492 | 1604 | 11737 | 710 | 8574 | 11646 | 3171 | 9030 | 765 | 5680 | 10955 | 6592 |
| | 2624 | 9114 | 5576 | 5196 | 5912 | 1980 | 11275 | 1119 | 3929 | 9810 | 12099 | 11930 |
| | 4546 | 3504 | 1950 | 7696 | 374 | 1703 | | | | | | |
| 373: | 7477 | 1676 | 4448 | 2400 | 6045 | 6940 | 9923 | 8913 | 10513 | 634 | 11746 | 6446 |
| | 4371 | 1018 | 4026 | 11604 | 5505 | 9219 | 4140 | 6427 | 12025 | 3605 | 11128 | 9362 |
| | 1483 | 5027 | 10847 | 2044 | 12343 | 3560 | 430 | 11220 | 12146 | 12424 | 3286 | 12461 |
| | 10479 | 10990 | 5652 | 5385 | 6086 | 4704 | 7769 | 7985 | 2785 | 6367 | 10591 | 10900 |
| | 11878 | 12536 | 12559 | 12067 | 10046 | 3157 | 5450 | 8144 | 9617 | 1987 | 2822 | 10312 |
| | 2196 | 5369 | 10124 | 11903 | 12289 | 2362 | 12010 | 6620 | 5204 | 7989 | 4885 | 877 |
| | 4170 | 2585 | 8540 | 4380 | 12092 | 3820 | 1508 | 3056 | 6337 | 5302 | 3096 | 2060 |
| | 202 | 4492 | 1604 | 11737 | 710 | 8574 | 11646 | 3171 | 9030 | 765 | 5680 | 2624 |
| | 9114 | 6592 | 10955 | 5196 | 5912 | 1980 | 11275 | 9810 | 12099 | 4821 | 10994 | 8060 |
| | 1531 | 6262 | 4632 | 1058 | 2716 | 7317 | 4825 | 3731 | 5786 | 414 | 5576 | 1000 |
| | 9714 | 1854 | 2489 | 4638 | 4485 | 11930 | 3504 | 1950 | 7696 | 374 | 372 | 1703 |
| 374: | 7477 | 1676 | 4448 | 2400 | 6045 | 6940 | 9923 | 11995 | 8913 | 10513 | 634 | 7969 |
| | 11746 | 6446 | 4371 | 1018 | 4026 | 10874 | 11604 | 5505 | 9219 | 4140 | 6427 | 12025 |
| | 3605 | 1669 | 10847 | 5027 | 11128 | 9362 | 1483 | 2044 | 12343 | 3560 | 430 | 11220 |
| | 12146 | 10479 | 4704 | 7769 | 5385 | 12424 | 3286 | 12461 | 10990 | 7985 | 5652 | 6086 |
| | 1435 | 12067 | 2785 | 6367 | 3470 | 10591 | 2959 | 10900 | 2598 | 11878 | 12536 | 12559 |
| | 5450 | 10046 | 7073 | 3157 | 2834 | 5795 | 8144 | 9617 | 10146 | 1987 | 2822 | 2279 |
| | 2196 | 10312 | 5369 | 10124 | 11903 | 12289 | 4170 | 2362 | 12010 | 5204 | 6620 | 12092 |
| | 8540 | 9585 | 9431 | 3500 | 7989 | 1508 | 4885 | 877 | 2585 | 4380 | 3820 | 3056 |
| | 6337 | 5302 | 2060 | 3096 | 202 | 4492 | 1604 | 11737 | 710 | 8574 | 11646 | 3171 |
| | 9030 | 765 | 5680 | 6592 | 10955 | 2624 | 9114 | 5196 | 5912 | 1980 | 11275 | 5576 |
| | 9810 | 12099 | 11930 | 4546 | 3504 | 1950 | 7696 | 372 | | | | |
| 375: | 2393 | 3407 | 11789 | 2984 | 10140 | 7809 | 9834 | 6717 | 6156 | 10608 | 6364 | |
| 376: | 6589 | 11497 | 6836 | 11332 | 11382 | 5877 | 2213 | 2951 | 8215 | 1064 | 1057 | 8227 |
| | 8249 | 10551 | 3319 | 8195 | 8218 | 8221 | 9142 | 9661 | 8198 | 6116 | 10889 | 10520 |
| | 7399 | 3659 | 1734 | 5077 | 3510 | 5010 | 8351 | 9403 | 9244 | 483 | 4466 | 12167 |
| | 7031 | 4216 | 6477 | 4107 | 12150 | 6828 | 5305 | 12404 | 1567 | 7248 | 11614 | 7445 |
| | 3482 | 5079 | 2734 | 11033 | 3311 | 7402 | 4221 | 2223 | 1040 | 9289 | 2902 | 4438 |
| | 4600 | 4324 | 9168 | 2576 | 10382 | 7057 | 4976 | 1719 | 10894 | 10483 | 3361 | 9016 |
| | 5634 | 7496 | 1784 | 9760 | 12134 | 7188 | 8533 | 10833 | 1303 | 9336 | 4810 | 10290 |
| | 3800 | 11192 | 6040 | 852 | 586 | 9907 | 11699 | 8192 | 7242 | 2963 | 10418 | 12069 |
| | 9588 | 10604 | 2330 | 9242 | 6800 | 10782 | 7530 | 6261 | 8220 | 8962 | 7198 | 7306 |
| | 7697 | 2102 | 2085 | 9372 | 5757 | 3700 | 11432 | 3755 | 11635 | 3376 | 11831 | 9573 |
| | 10934 | 12553 | 9010 | 12441 | 2757 | 3093 | 11913 | 9725 | 3144 | 5892 | 10167 | 7130 |
| | 5337 | 3872 | 1625 | 4143 | 7507 | 3179 | 7982 | 2590 | 1203 | 1932 | 1225 | 6260 |
| | 1044 | 9032 | 498 | 499 | 9053 | 7023 | 8322 | 4927 | 3331 | 10393 | 8132 | 11728 |
| | 8655 | 7771 | 988 | 8909 | 1791 | 5413 | 2157 | 10976 | 1365 | 2511 | 3200 | 7167 |
| | 9986 | 11603 | 1041 | 6238 | 10166 | 11045 | 9704 | 9621 | 7669 | 2014 | 3281 | 11206 |
| | 2697 | 4067 | 5118 | 12401 | 5368 | 12284 | 9430 | 7136 | 12412 | 6387 | 9543 | 2522 |
| | 572 | 5119 | 1606 | 8394 | 10248 | 11146 | 10285 | 11719 | 6776 | 11309 | 11139 | 10439 |
| | 9245 | 11197 | 11729 | 2036 | 4759 | 9773 | 6077 | 5571 | 11810 | 10084 | 10857 | 8191 |
| | 9597 | 8037 | 3279 | 8225 | 797 | 976 | 977 | 979 | 999 | 5185 | 4166 | 9598 |
| | 4901 | 7135 | 2944 | 10328 | 8189 | 6781 | | | | | | |
| 377: | 10527 | 6513 | 5978 | 2596 | 1034 | 5101 | 1637 | 6141 | 6160 | 5334 | 12110 | |
| 378: | 11873 | 5442 | 7939 | 7815 | 10258 | 11591 | 11595 | 4116 | 4129 | 9663 | 4114 | 11588 |
| | 11586 | 11585 | 9690 | 11556 | 4111 | 11550 | 9687 | 4264 | 11554 | 5405 | 4130 | 3884 |
| | 9667 | 3491 | 11545 | 3879 | 11546 | 3875 | 9646 | 3873 | 4105 | 3881 | 4101 | 4086 |
| | 4084 | 3850 | 4082 | 4078 | 8588 | 8545 | 8589 | 9271 | 8300 | 5033 | 5035 | 5058 |
| | 1832 | 5005 | 9380 | 5056 | 2097 | 1720 | 7704 | 10811 | 2733 | 5138 | 3490 | 8251 |
| | 3805 | 2314 | 3573 | 5858 | 2753 | 2629 | 7295 | 557 | 3786 | 8599 | 9153 | 2012 |
| | 5249 | 12190 | 6141 | 5334 | 6162 | 7529 | 1731 | 10108 | 463 | 12105 | 5048 | 660 |
| | 7731 | 10191 | 9632 | 11189 | 6866 | 10706 | 4292 | 7977 | 437 | 10106 | 5124 | 1552 |
| | 2131 | 7651 | 11225 | 8644 | 1923 | 3585 | 6432 | 3532 | | | | |
| 379: | 8061 | 8064 | 7961 | 7964 | 404 | 3270 | 11215 | 3265 | 10192 | 11241 | 2078 | 11168 |
| | 7871 | 12087 | 4499 | 1544 | 1412 | 6268 | 8820 | 11677 | 914 | 5507 | 4996 | 4991 |
| | 4416 | 4419 | 1981 | 4347 | 3401 | 10092 | 11767 | 11031 | 12403 | 9799 | 8861 | 3101 |
| | 12558 | 10974 | 1155 | 1157 | 1162 | 1171 | 1167 | 10984 | 5609 | 5612 | 5156 | 5809 |
| | 5813 | 2069 | 10182 | 4042 | 3226 | 7512 | 6791 | 5053 | 6674 | 11820 | 11674 | 10970 |
| | 9466 | 11069 | 10506 | 2468 | 2442 | 2444 | 2291 | 2435 | 2288 | 2294 | 2296 | 1196 |
| | 12392 | 12414 | 12417 | 12418 | 12430 | 12435 | 12437 | 12453 | 8217 | 3323 | 4516 | 10500 |
| | 12038 | 2221 | 5834 | 3035 | 7400 | 2413 | 1207 | 2833 | 2850 | 4552 | 4556 | 3578 |
| | 5581 | 555 | 7183 | 552 | 7176 | 553 | 7179 | 549 | 7185 | 4518 | 4522 | 4539 |
| | 4541 | 3889 | 12474 | 5841 | 9042 | 5812 | 8199 | 8200 | 8494 | 469 | 4590 | 6355 |
| | 711 | 9845 | 9847 | 4984 | 7919 | 7432 | 7433 | 4285 | 11121 | 4503 | 7100 | 5439 |
| | 2561 | 5559 | 1944 | 1237 | 10062 | 2402 | 4735 | 6583 | 3891 | 5718 | 5725 | 5746 |
| | 9737 | 2946 | 2949 | 7280 | 5538 | 2658 | 2674 | 9057 | 8712 | 9135 | 5144 | 6967 |
| | 11626 | 10759 | 9277 | 11254 | 11258 | 10005 | 400 | 1615 | 4506 | 2137 | 6636 | 12186 |
| | 843 | 8021 | 3464 | 4335 | 12045 | 3009 | 6698 | 7468 | 1733 | 3239 | 4185 | 12228 |
| | 1011 | 4131 | 5279 | 11790 | 5453 | 5452 | 9366 | 10461 | 6840 | 5340 | 4790 | 9069 |
| | 4421 | 767 | 6182 | 2807 | 11421 | 6683 | 6090 | 4254 | 11473 | 4256 | 7225 | 11457 |
| | 6361 | 5623 | 1560 | 2751 | 6147 | 6148 | 6151 | 11409 | 11411 | 11415 | 9097 | 1310 |
| | 3111 | 4301 | 9508 | 7650 | 1796 | 11061 | 11104 | 11934 | 6530 | 9063 | 10105 | 4959 |
| | 7479 | 8137 | 11271 | 4864 | 11951 | 8624 | 8110 | 5704 | 4119 | 6365 | 9492 | 7963 |
| | 10912 | 2447 | 3204 | 3317 | 2521 | 2451 | 2450 | 2546 | 2514 | 3202 | 2487 | 2365 |
| | 3205 | 2363 | 3148 | 2396 | 3354 | 2480 | 4047 | 3352 | 3203 | 3175 | 3243 | 2484 |
| | 3262 | 3173 | 2419 | 2335 | 3380 | 3316 | 4051 | 3164 | 9189 | 11975 | 11977 | 12013 |
| | 7351 | 6499 | 6517 | 11125 | 10880 | 9640 | 9651 | 5762 | 5769 | 11996 | 11736 | 6987 |
| | 8226 | 3336 | 8224 | 1210 | 722 | 7083 | 11418 | 5972 | 9384 | 11424 | 6600 | 7623 |
| | 7528 | 10940 | 10803 | 8430 | 3662 | 3099 | 4359 | 5542 | 1686 | 11972 | 8902 | 2017 |
| | 2110 | 6669 | 6692 | 11541 | 4284 | 1473 | 4147 | 8879 | 8882 | 4376 | 8535 | 9364 |
| | 10522 | 7701 | 8652 | 11989 | 10456 | 4561 | 12185 | 1501 | 3769 | 9669 | 5220 | 5218 |
| | 9616 | 2405 | 12056 | 491 | 492 | 8338 | 819 | 2222 | 6325 | 1906 | 9158 | 6199 |
| | 421 | 5392 | 2403 | 3750 | 694 | 697 | 699 | 724 | 728 | 6340 | 3693 | 3696 |
| | 3955 | 7148 | 1166 | 2645 | 2409 | 8777 | 4990 | 8278 | 8279 | 8281 | 8304 | 8306 |
| | 8308 | 8312 | 947 | 4453 | 1206 | 10609 | 10755 | 11735 | 11539 | 11786 | 1546 | 10196 |
| | 7632 | 3082 | 3037 | 3110 | 3106 | 6524 | 3289 | 12083 | 12085 | 12082 | 490 | 6197 |
| | 12080 | 12079 | 2219 | 8066 | 6405 | 8894 | 7333 | 6385 | 10897 | 8930 | 1223 | 3912 |
| | 11910 | 1912 | 8891 | 3475 | 4698 | 2662 | 901 | 1194 | 1503 | 1507 | 8696 | 9203 |
| | 6471 | 566 | 6133 | 3248 | 11892 | 5459 | 3302 | 8553 | 4703 | 8621 | 851 | 4337 |
| | 4341 | 3675 | 2970 | 5601 | 10607 | 9524 | 12425 | 7307 | 9223 | 3918 | 736 | 729 |
| | 961 | 10464 | 6196 | 1543 | 2758 | 2971 | 5700 | 12211 | 2578 | 10438 | 3880 | 1539 |
| | 8865 | 8981 | 3738 | 12457 | 445 | 3540 | 11456 | 5703 | 4762 | 8680 | 2839 | 583 |
| | 4627 | 8732 | 5599 | 2623 | 6321 | 5659 | 10004 | 10168 | 8741 | 6218 | 2194 | 2214 |
| | 6554 | 6244 | 6555 | 2191 | 7472 | 8949 | 2896 | 6699 | 6703 | 3469 | 4902 | 9719 |
| | 1995 | 5537 | 3957 | 3964 | 11679 | 3719 | 424 | 8848 | 3586 | 11832 | 7055 | 10785 |
| | 9507 | 7643 | 10467 | 7642 | 10395 | 8808 | 9495 | 8628 | 9050 | 12192 | 9591 | 3681 |
| | 7246 | 11257 | 10605 | 9721 | 2379 | 1187 | 1674 | 10009 | 1108 | 6522 | 3363 | 3360 |
| | 1049 | 1051 | 1056 | 7756 | 7738 | 8897 | 2974 | 8924 | 2988 | 7438 | 832 | 4666 |
| | 11859 | 4702 | 2991 | 4355 | 8867 | 4968 | 8868 | 3897 | 8895 | 2967 | 4669 | 4697 |
| | 4674 | 4700 | 9415 | 6838 | 6549 | 2617 | 621 | 1831 | 10829 | 10808 | 4794 | 6627 |
| | 4776 | 7605 | 8694 | 12200 | 11056 | 11002 | 7868 | 3699 | 2610 | 419 | 418 | 10435 |
| | 936 | 2345 | 6762 | 12543 | 9549 | 10661 | 10689 | 10699 | 10719 | 1190 | 1165 | 1161 |
| | 1170 | 11929 | 11419 | 7988 | 3798 | 1218 | 1220 | 2721 | 1800 | 6167 | 1268 | 3178 |
| 380: | 7583 | 12223 | 3882 | 6192 | 10967 | 5287 | 11700 | 9299 | 12204 | 3071 | 9116 | 6233 |
| | 3626 | 6939 | 5957 | 2854 | 2852 | 10080 | 6245 | 9582 | 11285 | 9334 | 2429 | |
| 381: | 611 | 12004 | 6814 | 5320 | 1380 | 5991 | 7802 | 5112 | 6551 | 402 | 7832 | 382 |
| | 11138 | | | | | | | | | | | |
| 382: | 611 | 12004 | 5991 | 6551 | 7832 | | | | | | | |
| 383: | 4837 | 10247 | 4513 | 8974 | 9193 | 3250 | 11577 | 10663 | 2910 | 9363 | 1186 | |
| 384: | 11191 | 6118 | 591 | 9506 | 9370 | 8717 | 1055 | 9504 | 10360 | 6606 | 575 | 5878 |
| | 5911 | 3404 | 1764 | 7501 | 4369 | 4367 | 12240 | 12238 | 2227 | 7439 | 2003 | 7437 |
| | 7440 | 7521 | 11862 | 2334 | 12466 | 12137 | 5673 | 11590 | 12090 | 11766 | 7515 | 5121 |
| | 3935 | 6239 | 6232 | 836 | 6307 | 6289 | 6328 | 6310 | 9157 | 8674 | 2257 | 2828 |
| | 2851 | 6282 | 1990 | 10944 | 5126 | 5127 | 905 | 3876 | 907 | 8578 | 912 | 8514 |
| | 8899 | 12309 | 12059 | 10972 | 11683 | 4862 | 4099 | 11900 | 9518 | 9521 | 4985 | 9517 |
| | 6702 | 960 | 9819 | 958 | 965 | 963 | 2731 | 11043 | 10487 | 10486 | 11010 | 7682 |
| | 1759 | 9618 | 9619 | 5901 | 10142 | 7649 | 753 | 755 | 4752 | 6402 | 11686 | 1413 |
| | 656 | 5951 | 9563 | 795 | 8523 | 4826 | 11233 | 3115 | | | | |
| 385: | 12282 | 3816 | 6979 | 4487 | 8194 | 4174 | 1027 | 3606 | 9593 | 8612 | 10293 | 4967 |
| | 3304 | 6074 | 6908 | 8506 | 6658 | 7332 | 8682 | 9519 | 4399 | 4231 | 3446 | 3597 |
| | 11520 | 11676 | 11673 | 11704 | 11678 | 11714 | 11703 | 5090 | 10122 | 4379 | | |
| 386: | 3874 | 2573 | 7549 | 7517 | 9650 | 3332 | 1953 | 4431 | 11909 | 3497 | 10045 | 2551 |
| | 2545 | 9861 | 8988 | 4509 | 11527 | 10422 | 4863 | 8961 | 11568 | 5627 | 2724 | 9699 |
| | 5675 | 8512 | 2715 | 4144 | | | | | | | | |

### Example 6

This example illustrates the preparation and identification by screening of transgenic seeds and plants having enhanced agronomic traits using DNA encoding homologs identified in Example 7. Transgenic corn, soybean or cotton seed and plants with recombinant DNA encoding each of the homologs identified in Example 5 are prepared by transformation. The transgenic seed, plantlets and progeny plants are screened for nitrogen use efficiency, yield, water use efficiency, growth under cold stress and seed composition change. Transgenic plants and seed having at least one enhanced agronomic trait of this invention are identified.

### Example 7

This example illustrates the identification of consensus amino acid sequence for the proteins and homologs encoded by DNA that is used to prepare the transgenic seed and plants of this invention having enhanced agronomic traits.

ClustalW program was selected for multiple sequence alignments of the amino acid sequence of SEQ ID NO: 371 and 11 homologs. Three major factors affecting the sequence alignments dramatically are (1) protein weight matrices; (2) gap open penalty; (3) gap extension penalty. Protein weight matrices available for ClustalW program include Blosum, Pam and Gonnet series. Those parameters with gap open penalty and gap extension penalty were extensively tested. On the basis of the test results, Blosum weight matrix, gap open penalty of 10 and gap extension penalty of 1 were chosen for multiple sequence alignment. Attached are the sequences of SEQ ID NO: 371, its homologs and the consensus sequence, SEQ ID NO: 12608 at the end. The symbols for consensus sequence are (1) uppercase letters for 100% identity in all positions of multiple sequence alignment output; (2) lowercase letters for >=70% identity; symbol; (3) “X” indicated <70% identity; (4) dashes “-” meaning that gaps were in >=70% sequences.

| SEQ ID NO: | |
|---|---|
| 371 | MDIFDNSDLEYLVDEFH--ADFDDDEPFGEVDVTSESDSDFMDSDFDFELSESKTNNETS |
| 12300 | MDIFDNSDLEYLVDDFHGFSDSEDDEPFGEFDHKSEADSDFEDDLDPTQESD------TS |
| 6283 | MEHFNNDDLEYVVDEYYDVPDFAVEDTS---SDIVPELTSDVDSDFEDEFPTSNAKTDTT |
| 1573 | MEHFNNDDLEYVVDEYYDVPDFAVEDTS---SDIVPELTSDVDSDFEDEFPTSNAKTDTT |
| 8372 | MEHFNNDDLEYVVDEYYDVPDFAVEDTS---SDIVPELTSDVDSDFEDEFPTSNAKTDTT |
| 5306 | MEHFNNDDLEYVVDEYYDVPDFAVEDTS---SDIVPELTSDVDSDFEDEFPTSNAKTDTT |
| 9432 | ------------------------------------------------------------ |
| 533 | ------------------------------------------------------------ |
| 2320 | ------------------------------------------------------------ |
| 1142 | -------------------------------------------------MTISNTSSTSK |
| 1200 | ------------------------------------------------------------ |
| 7732 | -----------------MAHDLHDDLEFVSGDDDDYYLEFDHDPGHGFHTSAATSASQTL |
| 12608 | xxxxxxxxxxxxxxxxxxxxxxxxxxxx---xxxxxxxxxxxxxxxxxxxxxxxxxxxxx |

ALEARNGKDIQGIPWESLNYTRDRYRENRLLHYKNFESLFRSREELDKECLQVEKGKNFY
ALEARNGKDIQGIPWERLNYSRDQYRYKRLQQYKNFEILFRSRQDLDKECLQVEKGKHFY
ASEARNGKDIQGIPWERLNYSRDKYRETRLKQYKNYQNFSRSRHDLRKECLEVQKGETFY
ASEARNGKDIQGIPWERLNYSRDKYRETRLKQYKNYQNFSRSRHDLRKECLEVQKGETFY
ASEARNGKDIQGIPWERLNYSRDKYRETRLKQYKNYQNFSRSRHDLRKECLEVQKGETFY
ASEARNGKDIQGIPWERLNYSRDKYRETRLKQYKNYQNFSRSRHDLRKECLEVQKGETFY
-----------GIPWERLNYSRDKYRETRLKQYKNYQNFSRSRHDLRKECFEVQKGETFY
-----------GIPWERLNYSRDKYRETRLKQYKNYQNFSLSPHHLHKECFQVQKGQTFY
-----------GIPWERLNYSRDKYRETRLKQYKNYQNFSRSPHHLRKECFQVQKGQTFY
TIFRRNGKDIQGIPWERLNYSRDKYRETRLKQYKNYQNFSLSPHHLHKECFQVQKGQTFY
------------IPWERLQITRKDYRKARLEQYKNYENFPQSGELMDKLCKQVESSSKYY
IGALYFRTSRWTIPWERLNYSRNQYREMRLRQYKNYENLTMPRDGLEKECKQVERKDTFY
xxxxxxxxxxxgIPWErLnysRdxYRexRLxqYKNyxnfxxsxxxlxKeCxxVxkgxtfY
DFQFNTRLVKSTIAHFQLR----------------NLVWATSKHDVYFMNNYSLMHWSSL
DFQFNTRLVKSTIAHFQLR----------------NLLWATTKHDVYFMKNYSLMHWSSL
DFFFNTRLVKSTIVHFQLR----------------NLLWATSKHDVYFMQNYSVMHWSAL
DFFFNTRLVKSTIVHFQLLRQVXVSSLAGPNIMLRNLLWATSKHDVYFMQNYSVMHWSAL
DFFFNTRLVRXTLAGPNIMLR--------------NLLWATSKHDVYFMQNYSVMHWSAL
DFFFNTRLVKSTIVHFQLRPN----------IMLRNLLWATSKHDVYFMQNYSVMHWSAL
DFFFNTRLVKSTIVHFQLR----------------NLLWATSKHDVYFMQNYSVMHWSAL
DFFFNTRLVKSTIVHFQLRN----------------LLWATSKHDVYLMQNYSVMHWSAL
DFFFNTRLVKSTIVHFQLQLGRTX-------IMLRNLLWATSKHDVYLMQNYSVMHWSAL
DFFFNTRLVKSTIVHFQLLXRWNMSSLAGPYIMLRNLLWATSKHDVYLMQDYSVMHWSAL
EFQYNTRIVKPSILHFQLR----------------NLLWATSKHDVYFMSNSTVGHWSSL
DFHLNTRLVKSTIVHFQLR----------------NLLWATSKHDVYLMQNYSVMHWSSL
dFxfNTRlVkstixhfqlxxx----------xxxxnLlWATsKHDVYxMqnysvmHWSxL
LQRGKEVLNVAKPIVPSMKQHGSLSQSVSRVQISTMAVKDDLKLREGSKESLSVRKSTNL
LQRSKEVLNVAKPIVPTMKQPGLLSQSISRVQISTMAVKDDLIVAGGFQGELICKRINEP
LRRGKEVLNVAKPIIPTLKRPGFLAQPVSRVQISTMTVKENLMVAGGFQGELICKNLKHP
LRRGKEVLNVAKPIIPTLKRPGFLAQPVSRVQISTMTVKENLMVAGGX-SRVSLYNLKHP
LRRGKEVLNVAKPIIPTLKRPGFLAQPVSRVQISTMTVKENLMVAGGFQGELICKNLKHP
LRRGKEVLNVAKPIIPTLKRPGFLAQPVSRVQISTMTVKENLMVAGGFQGELICKNLKHP
LRRGKEVLNVAKPIIPTLKRPGFLAQPVSRVQISTMTVKENLMVAGGFQGELICKNLKHP
LQRSKEVLNVAKPIIPTLTHPGFLAQPVSRVQISTMTVKENLMVAGGFQGELICKNLKQP
LRRGKEVLNVAKPIIPTLKRPGFLAQPVSRVQISTMTVKENLMVAGGFQGELICKNLKHP
LQRSKEVLNVAKPIIPTLTHPGFLAQPVSRVQISTMTVKENLMVAGGFQGELICKVGLII
SHKMTDVLDFSGHVAPAKKHPGCALEGFTGVQVSTLAVNEGLLVAGGFQGELVCKSLGER
LQRGKEVLNVAGQLAPSQNVR--GAMPLSRVQISTMAVKGNLMVAGGFQGELICKYVDKP
lxrxkeVLnvakpixPtxkxpgxlaqpvsrVQiSTmxVkenLmvagGfqgelickxxxxp
RLLSALN-----------------------------------------------------
GVAFCTVLHRFX-NDITNSVDIYNAPSGSLRVITANNDCTVRVLDAXNFAFLNSFTL---
GVLFCGKITTDDNAITHAV-DVYSNPAGSLRVITANNDFQGRVFD---------------
GVLFCGKITTDDNAITNAV-DVYSNPAGSLRVITANNDFQVRVFDAENFASLGWFKYDWS
GVLFCGKITTDDNAITNAV-DVYSNPAGSLRVITANNDFQVRVFDAENFASLGCFKYDWS
GVLFCGKITTDDNAITNAV-DVYSNPAGSLRVITANNDFQVRVFDAENFASLGCFKYDWS
GVLFCGKITTDDNAITNAV-DVYRNPAGSEGNPA--------------------------
GVLFCGKITTDGNAITNAVXDVYRNPAGSLRVITAXNDSQASGFDAENFAS---------
GVLFCGKITTDDNAITNAV-DVYSNPAGSLRVITANNDFQVRVFDAENFASLGCFKYDWS
ISYFHSI-----------------------------------------------------
DVKFCTRTTLSDNAITNAM-DIHRSTSGSLRITVSNNDSGVREFDMERFQLLNHFRFNWP
GVAFCTNLTGNNNSITNAV-DIYQAPNGGTRITTANNDCVVRTFDTERFSLISHFAFPWS
gvxfcxxxtxxxnxitxax-dxyxxpxgsxrxxxxxndxxxxxxdxxxxxxxxxxxxxxx
VNNTSVSPDG--------------------------------------------------
VNNTSVSPDGKLLAVLGDSTECLIADANTGKITGSLKGHLDYSFSSAWHPDGQILATGNQ
VNNTSVSPDGKLLAVLGDSTECLIADANTGKITGSLKGHLDYSFSSAWHPDGQILATGNQ
------------------------------------------------------------
------------------------------------------------------------
VNNTSVSPDGKLLAVLGDSTECLIADANTGKITGSLKGHLDYSFSSAWHPDGQILATGNQ
------------------------------------------------------------
VNHTSVSPDKKLLAVVGDDRDALLVDSRNGKVTSTLVGHLDYSFASAWHLDGVTFATGNQ
VNNTSVSPDGKLLAVLGDSSDCLIADSQSGKEMARLKGHLDYSFSSAWHPDGRVVATGNQ
xxxxxxxxxxxxxxxxxxxxxxxxxxxxxxxxxxxxxxxxxxxxxxxxxxxxxxxxxxxx
------------------------------------------------------------
------------------------------------------------------------
------------------------------------------------------------
------------------------------------------------------------
DKTCRLWDIRNLSQSMAVLKGRMGAIRALRFTSDGRFLAMAEPADFVHIFDSHSGYEQGQ
DKTCRLWDIRNLSQSMAVLKGRMGAIRALRFTSDGRFLAMAEPADFVHIFDSHSGYEQGQ
------------------------------------------------------------
------------------------------------------------------------
DKTCRLWDIRNLSQSMAVLKGRMGAIRALRFTSDGRFLAMAEPADFVHIFDSHSGYEQGQ
------------------------------------------------------------
DKTCRVWDIRNPSTSLAVLRGNIGAIRCIRYSSDGRFLLFSEPADFVHVYSTAECYRKRQ
DRTCRVWDVRNMSRSVAVLEGRIGAVRGLRYSPDGRFLAASEPADFVHVYDAAAGYADAQ
xxxxxxxxxxxxxxxxxxxxxxxxxxxxxxxxxxxxxxxxxxxxxxxxxxxxxxxxxxxx
------------------------------------------------------*
------------------------------------------------------*
------------------------------------------------------*
------------------------------------------------------*
EIDLFGEIAGISFSPDTEALFVGIADRTYGSLLEFNRKRHYNYLDSF-------*
EIDLFGEIAGISFSPDTEALFVGIADRTYGSLLEFNRKRHYNYLDSF-------*
------------------------------------------------------*
------------------------------------------------------*
EIDLFGEIAGISFSPDTEALFVGIADRTYGSLLEFNRKRHYNYLDSF-------*
------------------------------------------------------*
EIDFFGEISGISLSPDD------ESLFVGVCDRVYASLLNYRLVHANGYLDSYM*
EIDLFGEIAGVAFSPAGNNGGGGEALFVSIADRTYGSLLEFHRRRRHGYLDCYV*
xxxxxxxxxxxxxxxxxxxxxxxxxxxxxxxxxxxxxxxxxxxxxxx-------*

The consensus amino acid sequence can be used to identify DNA corresponding to the full scope of this invention that is useful in providing transgenic plants, e.g., corn, soybean and cotton plants with transgenic cells expressing protein encoding DNA that impart an enhanced agronomic traits. For example, enhanced nitrogen use efficiency, enhanced yield, enhanced water use efficiency, enhanced growth under cold stress and/or improved seed compositions are imparted by the expression in the plants of DNA encoding a protein with amino acid sequence identical to the consensus amino acid sequence.

### Example 8

### Identification of Target Genes of Transcription Factors ABF3 and CBF3 Chemical Kinetics Models to Identify Regulator-Target Relationships

It has been shown both in mRNA blotting and microarray experiments that activation of regulators under stress conditions usually occurs earlier than that of its targets (Haake, 2002, Seki, 2002a). In eukaryotic cells, the effect of a regulator is usually achieved in multiple steps, including transcription of the regulator genes, transportation of the regulator mRNA(s) out of the nucleus, translation of the transcript(s), transportation of the regulator protein back to the nucleus, and the binding of the regulator protein to the promoter regions of its target genes to achieve transcriptional regulation. Noticeable timing difference exists among changes in concentrations of the regulator mRNA, the regulator protein, and the mRNAs of its targets. A chemical kinetics model naturally fits this context by taking into account of the time lags among these events.

Because the active level of the regulator protein is not measured directly in microarray experiments, the regulator protein concentration is treated as a hidden variable in our model to serve as the link between the measurable mRNA concentrations of a regulator and its target(s). More specifically, the regulator protein concentration can be modeled by the following chemical kinetic equation without considering post-translational regulation:

$$\frac{d {R}_{p}}{d t} = {K}_{tran} {R}_{m} - {K}_{p} {R}_{p} \qquad equation ( 1 )$$

where Rₚ is the regulator protein concentration; Rₘ is the regulator mRNA concentration; Kₜᵣₐₙ is the apparent rate of mRNA translation, and Kₚ is the turnover rate of the regulator protein. Accordingly, the time course of the target mRNA concentration can be modeled with the following equation

$$\frac{d {T}_{m}}{d t} = {B}_{t +} f ( {R}_{p} ) - {K}_{t} {T}_{m} \qquad equation ( 2 )$$

where Tₘ is the concentration of the target mRNA; B, is the basal transcription rate of the target gene; and Kₜ is the turnover rate of the target mRNA; f(Rₚ) measures the regulated transcription rate, which is different for activators and repressors. For activators, it has the following Taylor first order approximation when Rₚ is small (Chen et al., 1999).

$$f ( {R}_{p} ) = f ( {R}_{p} = 0 ) + \frac{d ( f ( {R}_{p} )}{d {R}_{p}} {\vert}_{Rp = 0} {R}_{p} \qquad equation ( 3 )$$

f(Rₚ=0) is equal to zero, assuming target gene transcription should not be activated when there is no regulator protein.

$$\frac{d ( f ( {R}_{p} )}{d {R}_{p}} {\vert}_{Rp = 0}$$

is the activation rate of regulator protein on the target gene. If it is replaced by parameter K_{act} for simplicity, f(Rₚ) takes the following form:

f(Rₚ)=K_{act}Rₚ equation (4)

The basal level target transcription rate should satisfy the following condition:

Bₜ+f(R_{pbasal})−KₜT_{mbasal}=0 equation (5)

Where R_{pbasal} and T_{mbasal} are the basal concentrations of the regulator protein and target mRNA, respectively.

Usually, what is reported in transcription profiling experiment is not the absolute concentration of mRNA, but rather a fold change compared to basal transcription level of that gene. Thus, we define relative changes of Rₘ and Tₘ as Rₘ′ and Tₘ′

Rₘ′=Rₘ/R_{mbasal}−1 equation (6)

Tₘ′=Tₘ/T_{mbasal}−1 equation (7)

Combining equation (1), (2), (4), (5), (6) and (7), and considering the fact that KₜᵣₐₙR_{mbasel}−KₚR_{pbaseal}=0, leads to the following second order ordinary differential equation:

$$\frac{{d}^{2} ( {T}_{m}^{′} )}{d {t}^{2}} + ( {K}_{t} + {K}_{p} ) \frac{d ( {T}_{m}^{′} )}{d t} + {K}_{t} {K}_{p} {T}_{m}^{′} = γ {R}_{m}^{′} \qquad equation ( 8 )$$

### Where γ=K_{act}KₜᵣₐₙR_{mbasal}/T_{mbasel}

Given all the model parameters, the relationship between the relative mRNA levels of regulator and its target, Rₘ′ and Tₘ′, is defined by Equation (8). In other words, for the target gene of a regulator, its relative mRNA level Tₘ′ has to satisfy equation (8), given the model parameters and the relative regulator mRNA level Rₘ′. It is interesting to note that the regulator protein concentration, a key variable in the original model equations, is not involved explicitly in the final equation relating the relative mRNA levels of regulator and target. To predict the target of a specific regulator, we can solve equation (8) to obtain the theoretical target behavior curve, and then find the genes with mRNA levels similar to the theoretical curve, which will be identified as the potential targets of that regulator.

In the case of transcript expression profiling experiments under stress conditions, the initial conditions should be the following:

$$\begin{matrix}{T}_{m}^{′} {\vert}_{t = 0} = 0 & equation ( 9 ) \\ \frac{d ( {T}_{m}^{′} )}{d t} {\vert}_{t = 0} = 0 & equation ( 10 )\end{matrix}$$

Because the target gene mRNA and the regulator protein should be at their basal levels at the onset of stress condition (t=0). It is apparent from equations (2) and (5) that initial condition (10) should be true.

To approximate Rₘ, a stepwise linear model can be fit as follows:

Rₘ′ᵢ(t)=αᵢ+βᵢt tᵢ≦t≦tᵢ₊₁ i=0, . . . ,n−1 equation (11)

Where tᵢ is i^{th} time point; and αᵢ and βᵢ are the parameters of stepwise linear function in each time interval, which are determined by the measured regulator mRNA levels at the two adjacent time points. Equation (8) has analytic solution:

Tmᵢ(t)′=Aᵢe^{-Kit}+Bᵢe^{-Kpt}+Cᵢ+Dᵢt tᵢ≦t≦tᵢ₊₁, i=0, . . . ,n−1 equation (12)

Where Dᵢ=βᵢγ/KₚKₜ and Cᵢ=[αᵢγ−(Kₚ+Kₜ)Dᵢ]/KₚKₜ
The contiguous restrictions on Tₘ′ are stated in the following equations:

$$\begin{matrix}\begin{matrix} {Tm}_{i}^{′} ( t ) = {Tm}_{i + 1}^{′} ( t ) , & when t = {t}_{i} i = 1 , … , n - 1.\end{matrix} & equation ( 13 ) \\ \begin{matrix}\frac{d ( {Tm}_{i}^{′} ( t ) )}{d t} = \frac{d ( {Tm}_{i + 1}^{′} ( t ) )}{d t} , & when t = {t}_{i} i = 1 , … , n - 1.\end{matrix} & equation ( 14 )\end{matrix}$$

After substituting equation (12) into equations (9), (10), (13) and (14), Aᵢ and Bᵢ can be obtained by solving sets of linear algebra equations, and are functions of αᵢ, βᵢ, γ, Kₜ and Kₚ.

Learning Model Parameters.

For each regulator and target pair, there are three parameters involved in equation (8), the target mRNA turnover rate Kₜ, the active regulator turnover rate Kₚ, and γ, which is equal to K_{act}KₜᵣₐₙR_{mbasal}/T_{mbasel}. K_{act} represents the strength of regulator protein effect on the target gene; Kₜᵣₐₙ is the translation rate of regulator mRNA. They lump together with the ratio of basal mRNA concentrations of regulator and target to form parameter γ, which determines the magnitude of the relative target mRNA level but not its shape. It is the parameters Kₜ and Kₚ that determine the shape of the relative target mRNA level, such as how fast the target gene responds to the regulator.

For gene expression experiments under stress conditions in plants, the kinetics model can be trained with known regulator-target pair reported in the literature (e.g., CBF and RD17 in Arabidopsis under cold stress) with a non-linear regression model. When the normalized expression profile of a target gene with its maximal response is considered, there is no need to keep γ as a free model parameter (γ1=nγ2 leads to Tₘ₁′=nTₘ₂′ when other parameters are kept the same in equations (8), (9) and (10)). Therefore, only two parameters Kₜ and Kₚ are estimated from the non-linear regression model, and are used to predict other regulators and their targets in plant stress response. The theoretical target mRNA expression profiles are calculated for all the genes annotated as transcription factors, and Pearson correlation coefficient is computed for each theoretical target profile and each observed expression profile in each stress condition. When high correlation in one or several stress conditions is found, the transcription factor could be one of the putative regulators of the corresponding gene.

Target Gene Prediction Using Promoter Motif Analysis.

As an additional line of evidence for regulator-target pair prediction, we used promoter motif analysis to correlate regulators and their potential targets. Differentially expressed genes under stress conditions measured in microarray experiments can be partitioned into certain number of clusters based on the similarity in their expression profiles. All known promoter motifs within 1500 base-pairs distance to the starting codon were extracted from AGRIS database (Davuluri, 2003) for each gene. The frequency of each promoter motif in each cluster is computed, and Fisher's Exact Test is conducted to test the over-representation of certain promoter motifs. Enriched promoter motifs for a given cluster are selected as putative regulator motifs when statistical significance meets certain cutoff value (e.g., p-value 0.05). When a transcription factor (or a family of transcription factors) is known to bind to the putative regulator motif, the transcription factor(s) should be the putative regulators of target genes with the regulator motif in that cluster.

Combining Evidences from Kinetics Models and Promoter Analysis.

Kinetics models and promoter analysis independently predict putative regulator-target pairs, we attempted to combine their results to enhance our ability to detect true regulator-target pairs. In our kinetics models, for each target gene only the transcription factors with a Pearson correlation coefficient higher than certain cutoff in at least one stress condition are considered as its potential regulators. It is possible that the same regulator regulates its target genes in different stress conditions. Therefore, it is reasonable to give a higher ranking for a regulator if its theoretical target profiles are correlated to those of certain gene in multiple conditions. Based on these ideas, a ranking score for each possible regulator-target pair is derived as follows:

$$score ( {r}_{i} , {t}_{j} ) = {\prod }_{k} {R}_{k} ( {r}_{i} , {t}_{j} ) / N \qquad equation ( 15 )$$

Where Rₖ(rᵢ,tⱼ) is the rank of Pearson correlation coefficient of the theoretical target profile of transcription factor rᵢ to that of gene tⱼ in stress condition k; N is the total number of transcription factors on DNA chip.

The rank of the scores for putative transcription factors should represent the likelihood of them being the true regulator for a specific gene. Similarly, the rank of p-value of motif enrichment is the indicator of the likelihood of a transcription factor(s) being the true regulator for a specific target. Lastly, we combine both rankings from kinetics model prediction and promoter analysis by defining a score for a given regulator-target pair as following:

$$L ( {r}_{i} , {t}_{j} ) = {\prod }_{m} {rank}_{m} ( {r}_{i} , {t}_{j} ) / N \\ m = 1 , 2 \qquad equation ( 17 )$$

Where L(tᵢ,tⱼ) can be viewed as the strength of transcription factor rᵢ to be the regulator of gene tⱼ; rank₁(rᵢ,tⱼ) and rank₂(rᵢ,tⱼ) are the rank of score (rᵢ,tⱼ) from kinetics model prediction, and the rank of p-value of regulator r, binding motifs enrichment for the cluster with gene tⱼ, respectively.

This method was applied to an Arabidopsis gene expression dataset measuring responses to various stress conditions (Seki et al., 2002a; Seki, et al., 2002b). In this experiment, wild-type Arabidopsis plants were subject to stress treatments for various periods (1, 2, 5, 10 and 24 hours), and extracted mRNA samples were hybridized to a cDNA microarray with ˜7000 full-length cDNAs. 493 genes were chosen for the analysis, as each of these genes was differentially regulated in at least one of the stress conditions. Table 8 shows the evidence of the predicted target genes of CBF3 in terms of evidence strength, whether evidence from kinetics model or enriched promoter analysis exists for each predicted target.

TABLE 8

| | | Evidence | | |
|---|---|---|---|---|
| | | strength | Kinetics | Promoter |
| SEQ ID NO | Target | (10⁻⁵) | model | Analysis |
| / | At1g01470 | 2.13333 | yes | yes |
| / | AtGolS3 | 2.13333 | yes | yes |
| / | RD17 | 2.13333 | yes | yes |
| / | ERD10 | 2.13333 | yes | yes |
| 175 | At1g21790 | 2.13333 | yes | yes |
| / | ERD7 | 2.13333 | yes | yes |
| / | cor15A | 2.13333 | yes | yes |
| / | FL3-5A3 | 4.26667 | yes | yes |
| / | kin2 | 4.26667 | yes | yes |
| / | cor15B | 6.4 | yes | yes |
| 176 | ERD4 | 26.66667 | yes | no |
| / | RD29A | 26.66667 | yes | no |
| / | At1g16850 | 53.33333 | yes | no |
| 177 and 178 | At1g78070 | 800 | no | yes |
| / | kin1 | 800 | no | yes |

Table 9 shows the evidence of the predicted target genes of ABF3 in terms of the evidence strength, whether evidence from kinetics model or enriched promoter analysis exists for each predicted target.

TABLE 9

| | | Evidence | | |
|---|---|---|---|---|
| | | strength | Kinetics | Promoter |
| SEQ ID NO | Target | (10⁻⁵) | model | Analysis |
| 179, 180 and 181 | At3g47340 | 1.26222 | yes | yes |
| 182 | At5g13170 | 1.26222 | yes | yes |
| 183 | At2g19900 | 1.26222 | yes | yes |
| 184 and 185 | At5g09530 | 2.52444 | yes | yes |
| 186 | At2g42790 | 2.52444 | yes | yes |
| 187 | At3g56200 | 2.52444 | yes | yes |
| 188 and 189 | At5g01520 | 2.52444 | yes | yes |
| 190 | At5g66780 | 3.78667 | yes | yes |
| 191 | At5g59320 | 3.78667 | yes | yes |
| 192 | AtHB7 | 5.04889 | yes | yes |
| / | RD29B | 7.57333 | yes | yes |
| 193 | RD20 | 7.57333 | yes | yes |

It has been shown that ABF3 and CBF3 confer stress tolerance to transgenic plants. Thus, the target genes of ABF3 and CBF3, identified by this invention, including SEQ ID NO: 368 through SEQ ID NO: 386, and their homologs, are particularly useful for producing transgenic plant cells in crop plants with enhanced stress tolerance.

### Example 9

### Identification of Amino Acid Domain by Pfam Analysis

The amino acid sequence of the expressed proteins that were shown to be associated with an enhanced trait were analyzed for Pfam protein family against the current Pfam collection of multiple sequence alignments and hidden Markov models using the HMMER software in the appended computer listing. The Pfam protein families for the proteins of SEQ ID NO: 194 through 386 are shown in Table 10. The Hidden Markov model databases for the identified patent families are also in the appended computer listing allowing identification of other homologous proteins and their cognate encoding DNA to enable the full breadth of the invention for a person of ordinary skill in the art. Certain proteins are identified by a single Pfam domain and others by multiple Pfam domains. For instance, the protein with amino acids of SEQ ID NO: 194 is characterized by three Pfam domains, i.e. PPDK_N, PEP-utilizer and PEP-utilizer_C.

TABLE 10

| PEP | | | | | | |
|---|---|---|---|---|---|---|
| SEQ | | | | | | |
| ID | | Pfam domain | | | | |
| NO | GENE ID | name | begin | stop | score | E-value |
| 194 | PHE0003351_PMON81242.pep | PPDK_N | 99 | 464 | 710.9 | 7.90E−211 |
| 194 | PHE0003351_PMON81242.pep | PEP-utilizers | 500 | 601 | 182.3 | 1.10E−51 |
| 194 | PHE0003351_PMON81242.pep | PEP-utilizers_C | 613 | 969 | 723.9 | 1.00E−214 |
| 195 | PHE0003351_PMON83625.pep | PPDK_N | 99 | 464 | 710.9 | 7.90E−211 |
| 195 | PHE0003351_PMON83625.pep | PEP-utilizers | 500 | 601 | 182.3 | 1.10E−51 |
| 195 | PHE0003351_PMON83625.pep | PEP-utilizers_C | 613 | 969 | 723.9 | 1.00E−214 |
| 196 | PHE0000207_PMON77878.pep | Pkinase | 1 | 259 | 343.1 | 4.40E−100 |
| 197 | PHE0000208_PMON77879.pep | Pkinase | 1 | 259 | 353.4 | 3.30E−103 |
| 198 | PHE0000209_PMON77891.pep | Pkinase | 1 | 259 | 354.9 | 1.20E−103 |
| 199 | PHE0000210_PMON77880.pep | Pkinase | 1 | 259 | 359.4 | 5.40E−105 |
| 200 | PHE0001329_PMON92878.pep | Pkinase | 12 | 266 | 354.3 | 1.80E−103 |
| 200 | PHE0001329_PMON92878.pep | NAF | 311 | 371 | 123.6 | 5.10E−34 |
| 201 | PHE0001425_PMON79162.pep | CAF1 | 19 | 252 | 368.1 | 1.30E−107 |
| 202 | PHE0001573_PMON92870.pep | GATase_2 | 2 | 162 | 55.5 | 3.10E−15 |
| 202 | PHE0001573_PMON92870.pep | Asn_synthase | 210 | 451 | 329.6 | 5.00E−96 |
| 203 | PHE0001664_PMON99280.pep | FAD_binding_4 | 69 | 213 | 83.4 | 6.30E−22 |
| 204 | PHE0001674_PMON79194.pep | Myb_DNA-binding | 25 | 70 | 36.3 | 9.90E−08 |
| 205 | PHE0002026_PMON96489.pep | Ammonium_transp | 36 | 459 | 628.5 | 5.10E−186 |
| 206 | PHE0002108_PMON92821.pep | CSD | 1 | 65 | 155.1 | 1.60E−43 |
| 207 | PHE0002109_PMON93856.pep | CSD | 1 | 67 | 144.8 | 2.10E−40 |
| 208 | PHE0002508_PMON92607.pep | CBFD_NFYB_HMF | 24 | 89 | 130.9 | 3.20E−36 |
| 209 | PHE0002650_PMON81832.pep | SRF-TF | 9 | 59 | 106.9 | 5.50E−29 |
| 209 | PHE0002650_PMON81832.pep | K-box | 73 | 172 | 118.4 | 1.90E−32 |
| 210 | PHE0002989_PMON95630.pep | Miro | 10 | 126 | 74.3 | 3.40E−19 |
| 210 | PHE0002989_PMON95630.pep | Ras | 11 | 173 | 288.8 | 9.30E−84 |
| 212 | PHE0003300_PMON95106.pep | MtN3_slv | 12 | 99 | 131.1 | 2.80E−36 |
| 212 | PHE0003300_PMON95106.pep | MtN3_slv | 133 | 219 | 134.9 | 2.00E−37 |
| 214 | PHE0003389_PMON94682.pep | p450 | 48 | 527 | 286.5 | 4.60E−83 |
| 215 | PHE0003614_PMON95111.pep | Pyridoxal_deC | 33 | 381 | 531.8 | 6.80E−157 |
| 216 | PHE0003684_PMON92807.pep | Myb_DNA-binding | 118 | 168 | 47.9 | 3.00E−11 |
| 217 | PHE0003684_PMON93378.pep | Myb_DNA-binding | 118 | 168 | 47.9 | 3.00E−11 |
| 218 | PHE0003853_PMON92602.pep | Cyclin_N | 46 | 171 | 72.6 | 1.10E−18 |
| 219 | PHE0003903_PMON98271.pep | TPP_enzyme_N | 44 | 220 | 302.9 | 5.50E−88 |
| 219 | PHE0003903_PMON98271.pep | TPP_enzyme_M | 241 | 390 | 157.3 | 3.70E−44 |
| 220 | PHE0003905_PMON99283.pep | Aldedh | 30 | 492 | 514.4 | 1.10E−151 |
| 221 | PHE0003907_PMON98066.pep | Ribosomal_L12 | 124 | 191 | 62.6 | 1.20E−15 |
| 222 | PHE0003908_PMON98064.pep | DnaJ | 31 | 93 | 128.9 | 1.30E−35 |
| 223 | PHE0003960_PMON95079.pep | CTP_transf_2 | 56 | 186 | 142.9 | 7.70E−40 |
| 224 | PHE0003967_PMON95088.pep | GST_N | 11 | 84 | 43.3 | 7.40E−10 |
| 228 | PHE0004023_PMON92446.pep | PHD | 198 | 248 | 54.9 | 2.40E−13 |
| 229 | PHE0004026_PMON93885.pep | Aa_trans | 44 | 438 | 409.4 | 4.80E−120 |
| 230 | PHE0004027_PMON93860.pep | FAD_binding_4 | 64 | 218 | 83.9 | 4.60E−22 |
| 231 | PHE0004028_PMON94697.pep | Alpha-amylase | 10 | 426 | −62.1 | 4.30E−06 |
| 232 | PHE0004034_PMON92631.pep | DUF1336 | 236 | 478 | 491.8 | 7.30E−145 |
| 234 | PHE0004047_PMON92619.pep | LIM | 11 | 68 | 53.4 | 7.00E−13 |
| 234 | PHE0004047_PMON92619.pep | LIM | 110 | 167 | 63.9 | 4.70E−16 |
| 235 | PHE0004047_PMON93388.pep | LIM | 11 | 68 | 53.4 | 7.00E−13 |
| 235 | PHE0004047_PMON93388.pep | LIM | 110 | 167 | 63.9 | 4.70E−16 |
| 236 | PHE0004068_PMON93663.pep | AWPM-19 | 1 | 125 | 287.6 | 2.20E−83 |
| 237 | PHE0004071_PMON93311.pep | RRM_1 | 105 | 174 | 77.3 | 4.40E−20 |
| 238 | PHE0004072_PMON93654.pep | MMR_HSR1 | 214 | 324 | 65.7 | 1.40E−16 |
| 239 | PHE0004072_PMON93669.pep | MMR_HSR1 | 214 | 324 | 65.7 | 1.40E−16 |
| 241 | PHE0004075_PMON92851.pep | MtN3_slv | 15 | 104 | 75.5 | 1.50E−19 |
| 241 | PHE0004075_PMON92851.pep | MtN3_slv | 137 | 223 | 97.2 | 4.50E−26 |
| 242 | PHE0004080_PMON93321.pep | peroxidase | 19 | 227 | 241 | 2.40E−69 |
| 243 | PHE0004084_PMON95141.pep | Phi_1 | 35 | 314 | 691.3 | 6.40E−205 |
| 244 | PHE0004093_PMON93332.pep | Dimerisation | 40 | 100 | 105.7 | 1.20E−28 |
| 244 | PHE0004093_PMON93332.pep | Methyltransf_2 | 104 | 350 | 317.5 | 2.20E−92 |
| 245 | PHE0004093_PMON94155.pep | Dimerisation | 40 | 100 | 105.7 | 1.20E−28 |
| 245 | PHE0004093_PMON94155.pep | Methyltransf_2 | 104 | 350 | 317.5 | 2.20E−92 |
| 247 | PHE0004144_PMON93842.pep | Cofilin_ADF | 16 | 143 | 152.4 | 1.10E−42 |
| 248 | PHE0004148_PMON92574.pep | Iso_dh | 28 | 412 | 521.5 | 8.40E−154 |
| 249 | PHE0004149_PMON92471.pep | HEAT_PBS | 115 | 143 | 23.8 | 0.00057 |
| 249 | PHE0004149_PMON92471.pep | HEAT_PBS | 155 | 181 | 37.8 | 3.50E−08 |
| 249 | PHE0004149_PMON92471.pep | HEAT_PBS | 186 | 212 | 22.4 | 0.0015 |
| 249 | PHE0004149_PMON92471.pep | HEAT_PBS | 260 | 287 | 16.8 | 0.074 |
| 250 | PHE0004149_PMON93899.pep | HEAT_PBS | 115 | 143 | 23.8 | 0.00057 |
| 250 | PHE0004149_PMON93899.pep | HEAT_PBS | 155 | 181 | 37.8 | 3.50E−08 |
| 250 | PHE0004149_PMON93899.pep | HEAT_PBS | 186 | 212 | 22.4 | 0.0015 |
| 250 | PHE0004149_PMON93899.pep | HEAT_PBS | 260 | 287 | 16.8 | 0.074 |
| 251 | PHE0004152_PMON93672.pep | AT_hook | 69 | 81 | 7.4 | 1.1 |
| 251 | PHE0004152_PMON93672.pep | DUF296 | 96 | 217 | 175.1 | 1.60E−49 |
| 252 | PHE0004155_PMON92626.pep | ADH_N | 41 | 152 | 176.1 | 8.20E−50 |
| 252 | PHE0004155_PMON92626.pep | ADH_zinc_N | 181 | 324 | 127.8 | 2.70E−35 |
| 253 | PHE0004156_PMON92623.pep | NPH3 | 135 | 364 | 219.3 | 7.80E−63 |
| 254 | PHE0004162_PMON92481.pep | AUX_IAA | 22 | 279 | 395.9 | 5.30E−116 |
| 255 | PHE0004164_PMON92465.pep | X8 | 29 | 115 | 168.4 | 1.70E−47 |
| 257 | PHE0004167_PMON93333.pep | APS_kinase | 108 | 264 | 363.4 | 3.20E−106 |
| 258 | PHE0004168_PMON93855.pep | FAD_binding_4 | 84 | 225 | 93 | 8.10E−25 |
| 258 | PHE0004168_PMON93855.pep | BBE | 476 | 534 | 120.1 | 5.70E−33 |
| 259 | PHE0004169_PMON92568.pep | Aldo_ket_red | 14 | 298 | 389.4 | 4.80E−114 |
| 260 | PHE0004184_PMON92565.pep | UIM | 214 | 231 | 14.4 | 0.29 |
| 260 | PHE0004184_PMON92565.pep | UIM | 298 | 315 | 24 | 0.00049 |
| 261 | PHE0004185_PMON92802.pep | p450 | 39 | 504 | 157.8 | 2.70E−44 |
| 262 | PHE0004188_PMON92803.pep | HSF_DNA-bind | 70 | 233 | 177.8 | 2.50E−50 |
| 263 | PHE0004190_PMON92801.pep | HLH | 175 | 213 | 9.2 | 0.014 |
| 264 | PHE0004208_PMON92834.pep | Myb_DNA-binding | 5 | 56 | 39.1 | 1.30E−08 |
| 264 | PHE0004208_PMON92834.pep | Myb_DNA-binding | 134 | 181 | 44.6 | 3.10E−10 |
| 265 | PHE0004215_PMON92827.pep | PBP | 14 | 170 | 30 | 1.50E−07 |
| 266 | PHE0004223_PMON92840.pep | Fasciclin | 40 | 179 | 11.7 | 0.00032 |
| 266 | PHE0004223_PMON92840.pep | Fasciclin | 217 | 353 | 104.9 | 2.20E−28 |
| 267 | PHE0004225_PMON94167.pep | Aldedh | 77 | 539 | 880.7 | 6.30E−262 |
| 268 | PHE0004226_PMON95114.pep | Aldedh | 77 | 539 | 873 | 1.30E−259 |
| 269 | PHE0004227_PMON92605.pep | UPF0057 | 5 | 55 | 76.3 | 8.50E−20 |
| 270 | PHE0004229_PMON92867.pep | UPF0057 | 4 | 54 | 96.4 | 8.00E−26 |
| 271 | PHE0004233_PMON92843.pep | HSF_DNA-bind | 60 | 236 | 255.5 | 1.00E−73 |
| 272 | PHE0004237_PMON93673.pep | HSP20 | 48 | 153 | 184.3 | 2.80E−52 |
| 273 | PHE0004243_PMON92621.pep | CBFD_NFYB_HMF | 22 | 87 | 122.5 | 1.10E−33 |
| 274 | PHE0004244_PMON92858.pep | CBFD_NFYB_HMF | 39 | 104 | 121 | 3.00E−33 |
| 275 | PHE0004245_PMON93813.pep | CBFD_NFYB_HMF | 25 | 90 | 129.2 | 1.00E−35 |
| 276 | PHE0004248_PMON94672.pep | CBFD_NFYB_HMF | 37 | 102 | 125.3 | 1.60E−34 |
| 278 | PHE0004250_PMON92881.pep | CBFD_NFYB_HMF | 25 | 90 | 119.7 | 7.80E−33 |
| 279 | PHE0004252_PMON92606.pep | CBFD_NFYB_HMF | 14 | 79 | 94.1 | 3.80E−25 |
| 280 | PHE0004253_PMON92874.pep | CBFD_NFYB_HMF | 7 | 71 | 83.8 | 5.00E−22 |
| 281 | PHE0004258_PMON93385.pep | Pkinase | 5 | 276 | 144.7 | 2.30E−40 |
| 282 | PHE0004258_PMON93806.pep | Pkinase | 5 | 276 | 144.7 | 2.30E−40 |
| 283 | PHE0004259_PMON93384.pep | Abhydrolase_3 | 95 | 319 | 302.4 | 7.70E−88 |
| 285 | PHE0004261_PMON93389.pep | Pkinase | 31 | 282 | 289.6 | 5.30E−84 |
| 285 | PHE0004261_PMON93389.pep | Pkinase_Tyr | 31 | 280 | 69.3 | 6.10E−20 |
| 286 | PHE0004261_PMON93655.pep | Pkinase | 31 | 282 | 289.6 | 5.30E−84 |
| 286 | PHE0004261_PMON93655.pep | Pkinase_Tyr | 31 | 280 | 69.3 | 6.10E−20 |
| 287 | PHE0004262_PMON92862.pep | Pkinase | 86 | 366 | 153.9 | 3.70E−43 |
| 287 | PHE0004262_PMON92862.pep | Pkinase_Tyr | 86 | 366 | 132.2 | 1.30E−36 |
| 288 | PHE0004262_PMON93360.pep | Pkinase | 86 | 366 | 153.9 | 3.70E−43 |
| 288 | PHE0004262_PMON93360.pep | Pkinase_Tyr | 86 | 366 | 132.2 | 1.30E−36 |
| 289 | PHE0004264_PMON92845.pep | PMEI | 25 | 174 | 138.8 | 1.40E−38 |
| 290 | PHE0004264_PMON93354.pep | PMEI | 25 | 174 | 138.8 | 1.40E−38 |
| 291 | PHE0004265_PMON92873.pep | Suc_Fer-like | 59 | 308 | 60.7 | 4.30E−15 |
| 292 | PHE0004265_PMON93807.pep | Suc_Fer-like | 59 | 308 | 60.7 | 4.30E−15 |
| 293 | PHE0004266_PMON92877.pep | Myb_DNA-binding | 298 | 348 | 46.6 | 7.80E−11 |
| 294 | PHE0004284_PMON93857.pep | U-box | 23 | 97 | 98.2 | 2.30E−26 |
| 295 | PHE0004285_PMON95136.pep | CBFD_NFYB_HMF | 61 | 126 | 123 | 7.70E−34 |
| 296 | PHE0004286_PMON93666.pep | ICL | 21 | 551 | 1239.3 | 0 |
| 297 | PHE0004287_PMON93344.pep | ICL | 21 | 552 | 1169.2 | 0 |
| 298 | PHE0004307_PMON94102.pep | RWP-RK | 196 | 247 | 90.7 | 4.00E−24 |
| 299 | PHE0004314_PMON93397.pep | zf-C3HC4 | 148 | 185 | 34.1 | 4.50E−07 |
| 300 | PHE0004321_PMON93811.pep | Redoxin | 64 | 228 | 4.9 | 0.0016 |
| 300 | PHE0004321_PMON93811.pep | GSHPx | 73 | 181 | 246.5 | 5.10E−71 |
| 301 | PHE0004321_PMON93834.pep | Redoxin | 64 | 228 | 4.9 | 0.0016 |
| 301 | PHE0004321_PMON93834.pep | GSHPx | 73 | 181 | 246.5 | 5.10E−71 |
| 302 | PHE0004325_PMON93818.pep | CcmH | 1 | 139 | 16.6 | 6.50E−09 |
| 303 | PHE0004335_PMON93850.pep | DZC | 158 | 193 | 81.9 | 1.80E−21 |
| 303 | PHE0004335_PMON93850.pep | DZC | 308 | 343 | 80.9 | 3.70E−21 |
| 304 | PHE0004336_PMON93858.pep | DZC | 179 | 214 | 78.3 | 2.20E−20 |
| 304 | PHE0004336_PMON93858.pep | DZC | 369 | 404 | 71.4 | 2.60E−18 |
| 306 | PHE0004348_PMON93810.pep | CSD | 1 | 65 | 136.8 | 5.50E−38 |
| 307 | PHE0004349_PMON93812.pep | CSD | 1 | 65 | 141.9 | 1.50E−39 |
| 308 | PHE0004350_PMON93826.pep | CSD | 1 | 66 | 148.4 | 1.70E−41 |
| 309 | PHE0004351_PMON93821.pep | CSD | 1 | 66 | 149.5 | 8.10E−42 |
| 310 | PHE0004352_PMON93824.pep | CSD | 2 | 68 | 151.2 | 2.50E−42 |
| 312 | PHE0004393_PMON94192.pep | efhand | 29 | 57 | 18 | 0.031 |
| 312 | PHE0004393_PMON94192.pep | efhand | 66 | 94 | 25.3 | 0.0002 |
| 312 | PHE0004393_PMON94192.pep | efhand | 110 | 138 | 24.2 | 0.00042 |
| 313 | PHE0004395_PMON94145.pep | C2 | 16 | 138 | 72.1 | 1.60E−18 |
| 313 | PHE0004395_PMON94145.pep | PLDc | 357 | 392 | 30.1 | 7.20E−06 |
| 313 | PHE0004395_PMON94145.pep | PLDc | 702 | 729 | 37.1 | 5.70E−08 |
| 314 | PHE0004396_PMON94137.pep | Orn_Arg_deC_N | 118 | 393 | 282.3 | 8.80E−82 |
| 314 | PHE0004396_PMON94137.pep | Orn_DAP_Arg_deC | 396 | 596 | 161.7 | 1.70E−45 |
| 315 | PHE0004417_PMON94190.pep | Spermine_synth | 13 | 256 | 516.1 | 3.40E−152 |
| 316 | PHE0004418_PMON94368.pep | Amino_oxidase | 18 | 504 | 275.8 | 7.90E−80 |
| 317 | PHE0004419_PMON95100.pep | Amidohydro_1 | 95 | 446 | 56.2 | 1.00E−13 |
| 317 | PHE0004419_PMON95100.pep | Amidohydro_3 | 95 | 444 | −49.9 | 0.00024 |
| 318 | PHE0004421_PMON95120.pep | AP2 | 52 | 118 | 92.2 | 1.40E−24 |
| 319 | PHE0004422_PMON95123.pep | AP2 | 58 | 123 | 78.5 | 1.90E−20 |
| 322 | PHE0004432_PMON94112.pep | Lactamase_B | 63 | 262 | 81.6 | 2.20E−21 |
| 322 | PHE0004432_PMON94112.pep | RMMBL | 400 | 440 | 33.6 | 6.10E−07 |
| 323 | PHE0004472_PMON94115.pep | Sina | 5 | 205 | 188 | 2.00E−53 |
| 324 | PHE0004472_PMON94126.pep | Sina | 5 | 205 | 188 | 2.00E−53 |
| 325 | PHE0004488_PMON95609.pep | Anti-silence | 1 | 155 | 392.9 | 4.40E−115 |
| 327 | PHE0004492_PMON95614.pep | NPH3 | 193 | 435 | 469.9 | 2.90E−138 |
| 328 | PHE0004545_PMON95117.pep | Ribosomal_L14 | 49 | 196 | 105.5 | 1.50E−28 |
| 329 | PHE0004574_PMON94433.pep | Transaldolase | 102 | 405 | 620.7 | 1.20E−183 |
| 329 | PHE0004574_PMON94433.pep | efhand | 444 | 472 | 21.3 | 0.0031 |
| 330 | PHE0004606_PMON95627.pep | Metallophos | 54 | 249 | 154 | 3.60E−43 |
| 331 | PHE0004620_PMON94189.pep | PFK | 6 | 281 | 515.1 | 7.30E−152 |
| 332 | PHE0004620_PMON94442.pep | PFK | 6 | 281 | 515.1 | 7.30E−152 |
| 333 | PHE0004622_PMON95621.pep | F-box | 2 | 49 | 43.2 | 8.10E−10 |
| 333 | PHE0004622_PMON95621.pep | LRR_2 | 150 | 175 | 41.5 | 2.60E−09 |
| 333 | PHE0004622_PMON95621.pep | FBD | 332 | 382 | 74.8 | 2.50E−19 |
| 334 | PHE0004626_PMON95101.pep | Aminotran_3 | 79 | 434 | 323.4 | 3.50E−94 |
| 335 | PHE0004630_PMON94367.pep | Iso_dh | 40 | 363 | 326.9 | 3.20E−95 |
| 336 | PHE0004634_PMON94385.pep | AP2 | 28 | 91 | 114.2 | 3.40E−31 |
| 337 | PHE0004640_PMON95066.pep | FAE1_CUT1_RppA | 75 | 365 | 539.9 | 2.40E−159 |
| 337 | PHE0004640_PMON95066.pep | Chal_sti_synt_C | 322 | 466 | 8.7 | 0.0003 |
| 337 | PHE0004640_PMON95066.pep | ACP_syn_III_C | 382 | 464 | 26.7 | 2.30E−08 |
| 338 | PHE0004645_PMON94655.pep | 14-3-3 | 5 | 241 | 304.8 | 1.50E−88 |
| 339 | PHE0004645_PMON94685.pep | 14-3-3 | 5 | 241 | 304.8 | 1.50E−88 |
| 342 | PHE0004650_PMON94686.pep | Skp1_POZ | 4 | 64 | 105.3 | 1.70E−28 |
| 342 | PHE0004650_PMON94686.pep | Skp1 | 112 | 190 | 173 | 6.90E−49 |
| 343 | PHE0004652_PMON94657.pep | UPF0005 | 31 | 247 | 55.1 | 2.20E−13 |
| 344 | PHE0004652_PMON94687.pep | UPF0005 | 31 | 247 | 55.1 | 2.20E−13 |
| 346 | PHE0004689_PMON95131.pep | Pkinase | 12 | 291 | 357 | 2.70E−104 |
| 347 | PHE0004691_PMON95129.pep | Spermine_synth | 33 | 278 | 501.4 | 9.60E−148 |
| 348 | PHE0004719_PMON94698.pep | zf-C3HC4 | 203 | 243 | 26.6 | 8.00E−05 |
| 349 | PHE0004719_PMON95089.pep | zf-C3HC4 | 203 | 243 | 26.6 | 8.00E−05 |
| 350 | PHE0004734_PMON94667.pep | KOW | 26 | 62 | 30.7 | 4.80E−06 |
| 350 | PHE0004734_PMON94667.pep | eIF-5a | 84 | 153 | 125.8 | 1.10E−34 |
| 351 | PHE0004735_PMON95116.pep | KOW | 26 | 62 | 32.2 | 1.70E−06 |
| 351 | PHE0004735_PMON95116.pep | eIF-5a | 84 | 153 | 120.3 | 5.10E−33 |
| 352 | PHE0004739_PMON95110.pep | Miro | 7 | 121 | 68.7 | 1.70E−17 |
| 352 | PHE0004739_PMON95110.pep | Ras | 8 | 179 | 270.9 | 2.30E−78 |
| 353 | PHE0004753_PMON95105.pep | Aldedh | 61 | 520 | 791.8 | 3.50E−235 |
| 355 | PHE0004770_PMON95122.pep | DUF1242 | 2 | 70 | 118.4 | 1.80E−32 |
| 357 | PHE0004774_PMON95147.pep | zf-A20 | 14 | 38 | 33.1 | 9.10E−07 |
| 357 | PHE0004774_PMON95147.pep | zf-AN1 | 92 | 132 | 68.2 | 2.50E−17 |
| 358 | PHE0004777_PMON95118.pep | RNA_pol_L | 6 | 83 | 75.9 | 1.20E−19 |
| 359 | PHE0004785_PMON95057.pep | Ribosomal_L18p | 26 | 172 | 251.4 | 1.70E−72 |
| 360 | PHE0004786_PMON95604.pep | Phi_1 | 35 | 314 | 691.3 | 6.40E−205 |
| 361 | PHE0004788_PMON95092.pep | DS | 53 | 369 | 587.4 | 1.30E−173 |
| 362 | PHE0004799_PMON95602.pep | DAO | 34 | 481 | −14.1 | 7.30E−05 |
| 362 | PHE0004799_PMON95602.pep | Amino_oxidase | 42 | 483 | 342.7 | 5.60E−100 |
| 363 | PHE0004841_PMON95636.pep | DNA_photolyase | 18 | 190 | 254.3 | 2.30E−73 |
| 363 | PHE0004841_PMON95636.pep | FAD_binding_7 | 223 | 501 | 503 | 3.20E−148 |
| 367 | PHE0004888_PMON95603.pep | Globin | 7 | 134 | 73 | 8.40E−19 |
| 367 | PHE0004888_PMON95603.pep | FAD_binding_6 | 156 | 263 | 29 | 4.80E−07 |
| 367 | PHE0004888_PMON95603.pep | NAD_binding_1 | 276 | 393 | 13.4 | 9.80E−05 |
| 369 | ERD4.pep | DUF221 | 295 | 710 | 245.3 | 1.20E−70 |
| 370 | At1g78070.2.pep | WD40 | 310 | 347 | 34.1 | 4.40E−07 |
| 372 | At3g47340.1.pep | GATase_2 | 2 | 161 | 99.6 | 8.60E−27 |
| 372 | At3g47340.1.pep | Asn_synthase | 209 | 450 | 344.3 | 1.80E−100 |
| 373 | At3g47340.3.pep | GATase_2 | 2 | 161 | 99.6 | 8.60E−27 |
| 373 | At3g47340.3.pep | Asn_synthase | 209 | 430 | 286.1 | 6.20E−83 |
| 374 | At3g47340.2.pep | GATase_2 | 2 | 161 | 99.6 | 8.60E−27 |
| 374 | At3g47340.2.pep | Asn_synthase | 209 | 450 | 344.3 | 1.80E−100 |
| 375 | At5g13170.1.pep | MtN3_slv | 12 | 99 | 135.1 | 1.80E−37 |
| 375 | At5g13170.1.pep | MtN3_slv | 134 | 220 | 135.4 | 1.40E−37 |
| 376 | At2g19900.1.pep | malic | 107 | 295 | 407.1 | 2.20E−119 |
| 376 | At2g19900.1.pep | Malic_M | 297 | 550 | 466.9 | 2.30E−137 |
| 379 | At2g42790.1.pep | Citrate_synt | 93 | 461 | 506.2 | 3.40E−149 |
| 380 | At3g56200.1.pep | Aa_trans | 21 | 426 | 106.3 | 8.10E−29 |
| 381 | At5g01520.1.pep | zf-C3HC4 | 146 | 183 | 26.1 | 0.00011 |
| 384 | At5g59320.1.pep | Tryp_alpha_amyl | 27 | 111 | 114.7 | 2.30E−31 |
| 385 | AtHB7.pep | Homeobox | 30 | 86 | 66.6 | 7.10E−17 |
| 385 | AtHB7.pep | HALZ | 87 | 131 | 39.2 | 1.30E−08 |
| 386 | RD20.pep | Caleosin | 54 | 227 | 469 | 5.30E−138 |

TABLE 11

| Pfam domain | accession | gathering | |
|---|---|---|---|
| name | number | cutoff | domain description |
| 14-3-3 | PF00244.9 | 25 | 14-3-3 protein |
| ACP_syn_III_C | PF08541.1 | −24.4 | 3-Oxoacyl-[acyl-carrier-protein (ACP)] |
| | | | synthase III C terminal |
| ADH_N | PF08240.2 | −14.5 | Alcohol dehydrogenase GroES-like domain |
| ADH_zinc_N | PF00107.16 | 23.8 | Zinc-binding dehydrogenase |
| AP2 | PF00847.9 | 0 | AP2 domain |
| APS_kinase | PF01583.9 | 25 | Adenylylsulphate kinase |
| AT_hook | PF02178.8 | 3.6 | AT hook motif |
| AUX_IAA | PF02309.6 | −83 | AUX/IAA family |
| AWPM-19 | PF05512.1 | 25 | AWPM-19-like family |
| Aa_trans | PF01490.7 | −128.4 | Transmembrane amino acid transporter protein |
| Abhydrolase_3 | PF07859.2 | 25.8 | alpha/beta hydrolase fold |
| Aldedh | PF00171.11 | −209.3 | Aldehyde dehydrogenase family |
| Aldo_ket_red | PF00248.10 | −97 | Aldo/keto reductase family |
| Alpha-amylase | PF00128.12 | −93 | Alpha amylase, catalytic domain |
| Amidohydro_1 | PF01979.8 | −37.4 | Amidohydrolase family |
| Amidohydro_3 | PF07969.1 | −65.5 | Amidohydrolase family |
| Amino_oxidase | PF01593.12 | −11.4 | Flavin containing amine oxidoreductase |
| Aminotran_3 | PF00202.10 | −207.6 | Aminotransferase class-III |
| Ammonium_transp | PF00909.10 | −144 | Ammonium Transporter Family |
| Anti-silence | PF04729.4 | 25 | Anti-silencing protein, ASF1-like |
| Asn_synthase | PF00733.10 | −52.8 | Asparagine synthase |
| BBE | PF08031.1 | 25 | Berberine and berberine like |
| C2 | PF00168.18 | 3.7 | C2 domain |
| CAF1 | PF04857.8 | −100.5 | CAF1 family ribonuclease |
| CBFD_NFYB_HMF | PF00808.12 | 18.4 | Histone-like transcription factor (CBF/NF-Y) |
| | | | and archaeal histone |
| CSD | PF00313.12 | −0.3 | ‘Cold-shock’ DNA-binding domain |
| CTP_transf_2 | PF01467.16 | −11.8 | Cytidylyltransferase |
| Caleosin | PF05042.3 | 25 | Caleosin related protein |
| CcmH | PF03918.4 | −30.8 | Cytochrome C biogenesis protein |
| Chal_sti_synt_C | PF02797.5 | −6.1 | Chalcone and stilbene synthases, C-terminal |
| | | | domain |
| Citrate_synt | PF00285.10 | −101.5 | Citrate synthase |
| Cofilin_ADF | PF00241.10 | −4.7 | Cofilin/tropomyosin-type actin-binding protein |
| Cyclin_N | PF00134.13 | −14.7 | Cyclin, N-terminal domain |
| DAO | PF01266.12 | −35.9 | FAD dependent oxidoreductase |
| DNA_photolyase | PF00875.8 | 26.1 | DNA photolyase |
| DS | PF01916.7 | −95.2 | Deoxyhypusine synthase |
| DUF1242 | PF06842.1 | 25 | Protein of unknown function (DUF1242) |
| DUF1336 | PF07059.2 | −78.2 | Protein of unknown function (DUF1336) |
| DUF221 | PF02714.5 | 25 | Domain of unknown function DUF221 |
| DUF296 | PF03479.4 | −11 | Domain of unknown function (DUF296) |
| DZC | PF08381.1 | 15.3 | Disease resistance/zinc finger/chromosome |
| | | | condensation-like region |
| Dimerisation | PF08100.1 | 18.1 | Dimerisation domain |
| DnaJ | PF00226.19 | −8 | DnaJ domain |
| F-box | PF00646.21 | 13.6 | F-box domain |
| FAD_binding 4 | PF01565.12 | −8.1 | FAD binding domain |
| FAD_binding 6 | PF00970.13 | −11.4 | Oxidoreductase FAD-binding domain |
| FAD_binding 7 | PF03441.4 | 25 | FAD binding domain of DNA photolyase |
| FAE1_CUT1_RppA | PF08392.1 | −192.7 | FAE1/Type III polyketide synthase-like |
| | | | protein |
| FBD | PF08387.1 | 25 | FBD |
| Fasciclin | PF02469.10 | 4 | Fasciclin domain |
| GATase_2 | PF00310.10 | −106.2 | Glutamine amidotransferases class-II |
| GSHPx | PF00255.10 | −16 | Glutathione peroxidase |
| GST_N | PF02798.9 | 14.6 | Glutathione S-transferase, N-terminal domain |
| Globin | PF00042.11 | −8.8 | Globin |
| HALZ | PF02183.7 | 17 | Homeobox associated leucine zipper |
| HEAT_PBS | PF03130.5 | 15 | PBS lyase HEAT-like repeat |
| HLH | PF00010.15 | 8.2 | Helix-loop-helix DNA-binding domain |
| HSF_DNA-bind | PF00447.7 | −70 | HSF-type DNA-binding |
| HSP20 | PF00011.10 | 13 | Hsp20/alpha crystallin family |
| Homeobox | PF00046.18 | −4.1 | Homeobox domain |
| ICL | PF00463.10 | −234 | Isocitrate lyase family |
| Iso_dh | PF00180.10 | −97 | Isocitrate/isopropylmalate dehydrogenase |
| K-box | PF01486.7 | 0 | K-box region |
| KOW | PF00467.18 | 29.1 | KOW motif |
| LIM | PF00412.11 | 0 | LIM domain |
| LRR_2 | PF07723.2 | 6 | Leucine Rich Repeat |
| Lactamase_B | PF00753.16 | 24.6 | Metallo-beta-lactamase superfamily |
| MMR_HSR1 | PF01926.11 | 31.2 | GTPase of unknown function |
| Malic_M | PF03949.4 | −143.9 | Malic enzyme, NAD binding domain |
| Metallophos | PF00149.17 | 22 | Calcineurin-like phosphoesterase |
| Methyltransf_2 | PF00891.7 | −103.8 | O-methyltransferase |
| Miro | PF08477.1 | 28 | Miro-like protein |
| MtN3_slv | PF03083.5 | −0.8 | MtN3/saliva family |
| Myb_DNA-binding | PF00249.19 | 2.8 | Myb-like DNA-binding domain |
| NAD_binding_1 | PF00175.10 | −3.9 | Oxidoreductase NAD-binding domain |
| NAF | PF03822.4 | 4.5 | NAF domain |
| NPH3 | PF03000.4 | 25 | NPH3 family |
| Orn_Arg_deC_N | PF02784.7 | −76 | Pyridoxal-dependent decarboxylase, |
| | | | pyridoxal binding domain |
| Orn_DAP_Arg_deC | PF00278.12 | 6.7 | Pyridoxal-dependent decarboxylase, |
| | | | C-terminal sheet domain |
| PBP | PF01161.9 | −20.6 | Phosphatidylethanolamine-binding protein |
| PEP-utilizers | PF00391.12 | 10 | PEP-utilising enzyme, mobile domain |
| PEP-utilizers_C | PF02896.7 | −173 | PEP-utilising enzyme, TIM barrel domain |
| PFK | PF00365.10 | −132 | Phosphofructokinase |
| PHD | PF00628.17 | 25.9 | PHD-finger |
| PLDc | PF00614.11 | 0 | Phospholipase D Active site motif |
| PMEI | PF04043.5 | 25 | Plant invertase/pectin methylesterase |
| | | | inhibitor |
| PPDK_N | PF01326.8 | −87 | Pyruvate phosphate dikinase, PEP/pyruvate |
| | | | binding domain |
| Phi_1 | PF04674.2 | 25 | Phosphate-induced protein 1 conserved region |
| Pkinase | PF00069.14 | −70.8 | Protein kinase domain |
| Pkinase_Tyr | PF07714.5 | 65 | Protein tyrosine kinase |
| Pyridoxal_deC | PF00282.9 | −158.6 | Pyridoxal-dependent decarboxylase conserved |
| | | | domain |
| RMMBL | PF07521.1 | 18.5 | RNA-metabolising metallo-beta-lactamase |
| RNA_pol_L | PF01193.12 | 16.9 | RNA polymerase Rpb3/Rpb11 dimerisation domain |
| RRM_1 | PF00076.11 | 20.7 | RNA recognition motif. (a.k.a. RRM, RBD, |
| | | | or RNP domain) |
| RWP-RK | PF02042.5 | 25 | RWP-RK domain |
| Ras | PF00071.11 | −69.9 | Ras family |
| Redoxin | PF08534.1 | −1 | Redoxin |
| Ribosomal_L12 | PF00542.8 | 25 | Ribosomal protein L7/L12 C-terminal domain |
| Ribosomal_L14 | PF00238.9 | −8 | Ribosomal protein L14p/L23e |
| Ribosomal_L18p | PF00861.12 | 25 | Ribosomal L18p/L5e family |
| SRF-TF | PF00319.8 | 11 | SRF-type transcription factor (DNA-binding |
| | | | and dimerisation domain) |
| Sina | PF03145.6 | −48.4 | Seven in absentia protein family |
| Skp1 | PF01466.8 | −2 | Skp1 family, dimerisation domain |
| Skp1_POZ | PF03931.5 | 14.9 | Skp1 family, tetramerisation domain |
| Spermine_synth | PF01564.6 | −93.8 | Spermine/spermidine synthase |
| Suc_Fer-like | PF06999.2 | −42.4 | Sucrase/ferredoxin-like |
| TPP_enzyme_M | PF00205.11 | −23.9 | Thiamine pyrophosphate enzyme, central domain |
| TPP_enzyme_N | PF02776.7 | −70 | Thiamine pyrophosphate enzyme, N-terminal |
| | | | TPP binding domain |
| Transaldolase | PF00923.9 | −49 | Transaldolase |
| Tryp_alpha_amyl | PF00234.10 | −4 | Protease inhibitor/seed storage/LTP family |
| U-box | PF04564.5 | 10.5 | U-box domain |
| UIM | PF02809.10 | 4.1 | Ubiquitin interaction motif |
| UPF0005 | PF01027.11 | −6.7 | Uncharacterised protein family UPF0005 |
| UPF0057 | PF01679.7 | 25 | Uncharacterized protein family UPF0057 |
| WD40 | PF00400.20 | 21.5 | WD domain, G-beta repeat |
| X8 | PF07983.3 | −28.8 | X8 domain |
| eIF-5a | PF01287.9 | 9.6 | Eukaryotic initiation factor 5A hypusine, |
| | | | DNA-binding OB fold |
| efhand | PF00036.20 | 17.5 | EF hand |
| malic | PF00390.8 | 25 | Malic enzyme, N-terminal domain |
| p450 | PF00067.11 | −105 | Cytochrome P450 |
| peroxidase | PF00141.12 | −10 | Peroxidase |
| zf-A20 | PF01754.6 | 25 | A20-like zinc finger |
| zf-AN1 | PF01428.6 | 0 | AN1-like Zinc finger |
| zf-C3HC4 | PF00097.13 | 16.9 | Zinc finger, C3HC4 type (RING finger) |

### Example 10

### Selection of Transgenic Plants with Enhanced Agronomic Traits

This example illustrates identification of plant cells of the invention by screening transgenic plants and seeds for an enhanced trait. Transgenic seed and plants, e.g., with transgenic corn cells in the plants prepared in Example 2, transgenic soybean cells in the plants prepared in Example 3, transgnic cotton cells in the plants prepared in Example 4, and transgenic cells in the plants prepared in Example 6, are screened for enhanced water use efficiency, enhanced cold tolerance, increased yield, enhanced nitrogen use efficiency, enhanced seed protein and enhanced seed oil as compared to control plants.

### A. Selection for Enhanced Nitrogen Use Efficiency

The physiological efficacy of transgenic corn plants (tested as hybrids) can be tested for nitrogen use efficiency (NUE) traits in a high-throughput nitrogen (N) selection method. The collected data are compared to the measurements from wildtype controls using a statistical model to determine if the changes are due to the transgene. Raw data were analyzed by SAS software. Results shown herein are the comparison of transgenic plants relative to the wildtype controls.

### (1) Media Preparation for Planting a NUE Protocol

Planting materials used: Metro Mix 200 (vendor: Hummert) Cat. #10-0325, Scotts Micro Max Nutrients (vendor: Hummert) Cat. #07-6330, OS 4⅓″×3⅞″ pots (vendor: Hummert) Cat. #16-1415, OS trays (vendor: Hummert) Cat. #16-1515, Hoagland's macronutrients solution, Plastic 5″ stakes (vendor: Hummert) yellow Cat. #49-1569, white Cat. #49-1505, Labels with numbers indicating material contained in pots. Fill 500 pots to rim with Metro Mix 200 to a weight of ˜140 g/pot. Pots are filled uniformly by using a balancer. Add 0.4 g of Micro Max nutrients to each pot. Stir ingredients with spatula to a depth of 3 inches while preventing material loss.

### (2) Planting a NUE Selection in the Greenhouse

(a) Seed Germination—Each pot is lightly atered twice using reverse osmosis purified water. The first watering is scheduled to occur just before planting; and the second watering, after the seed has been planted in the pot. Ten Seeds of each entry (1 seed per pot) are planted to select eight healthy uniform seedlings. Additional wild type controls are planted for use as border rows. Alternatively, 15 seeds of each entry (1 seed per pot) are planted to select 12 healthy uniform seedlings (this larger number of plantings is used for the second, or confirmation, planting). Place pots on each of the 12 shelves in the Conviron growth chamber for seven days. This is done to allow more uniform germination and early seedling growth. The following growth chamber settings are 25° C./day and 22° C./night, 14 hours light and ten hours dark, humidity ˜80%, and light intensity ˜350 μmol/m²/s (at pot level). Watering is done via capillary matting similar to greenhouse benches with duration of ten minutes three times a day.

(b) Seedling transfer—After seven days, the best eight or 12 seedlings for the first or confirmation pass runs, respectively, are chosen and transferred to greenhouse benches. The pots are spaced eight inches apart (center to center) and are positioned on the benches using the spacing patterns printed on the capillary matting. The Vattex matting creates a 384-position grid, randomizing all range, row combinations. Additional pots of controls are placed along the outside of the experimental block to reduce border effects.

Plants are allowed to grow for 28 days under the low N run or for 23 days under the high N run. The macronutrients are dispensed in the form of a macronutrient solution (see composition below) containing precise amounts of N added (2 mM NH₄NO₃ for limiting N selection and 20 mM NH₄NO₃ for high N selection runs). Each pot is manually dispensed 100 ml of nutrient solution three times a week on alternate days starting at eight and ten days after planting for high N and low N runs, respectively. On the day of nutrient application, two 20 min waterings at 05:00 and 13:00 are skipped. The vattex matting should be changed every third run to avoid N accumulation and buildup of root matter. Table 12 shows the amount of nutrients in the nutrient solution for either the low or high nitrogen selection.

TABLE 12

| | 2 mM NH₄NO₃ | 20 mM NH₄NO₃ |
|---|---|---|
| | (Low Nitrogen | (high Nitrogen |
| | Growth Condition) | Growth Condition) |
| Nutrient Stock | mL/L | mL/L |
| 1M NH₄N0₃ | 2 | 20 |
| 1M KH₂PO₄ | 0.5 | 0.5 |
| 1M MgSO₄•7H₂O | 2 | 2 |
| 1M CaCl₂ | 2.5 | 2.5 |
| 1M K₂SO₄ | 1 | 1 |

Note:
Adjust pH to 5.6 with HCl or KOH

(c) Harvest Measurements and Data Collection—After 28 days of plant growth for low N runs and 23 days of plant growth for high N runs, the following measurements are taken (phenocodes in parentheses): total shoot fresh mass (g) (SFM) measured by Sartorius electronic balance, V6 leaf chlorophyll measured by Minolta SPAD meter (relative units) (LC), V6 leaf area (cm²) (LA) measured by a Li-Cor leaf area meter, V6 leaf fresh mass (g) (LFM) measured by Sartorius electronic balance, and V6 leaf dry mass (g) (LDM) measured by Sartorius electronic balance. Raw data were analyzed by SAS software. Results shown are the comparison of transgenic plants relative to the wildtype controls.

To take a leaf reading, samples were excised from the V6 leaf. Since chlorophyll meter readings of corn leaves are affected by the part of the leaf and the position of the leaf on the plant that is sampled, SPAD meter readings were done on leaf six of the plants. Three measurements per leaf were taken, of which the first reading was taken from a point one-half the distance between the leaf tip and the collar and halfway from the leaf margin to the midrib while two were taken toward the leaf tip. The measurements were restricted in the area from ½ to ¾ of the total length of the leaf (from the base) with approximately equal spacing between them. The average of the three measurements was taken from the SPAD machine.

Leaf fresh mass is recorded for an excised V6 leaf, the leaf is placed into a paper bag. The paper bags containing the leaves are then placed into a forced air oven at 80° C. for 3 days. After 3 days, the paper bags are removed from the oven and the leaf dry mass measurements are taken.

From the collected data, two derived measurements are made: (1) Leaf chlorophyll area (LCA), which is a product of V6 relative chlorophyll content and its leaf area (relative units). Leaf chlorophyll area=leaf chlorophyll X leaf area. This parameter gives an indication of the spread of chlorophyll over the entire leaf area; (2) specific leaf area (LSA) is calculated as the ratio of V6 leaf area to its dry mass (cm²/g dry mass), a parameter also recognized as a measure of NUE.

### Nitrogen Use Field Efficacy Assay

Level I. Transgenic plants provided by the present invention are planted in field without any nitrogen source being applied. Transgenic plants and control plants are grouped by genotype and construct with controls arranged randomly within genotype blocks. Each type of transgenic plants are tested by 3 replications and across 5 locations. Nitrogen levels in the fields are analyzed in early April pre-planting by collecting 30 sample soil cores from 0-24″ and 24 to 48″ soil layer. Soil samples are analyzed for nitrate-nitrogen, phosphorus (P), Potassium (K), organic matter and pH to provide baseline values. P, K and micronutrients are applied based upon soil test recommendations.

Level II. Transgenic plants provided by the present invention are planted in field with three levels of nitrogen (N) fertilizer being applied, i.e. low level (0 N), medium level (80 lb/ac) and high level (180 lb/ac). Liquid 28% or 32% UAN (Urea, Ammonium Nitrogen) are used as the N source and apply by broadcast boom and incorporate with a field cultivator with rear rolling basket in the same direction as intended crop rows. Although there is no N applied to the 0 N treatment the soil should still be disturbed in the same fashion as the treated area. Transgenic plants and control plants are grouped by genotype and construct with controls arranged randomly within genotype blocks. Each type of transgenic plants is tested by 3 replications and across 4 locations. Nitrogen levels in the fields are analyzed in early April pre-planting by collecting 30 sample soil cores from 0-24″ and 24 to 48″ soil layer. Soil samples are analyzed for nitrate-nitrogen, phosphorus (P), Potassium (K), organic matter and pH to provide baseline values. P, K and micronutrients are applied based upon soil test recommendations.

### B. Selection for Increased Yield

Many transgenic plants of this invention exhibit improved yield as compared to a control plant. Improved yield can result from enhanced seed sink potential, i.e. the number and size of endosperm cells or kernels and/or enhanced sink strength, i.e. the rate of starch biosynthesis. Sink potential can be established very early during kernel development, as endosperm cell number and size are determined within the first few days after pollination.

Effective yield selection of enhanced yielding transgenic corn events uses hybrid progeny of the transgenic event over multiple locations with plants grown under optimal production management practices, and maximum pest control. A useful target for improved yield is a 5% to 10% increase in yield as compared to yield produced by plants grown from seed for a control plant. Selection methods may be applied in multiple and diverse geographic locations, for example up to 16 or more locations, over one or more plating seasons, for example at least two planting seasons to statistically distinguish yield improvement from natural environmental effects. It is to plant multiple transgenic plants, positive and negative control plants, and pollinator plants in standard plots, for example 2 row plots, 20 feet long by 5 feet wide with 30 inches distance between rows and a 3 foot alley between ranges. Transgenic events can be grouped by recombinant DNA constructs with groups randomly placed in the field. A pollinator plot of a high quality corn line is planted for every two plots to allow open pollination when using male sterile transgenic events. A useful planting density is about 30,000 plants/acre. High planting density is greater than 30,000 plants/acre, preferably about 40,000 plants/acre, more preferably about 42,000 plants/acre, most preferably about 45,000 plants/acre. Surrogate indicators for yield improvement include source capacity (biomass), source output (sucrose and photosynthesis), sink components (kernel size, ear size, starch in the seed), development (light response, height, density tolerance), maturity, early flowering trait and physiological responses to high density planting, for example at 45,000 plants per acre, for example as illustrated in Table 13 and 14.

TABLE 13

| Timing | Evaluation | Description | comments |
|---|---|---|---|
| V2-3 | Early stand | Can be taken any time after | |
| | | germination and prior to | |
| | | removal of any plants. | |
| Pollen shed | GDU to 50% shed | GDU to 50% plants shedding | |
| | | 50% tassel. | |
| Silking | GDU to 50% silk | GDU to 50% plants showing | |
| | | silks. | |
| Maturity | Plant height | Height from soil surface to | 10 plants per plot - Yield |
| | | flag leaf attachment (inches). | team assistance |
| Maturity | Ear height | Height from soil surface to | 10 plants per plot - Yield |
| | | primary ear attachment node. | team assistance |
| Maturity | Leaves above ear | visual scores: erect, size, | |
| | | rolling | |
| Maturity | Tassel size | Visual scores +/− vs. WT | |
| Pre-Harvest | Final Stand | Final stand count prior to | |
| | | harvest, exclude tillers | |
| Pre-Harvest | Stalk lodging | No. of stalks broken below | |
| | | the primary ear attachment. | |
| | | Exclude leaning tillers | |
| Pre-Harvest | Root lodging | No. of stalks leaning >45° | |
| | | angle from perpendicular. | |
| Pre-Harvest | Stay green | After physiological maturity | |
| | | and when differences among | |
| | | genotypes are evident: Scale | |
| | | 1 (90-100% tissue green)-9 | |
| | | (0-19% tissue green). | |
| Harvest | Grain Yield | Grain yield/plot (Shell | |
| | | weight) | |

TABLE 14

| Timing | Evaluation | Description |
|---|---|---|
| V8-V12 | Chlorophyll | |
| V12-VT | Ear leaf area | |
| V15-15DAP | Chl fluorescence | |
| V15-15DAP | CER | |
| 15-25 DAP | Carbohydrates | sucrose, starch |
| Pre-Harvest | 1st internode diameter | |
| Pre-Harvest | Base 3 internode diameter | |
| Pre-Harvest | Ear internode diameter | |
| Maturity | Ear traits | diameter, length, kernel |
| | | number, kernel weight |

Electron transport rates (ETR) and CO2 exchange rates (CER): ETR and CER are measured with Li6400LCF (Licor, Lincoln, Nebr.) around V9-R1 stages. Leaf chlorophyll fluorescence is a quick way to monitor the source activity and is reported to be highly correlated with CO₂ assimilation under varies conditions (Photosyn Research, 37: 89-102). The youngest fully expanded leaf or 2 leaves above the ear leaf is measured with actinic light 1500 (with 10% blue light) micromol m⁻² s⁻¹, 28oC, CO2 levels 450 ppm. Ten plants are measured in each event. There are 2 readings for each plant.

A hand-held chlorophyll meter SPAD-502 (Minolta-Japan) is used to measure the total chlorophyll level on live transgenic plants and the wild type counterparts a. Three trifoliates from each plant are analyzed, and each trifoliate were analyzed three times. Then 9 data points are averaged to obtain the chlorophyll level. The number of analyzed plants of each genotype ranges from 5 to 8.

When selecting for yield improvement a useful statistical measurement approach comprises three components, i.e. modeling spatial autocorrelation of the test field separately for each location, adjusting traits of recombinant DNA events for spatial dependence for each location, and conducting an across location analysis. The first step in modeling spatial autocorrelation is estimating the covariance parameters of the semivariogram. A spherical covariance model is assumed to model the spatial autocorrelation. Because of the size and nature of the trial, it is likely that the spatial autocorrelation may change. Therefore, anisotropy is also assumed along with spherical covariance structure. The following set of equations describes the statistical form of the anisotropic spherical covariance model.

$$C ( h ; θ ) = vI ( h = 0 ) + {σ}^{2} ( 1 - \frac{3}{2} h + \frac{1}{2} {h}^{3} ) I ( h < 1 ) ,$$

where I() is the indicator function, h=\sqrt{\dot{x}²+\dot{y}²}, and

\dot{x}=[cos(ρπ/180)(x₁−x₂)−sin(ρπ/180)(y₁−y₂)]/ωₓ

\dot{y}=[sin(ρπ/180)(x₁−x₂)+cos(ρπ/180)(y₁−y₂)]/ω_{y}

where s₁=(x₁, y₁) are the spatial coordinates of one location and s₂=(x₂, y₂) are the spatial coordinates of the second location. There are 5 covariance parameters, θ=(ν, σ², ρ, ωₙ, ωⱼ), where ν is the nugget effect, σ² is the partial sill, ρ is a rotation in degrees clockwise from north, ωₙ is a scaling parameter for the minor axis and ωⱼ; is a scaling parameter for the major axis of an anisotropical ellipse of equal covariance. The five covariance parameters that defines the spatial trend will then be estimated by using data from heavily replicated pollinator plots via restricted maximum likelihood approach. In a multi-location field trial, spatial trend are modeled separately for each location.

After obtaining the variance parameters of the model, a variance-covariance structure is generated for the data set to be analyzed. This variance-covariance structure contains spatial information required to adjust yield data for spatial dependence. In this case, a nested model that best represents the treatment and experimental design of the study is used along with the variance-covariance structure to adjust the yield data. During this process the nursery or the seed batch effects can also be modeled and estimated to adjust the yields for any yield parity caused by seed batch differences. After spatially adjusted data from different locations are generated, all adjusted data is combined and analyzed assuming locations as replications. In this analysis, intra and inter-location variances are combined to estimate the standard error of yield from transgenic plants and control plants. Relative mean comparisons are used to indicate statistically significant yield improvements.

### C. Selection for Enhanced Water Use Efficiency (WUE)

Described in this example is a high-throughput method for greenhouse selection of transgenic corn plants to wild type corn plants (tested as inbreds or hybrids) for water use efficiency and method for selection transgenic cotton plants for water use efficiency. This selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment. The hydration status of the shoot tissues following the drought is also measured. The plant height is measured at three time points. The first is taken just prior to the onset drought when the plant is 11 days old, which is the shoot initial height (SIH). The plant height is also measured halfway throughout the drought/re-water regimen, on day 18 after planting, to give rise to the shoot mid-drought height (SMH). Upon the completion of the final drought cycle on day 26 after planting, the shoot portion of the plant is harvested and measured for a final height, which is the shoot wilt height (SWH) and also measured for shoot wilted biomass (SWM). The shoot is placed in water at 40 degree Celsius in the dark. Three days later, the shoot is weighted to give rise to the shoot turgid weight (STM). After drying in an oven for four days, the shoots are weighted for shoot dry biomass (SDM). The shoot average height (SAH) is the mean plant height across the 3 height measurements. The procedure described above may be adjusted for +/−˜one day for each step given the situation.

To correct for slight differences between plants, a size corrected growth value is derived from SIH and SWH. This is the Relative Growth Rate (RGR). Relative Growth Rate (RGR) is calculated for each shoot using the formula [RGR %=(SWH−SIH)/((SWH+SIH)/2)*100]. Relative water content (RWC) is a measurement of how much (%) of the plant was water at harvest. Water Content (RWC) is calculated for each shoot using the formula [RWC %=(SWM−SDM)/(STM−SDM)*100]. Fully watered corn plants of this age run around 98% RWC.

Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA. The specified culture conditions are growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions, i.e. irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential, increased biomass, enhanced leaf extension rates, and improved fiber parameters.

### D. Selection for Growth Under Cold Stress

(1) Cold germination assay—Three sets of seeds are used for the assay. The first set consists of positive transgenic events (F1 hybrid) where the genes of the present invention are expressed in the seed. The second seed set is nontransgenic, wild-type negative control made from the same genotype as the transgenic events. The third set consisted of two cold tolerant and one cold sensitive commercial check lines of corn. All seeds are treated with a fungicide “Captan” (MAESTRO® 80DF Fungicide, Arvesta Corporation, San Francisco, Calif., USA). 0.43 mL Captan is applied per 45 g of corn seeds by mixing it well and drying the fungicide prior to the experiment.

Corn kernels are placed embryo side down on blotter paper within an individual cell (8.9×8.9 cm) of a germination tray (54×36 cm). Ten seeds from an event are placed into one cell of the germination tray. Each tray can hold 21 transgenic events and 3 replicates of wildtype (LH244SDms+LH59), which is randomized in a complete block design. For every event there are five replications (five trays). The trays are placed at 9.7 C for 24 days (no light) in a Convrion growth chamber (Conviron Model PGV36, Controlled Environments, Winnipeg, Canada). Two hundred and fifty millilters of deionized water are added to each germination tray. Germination counts are taken 10th, 11th, 12th, 13th, 14th, 17th, 19th, 21st, and 24th day after start date of the experiment. Seeds are considered germinated if the emerged radicle size is 1 cm. From the germination counts germination index is calculated.

The germination index is calculated as per:

Germination index=(Σ([T+1−nᵢ]*[Pᵢ−Pᵢ₋₁]))/T

Where T is the total number of days for which the germination assay is performed. The number of days after planting is defined by n. “i” indicated the number of times the germination had been counted, including the current day. P is the percentage of seeds germinated during any given rating. Statistical differences are calculated between transgenic events and wild type control. After statistical analysis, the events that show a statistical significance at the p level of less than 0.1 relative to wild-type controls will advance to a secondary cold selection. The secondary cold screen is conducted in the same manner of the primary selection only increasing the number of repetitions to ten. Statistical analysis of the data from the secondary selection is conducted to identify the events that show a statistical significance at the p level of less than 0.05 relative to wild-type controls.

TABLE 15

| | | | Germination Index | | | |
|---|---|---|---|---|---|---|
| | | | 1st Run | | 2nd Run | |
| PEP SEQ | | | % | P | % | P |
| ID | CONSTRUCT | Event | Change | value | Change | value |
| 266 | PMON92840 | MON810, ZM_M106115 | 28 | 0.079 | 15 | 0.233 |
| | PMON92840 | MON810, ZM_M107208 | 58 | 0.000 | 24 | 0.049 |
| | PMON92840 | MON810, ZM_M107212 | 36 | 0.026 | 34 | 0.006 |
| | PMON92840 | MON810, ZM_M107214 | 53 | 0.001 | 26 | 0.035 |
| | PMON92840 | MON810, ZM_M107221 | 29 | 0.072 | −5 | 0.663 |
| | PMON92840 | MON810, ZM_M107224 | 60 | 0.000 | 35 | 0.004 |
| | PMON92840 | MON810, ZM_M107228 | 39 | 0.017 | 30 | 0.016 |
| 284 | PMON92854 | MON810, ZM_M103991 | 28 | 0.070 | 9 | 0.364 |
| | PMON92854 | MON810, ZM_M104002 | 35 | 0.025 | 8 | 0.412 |
| | PMON92854 | MON810, ZM_M105195 | 27 | 0.082 | 10 | 0.321 |
| | PMON92854 | MON810, ZM_M105213 | 30 | 0.060 | 21 | 0.033 |
| | PMON92854 | MON810, ZM_M105218 | 74 | 0.000 | 49 | 0.000 |
| | PMON92854 | MON810, ZM_M105267 | 43 | 0.006 | 28 | 0.004 |
| | PMON92854 | MON810, ZM_M106123 | −1 | 0.965 | 30 | 0.002 |

(2) Cold Shock assay—The experimental set-up for the cold shock assay is the same as described in the above cold germination assay except seeds were grown in potted media for the cold shock assay.

The desired numbers of 2.5″ square plastic pots are placed on flats (n=32, 4×8). Pots were filled with Metro Mix 200 soil-less media containing 19:6:12 fertilizer (6 lbs/cubic yard) (Metro Mix, Pots and Flat are obtained from Hummert International, Earth City, Mo.). After planting seeds, pots are placed in a growth chamber set at 23° C., relative humidity of 65% with 12 hour day and night photoperiod (300 uE/m2-min). Planted seeds are watered for 20 minute every other day by sub-irrigation and flats were rotated every third day in a growth chamber for growing corn seedlings.

On the 10^{th} day after planting the transgenic positive and wild-type negative (WT) plants are positioned in flats in an alternating pattern. Chlorophyll fluorescence of plants is measured on the 10^{th} day during the dark period of growth by using a PAM-2000 portable fluorometer as per the manufacturer's instructions (Walz, Germany). After chlorophyll measurements, leaf samples from each event are collected for confirming the expression of genes of the present invention. For expression analysis six V1 leaf tips from each selection are randomly harvested. The flats are moved to a growth chamber set at 5° C. All other conditions such as humidity, day/night cycle and light intensity are held constant in the growth chamber. The flats are sub-irrigated every day after transfer to the cold temperature. On the 4^{th} day chlorophyll fluorescence is measured. Plants are transferred to normal growth conditions after six days of cold shock treatment and allowed to recover for the next three days. During this recovery period the length of the V3 leaf is measured on the 1^{st} and 3^{rd} days. After two days of recovery V2 leaf damage is determined visually by estimating percent of green V2 leaf.

Statistical differences in V3 leaf growth, V2 leaf necrosis and fluorescence during pre-shock and cold shock can be used for estimation of cold shock damage on corn plants.

(3) Early seedling growth assay—Three sets of seeds are used for the experiment. The first set consists of positive transgenic events (F1 hybrid) where the genes of the present invention are expressed in the seed. The second seed set is nontransgenic, wild-type negative control made from the same genotype as the transgenic events. The third seed set consists of two cold tolerant and two cold sensitive commercial check lines of corn. All seeds are treated with a fungicide “Captan”, (3a,4,7,a-tetrahydro-2-[(trichloromethly)thio]-1H-isoindole-1,3(2H)-dione, Drex Chemical Co. Memphis, Tenn.). Captan (0.43 mL) was applied per 45 g of corn seeds by mixing it well and drying the fungicide prior to the experiment.

Seeds are grown in germination paper for the early seedling growth assay. Three 12″×18″ pieces of germination paper (Anchor Paper #SD7606) are used for each entry in the test (three repetitions per transgenic event). The papers are wetted in a solution of 0.5% KNO₃ and 0.1% Thyram.

For each paper fifteen seeds are placed on the line evenly spaced down the length of the paper. The fifteen seeds are positioned on the paper such that the radical would grow downward, for example longer distance to the paper's edge. The wet paper is rolled up starting from one of the short ends. The paper is rolled evenly and tight enough to hold the seeds in place. The roll is secured into place with two large paper clips, one at the top and one at the bottom. The rolls are incubated in a growth chamber at 23° C. for three days in a randomized complete block design within an appropriate container. The chamber is set for 65% humidity with no light cycle. For the cold stress treatment the rolls are then incubated in a growth chamber at 12° C. for twelve days. The chamber is set for 65% humidity with no light cycle.

After the cold treatment the germination papers are unrolled and the seeds that did not germinate are discarded. The lengths of the radicle and coleoptile for each seed are measured through an automated imaging program that automatically collects and processes the images. The imaging program automatically measures the shoot length, root length, and whole seedling length of every individual seedling and then calculates the average of each roll.

After statistical analysis, the events that show a statistical significance at the p level of less than 0.1 relative to wild-type controls will advance to a secondary cold selection. The secondary cold selection is conducted in the same manner of the primary selection only increasing the number of repetitions to five. Statistical analysis of the data from the secondary selection is conducted to identify the events that show a statistical significance at the p level of less than 0.05 relative to wild-type controls.

### (4). Cold Field Efficacy Trial

This example sets forth a cold field efficacy trial to identify gene constructs that confer enhanced cold vigor at germination and early seedling growth under early spring planting field conditions in conventional-till and simulated no-till environments. Seeds are planted into the ground around two weeks before local farmers are beginning to plant corn so that a significant cold stress is exerted onto the crop, named as cold treatment. Seeds also are planted under local optimal planting conditions such that the crop has little or no exposure to cold condition, named as normal treatment. The cold field efficacy trials are carried out in five locations, including Glyndon Minn., Mason Mich., Monmouth Ill., Dayton Iowa, Mystic Conn. At each location, seeds are planted under both cold and normal conditions with 3 repetitions per treatment, 20 kernels per row and single row per plot. Seeds are planted 1.5 to 2 inch deep into soil to avoid muddy conditions. Two temperature monitors are set up at each location to monitor both air and soil temperature daily.

Seed emergence is defined as the point when the growing shoot breaks the soil surface. The number of emerged seedling in each plot is counted everyday from the day the earliest plot begins to emerge until no significant changes in emergence occur. In addition, for each planting date, the latest date when emergence is 0 in all plots is also recorded. Seedling vigor is also rated at V3-V4 stage before the average of corn plant height reaches 10 inches, with 1=excellent early growth, 5=Average growth and 9=poor growth. Days to 50% emergence, maximum percent emergence and seedling vigor are calculated using SAS software for the data within each location or across all locations.

E. Screens for Transgenic Plant Seeds with Increased Protein and/or Oil Levels

This example sets forth a high-throughput selection for identifying plant seeds with improvement in seed composition using the Infratec 1200 series Grain Analyzer, which is a near-infrared transmittance spectrometer used to determine the composition of a bulk seed sample. Near infrared analysis is a non-destructive, high-throughput method that can analyze multiple traits in a single sample scan. An NIR calibration for the analytes of interest is used to predict the values of an unknown sample. The NIR spectrum is obtained for the sample and compared to the calibration using a complex chemometric software package that provides a predicted values as well as information on how well the sample fits in the calibration.

Infratec Model 1221, 1225, or 1227 with transport module by Foss North America is used with cuvette, item #1000-4033, Foss North America or for small samples with small cell cuvette, Foss standard cuvette modified by Leon Girard Co. Corn and soy check samples of varying composition maintained in check cell cuvettes are supplied by Leon Girard Co. NIT collection software is provided by Maximum Consulting Inc. Software. Calculations are performed automatically by the software. Seed samples are received in packets or containers with barcode labels from the customer. The seed is poured into the cuvettes and analyzed as received.

TABLE 16

| | |
|---|---|
| Typical sample(s): | Whole grain corn and soybean seeds |
| Analytical time to run method: | Less than 0.75 min per sample |
| Total elapsed time per run: | 1.5 minute per sample |
| Typical and minimum sample | Corn typical: 50 cc; minimum 30 cc |
| size: | Soybean typical: 50 cc; minimum 5 cc |
| Typical analytical range: | Determined in part by the specific |
| | calibration. |
| | Corn - moisture 5-15%, oil 5-20%, |
| | protein 5-30%, starch 50-75%, and |
| | density 1.0-1.3%. |
| | Soybean - moisture 5-15%, oil 15-25%, |
| | and protein 35-50%. |

TABLE 17
Transgenic corn plants have an increased oil level in seeds

| PEP | | | 2004 Data | | 2005 Data | |
|---|---|---|---|---|---|---|
| SEQ | | | Oil | | Oil | |
| ID NO | Event | Construct | Delta | Pvalue | delta | P value |
| 231 | ZM_S90572 | PMON17730 | 0.18 | 0.22 | 0.25 | 0.04 |
| | ZM_S90588 | PMON17730 | N/A | N/A | 0.10 | 0.33 |
| | ZM_S90610 | PMON17730 | N/A | N/A | −0.03 | 0.78 |
| | ZM_S90614 | PMON17730 | 0.26 | 0.08 | 0.47 | 0.00 |
| | ZM_S90622 | PMON17730 | −0.03 | 0.82 | 0.31 | 0.00 |

TABLE 18
Transgenic corn plants have an increased protein level in seeds

| PEP | | | | | | | | | | |
|---|---|---|---|---|---|---|---|---|---|---|
| SEQ | | | 1st Inbred protein trial | | | | 2nd Inbred protein trial | | | |
| ID | | | Mean | Mean | | | Mean | Mean | | |
| NO | Construct | Event | transgenic | control | Delta | Pvalue | transgenic | control | Delta | Pvalue |
| 208 | PMON92607 | ZM_M106133 | 14.06 | 10.39 | 3.67 | 0 | 11.91 | 10.06 | 1.84 | 0.0023 |
| | | ZM_M106129 | 16.46 | 10.39 | 6.07 | 0 | 16.00 | 10.06 | 5.94 | 0 |
| | | ZM_M105269 | 15.80 | 10.39 | 5.41 | 0 | 14.89 | 10.06 | 4.83 | 0 |
| | | ZM_M105268 | 14.07 | 10.39 | 3.67 | 0 | 12.81 | 10.06 | 2.74 | 0 |
| | | ZM_M104742 | 12.57 | 10.39 | 2.18 | 0.0064 | 12.52 | 10.06 | 2.46 | 0 |
| | | ZM_M104740 | 14.45 | 10.39 | 4.06 | 0 | 12.93 | 10.06 | 2.86 | 0 |
| | | ZM_M104403 | 12.90 | 10.39 | 2.51 | 0.0017 | 12.60 | 10.06 | 2.53 | 0 |
| | | ZM_M104399 | 13.21 | 10.39 | 2.82 | 0.0004 | 14.04 | 10.06 | 3.98 | 0 |
| | | ZM_M104398 | 14.91 | 10.39 | 4.51 | 0 | 12.79 | 10.06 | 2.73 | 0 |
| | | ZM_M104396 | 12.13 | 10.39 | 1.74 | 0.0289 | 12.90 | 10.06 | 2.83 | 0 |
| | | ZM_M104385 | 13.12 | 10.39 | 2.72 | 0.0007 | 12.89 | 10.06 | 2.82 | 0 |
| | | ZM_M104371 | 12.23 | 10.39 | 1.83 | 0.0213 | 12.18 | 10.06 | 2.12 | 0.0004 |
| | | ZM_M104369 | 13.41 | 10.39 | 3.01 | 0.0002 | 11.35 | 10.06 | 1.29 | 0.0309 |
| | | ZM_M103621 | 12.26 | 10.39 | 1.86 | 0.0191 | 10.76 | 10.06 | 0.69 | 0.2425 |
| | | ZM_M106138 | 13.74 | 10.39 | 3.35 | 0 | 13.34 | 10.06 | 3.28 | 0 |

TABLE 19
Transgenic soybean plants have an increased seed oil level

| PEP | | | | | | | | | |
|---|---|---|---|---|---|---|---|---|---|
| SEQ | | | | seed oil content | | | seed protein content | | |
| ID | | | | control | transgenic | | control | transgenic | |
| NO | construct | event | run | mean | mean | delta | mean | mean | delta |
| 231 | PMON94697 | construct | 1 | 20.0 | 19.7 | −0.3 | 42.0 | 42.9 | 0.9 |
| | | analysis | 2 | 19.6 | 20.2 | 0.6* | 43.3 | 43.6 | 0.3 |
| | | | 3 | 19.9 | 20.6 | 0.7* | 42.5 | 43.1 | 0.6 |
| | | GM_A79833 | 1 | 20.2 | 19.7 | −0.3 | 42.0 | 42.9 | 0.9 |
| | | | 2 | 19.6 | 19.9 | 0.3 | 43.3 | 43.5 | 0.2 |
| | | GM_A79838 | 2 | 19.6 | 19.8 | 0.2 | 43.3 | 45.2 | 1.9* |
| | | GM_A79839 | 2 | 19.6 | 20.4 | 0.8* | 43.3 | 43.7 | 0.4 |
| | | GM_A79857 | 2 | 19.6 | 19.9 | 0.3 | 43.3 | 43.7 | 0.4 |
| | | GM_A79859 | 2 | 19.6 | 20.6 | 1.0* | 43.3 | 43.7 | −0.6 |
| | | GM_A79894 | 2 | 19.6 | 20.4 | 0.8* | 43.3 | 43.1 | −0.2 |
| | | GM_A79896 | 2 | 19.6 | 19.8 | 0.2 | 43.3 | 44.4 | 1.1* |
| | | GM_A79914 | 2 | 19.6 | 20.9 | 1.3* | 43.3 | 42.7 | −0.6 |
| | | | 3 | 19.9 | 20.6 | 0.7 | 42.5 | 43.4 | 0.9 |
| | | GM_A79934 | 3 | 19.9 | 20.3 | 0.4* | 42.5 | 43.3 | 0.8* |
| | | GM_A79936 | 3 | 19.9 | 21.0 | 1.1* | 42.5 | 42.5 | 0.0 |

Data point with “*” indicate a statistically significant delta (the difference between transgenic and control plants).
Seed protein or oil is measured as a percentage of total seed composition.

1. A plant cell with stably integrated, recombinant DNA comprising a promoter that is functional in plant cells and that is operably linked to DNA from a plant, bacteria or yeast that encodes a protein having at least one domain of amino acids in a sequence that exceeds the Pfam gathering cutoff for amino acid sequence alignment with a protein domain family identified by a Fasciclin Pfam, the Pfam gathering cuttoff for said protein domain families is 4; wherein said plant cell is selected from a population of plant cells with said recombinant DNA by screening plants that are regenerated from plant cells in said population and that express said protein for an enhanced trait as compared to control plants that do not have said recombinant DNA; and wherein said enhanced trait is selected from group of enhanced traits consisting of enhanced water use efficiency, enhanced cold tolerance, increased yield, enhanced nitrogen use efficiency, enhanced seed protein and enhanced seed oil.

2. A plant cell with stably integrated, recombinant DNA comprising a promoter that is functional in plant cells and that is operably linked to DNA from a plant, bacteria or yeast that encodes a protein having at least one domain of amino acids in a sequence that exceeds the Pfam gathering cutoff for amino acid sequence alignment with a protein domain family identified by a Pfam name in the group of Pfam names consisting of FAD_binding_{—}4, MtN3_slv, Homeobox, FAD_binding_{—}6, RWP-RK, PMEI, FAD_binding_{—}7, RRM_{—}1, Transaldolase, RNA_pol_L, WD40, U-box, Cyclin_N, Skp1, Redoxin, DZC, PBP, TPP_enzyme_M, CBFD_NFYB_HMF, TPP_enzyme_N, PFK, Caleosin, Iso_dh, Ribosomal_L18p, Metallophos, zf-A20, Ras, BBE, NAF, PLDc, DUF1242, Pkinase, C2, p450, Pyridoxal_deC, FBD, UPF0005, HEAT_PBS, GST_N, PEP-utilizers, Alpha-amylase, Amino_oxidase, SRF-TF, Phi_{—}1, Malic_M, Tryp_alpha_amyl, GSHPx, Miro, HSF_DNA-bind, DNA_photolyase, Sina, CTP_transf_{—}2, Abhydrolase_{—}3, Chal_sti_synt_C, ACP_syn_III_C, ADH_zinc_N, CSD, Globin, GATase_{—}2, Amidohydro_{—}1, HLH, HALZ, Amidohydro_{—}3, Lactamase_B, HSP20, DAO, DUF296, AT_hook, A WPM-19, Dimerisation, Suc_Fer-like, Methyltransf_{—}2, Aminotran_{—}3, PHD, MMR_HSR1, Aldo_ket_red, zf-AN1, malic, Fasciclin, UPF0057, DUF221, Pkinase_Tyr, DnaJ, Cofilin_ADF, Orn_Arg_deC_N, Skp1_POZ, Asn_synthase, K-box, LRR_{—}2, Ribosomal_L12, Ammonium_transp, Ribosomal_L14, KOW, DUF1336, DS, Aa_trans, CcmH, peroxidase, eIF-5a, Aldedh, PEP-utilizers_C, ADH_N, UIM, NAD_binding_{—}1, zf-C3HC4, Spermine_synth, AUX_IAA, LIM, Anti-silence, X8, Citrate_synt, 14-3-3, RMMBL, efhand, NPH3, CAF1, ICL, FAE1_CUT1_RppA, Orn_DAP_Arg_deC, PPDK_N, Myb_DNA-binding, AP2, F-box, and APS_kinase wherein the Pfam gathering cuttoff for said protein domain families is stated in Table 11; wherein said plant cell is selected from a population of plant cells with said recombinant DNA by screening plants that are regenerated from plant cells in said population and that express said protein for an enhanced trait as compared to control plants that do not have said recombinant DNA; and wherein said enhanced trait is selected from group of enhanced traits consisting of enhanced water use efficiency, enhanced cold tolerance, increased yield, enhanced nitrogen use efficiency, enhanced seed protein and enhanced seed oil.

3. A plant cell of claim 2 wherein said protein has an amino acid sequence with at least 90% identity to a consensus amino acid sequence in the group of consensus amino acid sequences consisting of the consensus amino acid sequence constructed for SEQ ID NO: 194 and homologs thereof listed in Table 7 through the consensus amino acid sequence constructed for SEQ ID NO:386 and homologs thereof listed in Table 7.

4. A plant cell of claim 2 wherein said protein is selected from the group of proteins identified in Table 1.

5. A plant cell of claim 2 further comprising DNA expressing a protein that provides tolerance from exposure to an herbicide applied at levels that are lethal to a wild type of said plant cell.

6. A plant cell of claim 5 wherein the agent of said herbicide is a glyphosate, dicamba, or glufosinate compound.

7. A transgenic plant comprising a plurality of the plant cell of claim 1.

8. A transgenic plant of claim 7 which is homozygous for said recombinant DNA.

9. A transgenic seed comprising a plurality of the plant cell of claim 1.

10. A transgenic seed of claim 9 from a corn, soybean, cotton, canola, alfalfa, wheat or rice plant.

11. A transgenic pollen grain comprising a haploid derivative of a plant cell of claim 1.

12. A method for manufacturing non-natural, transgenic seed that can be used to produce a crop of transgenic plants with an enhanced trait resulting from expression of stably-integrated, recombinant DNA comprising a promoter that is (a) functional in plant cells and (b) is operably linked to DNA from a plant, bacteria or yeast that encodes a protein having at least one domain of amino acids in a sequence that exceeds the Pfam gathering cutoff for amino acid sequence alignment with a protein domain family identified by a Pfam name in the group of Pfam names consisting of FAD_binding_{—}4, MtN3_slv, Homeobox, FAD_binding_{—}6, RWP-RK, PMEI, FAD_binding_{—}7, Transaldolase, RNA_pol_L, WD40, U-box, Cyclin_N, Skp1, Redoxin, DZC, PBP, TPP_enzyme_M, CBFD_NFYB_HMF, TPP_enzyme_N, PFK, Caleosin, Iso_dh, Ribosomal_L18p, Metallophos, zf-A20, Ras, BBE, NAF, PLDc, DUF1242, Pkinase, C2, p450, Pyridoxal_deC, FBD, UPF0005, HEAT_PBS, GST_N, PEP-utilizers, Alpha-amylase, Amino_oxidase, SRF-TF, Phi_{—}1, Malic_M, Tryp_alpha_amyl, GSHPx, Miro, HSF_DNA-bind, DNA_photolyase, Sina, CTP_transf_{—}2, Abhydrolase_{—}3, Chal_sti_synt_C, ACP_syn_III_C, ADH_zinc_N, CSD, Globin, GATase_{—}2, Amidohydro_{—}1, HLH, HALZ, Amidohydro_{—}3, Lactamase_B, HSP20, DAO, DUF296, AT_hook, A WPM-19, Dimerisation, Suc_Fer-like, Methyltransf_{—}2, Aminotran_{—}3, PHD, MMR_HSR1, Aldo_ket_red, zf-AN1, malic, Fasciclin, UPF0057, DUF221, Pkinase_Tyr, DnaJ, Cofilin_ADF, Orn_Arg_deC_N, Skp1_POZ, Asn_synthase, K-box, LRR_{—}2, Ribosomal_L12, Ammonium_transp, Ribosomal_L14, KOW, DUF1336, DS, Aa_trans, CcmH, peroxidase, eIF-5a, Aldedh, PEP-utilizers_C, ADH_N, UIM, NAD_binding_{—}1, zf-C3HC4, Spermine_synth, AUX_IAA, LIM, Anti-silence, X8, Citrate_synt, 14-3-3, RMMBL, efhand, NPH3, CAF1, ICL, FAE1_CUT1_RppA, Orn_DAP_Arg_deC, PPDK_N, Myb_DNA-binding, AP2, F-box, and APS_kinase; wherein the gathering cutoff for said protein domain families is stated in Table 11; and wherein said enhanced trait is selected from the group of enhanced traits consisting of enhanced water use efficiency, enhanced cold tolerance, increased yield, enhanced nitrogen use efficiency, enhanced seed protein and enhanced seed oil, said method for manufacturing said seed comprising:
(a) screening a population of plants for said enhanced trait and said recombinant DNA, wherein individual plants in said population can exhibit said trait at a level less than, essentially the same as or greater than the level that said trait is exhibited in control plants which do not express the recombinant DNA,
(b) selecting from said population one or more plants that exhibit the trait at a level greater than the level that said trait is exhibited in control plants,
(c) verifying that said recombinant DNA is stably integrated in said selected plants,
(d) analyzing tissue of a selected plant to determine the production of a protein having the function of a protein encoded by nucleotides in a sequence of one of SEQ ID NO:1-193; and
(e) collecting seed from a selected plant.

13. A method of claim 12 wherein plants in said population further comprise DNA expressing a protein that provides tolerance to exposure to an herbicide applied at levels that are lethal to wild type plant cells, and wherein said selecting is effected by treating said population with said herbicide.

14. A method of claim 13 wherein said herbicide comprises a glyphosate, dicamba, or glufosinate compound.

15. A method of claim 12 wherein said selecting is effected by identifying plants with said enhanced trait.

16. A method of claim 12 wherein said seed is corn, soybean, cotton, alfalfa, wheat or rice seed.

17. A method of producing hybrid corn seed comprising:
acquiring hybrid corn seed from a herbicide tolerant corn plant which also has stably-integrated, recombinant DNA comprising a promoter that is (a) functional in plant cells and (b) is operably linked to DNA that encodes a protein having at least one domain of amino acids in a sequence that exceeds the Pfam gathering cutoff for amino acid sequence alignment with a protein domain family identified by a Pfam name in the group of Pfam names consisting of FAD_binding_{—}4, MtN3_slv, Homeobox, FAD_binding_{—}6, RWP-RK, PMEI, FAD_binding_{—}7, RRM_{—}1, Transaldolase, RNA_pol_L, WD40, U-box, Cyclin_N, Skp1, Redoxin, DZC, PBP, TPP_enzyme_M, CBFD_NFYB_HMF, TPP_enzyme_N, PFK, Caleosin, Iso_dh, Ribosomal_L18p, Metallophos, zf-A20, Ras, BBE, NAF, PLDc, DUF1242, Pkinase, C2, p450, Pyridoxal_deC, FBD, UPF0005, HEAT_PBS, GST_N, PEP-utilizers, Alpha-amylase, Amino_oxidase, SRF-TF, Phi_{—}1, Malic_M, Tryp_alpha_amyl, GSHPx, Miro, HSF_DNA-bind, DNA_photolyase, Sina, CTP_transf_{—}2, Abhydrolase_{—}3, Chal_sti_synt_C, ACP_syn_III_C, ADH_zinc_N, CSD, Globin, GATase_{—}2, Amidohydro_{—}1, HLH, HALZ, Amidohydro_{—}3, Lactamase_B, HSP20, DAO, DUF296, AT_hook, A WPM-19, Dimerisation, Suc_Fer-like, Methyltransf_{—}2, Aminotran_{—}3, PHD, MMR_HSR1, Aldo_ket_red, zf-AN1, malic, Fasciclin, UPF0057, DUF221, Pkinase_Tyr, DnaJ, Cofilin_ADF, Orn_Arg_deC_N, Skp1_POZ, Asn_synthase, K-box, LRR_{—}2, Ribosomal_L12, Ammonium_transp, Ribosomal_L14, KOW, DUF1336, DS, Aa_trans, CcmH, peroxidase, eIF-5a, Aldedh, PEP-utilizers_C, ADH_N, UIM, NAD_binding_{—}1, zf-C3HC4, Spermine_synth, AUX_IAA, LIM, Anti-silence, X8, Citrate_synt, 14-3-3, RMMBL, efhand, NPH3, CAF1, ICL, FAE1_CUT1_RppA, Orn_DAP_Arg_deC, PPDK_N, Myb_DNA-binding, AP2, F-box, and APS_kinase;
(a) wherein the gathering cuttoff for said protein domain families is stated in Table 11;
(b) producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA;
(c) selecting corn plants which are homozygous and hemizygous for said recombinant DNA by treating with an herbicide;
(d) collecting seed from herbicide-treated-surviving corn plants and planting said seed to produce further progeny corn plants;
(e) repeating steps (c) and (d) at least once to produce an inbred corn line;
(f) crossing said inbred corn line with a second corn line to produce hybrid seed.

18. A method of selecting a plant comprising cells of claim 1 wherein an immunoreactive antibody is used to detect the presence of said protein in seed or plant tissue.

19. Anti-counterfeit milled seed having, as an indication of origin, a plant cell of claim 1.

20. A method of growing a corn, cotton or soybean crop without irrigation water comprising planting seed having plant cells of claim 2 which are selected for enhanced water use efficiency.
